SEQUENCE LISTING (136 sequences)

SEQ ID NO: 1 - protein, 257 aa, synthetic construct
MASLENQIILITGASSGIGTACAKIFAGAGAKLILAARRLERLQQLADILTQDFNTEVHLLELDVRDRSAVESAISNLPASWSDIDILINNAGLSRGLDKLHEGSFTDWEEMIDTNIKGLLYLSRYVVPGMVSRGRGHVVNLGSIAGHQTYPGGNVYCATKAAVRAISEGLKQDLLGTPVRVTSVDPGMVETEFSQVRFHGNAQRANQVYQGVTPLTPDDVADVIFFCVTRSPHVNINEVVLMPVDQASATLVNRRT

SEQ ID NO: 2 - protein, 267 aa, synthetic construct
MLKVDTLKISSQQVEAFERDGVICVKNALDDIWVERLRTAVDRNISIPGPLEEKNAPRPEGSVEHASSLWLVDADFRALAFESPLPTLAAQVLKSEKLNFLADGFFVKKPKTNGHIGWHNDLPYWPVQGWQCCKIWLPLDTVKQENGRLEYIKGSHQWGKELRERSNPSWFVEPEPHEILSWDMEAGDCLIHHFLTIHHSVTNISSTQRRAIVTNWTGDDVTYYQRPKAWPFKPLEEIDLPEFNSFKTKKVGEPIDCDIFPRVEVFR

SEQ ID NO: 3 - protein, 267 aa, synthetic construct
MLKLELPKITLQEIEAFEQDGVICVKNVLDNIWVERMRKAVDKNISIAGPLEVKGISKPEGNVEHTNSLWLVDADFRALVFESPLATLAAQILKSTKLNFLADGFFVKQPKATSRVGWHNDLPYWPIQGWQCCKIWLALDKVNQQNGRLEYIKGSHRWGKELREDSNPAWFSQPESHELLSWDMEPGDCLVHHLLTIHHSVTNISSTQRRAVVTNWTGDDVTYYPRPKAWPFRPLDEIDIPEFDSLKAKKPGEPIDCDMFPKIKWHR

SEQ ID NO: 4 - protein, 286 aa, synthetic construct
MLIANSSKISRQEVENFKRDGVICLKNVVDDYWVERMRKAVDRNLLNSNGVRGRKLKTGDVVHDYGLWLKDNDFRDLVFKSPLARVAAQIMESETINFLCDGFFVKKAKADSHVGWHNDLPYWPVKGWKCCKIWLALDPVNQENGRLEYIKGSHLWNKDLRENSNVSWFSEPSYSDILYWDMEPGDALVHHFQTIHHSIGNTTYKSRRAIVTNWTGDDVVYDPSPQTWPFQPIEEIGISEFNSLDTLRSGESIDCEIFPKIDLTPSPSPTSRGEQNPNFLKFPHRL

SEQ ID NO: 5 - protein, 267 aa, synthetic construct
MLKVDTSKITTQQVEAFERDGVICVKNVLDDIWVERMRRAVDKNVLIPGPLEVKGIPRAEGHVEHTSSLWLTDADFRALAFESPLATLTAQVLKSKKLNFLGDGFFVKKPKGETGVGWHNDKSYWPIQGWQCCKIWLALDSVNQENGKLEYIKASHLWGKELREASDPSWFVEPEPHEIISWDMEPGDCLVHHFMTIHHSVRNTSSTRRRAVVINWTGDDVTYERRPNAWPFRPLEEIDIPEFESLKAKKSGEPIDCDIFPRVELHR

SEQ ID NO: 6 - protein, 272 aa, synthetic construct
MLKVDTPKISPQQVEAFERDGVICVKNALDDIWIERMRKAVDKNISIPGPLEGKNTPKKEASAEHTSSLWLVDADFRALAFESPLPKLAVGVLKSEKLNFLADGFFVKRPEANGRIGWHNDLPYWPVQGWQCCKIWLALDTVKQENGRLEYIKGSHQWGRELRERSNPSWFVEPEPHEILSWDMEAGDCLIHHFLTIHHSVTNKSSTQRRAIVTNWTGDDVTYYQRPKAWPFKPLEEIDLPQFNSLKTKKFGEPIDCDIFPRVEVHRHRTHI

SEQ ID NO: 7 - protein, 267 aa, synthetic construct
MLKIDTLKISLQQIEAFERDGVICLRNVLDESWVERMRTAVDKNVSIPGPLEVKGISRPEASVEHTSSLWLVDPDFRALVFESPLSTIAAQLLRSEKLNFLADGFFVKKPKATSRVGWHNDLPYWPIQGWQFCKIWLALDNVNEENGRLEYIKGSHQWGKELREDSNPSWFVEPEPHELLSWDMEPGDCLVHHLLTIHHSVTNISSRQRRAVVTNWTGDDVTYYPRLKAWPFRPLEEIDLPEFNSLKTKKTGEQIDCYMFPPIQLHR

SEQ ID NO: 8 - protein, 267 aa, synthetic construct
MLKVDTQKISPQQVEAFERDGVICVKNAVDDIWVERMRTAVDKNISIPGPLEDKNVPKPQGSAEHASSIWLIDADFRALAFESPLPTLAAQVLKSKKLNFLADGFFVKKPESNGRIGWHNDLPYWPVQGWQCCKIWLALDTVKQENGRLEYIKGSHQWGKELRERSNPSWFIEPEPHEILSWDMEAGDCLIHHFLTIHHSVTNISSTQRRAIVTNWTGDDVTYYQRPKAWPFKPLEEIDLPEFNSLKTKKSGEPIDCDIFPRVQVHR

SEQ ID NO: 9 - protein, 267 aa, synthetic construct
MLKLDLPKITLQEIEAFEQDGVICVKNVLDNIWVERMRKAVDKNLSIAGPLEVKGITKPEGNVEHSNSLWLVDTDFRALVFESPLANLAAQFLKSTKLNFLADGFFVKQPKASSRVGWHNDLPYWPIQGWQCCKIWLALDKVNQQNGRLEYIKGSHRWGKELREDSNPSWFSEPEPHELLSWDMEPGDCLVHHLLTIHHSVTNISSTKRRAVVTNWTGDDVTHYPRPKAWPFRPLDEIDIPEFDSLKAKKPGEPIDCDMFPKIKWHR

SEQ ID NO: 10 - protein, 266 aa, synthetic construct
MLKIDTSKISFQQIGAFERDGVICLRNVLDENWVERMRTAVDKNVSINGPLEAKGISRAEASVEHTSSLWLVDPDFRALVFESPLSTIAAQLLQSEKLNFLADGFFVKKPKATSRVGWHNDLPYWPIQGWQCCKIWLALDHVNEKNGRLEYIKGSHKWGKELREDSNPLWFVEPEPHELLSWNMEPGDCLVHHLLTIHHSVTNISSTQRRAVVTNWTGDDVTYYPRPKAWPFRSVEEIDLPEFNSLKTKKTGEPIDCDMFPQVQLH

SEQ ID NO: 11 - protein, 267 aa, synthetic construct
MLKVDTRKISHQQVEAFERDGVICVKNAVDDIWVQRMRTAVDKNVLIPGPLEEKNAPKPEASAEHTSNLWLVDADFRALAFESPLPTLAVQVLKSKKLNFLADGFFVKKPKSNSRIGWHNDLPYWPIQGWQCCKIWLALDTVNQENGRLEYIKGSHRWGKELRERSNPSWFVEPKPHEILSWDMEAGDCLIHHFLTIHHSVTNISSRQRRAVVTNWTGDDVTYYQRPKAWPFKSIEEIDLPQFNSFKTKKSGEPLDCDIFPRIEVHR

SEQ ID NO: 12 - protein, 368 aa, synthetic construct
MPVLRILHLVGSAQDDFYCDLSRLYAQDCLAAMAELPYDSAIAYITPDGQWRFPRSLSREDIAQAKPMPVSEAIEFIAAQNIDIVLPQMFCIPGMTYYRALFDLLEIPYIGNTPDLMAITAHKARTKAIVEAAGVKVPRGEVLRRGDVPTITPPVVIKPVSSDNSLGVTLVKDAAEYEAALEKAFEHGDEAIVETFIEGREVRCGIIVKDGELIGLPLEEYLIDSQEKPIRTYADKLKKTDDGSLGFAAKGNNKSWILDPNDPITQKVQEVAKKCHQALGCRHYSLFDFRIDSQGQPWFLEAGLYCSFAPKSVISSMAKAVGIPLNELLTIAIAETLGSNKYSDRISVVEINEPSKTPRKERELSQMI

SEQ ID NO: 13 - protein, 442 aa, synthetic construct
MSGVRVHRIWDAGPGRTVAALAALCATLPVDLAVVLVALLVGRQPPRGRLPAEARRTVLLNGGKMTKALQLARSFHLAGHRVILVESAKYRWTGHRFSRAVDAFYCVPEPGTPGYAPALLNIVRYENVDVYVPVSSPAGSVPDAVARELLDGACDVVHSDAKTVQLLDDKAEFASTAASLSLQVPDSHRITDARQVADFPFPPGRSYILKRIAYNPVGRMNLTRLSAATPDRNAAYARSLSISEDDPWILQEFIEGREYCTHGTARSGRLQVYGCCESSAAQVNYRSVDKPEIRRWVETFVKNLNLSGQVSFDFIEAHDGQVYAIECNPRTHSAITMFHDHPDLAAAYLNDGHPLITPKHNSRPTYWIYHELWRLLRHPGRLGRLATILRGTDAIFTGWDPVPYLMVHHLQIPALLWANLRVGKGWSRIDFNIGKLVENGGD

SEQ ID NO: 14 - protein, 429 aa, synthetic construct
MGRTLATLVVLFGTLPFDLALVLVALLAGRRPSRGRLPAQARRTILLNGGKMTKALQLARSFHLAGHRVILVESEKYRWTGHRFSRAVDAFYCVPEPTEPGYALALLDIVRYENVDVYVPVSSPAGSVPDAVARELLDGACDVVHSDAKTVQLLDDKAEFASTAASLSLRVPDSHRITDARQVVDFAFPAGRSYILKRIAYDPVGRMNLTRLSGATPDHNAAYARSLPISEDDPWILQEFIEGREYCTHGTARSGRLQVYGCCESSSAQVNYRNVDKPEIRRWVETFVKNLNLSGQVSFDFIEARDGQVYAIECNPRTHSAITMFHDHPDLAAAYLDDNHPLITPNDGARPTYWIYHELWRLLRHRGRISRLVTMLRGKDAIFAGWDPMPYLMVHHLQIPALLWANLRAGKGWSRIDFNIGKLVENGGD

SEQ ID NO: 15 - protein, 435 aa, synthetic construct
MREVFQAKTIGTLALLQVVLPLNLALTTFALLRGVFVAPPPVAVAAQRKTILVSGGKMTKALQLARSFHAAGHRVVLVESSKYRFNGHRFSRAVDRFYTVPAPDSDNYAVALLAVVRAEEVDVYVPVCSPVASYYDALAKDQLSPHCEVLHCDADMVARLDDKYEFFALVASLGLSTPETHRVTAPGQVEEFDFTGTDYILKSIPYDPVHRRDMTTVPRPTATETTTYARSKPITEATPWIMQEFVRGQEYCTHSLVRDGAVQVFCCCESSAFQINYRMVDKPEIEEWVGEFAQRLNLTGQVSFDFIQGDDGRLHAIECNPRTHSAITMFYDHPDLARAYLERGVPVVKPLPHSKPTYWIYHELWRLVTQRGGRAHRLAVIAQGKDAIFDWDDPLPFLLVHHLQIPSLLLSNLLRRKGWTGIDFNIGKLIEAAGD

SEQ ID NO: 16 - protein, 530 aa, synthetic construct
MCRVETRPQVGEHAGMESVPLKAAEGGLVEERKAFLPQSYSLWKDSIEGRLWSLLTLFGLFISSPFLFAFVALSVLSAVVRKLLRLPAARKLPEGSNKGRGRTALVTGGKMTKSLDVCRHLKNEGFRVILTETPRYWMSASRFSSAVDKFVVLPVAPETHPEGYVEALRNLFEKENVSLFAPVCSPFSSLYDAKAAESLPEGAISWSLPAEMVQQLDDKVEFARMAKEVGLPVPDTLRVESKEEVRRFNSELAEKWRRDSSSAIASGAEKKKTDCRRYILKTLDYDPMRRLDLFTLPCGPKELEKYLDETTISPDRPWLVQEFLEGREYSSCALSWKGKLLAFTDNEAVISCYNFKYAGRDKIQEWVRVFCEKYQLSGVICVDFFERADGTQLAIECNPRFSSNMTAFYNNPRLGAAMADPDLALRSGVTETPLPSSKESNWTLVDLYFHSYTQMMKNPLAAFTAAAGLLLVSEETKEKQDAYWAPEDPLPSLALHCFHMPALLVRNVWDGRKWAKIDFCIGKMTEENGD

SEQ ID NO: 17 - protein, 397 aa, synthetic construct
EVKPNGKVAIVSGGKMTKAYVIARQLKAQGCRVVLLETSKYWMVASRASNCVDRFAMVPLPEKDLAGYLDAVRALAIEEKADLFIPVTSPAASEYEAQVAPVLPAGCVSWSLDLETVRDLDDKTAFCSSAERLGLPAPRSHRVASDEEAHAFNEKLLAEAATATAGAETRYILKSLAYDSMHRLDLFTLPCAPDNPWIIQTFVVGDEYSTCALVKEGRLLAFTDNRACLSCFNYTPARSEALRSWVRDFCAARRLSGVVCIDFIVDAQSGTPYAIECNPRFSSNVLNLFWNPPFGGALFRPHKGGGVEAFFWPPPPPPPLQIWALLSKRPFSLRSAGALLSTVATKKDAYFDVADPLPFIAHLFVHIPALLARNLSTGNKWAKIDPCIGKLTEENGD

SEQ ID NO: 18 - protein, 480 aa, synthetic construct
MLLPQSITPTMQIFAVFQNLGTLLLLAIAFPFNCIVVLTALLWNLVSKPFRDRGILPVHPKNIMLTGGKMTKALQLARSFHMVGHRVVLVETHKYWLTGHRFSNAVDRFYTVPAPEKDPEAYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATPAETAAFVNSLPISPEKPWIMQEFIPGQEYCTHSTVRNGELRMHCCCESSAFQVNYENVDKPEILAWVRHFVKELGITGQASFDFIQAEDGNVYAIECNPRTHSAITMFYNHPGVADAFCRDVTCNTSTSRAGLLNSSFINNISGEPAPTIYPLQPLSTSKPTYWTYHELWRLTGIRSFPQLQTWCKNILRGKDAIFAIDDPLPFLMVHHWQIPLLLLDNLRRLKGWIRIDFNIGKIVELGGD

SEQ ID NO: 19 - protein, 465 aa, synthetic construct
MTQSISFSSPVPATPPISVKARFIALFQNLGTLTLLLLALPVNAVIVVISLVWNSLTRLFSTQQTTVARSKNILISGGKMTKALQLARSFGAAGHRVVLIETHKYWLSGHRFSNAVSRFYTTPTPQYDPEAYIQTLIDIVKRENIDVYVPVTSPVASYYDSLAKPALSPYCEVLHFDADVTKMLDDKFAFSEKARDLGLSVPKSFKITNPEQVLNFDFSQETRKYILKSIPYDSVRRLDLTKLPCDTLEETAAFVKSLPISPEKPWIMQEFIPGKEFCTHSTVRNGELRLHCCSESSAFQVNYENVENPEIQAWVKHFVNGLGFTGQVSFDFIQTDDGKVYAIECNPRTHSAITMFYNHPQVSDAYLGTEPLTEPLQPLPNSKPTYWLYHEVWRLTGIRSFSQLQNWVRNIFRGTDAIYKLHDPLPFLTVHHWQIPLLLLNNLWQLRGWTKIDFNIGKLVEFGGD

SEQ ID NO: 20 - protein, 471 aa, synthetic construct
MLCPYERLVFCLKEKLMTQSIPLSFSQPTTPLTVVKTKIVALFKTLGTLALLLLALPLNGFVVLISLLWVIVRNPFTKPTAVAAHPQNILVSGAKMTKALQLARSFHAAGNRVILIEGHKYWLSGHRFSNAVSRFYTVPAPQDDPESYTQALLEIVKKEKIDVYIPVCSPVASYYDSLAKPVLSEYCEVFHFDADITAMLDDKFAFTDQARSLGLSVPKSFKITDPEQIINFDFSQETRKYIIKSISYDSVRRLNLTKLPCDTPEETAAFVRSLPISPEKPWIMQEFIPGKELCTHSTVRDGELRLHCCSNSSAFQINYENVENPQIREWVQHFVKSLRLTGQVSFDFIQAEDGTVYAIECNPRTHSAITMFYNHPGVAQAYLGKTPQAAPLEPLADSKPTYWLYHEIWRLTSIRSWKHLQTWFKNLVRGTDAIYSMDDPIPFLTLHHWQITLLLLQNLQQLKGWVKIDFN

SEQ ID NO: 21 - protein, 465 aa, synthetic construct
MAQSISLSLPSSTTPSTGVRVKIVALFKTLGTLTLLLIALPFNALIVLIALLWGIARSPFTKKAVVAANPQTILVSGAKMTKALQLARSFHAAGHRVILIEGHKYWLSGHRFSQAVSRFYTVPAPQSDPEAYIQALVEIVKKEKVDIYVPVCSPVASYYDSLAKPTLSEYCEVFHFDADITKMLDDKFAFTDKARSLGLSVPKSFKITDPQQVINFDFSQETRKYILKSIAYDSVRRLDLTKLPCDSPEETAAFVNSLPISPENPWIMQEFIPGKEFCTHSTVRDGELRLHCCCHSSAFQINYENVENPQIREWVQQFVKSLRLTGQVSFDFIQAEDGTVYAIECNPRTHSAITMFYNHPGVAEAYFGKTPLAAPLEPLASSKPTYWIYHEIWRLTNIRSWKQLQTRLNILFRGTDAIFRLNDPVPFLTLHHWQIPLLLLQNLQKLKGWVKIDFNIGKLVELGGD

SEQ ID NO: 22 - protein, 457 aa, synthetic construct
MAQSISLSSPAKTHAPGISASSLKTLGTLTLLLLALPLNASLVLVALLLKSLRPQNVTTEEPKNILISGGKMTKALQLARSFHEQGHRVILLEAHKYWLTGHRFSFAVNKFYTVEAPEKDPEGYIQSLVNIVEKENIDVYVPVCSPVASYYDSLAKKALPQCEVIHCDAEMTQMLDDKYAFAQTAQSFGLSVPKSFKITEPEQVINFDFSQEKRKYILKSIPYDSVRRLDLTKLPCDTPEATAAFVRSLPISPEKPWIMQEFIPGKEYCTHSTVRNGVITLHCCCESSAFQVNYENVDNPKIFEWVSRFVKELGITGQVSFDFIEAEDGNIYAIECNPRTHSAITMFYNHPGVADAYLGTGSNLAEPIQPKSTSKPTYWTYHEVWRLITTRSWSDFVYRFKIITHGKDAIFSWQDPLPFLMNPHWQIFLLLIQNLQKNRGWVRIDFNIGKLVELGGD

SEQ ID NO: 23 - protein, 465 aa, synthetic construct
MAQSLPLTSAGGATSPTAFVAQVKALFQNIATLTILLLVLPINAAIVLTSLFWSRVSRFVRPQTVVAANRKNILISGGKMTKALQIARSFHAAGHRVVLIETHKYWLSGHRFSDAISRFYTTPTPQYDPEAYIQALLDIVKKENIDVYVPVTSPVASYYDSLAKPALSPYCEVFHFDADVTQMLDDKFAFSEKARSFGLSVPKSFKITNPEQVLNFDFSGETRKYILKSIPYDSVRRLDLTKLPCDTPEETAAFVRSLPISPEKPWIMQEFIPGKEFCTHSTVKNGELRLHCCAESSAFQVNYENVENPKIQEWVRHFVKELGITGQVSFDFIQAEDGTVYAIECNPRTHSAITMFYNHPDVADAYLSEEPFTEPLVPLPNSKPTYWTYHEVWRLTGIHSFAQLQTWIRNFLQGTDAIYQLDDPLPFLMVHHWQIPLLLLNNLRQLKGWTKIDFNIGKLVEIGGD

SEQ ID NO: 24 - protein, 442 aa, synthetic construct
MRKYIFVVFQNLGTLVLLAIAFPLNCIVVLTSLLWNFLKQPFNKSIVVNPNSKNILIAGARMTKTLQLARSFHAAGHRVIIIDIEKFWSSGNKYSNSVAGFYTVPDPSKDLEGYVESLHAIAKTEKIDFFIPVAIFSVIHYDQGQPPLPDFVEFFHFDADVTKILDDKFAFAETARSFGLSVPKSFKITHPEQVINFDFSHEKRKYILKSIPYDQIRRLNLTKLPCATSAETAAFVNSLPISEENPWIMQEFIPGKEYCTHTTARDGESRMYCCCESSAFQVNYENVDQQEIMQWATHFTKELGKTGQLSFDFIQAEDGTVYAIECNPRTHSAITMFYNHPGVADAYLGKEPLAESLQPLADSKPTYWLYHEVWRLNEIRNFEQLQTWVRNIRRGKEAIFEVSDPLPFLMVHHWQIPLLILDNLRRLKGWIRIDFNMGELIE

SEQ ID NO: 25 - protein, 459 aa, synthetic construct
MTQSISVASPAPKTQSVPLGLRISALWKNVGTLALLLLVLPINAVIVLVSLLLGHQSQAIATEPKNILISGAKMTKALQLARSFHAAGHRVVLVETHKYWLTGHRFSKAVSRFYTVPTPQSDPEAYTQALLDIVKTENIDVYVPVCSPIASYYDSLAKPVLSKFCEVFHCDADVTQMLDDKYAFAEKARSLGLSVPKSFKITDPEQILNFDFSQEKRQYILKSIPYDSVRRLDLTKLPCETPEATADFVNSLPISPQKPWIMQEFIPGKEYCTHSTVRNGELRMHCCCESSAFQVNYENVDHPQILEWVRHFVKALGITGQVSFDFIQAEDGTIYAIECNPRTHSAITMFYNHPHVADAYLSEIPQLEPIQPLTNSKPTYWTYHEIWRLTGIRSFSQLQTWLKTFFGGKDAIYCFSDPLPFLTVHHWQIPLLLLQNLQQLKGWIRIDFNIGKLVEFGGD

SEQ ID NO: 26 - protein, 481 aa, synthetic construct
MLLPQSITPTMQIFAVFQNLGTLLLLAIAFPFNCIVVLTALLGNLVSKPFRDRGILPVSHPKNIMLTGGKMTKALQLARSFHMVGHRVVLVETHKYWLTGHRFSNAVDRFYTVPAPEKDPEGYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATPAETAAFVNSLPISPEKPWIMQEFIPGQEYCTHSTVRNGELRMHCCCESSAFQVNYENVDKPEILAWVRHFVKELGITGQASFDFIQAEDGNVYAIECNPRTHSAITMFYNHPGVADAFCRDVTCNTSTSRAGLLNSSFINNISGEPAPTIYPLQPLSTSKPTYWTYHELWRLTGIRSFPQLQTWCKNILRGKDAIFAIDDPLPFLMVHHWQIPLLLLDNLRRLKGWIRIDFNIGKIVELGGD

SEQ ID NO: 27 - protein, 464 aa, synthetic construct
MAQSISVSSSPAIPSFPSETKIAVIIQNLLTLALLLLALPINATIVLVTLLWHTISRPFQQPATKAANPKNILISGGKMTKALQLARSCNAAGHRVVLIETHKYWLSGHRFSQAVDKFYTVPAPQENPERYTQALIDIIKQENIDVYIPVTSPLGSYYDSLAKPLLSKYCEVFHFDADITERLDDKFAFAETARSLGLSVPKSFKITKAEQVLNFDFSQESRKYILKSIPYDSVRRLDLTKLPCATPEETAAFVRSLPISPEKPWIMQEFIPGKEFCTHSTVRDGELRLHCCCESSAFQVNYENVENSQIREWVRHFVKELKLTGQVSFDFIQAEDGKVYAIECNPRTHSAITTFYDHPQVAQAYLDNEPMAQTLQPLPSSKPTYWTYHEVWRLTGIRSLTQFKKWIANIWRGTDAIYKSDDPLPFLMVHHWQIPLLLIKNLRQLKGWTRIDFNIGKLVELGGD

SEQ ID NO: 28 - protein, 448 aa, synthetic construct
MAQLQSIQASIFAVLQNLGTLALLMIAFPFNCIVVLLSLLLNFLSRPFHKPVILTKNPRNIMIAGARMTKTLQLARSFHAAGHRVILVDTEKFWLSGNQFSHAVAGFYTVPDPHKDLEGYTQALRAIAKKENIDFFIPVAIFAVIYYDSMSQHQLFDCCEVFHFNADVTKMLDDKFAFAEKARSLSLSVPKSFKITAPEQILNFDFSNEKRKYILKSIPYDAVRRLNMTLLPCDTPEQTAAFVKSLPISEEKPWIMQEFIPGKEYCTHSTVRDGKQTIYCCCESSAFQVNYENVDKPEILQWVNHFVKELGLTGQISFDFIQAVDGTVYVIECNPRTHSAITMFYNHPGVADAYLSKQPLAEPLQPLSDSKPTYWLYHEVWRLNEIRSLKQLQTWIKNILRGKDAIFTVNDPLPFLMVHHWQIPLLLLDNLRRLKGWIRIDFNPLLSL

SEQ ID NO: 29 - protein, 448 aa, synthetic construct
MTNSLILAVLQNLGTLTLLAIAFPFNLTVVVVALVWDSLTRPFQNPKVANPNPKTIMLTGGKMTKSLQLARSFYADGHRVILVESHKYWLVGHRFSRAVDRFYTVPAPNKDPDGYMEGLLAIAKQENVDVYVPVCSPVASYYDSLAKPVLSGCCEVFHFDPDVTQLLDDKFAFAQKAREFGLSVPKSFKITDPQQVIDFDFRGEKRKYILKSIPYDSVRRLNLTKLPCKTPSETAAFVKSLPISEDKPWIMQEFIPGKEYCTHSTVRNGELRLHCCCESSAFQVNYENVDQPDILQWVSRFVQGLNLTGQASFDFIKTEDGIVYAIECNPRTHSAITMFYNHPGVAEAYLSDTPLPEPLQPLPESKPTYWLYHEVWRLNEIRSFGDIRRWFKTVFGGKDAIFQVNDPLPFLMVHHWQIPLLLLDNLRRMQGWIRIDFNIGKLVELGGD

SEQ ID NO: 30 - protein, 461 aa, synthetic construct
MAQSISFDSSPATPSLGLETKIAAIIQNILTLALLLLALPINAIIVCIALVLGTIFRPQTTKTSNPKNILISGGKMTKALQLARSFHADGHRVVLLETHKYWLTGHRFSQAVDKFYTTPAPQKKPEDYIKALVDIVKRENIDVYIPVTSPVGSYYDSLAKPELSHHCEVFHFDAEITQMLDDKFAMAEKARSLGLSVPKSFKITSGEQVINFDFSRETRKYILKSIAYDSVRRLDLTKLPCATPEETAAFVRKLPISPEKPWIMQEFIPGKEFCTHSTVRDGEIRLHCCCESSAFQVNYENIENPQILEWVRHFVKELKLTGQISFDFIQTEDGQVYAIECNPRTHSAITTFYNHPQVAEAYIGKQPMAETLQPLATSKPTYWTYHEIWRLTGIRSFTQLKTWLKNIWRGTDAILQLHDPLPFLMVHHWQIPLLLLNNLRQLKGWTRIDFNIGKLVEFGGD

SEQ ID NO: 31 - protein, 439 aa, synthetic construct
MLNKLIAALQNLLTLTALLITLPINLAIVLIASLIGLFQRETIPQSNSPKRILITGGKMTKALQLARSFHAAGHFVVLVETQKYWLTGHQFSNAVDRFYTVPAPKQDSEAFIQALVDIVQRENIDFFVPVTSPIESYYCSLAKPELSKYCEVLHFDVGITQLLDDKFELSEKARSLNLTAPKTYRITDPQQVLDFEFDSSQYILKSIAYNSVHRLDMTKYPLESKAAMKAHLATLPISEDNPWILQEFISGQEYCTHSTVRDGKVRLHCCAKSSAFQVNYEQVENSEIQAWVTTFVKALNLSGQISFDFIESSSGEVYAIECNPRTHSAITMFYNHPDVAKAYLGEPLTVEPIQPLPTSKPTYWTYHEVWRLITGDRPLYRLQTILHGKDAILQTSDPIPFLMVHHWQIPLLLLNNLRHLKGWVRIDFNIGKLVELGGD

SEQ ID NO: 32 - protein, 481 aa, synthetic construct
MLLPQSITPTMQIFAVFQNLGTLLLLAIAFPFNCIVVLTALLWNLVSKPFRDRGILPVSHPKNIMLTGGKMTKALQLARSFHMVGHRVVLVETHKYWLTGHRFSNAVDRFYTVPAPEKDPEAYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATPAETAAFVNSLPISPEKPWIMQEFIPGQEYCTHSTVRNGELRMHCCCESSAFQVNYENVDKPEIIAWVRHFVKELGITGQASFDFIQAEDGNVYAIECNPRTHSAITMFYNHPGVADAFCRDVTCNTSTSRAGLLNSSFINNISGEPAPTIYPLQPLSTSKPTYWTYHELWRLTGIRSFPQLQTWCKNILRGKDAIFAIDDPLPFLMVHHWQIPLLLLDNLRRLKGWIRIDFNIGKIVELGGD

SEQ ID NO: 33 - protein, 449 aa, synthetic construct
MAQLQSIQASIFAVLQNLGTLALLMIAFPFNCIVVLLSLLLNFLSRPFHKPVILTKNPRNIMIAGARMTKTLQLARSFHAAGHRVILVDTEKFWLSGNQFSHAVAGFYTVPDPHKDLEGYTQALRAIAKKENIDFFIPVAIFAVIYYDLMSQHPLFDCCEVFHFNADVTKMLDDKFAFAEKARLLSLSVPKSFKITAPEQILDFDFSNEKRKYILKSIPYDAVRRLNMTLLPCDTPEQTAAFVKSLPISEEKPWIMQEFIPGKEYCTHSTVRDGKQTIYCCCESSAFQVNYENVDKPEILQWVNHFVKELGLTGQISFDFIQAVDGTVYAIECNPRTHSAITMFYNHPGVADAYLSKQPLAEPLQPLSDSKPTYWLYHEVWRLNEIRSLKQLQTWVKNILRGKDAIFTVNDPLPFLMVHHWQIPLLLLDNLRRLKGWIRIDFNIGELIE

SEQ ID NO: 34 - protein, 465 aa, synthetic construct
MAQSISLSLAKSPGSSTGVWVKLVALFKTLGTLTLLLIALPFNALIVLISLLWGFVRSPFRQKAVVADHPQTILVSGAKMTKALQLARCFHAAGHRVILIEGHKYWLSGHRFSKAVSGFYTVPAPELDPLGYIQALVEIVKKEKVDVYVPVCSPVASYYDSLAKPALSEYCEVFHFDADVTKMLDDKFAFTDQARSLGLSVPKSFKITDHQQVINFDFSQETHKYILKNIAYDSVRRLNLTKLPCDTPEETAAFVNSLPISEENPWIMQEFIPGKELCTHSTVRDGELRLHCCSDSSAFQINYENVENPQIREWVQHFVKSLALTGQVSFDFIQAESGTVYAIECNPRTHSAITMFYNHPGVAEAYLGKTPLTDLTEPLANSKPTYWIYHEIWRLTGIRSWKQLQTSINTLAQGTDAVYQLDDPIPFLTLHHWQIPLLLLKNLQQLKGWVKIDFNIGKLVELGGD

SEQ ID NO: 35 - protein, 465 aa, synthetic construct
MAQSISLSLPESTTPATSVGVKIAALFKTLGTLTLLLIALPFNALIVLIALLWGIVRSPFTKKAVVAAHSQTILVSGAKMTKALQLARSFHAAGHRVILIEGHKYWLSGHRFSQAVSRFYTVPAPQSDSEGYIQALVEIVKQEKVDIYVPVCSPIASYYDSLAKPALSEYCEVFHFDADITKMLDDKFAFTDKARSLGLSVPKSFKITDPQQVINFDFSQETRKYILKSIAYDSVRRLDLTKLPCNTSEETAAFVNSLPISPENPWIMQEFIPGKEFCTHSTVRDGELRLHCCCHSSAFQINYENVENPQICEWVQQFVKSLQLTGQVSFDFIQAEDGSVYAIECNPRTHSAITMFYNHHGVADAYFGKTPLAAPLEPLASSKPTYWIYHEIWRLTGIRSWKQLQTSVNTLLRGTDAIYNLNDPVPFLTLHHWQIPLLLLKNLQQLKGWVKIDFNIGKLVELGGD

SEQ ID NO: 36 - protein, 469 aa, synthetic construct
MAQSSVSVSASQPIAPPTSIGMRFFALFQNLATLTLLLLALPINATIVLTTLLLNILTSPFQKKQTTVVATEKKNILISGGKMTKALQLARFFHSAGHRVILTETHKYWLSGHRFSQSVDKFYTTPVPQKDSQAYTQALIDIINKEGIDIYIPVTSPIASYYDSLAKPALSEYCEVFHIDAATCEMLDDKFAFSEKARSFGLSIPKCFKITNPEQVINFDFSGETRKYILKSIPYDSVRRLDLTKLPCDTPEETEAFVRSLPISPQKPWIMQEFIPGKEYCTHSTVRDGVMRLHCCCESSAFQVNYENVENPKIREWVTHFVKELGVTGQLSFDFIEAEDGNVYAIECNPRTHSAITIFHDQLQQAANAYLSKEPIAAPLQALPNSKPTYWTYHEFWRLNEIRSLSQLGNWIKNMLRGTDAIYTFDDCLPFLMVHHWQIPVLLLKNLSKLKGWTRIDFNIGKLVELGGD

SEQ ID NO: 37 - protein, 465 aa, synthetic construct
MAKSVSLSLAKSTTPSTDVRLKLVALFKTLGTLTLLLIALPFNGLIVLIALLWGIVQWPLRKKALVAADPRTVLVSGGKMTKALQLARCFHGAGHRVILIETHKYWLSGHKFSRAVSAFYTVPSPQSDPEGYIQSLVAIVKKEKVDFYVPVCSPVASYYDSLAKPALSAYCEVFHFDADITKMLDDKFAFTEQGRSLGLSVPKSFQITDPQQVINFDFSQETRKYILKNIAYDSVRRLNLTKLPCNTPEETAAFVNSLPISAQNPWIMQEFIPGKELCTHSTVRDGELRLHCCSNSSAFQINYQNVENPQIRQWVQQFVKSLGLTGQVSFDFIQAEDGTVYAIECNPRTHSAITMFYNHPGVADAYLGKTPQAAPVEPLANSKPTYWLYHEIWRLTGIRSWKQLQTSVNTLVGGTDAIFCFDDPVPFLTLYHWQIPLLLLKNLQDLKGWVKIDFNIGKLVELDGD

SEQ ID NO: 38 - protein, 464 aa, synthetic construct
MAQSISVSSSPAIPSFPSETKIAVIIQNLLTLALLLLALPFNATIVLVTLLWHTISRPFQQATTKTANPKNVLISGAKMTKALQLARSFNAAGHRVVLIETHKYWLSGHRFSQAVDKFYTVPAPQENPERYTQALIDIIKQENIDVYVPVTSPLGSYYDSLAKPMLSNYCEVFHFDADITQKLDDKFAFAETARSLGLSVPKSFKITSAEQVLNFDFSQESRKYILKSIPYDSVRRLDLTKLPCATPEETAAFVKSLPISPEKPWIMQEFIPGKEFCTHSTVRNGELRLHCCCESSAFQVNYENVENSQIREWVRHFVKEQKLTGQVSFDFIQAEDGRVYAIECNPRTHSAITTFYDHPQVAQAYLDKEPMAETLQPLPTSKPTYWTYHEVWRLTGIRSFTQLKKWIANIWRGTDAIYKPDDPLPFLMVHHWQIPLLLLKNLRQLKGWTRIDFNIGKLVELGGD

SEQ ID NO: 39 - protein, 437 aa, synthetic construct
MLALFQNLGTLLLLALAFPFNCIVVLVALLTKPKLPQATVAKAQNILISGGKMTKALQLARSFYAAGHRVVLIETDKYWLTGHRFSRAVDAFYTVPAPQKDPEAYIQALVNIAKKENIDVYIPVCSPISSYYDSLAKPALAGCCEVFHFDADITKMLDDKFAFAQTAQSFGLSVPKSYKITHPQQVLDFDFSTEQNKYILKSIPYDSVRRLNLTKLPCNTRAETAAFVNSLPISEEKPWIMQEFITGKEYCTHSTVRDGELRLHCCCESSAFQVNYENVDQPEILQWVSHFVKQLGVTGQASFDFIRAENGNIYAIECNPRTHSAITMFYNHPGVASAYLSSQPLKPLQPLTDSKPTYWLYHEVWRLNEIRSLQQLQTWFKNIRRGKESIFAFNDPLPFLMVHHWQIPLLLLDNLRRLAGWIRIDFNIGKLVEFGGD

SEQ ID NO: 40 - protein, 440 aa, synthetic construct
MFSTTFKSLGTLALLKLALPFNLTLVLIASIINIFSTPFKIKKKPNINSKTVLLTGGKMTKALQLARSFYSAGHRVILVETHKYWLSGHRFSVAVDKFFTIPDPVKDKEGYIDGLLDIVKRENVDIFIPVSSPVASYYDSVAKMVLSPYCKVLHFDVEMTLVLDDKASLCQKASSLGLTSPASYLITDVQEILDFDFSKNNHKYILKSIKYDSVYRLNMTQFPFEGMEEYVRSLPISEENPWVMQQFITGQEYCTHSTVLNGKIRLHCCSMSSHFQVNYEHVDNQKIYEWVEEFVGKLNLTGQISFDFIQTDDGTVYPIECNPRTHSAISMFYNHPLVADAYLNDGDDAPITPLESSKPTFWTYHELWRLTEVRSPQDLSQWWQKVTKGQDGIFSWQDPLPFLMVHHWQIPLLLFGNLIKLKPWVKIDFNIGKLVESAGD

SEQ ID NO: 41 - protein, 459 aa, synthetic construct
MAQSISFDSSPATPSLGLETKIAAIIQNILTLTLLLLALPINTAIVFIYLVVGAIFRPQTSKTSNPKNILISGGKMTKSLQLARSFHAPGHRVVLVETHKYWLTGHRFSQAVDKFYTTPAPQKDPEAYIQALEEIVKRENIDVYIPVTSPVGSYYDSLAKPKLSPHCEVLHFDAEITQMLDDKFAMAEKARSLGLSVPKSFKITSSEQVINFDFSGETRKYILKSIPYDSVRRLDLTKLPCATPEETAAFVRNLPISPEKPWIMQEFIPGKEFCTHSTVRDGEIKLHCCCESSAFQVNYENVENPQILEWVKHFVKELKLTGQISFDFIQTEDGQVYAIECNPRTHSAITAFYNHPLVAEAYIGSVTETLQPLSTSKPTYWTYHEVWRLTGIRSFTQLKTWLHNIWRGTDAILKLDDPLPFLMVHHWQIPLLLLNNLRQLKGWTRIDFNIGKLVELGGD

SEQ ID NO: 42 - protein, 442 aa, synthetic construct
MRKHIFVVFQNLGTLVLLAIAFPLNCIVVLTSLLWSFIKQPFNKSIVVNPNSKNILIAGARMTKTLQLARSFHAAGHRVIIIDIEKYWLSGNKYSNSVAGFYTVPDPSKDLEGYVETLHAIANTEKIDFFIPVAIFSVIHYDQGKPPLPDCVEFFHFDADVTKILDDKFAFAETARSFGLSVPKSFKITDPEQVLNFDFSQEKRKYILKSIPYDQVRRLNLTKLPCDTKSETAAFVKSLPISEENPWIMQEFIPGKEYCTHTTARDGESRMYCCCESSAFQVNYENVDQREIMQWASHFTKELGKTGQLSFDFIQAEDGTVYAIECNPRTHSAITMFYNHPGVADAYLGKEPLAESLQPLPDSKPTYWLYHEVWRLNEIRSFKQLQTWVRNIRRGKEAIFEVSDPLPFLMVHHWQIPLLILDNLRRLKGWIRIDFNMGELIE

SEQ ID NO: 43 - protein, 455 aa, synthetic construct
MKSRQTPRERTFALLKSLGTLSLLLLAFPFSLSAVVGALLWSSLASLFQKRRVQAEPKRILLTGAKMTKCLTLARSFHAAGHQVVMVETHKYWLSGNRFSNCVEAFYTVPAPQHDAEGYIQGLLNIVKQEKIDMFIPVSSPVASYYDSLAKPALSPYCEVFAFDAETTKLLDNKFTFNQKAHSVGLSAPKTFLITNPEQVLNFDFAADGSQYILKSIAYDSINRLALLKLPCAPQKMAEYVRSLPISEENPWIMQEFLKGQEYCTHAVVRDGKLLLYACSKSCDFLVNYEHDYNPAILDWVTRFVKELNLTGQICLDFIQAEDGTVYPIECNPRTSTCITMFHDQPKVVADAYLSSGAQASKEPVQPLPDSKPTYWTFHELWRLLTKVKSWKDLQYRLGIIFNGVDPVFHPRDPLPFLGVNHWQIPLLILNNVRQLKGWERIDFNIGKLVQLGGD

SEQ ID NO: 44 - protein, 455 aa, synthetic construct
MQSGQTTSERTFALLKSLGTLTLLLLAFPFSLSVVVGALLWSSLTSLFQKRRVQVEPKRILLTGAKMTKCLTLARSFHAAGHQVFMVETKKYWLSGNQFSNCVEALYTVPAPQHDAEGYIQGLLNIVKQEKIDMFIPVSSPVASYYDSLAKPALSPYCEVFAFDAETTKLLDNKFTFNQKAHSVGLSAPKTFLITNPEQVLNFDFAADGSQYILKSIAYDSINRLALLKLPCAPEKMAEYVHSLPISAENPWIMQEFLKGQEYCTHAVVRDGKLLLYACSKSCDFLVNYEHDYNPAILDWVTRFVKELNLTGQICLDFIQAEDGTVYPIECNPRTSTCITMFHDQPKVVADAYLSSSAQAPKEPVQPLPESKPTYWTFHELWRLLTKVKSWKDLQYRLGIIFNGVDPVFHPRDPLPFLGVNHWQIPLLILNNVRQLKGWERIDFNIGKLVQLGGD

SEQ ID NO: 45 - protein, 465 aa, synthetic construct
MAHSISLSSRPATPAISIKALLVALFQNLGTLTILLLVLPINAAIVLISLLWSRLSSPWRSQKAVVATHRKNILISGGKMTKALQLARSFHAAGHRVVLIETHKYWLSGHRFSNAVSRFYTTPTPQHNPEAYIQALLDIVKREKIDVYVPVTSPVASYYDSLAKPALSPYCEVFHFDADVTQMLDDKFAFSEKARALGLSVPKSFKITNPEQVINFDFSQETRKYILKSIPYDSVRRLDLTKLPCDTPEETAAFVRSLPISPEKPWIMQEFIPGKEFCTHSTVKNGELRLHCCSESSAFQVNYENIENPKIQKWVTHFVKELGITGQISFDFIQAEDGTVYAIECNPRTHSAITMFYNHPQVADAYLSQEAFTEPQEPLPNSKPTYWTYHEVWRLTGIRSFAQLQTWIRNFLRGKDAIYQVDDPLPFLMVHHWQIFLLLLDNLRQFRGWTRIDFNIGKLVELGGD

SEQ ID NO: 46 - protein, 450 aa, synthetic construct
MQIFAVFQNLGTLLLLAIAFPFNCIVVLTALFWNLVSKPFRDRGILPVSHPKNIMLTGGKMTKALQLARSFHMVGHRVVLVETHKYWLTGHRFSNAVDRFYTVPAPEKDPEGYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATQAETAAFVNSLPISPEKPWIMQEFIPGQEYCTHSTVRNGELRMHCCCESSAFQVNYENVDKPEILAWVRHFVKELGITGQASFDFIQAEDGNVYAIECNPRTHSAITMFYNHPGVADAFCRDVTCNVSTLYPLQPLSTSKPTYWTYHELWRLTGIRSFPQLQTWFKNILRGKDAIFAIDDPLPFLMVHHWQIPLLLLDNLRRLKGWIRIDFNIGKIVELGGD

SEQ ID NO: 47 - protein, 454 aa, synthetic construct
MALILFVQGRAYALFQNLGTLILLLIVLPFNFLKVIPSLLWNFISQPFQKKVVAENPKNILITGAKMTKCLQLARSFHAAGHKVFLLEANKYWLSGNRFSNAVTGFYTLPFPQKDWEGYSQGLLEIIKKEKIDVFIPVSSPAGSYYESLAKPLISEHCEVLHFDAEITQLLDNKFTFIEKAKSFGLSVPKSFLITNPEQVLNFDFATDGSKYILKSIPYDSVRRLDMTKLPMNSKAEMEEFVNSLPISEQRPWIMQEFVKGKEYCTHSTVRKGKVRLYCCCESSEFQVNYHHVDRPQIYQWVEKFVRELNITGQISFDFIQTEDGRVYPIECNPRTHSAITTFYDHPGVADAYLKDSKDENEASLIPLPNSKPTYWTYHELWRLTGIRSLGQLKTWINRIFQGTDGIFQINDPLPFLMVHHWQIPLLLLGNLQKLKGWVRIDFNIGKLVELGGD

SEQ ID NO: 48 - protein, 454 aa, synthetic construct
MGLISGSQKPIYTVLQNLGTLTLLLSVLPFNLLKVLPALLWNFLSKPFQKKLVVENSKNIILTGAKMTKCLQLARSFQAAGHKVFMLETDKYWLSGNRFSNSVTGFYTVPNPKKDWNGYCQKLLDIVKKENIDVFIPVSSAVLNYYESLVKPILSEYCEVLHFDVEITKLLDNKFTFIEKAKSFGLTVPKSFLITKPEQIINFDFATDGSQYILKSIPYDSVRRLNMTKLPMKSVQEMSNFVKSLPINQEKPWIMQEFVKGKEYCTHSTVRKGQIRLHCCCESSEFQVNYEHVDHPQIYEWIEKFVKELNLTGQISFDFIQTEDNRVYPIECNPRTHSAITTFYNHPEVADAYLNDSQNDNESPITPLSNSKPTYWTYHELWRLTAIRSWEQLKAWSKKITAGTDSIFQFNDPLPFLMVHHWQIPLLLLENLKKLKGWVMIDFNIGKLVELEED

SEQ ID NO: 49 - protein, 440 aa, synthetic construct
MFLTTFKSLGTLALLKLALPFNLTLVLIASIINIFSNPFKIKKKPNINSKTVLLTGGKMTKALQLARSFHSAGHRVILVETHKYWLSGHRFSVAVDKFFTMPNPVKDKEGYIDGLLDIVKRESVDIFIPVSSPVASYYDSVAKMVLSPYCEVLHFDVEMTLVLDDKANLCKKASSLGLTSPASYLITNVQEILDFDFSKNNHKYILKSIKYDSVYRLNMTQFPFEGMEEYVRSLPISEENPWVMQQFITGQEYCTHSTVRNGKIRLHCCSESSHFQVNYKHIDNQKIYEWVEEFVGKLNLTGQISFDFIQTDDGTVYPIECNPRTHSAISMFYNHPLVADAYLNDGDDAPITPLESSKPTFWTYHELWRLTEVRSPQDLSQWWQKVTKGQDGIFSWQDPLPFLMVHHWQIPLLLFGNLMKLKPWVKIDFNIGKLVESAGD

SEQ ID NO: 50 - protein, 459 aa, synthetic construct
MTQSISVASVGQTTQSVTLGLRISALFKNLATLALLLLVLPINAAIVLVSLLLGSQSQAIATEPKNILISGGKMTKALQLARSFHAAGHRVVLVETHKYWLTGHRFSKAVSRFYTLPTPQSDPEAYTQALLDIVQKENIDVYVPVCSPVASYYDSLAKPVLSKYCEVFHCDADVTQMLDDKYAFVEKARSLGLSVPKSFKITDPEQVSNFDFSQEKRKYILKSIPYDSVRRLDLTKLPCETPEATADFVNSLPISSQKPWIMQEFIPGKEFCTHSTVRNGELRMHCCCESSAFQVNYENVDHPQILEWVRHFVKALGITGQVSFDFIEAQDGTIYAIECNPRTHSAITMFYNHPDVANAYLSEIPQVEPIQPLINSKPTYWTYHEIWRLTGIRSFSQLQTWLKNFFGGKDAIYSLSDPLPFLTVHHWQIPLLLLQNLQQLKGWIRIDFNIGKLVEFGGD

SEQ ID NO: 51 - protein, 458 aa, synthetic construct
MAQSLPLSSAPATPSLPSQTKIAAIIQNICTLALLLLALPINATIVFISLLVFRPQKVKAANPQTILISGGKMTKALQLARSFHAAGHRVVLVETHKYWLTGHRFSQAVDKFYTVPAPQDNPQAYIQALVDIVKQENIDVYIPVTSPVGSYYDSLAKPELSHYCEVFHFDADITQMLDDKFALTQKARSLGLSVPKSFKITSPEQVINFDFSGETRKYILKSIPYDSVRRLDLTKLPCATPEETAAFVRSLPITPEKPWIMQEFIPGKEFCTHSTVRNGELRLHCCCESSAFQVNYENVNNPQITEWVQHFVKELKLTGQISFDFIQAEDGTVYAIECNPRTHSAITTFYDHPQVAEAYLSQAPTTETIQPLTTSKPTYWTYHEVWRLTGIRSFTQLQRWLGNIWRGTDAIYQPDDPLPFLMVHHWQIPLLLLNNLRRLKGWTRIDFNIGKLVELGGD

SEQ ID NO: 52 - protein, 465 aa, synthetic construct
MAQSISLSLPESTTPSTSAGVKIVALFKTLGTLTLLLIALPFNALIVLIALLWGIVRRPFTKKAAVAAHPQTILVSGAKMTKALQLARSFHAAGHRVILIEGHKYWLSGHRFSKAVSRFYTVPAPQKDPEGYIQALVEIVKKEKVDVYVPVCSPVASYYDSLAKPALSEYCEVFHFDADITKMLDDKFAFTDKARSLGLSVPKSFKITDPQQVINFDFSQETRKYILKSIAYDSVRRLDLTKLPCDTPEETAAFVNSLPISSENPWIMQEFIPGKEFCTHSTVRDGELRLHCCCNSSAFQINYENVENPQIREWVQQFVKSLRLTGQVSFDFIQAEDGTVYAIECNPRTHSAITMFYNHPGVADAYLGKTPLAAPLEPLASSKPTYWIYHEIWRLTGIRSWKQLQTSINTLLRGTDAICCLDDPVPFLTLHHWQIPLLLLKNLQQLKGWVKIDFNIGKLVELGGD

SEQ ID NO: 53 - protein, 465 aa, synthetic construct
MAQSISLSLPQSTTPSTGVKVKIVALFKTLGTLTLLLIALPFNALIVLISLLWGIGRSPFTKKAVVATHPQTILVSGAKMTKALQLARSFHAAGHRVILIEGHKYWLSGHRFSKAVSRFYTVPAPQEDPEGYIQALVEIVKQEKVDVYVPVCSPVASYYDSLAKPALSEYCEVFHFDADITKMLDDKFAFTDRARSLGLSVPKSFKITDPQQVINFDFSQEIRKYILKSISYDSVRRLDLTKLPCDTPEQTAAFVNSLPISPEKPWIMQEFIPGKELCTHSTVRNGELRLHCCSNSSAFQINYENVENPRIREWVQHFVKSLGLTGQVSFDFIQAEDGTTYAIECNPRTHSAITMFYNHSGVANAYFGKTLLDAPLEPLASSKPTYWIYHEIWRLTGIRSWKQLQTSVNTIVRGTDAIYCLDDPVPFLTLYHWQIPLLLLKNLQQLKGWVKIDFNIGKLVELGGD

SEQ ID NO: 54 - protein, 462 aa, synthetic construct
MAQSISLSLPESTTPSTGIRIKIVALFKTLGTLTLLLIALPINALIVLLSLLWSILFTKKPAVAAHPQTILVSGGKMTKALQLARSFHAAGHRVILVEGHKYWLSGHRFSNAVSRFYTVPAPQDDPEGYIQALLEIVKKEKVDIYVPVCSPVASYYDSLAKPSLSAYCEVFHFDAEITKMLDDKFAFTDQARSLGLSVPKSFKITDAEQVINFDFSKETRKYIIKSISYDSVRRLNLTKLPCDTPEETAAFVKSLPISPEKPWIMQEFIPGKELCTHSTVRDGELRLHCCSDSSAFQINYENVENPQIRQWVQHFVKSLGLTGQVSFDFIQAEDGTAYAIECNPRTHSAITMFYNHPGVAEAYFGKTLLAAPLEPLADSKPTYWIYHEIWRLTGIRSAKQLQTWFQRLVRGTDAIYQINDPIPFLTLHHWQITLLLLQNLQKLKGWVKIDFNIGKLVELGGD

SEQ ID NO: 55 - protein, 463 aa, synthetic construct
MAQSIPFDSASPTPQVSWGVRISALWKTVGTLLLLFLALPVNASIVLISLLWGIFSKPFEKRVVAAAPKNILISGGKMTKALQLARSFHAAGHRVVLVESHKYWLTGHQFSNAVSVFYTVSPPEKDPEGYTQQLLDIVKKERIDVYVPVCSPVASYYDSLVKPALSQHCEVFHCDAEITQMLDDKYAFSEKARSFGLSVPKSFKITNPEQVINFDFSQEKRKYILKSIPYDSVRRLNLTKLPCDTPEETAAFVRSLPISPEKPWIMQEFIPGKEFCTHSTVRNGELRLHCCCESSAFQVNYENVNNPQILEWVKHFIKEMGITGQVSFDFIQTEDGTVYAIECNPRTHSAITMFYNHPGVADAYLGKIPLPEPLQPLADSKPTYWLYHEIWRLTGIRSLSQFWTWLKNLMRGKDAIYQLNDPLPFLTVPHWQITLLLLQNLRQLRGWVKIDFNIGKLVELGGD

SEQ ID NO: 56 - protein, 455 aa, synthetic construct
MQSGQTIRERTFASLKSLGTLTLLLLAFPFSLSVVVGALLWSSLTSLFQKHRVQVKPKRILLTGAKMTKCLTLARSFHAAGHQVFMVETKKYWLSGNQFSNCVEALYTVPAPQHDAEGYIQGLLNIVKQEKIDMFIPVSSPVASYYDSLAKPALSPYCEVFAFDAETTKLLDNKFTFNQKAHSVGLSAPKTFLITNPEQVLNFDFATDGSQYILKSIAYDSINRLALLKLPCAPATMAKYVHSLPISEENPWIMQEFLKGQEYCTHAVVREGKLMLYACSKSCDFLVNYEHDYNPAILDWVTRFVKALNLTGQICLDFIQAEDGTVYPIECNPRTSTCITMFHDQPKVVADAYLSSSASILKEPVQPLPDSKPTYWTFHELWRLITKVKSWQDLQYRLGIIFNGVDPVFHPRDPLPFLGVNHWQIPLLILNNVRQLKGWERIDFNIGKLVQLGGD

SEQ ID NO: 57 - protein, 454 aa, synthetic construct
MLIILFIQNHAYALFQNLSTFLLLTLLLPFNLLKILPVVLWNILTPIRAKPPGYEKPKNILITGAKMSKSLQLARSFNGSGHRVFLLEIHKYWLSGNRFSNAIKGFYTVPNPQKDWDGYQQAVLEIVQKENINLFIPVSSPAGSYDESRLKPILSPYCEVFHFNLDITELLDNKFTFIEKAKSLGLSVPQSFLITDSKQILDFDFAQDGSRYILKSIPYDSVRRLDMTKLPMKSEQEMEEFVKKLPITEDKPWIMQEFVQGKEYCTHSTVRKGKIRLHCCCESSEFQVNYDHVEEPEIYQWVETFVRALNLTGQISFDFIKTEDGQVYPIECNPRTHSAITTFHDHPGVADAYLKDAEDETESPIFPLPDSKPTYWTYHELWRVTEIRSFGQFQAWIKRITEGTDGIFQLNDPLPFLMVHHWQIPLLLLQNLKKMKGWVRIDFNIGKLVELDGD

SEQ ID NO: 58 - protein, 472 aa, synthetic construct
MGQSISLSLPQSPTSSTSVRVKIIALFKTLGTLTLLLIALPFNALIVLISLLWGIVRWTLPRRRRSLFTKNVVAAHPQTILVSGAKMTKALQLARSFHAAGHRVILIEGHKYWLSGHRFSKAVSRFYTVLAPQSDLEGYIQALVEIVKKEKVDVYVPVSSPVSSYYESLAKAALSEYCEVFHFDPDITKMLDDKFALTDRARSLGLSVPKSFKITDPQQVINFDFSQETRKYILKSIDYDSVRRLNLTKLPCDTPEETAAFVNSLPISPEKPWIMQEFIPGKELCTHSTVRDGELRLHCCSDSSAFQINYENVENPQIREWVQHFVKSLALTGQVSFDFIQAQDGTVYAIECNPRTHSAITMFYNHPGVADAYLGKTPLAAPLEPLASSKPTYFIYHEIWRLTGIRSWKQLQTSVNTLVRGTDAIYSLDDPIPFLTLHHWQIPLLLLKNLQQLKGWVKIDFNIGKLVELGGD

SEQ ID NO: 59 - protein, 463 aa, synthetic construct
MPQSISLTSSPTINQVNNKSVDISSSLKTLGTLTLLLLALPVNATLVLVALLLNSLRPRNITTAANPKNILISGGKMTKALQLARSFHNAGHRVVLLEAHKYWLTGHRFSFAVNKFYTVEAPEKDPEGYVQSLVDIVNKENIDVYVPVCSPVASYYDSLAKKALSSQCEVIHCDALTTQMLDDKYAFTETARGFGLSVPKSFKITDPEQVINFDFSQEKRKYILKSIPYDSVRRLDLTKLPCDTPEATAAFVRSLPISPEKPWIMQEFIPGKEYCTHSTVRNGEITLHCCCESSAFQVNYAQVDNPQIFEWVRHFLKQLGITGQVSFDFIEAEDGTVYAIECNPRTHSAITMFYNHPGVADAYLGTLNNLEEPIQPLPTSKPTYWIYHEMWRLINAGSWSKFVERLQIITRGTDAIFSWQDPLPFLMNPHWQIFLLLIQNLQKNRGWIRIDFNIGKLVELGGD

SEQ ID NO: 60 - protein, 450 aa, synthetic construct
MFLQAKIWAFFQNIGTLTLLLLALPFNAIVVLPCLLWSWIAKLFQKKVVAANPKNILITGGKMTKALQLARCFHAAGHTVFLVETHKYWLSGHRFSRAVKGFFTVPAPEKHANGYCQGLLDIVKQEKIDVFIPVSSPVASYYDSIAKSLLSPHCEALTFDAEITEMLDNKFTFCQKARELGLTAPKAFLITDPEQVLNFDFAADGSRYILKSIAYNSVYRLDLTKLPMSSKEQMASFVKGLPISESQPWIMQEFISGQEYCTHSTVRNGIVRLHCCSQSSPFQVNYEQVDNQNIFQWVQQFVKALNLTGQISLDVIQTKDGKVYPVECNPRTHTAIAMFYNHPGVADAYILDSKDAREPPIQPLPESKPTYWTYHELWRLTGIRSWGQLKGWFNKIIKGTDGIFQVNDPLPFLMVHHWQIPLLLLNNMRKFKGWVKIDFNIGKLVELGGD

SEQ ID NO: 61 - protein, 465 aa, synthetic construct
MAQSISLSLPQSPTSSTGIKVKLVALFNTLGTLTLLLIALPFNALIVLISLLWGIVSSPFTKKAVVAAHPQTILVSGAKMTKALQLARSFHAAGHRVILIEGNKYWLSGHRFSKAVSRFYTVPAPQEDPEGYIQALVEIVKREKVDVYVPVCSPVASYYDSLAKPLLSEYCEVFHFDPDITKMLDDKFAFTDRARSLGLSVPKSFKITDPQQVINFDFSQETRKYILKSIDYDSVRRLNLTKLPCDTPEETAAFVNSLPISAEKPWIMQEFIPGKELCTHSTVRNGELRLHCCSNSSAFQINYENVENPQIREWVQHFVKSLALTGQVSFDFIQAEDGTAYAIECNPRTHSAITMFYNHPGVADAYLGKTPLAAPLEPLASSKPTYFLYHEIWRLTGIRSWKQLQTSVNTLVRGTDAIYSLDDPIPFLTLHHWQIPLLLLKNLQQLKGWVKIDFNIGKLVELGGD

SEQ ID NO: 62 - protein, 469 aa, synthetic construct
MLMGFFEGEFMTQSISVASPAPKTQSVPLGFRISALWKNVGTLALLLLVLPINAVIVLVSLLLGHQSQAIATEPKNILISGGKMTKALQLARSFHAAGHRVVLVETHKYWLTGHRFSKAVSRFYTLPTPQSDPKAYTQALLDIVKKENIDVYVPVCSPVASYYDSLAKPVLSKYCEVFHCDADVTQMLDDKYAFAEKARSLGLSVPKSFKITDPEQVINFDFSQEKRQYILKSIPYDSVRRLDLTKLPCETPEVTADFVNSLPISPQKPWIMQEFIPGKEFCTHSTVRNGELRMHCCCESSAFQVNYENVDHPQILEWVRHFVKELGITGQVSFDFIQAEDGTIYAIECNPRTHSAITMFYNHPSVADAYLSEIPQLEPIQPLFNSKPTYWIYHEIWRLTGIRHWSQLQTWLKNFFGGKDAIYSFSDPLPFLTVHHWQIPLLLLQNLQQLKGWLRIDFNIGKLVEFGGD

SEQ ID NO: 63 - protein, 454 aa, synthetic construct
MGLISRSQKPVYIALQNLGILTLLLSVLPFNLLKVLPAVLWNFISKPFQKKVVAENSKNIILTGAKMTKCLQLARSFQVAGHKVFMLETDKYWLSGNRFSNTVTGFYTVPNPKKNWNGYCQELLDIVKREDIDVFIPVSGAALNYYESLIKPILSEHCEVLHFDIEITKLLDNKFTFIEKAKSFGLAVPKSFLITNPEQILNFDFPADGGQYILKSIPYDSVRRLDMRKLPMKSAQEMKDFVNSLPISEEKPWIMQEFVKGKEYCTHSTVRKGQIRLHCCCESSEFQVNYEHVNHPQIYEWVETFVKELNLTGQISFDFIQTEDNRVYPIECNPRTHSAITTFYNHPEVADAYLNDSQDDNESPLIPLPNSKPTYWIYHELWRLTAIRSWEQLKDWIKKITAGTDSIFQFNDPLPFLMVHHWQIPLLLLDNLKKLKGWVMIDFNIGKLVELEED

SEQ ID NO: 64 - protein, 464 aa, synthetic construct
MTQSISLSLPESTTPSVGIKVKILALFKTLGTLSLLLVALPFNVLIVLISLLWGIVRVPFTKNVVATHSQTILVSGAKMTKALQLARSFHADGHRVILIESHKYWLSGHRFSKAVSRFYTVPSPQKDPESYIQALIEIVKKEKVDVYVPVCSPVASYYDSLAKPALSEYCEVFHFNADITKMLDDKFAFTQKARALGLSVPKSFKITDPQQVINFDFSQETRKYILKSINYDSVRRLNLTKLLCDTPEETAAFVKSLPISPETPWIMQEFIPGKEFCTHSTVRDGELRLHCCCHSSAFQINYENVENPQIREWVQHFVKSLGLTGQVSFDFIQAEDGTVYAIECNPRTHSAITMFYNHPGVAEAYFGKIPLPAPVEPLATSKPTYWTYHEIWRLTGIRSWKQLQTAIKTIFQGTDAIYCLDDPLPFLTLHHWQIPLLLLQNLQQLKGWVKIDFNIGKLVELGGD

SEQ ID NO: 65 - protein, 458 aa, synthetic construct
MAQSLSLSSSHATPSIPWQTRVAAILQNIGTLTLLLLALPINASIVFISWLIFRPQKVKAANPQNILISGGKMTKALQLARSFHAAGHRVVLLETHKYWLTGHRFSVAVDKFYTVPAPQENPQAYIQALVDIVKQENIDVYVPVTSPAGSYYDSLAKPELSRYCEVFHFDADITQMLDDKFALVEKARSLGLSVPKSFKITSPEQVINFDFSGESRKYILKSIPYDSVRRLDLTKLPCATPEETAAFVRTLPISQEKPWIMQEFIPGKEFCTHSTVRDGELRLHCCCESSAFQVNYENVDNPQIREWVRRFVKELKLTGQISFDFIQAEDGTVYAIECNPRTHSAITTFYDHPQVAQAYLSKETTAETLQPLATSKPTYWTYHEVWRLTGIRSLTQLGRWLGNIWRGTDAIYQPGDPLPFLMVHHWQIPLLLLNNLRRLKGWTRIDFNIGKLVELGGD

SEQ ID NO: 66 - protein, 464 aa, synthetic construct
MLETVSVAAMPSERETNTGNRRFPTAFKTIATLILLLLVMPLNLALTAIALLRSIIIKPFQSRSTTATPQTILISGGKMTKALQLARSFHQAGHRVILVETEKYWLTGHRYSRAVDRFYTVPNPQTEEYPQALLKIVRQEGVNVYVPVCSPVASYYDAEVKRVLSGHCTVMHVDVETLQRLDDKYEFATAAQALGLPVPKSYRITNPQQVIDFDFSDAQRKYIIKSIPYDSVRRLDLTKLPCETPAETAAFVNSLPISESKPWIMQEYIPGQEFCTHSTVRNGHLQLHCCCKSSAFQVNYENVDRPDIENWIRQFAKSLNLTGQVSFDFIQAADDGEIYAIECNPRTHSAITMFYNHPDVAKAYLEPDPLPQTVQPLASSRPTYWIYHEIWRLVTHLSSPKLVSERLKIIAQGKDAIFDWDDPLPFLMVHHWQIPLLLWGNLQNPKEWIRIDFNIGKLVEIGGD

SEQ ID NO: 67 - protein, 347 aa, synthetic construct
SRSVDRFYTVPKPQEKDYIDALLEIVQREGVDVYIPVCSPVASYYDALAKQVLSKYCEVMHFDPELVQKLDDKSEFSAIATSLGLAVPDSYRITDTQQILDFDFAKQAHTYILKSIPYDSLRRLNLTQLPCETPQQTAAFVEQLPICESNPWIMQAFITGQEYCTHSTVRNGELQLHCCCESSAFQINYEMVDKPEIEAWVRKFVSSLKLTGQVSFDFIQTRDGGVYAIECNPRTHSAITMFYNHPDVARAYLESDFPLIKPLESSRPTYWIYHEIWRLVTQPTQIGQRLKIIASGKDAIFDWADPLPFLMVHHAQIPWLLLENLRQLKGWMRIDFNIGKLVEPAGD

SEQ ID NO: 68 - protein, 455 aa, synthetic construct
MAQVQPIKARIFAVFQNLGTLALLAIAFPINCIVVLASLLWNFCSRPFSKQGVSTLNPKNILIGGGKMTKTLQLARLFHAAGHRVILFDSEKFRFSGYRFSNAVDRFYTVPDPQTDLEGYTQALRAIAKQENIDIFIPVGIFAGGYFDSQRQPVLSGCCELFHFDADTMKMLDNKFTFGEIARSFGLSVPKTFLITDPEQVLQFDFANEKNKYILKSIVYDSVYRLDMTKLPMESQEKMAAHVNSLPIRKDNPWILQEFISGKEYCTHSTVRNGELTVHCCCESSAFQVNYENVDHPEIMQWVSRFVKELKLSGQISFDFMQAEDGTLYAIECNPRTHSAITMYYNHPDLADAYLSAERRNYALPLQPLPDSKPTYWLYHEVWRLNEIRSLKQLQTWFKNIWRGKDAIFEVNDPLPFLMVHHCYIPLLLLDSLRKLKGWVRIDFNIGKLVQLEGD

SEQ ID NO: 69 - protein, 463 aa, synthetic construct; identical to SEQ ID NO: 59

SEQ ID NO: 70 - protein, 464 aa, synthetic construct
MAQSISVSSSPAMPSLAVETKIAVIIQNILTLALLLLALPINATIVVVTLLWCNISRPFQHSATKAANPKNILISGGKMTKALQLARSFNAAGHRVVLIETHKYWLSGHRFSQAVDKFYTVPAPQENPECYTQALIDIIKQENIDVYIPVTSPLGSYYDSLAKPLLSEYCEVFHFDADITQKLDDKFAFAETARSLGLSAPKSFKITSAEQVLNFDFSQESRKYILKSIPYDSVRRLDLTKLPCATPEETAAFVRSLPISPEKPWIMQEFIPGKEFCTHSTVRDGELRLHCCCESSAFQVNYENVENSQIREWVRHFVKELKLTGQISFDFIQAEDGRVYAIECNPRTHSAITTFYDHPKVAQAYLDKEPMAETLQPLPTSQPTYWTYHEVWRLTGIRSFTQLKKWIANIWRGTDAIYKSDDPLPFLMVHHWQIPLLLIDNLRRLKGWTRIDFNIGKLVELGGD

SEQ ID NO: 71 - protein, 458 aa, synthetic construct; identical to SEQ ID NO: 51

SEQ ID NO: 72 - protein, 459 aa, synthetic construct
MTQSISVASVGQTTQSVTLGLRISALFKNLATLALLLLVLPINAVIVLVSVLLGSQSQAIATEPKNILISGGKMTKALQLARSFHAAGHRVVLVETHKYWLTGHRFSKAVSRFYTLPTPQSDPQAYTQALLDIVKKESIDVYVPVCSPVASYYDSLAKPVLSKYCEVFHCDADVTQMLDDKYAFAEKARSLGLSVPKSFKITDPEQVINFDFSQEKRQYILKSIPYDSVRRLDLTKLPCETPQATADFVNSLPISPQKPWIMQEFIPGKEYCTHSTVRNGELRMHCCCESSAFQVNYENVDHPQILEWVRHFVKALGITGQVSFDFIEAEDGTIYAIECNPRTHSAITMFYNHPDVANAYLSEIPQVEPIQPLTNSKPTYWTYHEIWRLTGIRSFSQLQTWVKNFFGGKDAIYSLSDPLPFLAVHHWQIPLLLLQNLQQLKGWIRIDFNIGKLVEFGGD

SEQ ID NO: 73 - protein, 450 aa, synthetic construct
MFLQAKIWAFFQNIGTLTLLLLALPFNAIVVLPCLLWSWIAKLFQKKVVAANPKNILITGGKMTKALQLARCFHAAGHTVFLVETHKYWLSGHRFSRAVKGFFTVPAPEKHANGYCQGLLDIVKQEKIDVFIPVSSPVASYYDSIAKSLLSPHCEALTFDAEITEMLDNKFTFCQKARELGLTAPKAFLITDPEQVLNFDFAADGSRYILKSIAYNSVYRLDLTKLPMSSKEQMASFVKGLPISESQPWIVQEFISGQEYCTHSTVRNGIVRLHCCSQSSPFQVNYEQVDNQKIFQWVQQFVKALNLTGQISLDVIQTKDGKVYPVECNPRTHTAIAMFYNHPGVADAYLLDSKDAREPPIQPLPESKPTYWTYHELWRLTGIRSWGQLKGWFNKIIKGTDGIFQVNDPLPFLMVHHWQIPLLLLNNMRKFKGWVKIDFNIGKLVELGGD

SEQ ID NO: 74 - protein, 423 aa, synthetic construct
MFPINLTLVITAFLTNLITLPFPKKITYENSKNILLTGGKMTKSLQLARSFHRAGHKVFMVETHKYWLSGHQYSKAVKKFLTVPAPEKDPEGYCQSLLDIVKREKIDVFIPVSSPVASYYDSLAKPILSPYCEVFHFDTEMTKTLDDKFSLCEQARVLGLTAPKVFLITSPGEIINFDFSQEQNPYIIKSIQYDSVTRLDMTKFPFEGMKEYVKKLPISKERPWVMQEFIKGQEYCTHSTVRDGEIRLHCCSKSSPFQVNYEQVDNPEIFQWVQKFVKELNLTGQISFDFMQTEDGKVYPIECNPRTHTAITMFYDHPGLADAYLEPGKNQPHIEPLPTSKPTYWLYHELWRITGIRSFNDLTNWLNKVIKGKDAMLDKDDPLPFLMVHHWQIVLLLLQNMVKLKGWVRIDFNIGKLVEIGGD

SEQ ID NO: 75 - protein, 454 aa, synthetic construct
MLIILFIQNRAYALFQNLSTFLLLTLLLPFNLLKILPALLWNILTSIRAKLPGDEKPKNILITGAKMSKSLQLARSFNGAGHRVFLLETHKYWLSGNRFSNAIKDFYTVPNSEKNWDGYQQAVLEIVQKENINLFIPVSSAAGSYDESRLKAILSPYCEVFHFDLDITELLDNKFTFIEKAKNLGLSVPKSFLMTDSKQILDFDFVQDGSRYILKSIPYDSVRRLDMTKLPMKSEQEMEEFVKELPITEDKPWIMQEFVQGKEYCTHSTVRKGKIRLYCCCESSEFQVNYNHVEEPEIYQWVKTFVRALNLTGQISFDFIKTEDGQVYPIECNPRTHSAITTFHDHPGVADAYLKDVEDETKSPIFPLPDSKPTYWTYHELWRLTQIRSFGQFKAWIKRMIEGTDGIFQPHDPLPFLMVHHWQIPLLILQNLKTMKGWVRIDFNIGKLVELDGD

SEQ ID NO: 76 - protein, 464 aa, synthetic construct
MAQSISVDSSPAIPSLASETKIAVIIQNILTLALLLLALPINATIVLVTLFWGTILRPFQHSATKTANPKNILISGGKMTKALQLARSFHAAGHKVVLLETHKYWLTGHRFSQAVDKFYTVPAPQENPESYTQALIDIIKQENIDVYIPVTSPLGSYYDSLAKPLLSRHCEVFHFDVDITQNLDDKFEFAQKARSLNLSAPKSFKITSAEQVLNFDFSQESRKYILKSIPYDSVRRLDLTKLPCATPEETAAFVRSLPISPEKPWIMQEFIPGKEFCTHSTVRDGELRLHCCCESSAFQVNYENVENSQIREWVRHFVKELKLTGQISFDFIQAEDGAVYAIECNPRTHSAITTFYDHPKVAQAYLDQEPMAETLQPLPTSKPTYWTYHEVWRLTGIRSFTQLQKWLANIGRGTDAIYKLDDPLPFLMVHHWQIPLLLLNNLLRLKGWTRIDFNIGKLVELGGD

SEQ ID NO: 77 - protein, 469 aa, synthetic construct
MAQSSIPVLSSQTATHTISLGRRFVALVQNLATLTALLLALPINATIVFISLVLKILISPFQKEQTTVTTAERKNILISGGKMTKALQLARFFHAAGHRVVLTETHKYWLSGHRFSQAVDKFYTTPVPQKDSQIYTQALIDIVNKENIDIYIPVTSPIASYYDALAKQTLSEYCEVFHIDAATCEMLDDKFAFSEKARSFGLSVPKSFKITNPEQVLNFDFSGETRKYILKSIPYDSVRRLDLTKLPCDTPEETEAFVRSLPISPQKPWIMQEFIPGKEYCTHSTIRDGVVRLHCCCESSAFQVNYENVENAKIREWVTHFVKELGVTGQLSFDFIEAEDGNVYAIECNPRTHSAITIFHDQLQPAANAYLSKEPIKEPLQALINSKPTYWTYHEFWRLNEIRSFSQLGNWIKNMLQGTDAIYTFDDSLPFLMVHHWQIPLLLLKNLFKLKGWTRIDFNIGKLVESGGD

SEQ ID NO: 78 - protein, 465 aa, synthetic construct
MAQSISLTESQTTVKPLAVWGKINALLKNLGTLVLLLVALPINATIVLVSLLWNLLAKPFQKEQTVAGDRKNILISGAKMTKALQLARSFHAAGHRVVLLETHKYWLSGHRFSKAVDNFYTTPVPQRDPQAYTQALIDIIEKENIDVYIPVTSPIASYYDSLAKPVLSQYCEVFHFDAAVTQMLDDKFAFSEKARSLGLSVPKSFKITSPEQVLNFDFSQETRKYILKSIPYDSVRRLDLTKLPCDTPEQTEAFVRSLPISAQKPWIMQEFIPGKEFCTHSTVRDGEIRLHCCCESSAFQVNYEHVEHPQISEWIARFVKGLGITGQISFDFIQAEDGSVYAIECNPRTHSAITTFHDRPEVAQAYLGKEAMTEPLQPLPSSKPTYWLYHEVWRLTSIRSLAQLRTWIRNIWRGTDAIYKLDDPLPFLMLHHWQIPLLLLNNLWRLKGWTRIDFNIGKLVELGGD

SEQ ID NO: 79 - protein, 453 aa, synthetic construct
MAQLLFVRTPSFTMLKSLGTLTLLLIAFPINSIVVLTSLLWGLLSRPFQKQPLPADNQKTAMFTGGKMTKALQLARSFHAAGHRVILVETHKYWLTGHRFSNAVDRFYTIPAPQKDPEGYTQALLNIAKQENVDIYIPVCSPVSSYYDSLAKPALSGCCEVFHFDADITKMLDDKFAFSEKARALGLSVPKSFKITNPEQVLNFDFSNETRKYILKSIPYDSVRRLNLTKLPCDTPEETAAFVKSLPISEEKPWIMQEFIPGQEYCTHSTVRDGELRLHCCCESSAFQVNYENVDQPEIMKWVSHFVKELKLTGQASFDFIQAEDGAIYAIECNPRTHSAITMFYNHPGVADAYLGKEPLAEPLQPLPDSKPTYWLYHEIWRLNEIRSWSQLQTWMNNLLRGTDAIFDVNDPLPFLTVHHWQIPVLLLDNLRKLRGWVRIDFNIGKLVESGGD

SEQ ID NO: 80 - protein, 461 aa, synthetic construct
MAQSISLSLPQSTTPSKGVRLKIAALLKTIGTLILLLIALPLNALIVLISLMCRPFTKKPAVATHPQNILVSGGKMTKALQLARSFHAAGHRVILIEGHKYWLSGHRFSNSVSRFYTVPAPQDDPEGYTQALLEIVKREKIDVYVPVCSPVASYYDSLAKSALSEYCEVFHFDADITKMLDDKFAFTDRARSLGLSAPKSFKITDPEQVINFDFSKETRKYILKSISYDSVRRLNLTKLPCDTPEETAAFVKSLPISPEKPWIMQEFIPGKELCTHSTVRDGELRLHCCSNSSAFQINYENVENPQIQEWVQHFVKSLRLTGQISLDFIQAEDGTAYAIECNPRTHSAITMFYNHPGVAEAYLGKTPLAAPLEPLADSKPTYWIYHEIWRLTGIRSGQQLQTWFGRLVRGTDAIYRLDDPIPFLTLHHWQITLLLLQNLQRLKGWVKIDFNIGKLVELGGD

SEQ ID NO: 81 - protein, 453 aa, synthetic construct
MTKLQPIKARIIAVFQNLGTLLLLAIAFPINCSVVLVSLLWNFFSRPSHKQVVLTENPKNILIGGGRMTKTLQLARSFHAAGHRVILVDIDKYWLSGHRFSRAVAGYYTVPAPQKDLEGYTQALRAIAKKENIDFFIPVAIFAVSYFDSKGEPVLSGCCEIFHFDADITKMLDDKFAFAEKARSLGLSVPKSFKITDPEQVLNFDFSQEKRKYILKSIPYDCLRRLNMTKLPCDTFDMTAEFVKSLPISEEKPWIMQEFIPGKEYCTHSTVRDGELRLYCCCESSAFQVNYENVDRPEIRQWVQQFVQEVGLTGEISFDIIQADDGTVYPIECNPRTHSAITMFYNHPGVANAYLNKEPLVEPLQPLADSKPTYWLYHEVWRLTGIRSLKQLQTWIRNILRGKEAIFSVSDPLPFMMVHHWQIPLLLLDNLRRLKGWVRIDFNLGELIESEEY

SEQ ID NO: 82 - protein, 464 aa, synthetic construct
MAQSISFSSAPATPSVPSTSKIAAIFPNIGTLTLLLLALPINASIVLITLLLRAILRPFQPSAVKAANPKNILISGGKMTKALQLARSFHAAGHRVVLLETHKYWLTGHQYSQAVDKFYTVSAPQENPERYTQALVDIIKQENIDVYIPVTSPLGSYYDSLAKPELSRYCEVFHFDADITQMLDDKYELAQTARSLGLSVPKSFKITSAEQVLNFDFSGETRKYILKSIPYDSVRRLDLTKLPCATPEETAAFVRSLPISPEKPWIMQEFIPGKEFCTHSTVRNGELRLHCCCESSAFQVNYENVENPQILEWVKHFVKELKLTGQISFDFIQAEDGKVYAIECNPRTHSAITTFYDHPKVAEAYLSQEATTETLQPLPTSKPTYWTYHEVWRLTGIRSFKQLKTWIVNIWRGTDAIYKFDDPLPFLMVHHWQIPLLLLKNLRQLKGWTRIDFNIGKLVELGGD

SEQ ID NO: 83 - protein, 435 aa, synthetic construct
MFQNLGTLVLLAIAFPLNCIVVLTSLLWSFIKQPFNKSIVVNPNSKNILIAGARMTKTLQLARSFHAAGHRVIIIDIEKYWLSGNKYSNSVAGFYTVPDPSKDLEGYVETLHAIANTEKIDFFIPVAIFSVIHYDQGKPPLPDCVEFFHFDADVTKILDDKFAFAETARSFGLSVPKSFKITDPEQVLNFDFSQEKRKYILKSIPYDQVRRLNLTKLPCDTKSETAAFVKSLPISEENPWIMQEFIPGKEYCTHTTARDGESRMYCCCESSAFQVNYENVDQREIMQWASHFTKELGKTGQLSFDFIQAEDGTVYAIECNPRTHSAITMFYNHPGVADAYLGKEPLAESLQPLPDSKPTYWLYHEVWRLNEIRSFKQLQTWVRNIRRGKEAIFEVSDPLPFLMVHHWQIPLLILDNLRRLKGWIRIDFNMGELIE

SEQ ID NO: 84 - protein, 454 aa, synthetic construct; identical to SEQ ID NO: 47

SEQ ID NO: 85 - protein, 443 aa, synthetic construct
MKEQIFIVFQNLGTLVLLAIAFPFNCIVVLTSLVWNFIKQPFSQSIVVNPNSKNILIAGARMTKTLQLARSFHAAGHRVIIIDIEKFWSSGNKYSNSVAGFYTVPDPSKDLEGYVESLHAIAKKEKIDFFIPVAIFSVIHYDSQGKPPLPDDVEFFHFDADVTKILDDKFAFAETARSFGLSVPKSFKITDPEQVLNFDFSQEKRKYILKSIPYDQVRRLNLTKLPCDTPSQTAAFVKTLPISEEKPWIMQEFIPGKEYCTHTTARDGESRMYCCCESSAFQVNYENVDQPEIMQWASHFTKELGKTGQLSFDFIQAEDGTVYAIECNPRTHSAITMFYNHPGVADAYLGKEPLAESLQPLSDSKPTYWLYHEVWRLNEIRSFKQLQTWVRNIRRGKEAIFEVSDPLPFLMVHHWQIPLLILDNLRRLKGWIRIDFNMGELID

SEQ ID NO: 86 - protein, 458 aa, synthetic construct; identical to SEQ ID NO: 51

SEQ ID NO: 87 - protein, 456 aa, synthetic construct
MIDTVSLNKSLAEKGFGRREIGVIGRNLATLGLLLLVLPINLLLTGVGLISRVSLRNPISQKTILISGGKMTKALLIARRFHAAGHRVILIESHKYWLTGHRFSNAVNKFYTVPAPEKNPSAYIQALLDIIKREKVDLYVPVCSPVASYYDALVKSEMGFLTQVFHCDPEMVKMLDDKFTFAETARKLGLSVPKSFLITHPHQVINFDFQKETRPYILKSIRYDSVRRLDLTKLPCETPEATERFVRSLPISPENPWIMQEFIPGQEYCTHSTVKNGELRMHCTSKSSAFQVNYENIDHPRIQSWVSKFVKELGITGQVSFDFIETEDGEVYAIECNPRTHSAITMFYNHPRVADAYLDEGVWEQPIQPLPDSKPTYWLYHEIWRLTGIRSWKDLQYRWKVLSTGVDAIYSLDDPLPFLMVHHWQIPLLLWQNLLQLRGWVRIDFNIGKLVELGGD

SEQ ID NO: 88 - protein, 445 aa, synthetic construct
MQKMFAIFQNLGTLTLLAIAFPFNCIVVLSALVWNLISQPFQKQVVFNPDAKNILIGGGRMTKTLQLARSFHAAGHRVILFDIDKNWFSGYRFSNAVAGFYTVPDPIKDLEGYTIALRAIAKQENIDFFVPVGIFANDYFDSKRQPVLSGCCETFHFDADTMKMLDNKFTFTQKARSLSLSVPKAYLITDPEQVLKFDFSNEKNKYILKSIVYDPVFRLDLTKLPMESLEKMAIHVRNLPISKDNPWILQEFITGQEYCTHSTVRNGELTVHCCCESSAFQVNYENVDKPEILQWVSHFVKELQLTGQISFDFIQAEDGTIYAIECNPRTHSAITMYYNHPGLADAYLGQKPLAELLQPLPDSKPTYWLYHEVWRLNEIRSLKQLQTWFKNILRGKDAIFDVNDPLPFLMVHHWHIPLLLLDNLQKLKGWVRIDFNIGKIVQVSD

SEQ ID NO: 89 - protein, 464 aa, synthetic construct
MDNLFNSSADSSSLSKGWLRSIQGSSLKTLGTLLLLLLMLPFNLALTLTALVWSWVWPFRKRVIASNPKTVMISGGKMTKALQLARSFYMAGHRVILVETHKYWLVGHRYSWAVDRFYTIPDPKQDTEGYLQGLLDIAQKEQVDLYVPVCSPVASYYDALAKELLAQQCDVFHEDAKTVQQLDDKYQFAQAATNLGLTVPKSFKITHPQQVLDFDFSKETHPYIIKSIPYDSVNRLNLTKLPCASRQDTEMFVNSLPISETKPWVMQEFITGQEYCVHSTVKNGELRVYCCCESSAFQVNYEAVDIPEIKQWVTQFVQGMKLTGQMSFDFIRTPTGEVYAIECNPRTHSAITLFYNHPDLAKAYLDPEPFSEPLEPLASARPTYWTYHEFWRLVTHLSSLQEVAYRLGILFKGKDAIFSWNDPLPFLMVHGWQIPLLLLKSLRQGKDWIRIDFNIGKLVQMGGD

SEQ ID NO: 90 - protein, 447 aa, synthetic construct
MTQIFFVSGRGSAVLQNLGTLVLLLFLLPFNLIAVAFSAVINIFSGSKQRLTKTDVPKRILITGAKMTKALQLARSFHQRGHEVYLVETHKYWLSGHRFSRAVKGFFTVPTPEKEPDAYCQRLLEIVQQKNIDVFIPVSSPIASYYDSLAKKILEPDCEAIHFDPEITAMLDDKYAFCTKAKELGLSAPKVFCFTSPQQVIDFDFESDGSQYIVKSIPYDSVRRLDLTKLPFEGMESYLRSLPISSEKPWVMQEFIRGQEYCFHATVRKGKIRLHCCSQSSPFQVNYEQVDNPAIYQWVEKFVRELNLTGQICFDMIQTPDGTVYPIECNPRLHSAITMFHDHPGVADAYLLDGEQAITPLPDSKPTYWTYHELWRLLQVRSLSELQAWWHKVSRGTDAILQGDDPLPFLMLHNWQIPLLLLDNLRRLKGWIRIDFNIGKLVELEGD

SEQ ID NO: 91 - protein, 464 aa, synthetic construct
MTQSISLSLPESTTPSTGIKVKIVALFKTLGTLTLLLIALPFNVLIVLISLLWGIVRVPFTKNVVATHPQTILVSGAKMTKALQLARSFHADGHRVILIEGHKYWLSGHRFSKAVSRFYTVPAPQSDPEGYIQALIEIVKKEKVDVYVPVCSPVASYYDSLAKPALSEYCEVFHFDADITKMLDDKFAFTEKARSLGLSVPKSFKITDPQQVINFDFSQETRKYILKSINYDSVRRLNLTKLPCDTPEQTAAFVKSLPISPETPWIMQEFIPGKEFCTHSTVRDGELRLHCCCHSSAFQINYENVENPQIQAWIQHFVKSLRLTGQVSFDFIQAEDGQVYAIECNPRTHSAITMFYNHPGVAEAYFGKTPLAAPLEPLPSSKPTYWTYHEIWRLTGVRSWKQLQTRLNILLRGTDAIYCLDDPIPFLTLHHWQIPLLLLQNLQQLKAWVKIDFNIGKLVELGGD

SEQ ID NO: 92 - protein, 462 aa, synthetic construct
MAQSISLSVPKSTTPSTGVSIKIVALFKTLGTLTLLLIALPINAFIVLLSLLWGILFTKKPAVAAHPQNILVSGGKMTKALQLARSFHAAGHRVILIEGHKYWLSGHRFSNAVSRFYTVPAPQDDPQGYTQALLEIVKQEKIDIYVPVCSPVASYYDSLAKPALSEYCEVFHFDADITKMLDDKFAFTDQARSLGLSVPKSFKITDPEQVINFDFSKETRKYILKSISYDSVRRLNLTKLPCDTPEETAAFVNSLPISPEKPWIMQEFIPGKELCTHSTVRDGELRLHCCSDSSAFQINYENVENPQIREWVQHFVKSLGLTGQVSFDFIQAEDGTAYAIECNPRTHSAITMFYNHPGVAEAYFGKTPLAAPLEPLADSKPTYWVYHEIWRLTGIRSGKQLQTWFARLVRGTDAIYKIDDPLPFLTLHHWQIALLLLQNLQQLKGWVKIDFNIGKLVELGGD

SEQ ID NO: 93 - protein, 442 aa, synthetic construct; identical to SEQ ID NO: 42

SEQ ID NO: 94 - protein, 464 aa, synthetic construct
MAQSISVSSSPAIPSFPSETKIAVIIQNLLTLALLLLALPINAAIVLVTLLWHTISRPFQQPATKAANPKNILISGGKMTKALQLARSCAAAGHRVILIETHKYWLSGHRFSQAVDKFYTVPAPQENPERYTQALIDIIKQENIDVYIPVTSPLGSYYDSLAKPLLSEYCEVFHFDIDITEKLDDKFAFAETARSLGLSVPKSFKITSAEQVLNFDFSQESRKYILKSIPYDSVRRLDLTKLPCATPEETAAFVRSLPISPDKPWIMQEFIPGKEFCTHSTVRDGELRLHCCCESSAFQVNYENVENSQIREWVRHFVKELKLTGQVSFDFIQAEDGRVYAIECNPRTHSAITTFYDHPQVAQAYLDNEPMAETLQPLPSSKPTYWTYHEVWRLTGIRSFTQLKKWIANIWRGTDAIYKPDDPLPFLMVHHWQIPLLLLKNLRQIKGWTRIDFNIGKLVELGGD

SEQ ID NO: 95 - protein, 459 aa, synthetic construct
MTQSISVASVGQTTQSVTLGLRISALFKNLATLALLLLVLPINAAIVLVSLLLGSQSQAIATEPKNILISGGKMTKALQLARSFHAAGHRVVLVETHKYWLTGHRFSKAVSRFYTLPTPQSDPEAYTQALLDIVQKESINVYVPVCSPVSSYYDSLAKPVLSKYCEVFHCDADVTQMLDDKYAFAEKARSLGLSVPKSFKITDPKQVINFDFSQEKRKYILKSIPYDSVRRLDLTKLPCESPEATADFVNSLPISSQKPWIMQEFIPGKEFCTHSTVRNGELRMHCCCESSAFQVNYENVDHPQILEWVRHFVKALGITGQVSFDFIEAQDGTIYAIECNPRTHSAITMFYNHPDVANAYLSEIPQVEPIQPLINSKPTYWTYHEIWRLTGIRSFSQLQTWVKNFFGGKDAIYSLSDPLPFLTVHHWQIPLLLLQNLQQLKGWIRIDFNIGKLVEFGGD

SEQ ID NO: 96 - protein, 463 aa, synthetic construct; identical to SEQ ID NO: 55

SEQ ID NO: 97 - protein, 465 aa, synthetic construct
MTQSISFSSPVPATPPFCVKTRFIALFQNLGALTLLLLALPINVAIVLISLIWSFLSRLFSTQETTVAGAKNILISGGKMTKALQLARFFSAAGHRVVLIETHKYWLSGHRFSNAVSRFYTTPTPQDEPEEYIQTLVDIVKRENIDVYVPVTSPVASYYDSLAKPALSPYCEVLHFDADVTKMLDDKFAFSEKARALGLSVPKSFKITNPEQVLNFDFSQETRKYILKSLPYDSVRRLDLTKLPCNTPEETAAFVKSLPISLEKPWIMQEFIPGKEFCTHSTVRNGDLKLHCCSESSAFQVNYENVKNPKIQEWVRHFVKGLGLTGQVSFDFIQADDGKVYAIECNPRTHSAITMFYNHPQVADAYLGTEPLAEPLAPVPNSKPTYWLYHEVWRLTGIRSFAQLQSWIRNILRGTDAIYELHDPLPFLMVHHWQIALLLLNNLRQLKGWTKIDFNIGKLVELGGD

SEQ ID NO: 98 - protein, 442 aa, synthetic construct
MRKHIFVVFQNLGTLVLLALAFPLNSIVVLTSLLWNFLKQPFSKSIVVNPNSKNILIAGARMTKTLQLARSFHAAGHRVIIIDIEKFWSSGNKYSNSVAGFYTVPDPSKDLEGYVETLHAIAKTEKIDFFIPVAIFSVIHYDRGKPPLPDFCEFFHFDADVTKSLDDKFAFAETARSFGLSVPKSFKITNPEQVLNFDFSQEKRKYILKSIPYDQIRRLNLTKLPCDTQSETAAFVKSLPISEENPWIMQEFIPGKEYCTHTTARDGESRMYCCCESSAFQVNYENVDRLEIMEWASHFTKQLGKTGQLSFDFIQAEDGTVYAIECNPRTHSAITMFYNHPGVADAYLGKNPLAESLQPLGDSKPTYWLYHEVWRLNEIRSFKQLQTWLRNIRRGKEAMFEVSDPLPFLMVHHWQIPLLILDNLRRLKGWIRIDFNMGELIE

SEQ ID NO: 99 - protein, 449 aa, synthetic construct
MVELQFIKARIFAVFRNLGTLALLAIAFPFNCIVVLAALLWNFFTRPFQKQVVLSENPKNILIGGGRMTKTLQLARSFHAAGHRVILVDIHKYWLSGHRFSKAVAGYYTVPEPQKDLEGYTQALRAIAKKENIDFFIPVAIFAVSYFDPQNKPVLAGCCEIFHFDGEVTKMLDDKFAFAEKARSFGLSVPKSFKITAPEQVLNFDFSQEKNKYILKSIPYDSVRRLNMTKLPCDTTEQTAAFVKSLPISEENPWIMQEFIPGQEYCTHSSLRNGELRLHCCCESSAFQVNYENVDKPEIMQWVSHFVKELGLTGEASFDIIQAVDGTVYPIECNPRTHSAITMFYNHPGVADAYLGKEPLAEPLQPLPDSKPTHWLYHEVWRLTGIRSLKQLQTWVRNILRGKDAIFEVHDPLPFLMVHHWQIPLLLLDNLRRLKGWIRIDFNLGELIE

SEQ ID NO: 100 - protein, 472 aa, synthetic construct
MHFNCGAEKLMAQSISLSLPKSTTPSTGVRIKIVALFKTLGTLTLLLIALPINAFIVLLSLLWSIPFTKKPAVAAHPQNILVSGGKMTKALQLARSFHAAGHRVILVEGHKYWLSGHRFSKAVSRFYTVPAPQDDPEGYTQALLEIVKQEKIDIYVPVCSPIASYYDSLAKPALSEYCEVFHFDADITKMLDDKFAFTDQARSLGLSVPKSFKITDPEQVINFDFSKETRKYILKSISYDSVRRLNLTKLPCDTPEETAAFVNSLPISPEKPWIMQEFIPGKELCTHSTVRDGELRLHCCSDSSAFQINYENVENPQIREWVQHFVKSLGLTGQVSFDFIQAEDGTAYAIECNPRTHSAITMFYNHPSVAEAYFGKTPLAAPLEPLADSKPTYWVYHEIWRLTGIRSGKQLQTWFTRLVRGTDAIYKIDDPLPFLTLHHWQIALLLLQNLQQLKGWVKIDFNIGKLVELGGD

SEQ ID NO: 101 - protein, 423 aa, synthetic construct
MFPINLTLVITAFLTNLITLPFQKKITYENPKNILLTGGKMTKSLQLARSFHRAGHKVFMVETHKYWLSGHQYSKAVKKFLTVPAPEKDPEGYCQSLLDIVKREKIDVFIPVSSPVASYYDSLAKPILSPYCEVFHFDTEMTKTLDDKFSLCEQARVLGLTAPKVFLITSPGEIINFDFSQEQNPYIIKSIQYDSVTRLDMTKFPFEGMKEYVKKLPISKERPWVMQEFIKGQEYCTHSTVRDGEIRLHCCSKSSPFQVNYEQVDNPEIFQWVQKFVKELNLTGQISFDFMQTEDGKVYPIECNPRTHTAITMFYDHPGLADAYLEPGKNQPHIEPLPTSKPTYWLYHELWRITGIRSFNDLTNWLNKVIKGKDAMLDKDDPLPFLMVHHWQIVLLLLQNMVKLKGWVRIDFNIGKLVEIGGD

SEQ ID NO: 102 - protein, 458 aa, synthetic construct; identical to SEQ ID NO: 51

SEQ ID NO: 103 - protein, 465 aa, synthetic construct
MAQSISLSLGNSPTSSTGVWVKLVALFKTLGTLTLLLIALPFNALIVLISLLWGFVRSPFRQKAVVAEHPQTILVSGAKMTKALQLARCFHAAGHRVILIEGHKYWLSGHRFSKAVSGFYTVPAPQLDPEAYIQALVDIVEKEQVDVYVPVCSPVASYYDSLAKPALSEYCEVFHFDADVTKMLDDKFAFTAQARSLGLSVPKSFKITDTQQVINFDFSQETHKYILKNIAYDSVRRLNLTKLPCDTPEETAAFVNSLPISEENPWIMQEFIPGKELCTHSTVRDGELRLHCCSDSSAFQINYENVENTQIREWVQHFVKSLALTGQISFDFIQAESGTVYAIECNPRTHSAITMFYNHPGVAEAYLGKTTLDAPLEPLTNSKPTYWIYHEIWRLTGIRSWKQLQTAVNTLLRGTDAIFQLNDPVPFLTLHHWQIPLLLLKNLQQLKGWVKIDFNIGKLVELDGD

SEQ ID NO: 104 - protein, 461 aa, synthetic construct
MIIHMAQSISLSSPAKTHAPGISASSLKTLGTLTLLLLALPLNASLVLVALLLKSLRPQNFTTEKPKNILISGGKMTKALQLARSFHNAGHRVILLEAHKYWLTGHRFSSAVNKFYTVEAPEKDPEGYIQSLVDIVEKENIDVYVPVCSPVASYYDSLAKKALPQCEVIHCDAEMTQMLDDKHAFAQTAQSFGLSVPKSFKITDPEQVINFDFSQEKRKYILKSIPYDSVRRLDLTRLPCDTPEATAAFVRSLPISSEKPWIMQEFIPGKEYCTHSTVRNGVITLHCCCESSAFQVNYENVDNPKIFEWVSRFVKELGITGQVSFDFIEAEDGNIYAIECNPRTHSAITMFYNHPGVADAYLGTGNNLAEPIQPKFTSKPTYWTYHEIWRLFNTRSWSDFVYRFKIIKHGKDAIFSWQDPLPFLMNPHWQIFLLLIQNLQKNRGWIRIDFNIGKLVELGGD

SEQ ID NO: 105 - protein, 439 aa, synthetic construct
MGNGALAENLKEDDKTVIWRPHEEKYRTSEWYTGSGQITTADEGLSFEVTAVYQLKSEVKVVKDIFAISNHTLANIYRPRSRCIAVVDQTVAELYGEKIEGYFQAQEIPLELMVIRAWESDKTPETVHRILAFLGKDGCDVSRNEPVLVIGGGVLSDVAGLACALQHRRTPYIMIGTTIIAAIDAGPSPRTCTNGTQFKNSIGVYHPPVLTLVDRQFFSTLDMGHIRNGMAEIIKMAVTDDKELFELLEQYGQELIKTRFATIDASEELEKIADLIIYKALYAYMKHEGTNMFETYQDRPHAYGHTWSPRFEPAVKLMHGHAVTIGMAFGATLAQELGWLSQEECQRIINLSSKLGLSVFHPILEDVQIMVDGQKNMRRKRGDGGLWAPLPTTIGACDYVQEVEPELLNQAVVAHKKYCSQLPHEGAGEQMYLSDLGLE

SEQ ID NO: 106 - protein, 403 aa, synthetic construct
MSNLQAQVVAGDRSFRVEGYERIEYDLIYVDGVFAIENTELADSYRPYGRALMVVDEAVHDIYGDRISAYFDHHEIALTVVPVHIAETAKSLETFERIVGEFDAFGLVRTEPVLVVGGGLTTDVAGFACASYRRNTPYIRIPTTLIGLIDASVSIKVAVNYGKHKNRLGAYHASQKVLLDFSFLGTLPEDQVRNGMAELIKISVVGNLEIFEMLEQYGPELLRTRFGHLDGTAELRSVADKLTYSAIATMLELEAPNLHEIDLDRVIAFGHTWSPTLELTPPAPFFHGHAINIDMALSTTVAEQRGHLSTADRDRVLGVMSSIGLALDSPYLTPELLSEATASILKTRDGILRAAVPDPIGTCRFLNDLDAAELADVLTLHKKICLDFPRAGEGLDMFTAPTP

SEQ ID NO: 107 - protein, 410 aa, synthetic construct
MAGIKATFTSTDCAFHIQGYEKIDFSLLYVNGAFKIGNPEIAESYAPFRRCLMVIDQTVYGLYRQQIDQYFAHYQIDLTVFQVSIKEPEKTLRTFEKIVDAFADFGLVRKEPVLVVGGGLTTDVAGFACSAYRRKTNYIRVPTSLIGLIDASVAIKVAVNHGKLKNRLGAYHASQKVILDFSFLGTLPIDQIRNGMAELIKIAVVGNQEIFELLEEHGAALLHSRFGYLNGTPELQAVGHRLTYKAIQAMLELEVPNLHELDLDRVIAYGHTWSPTLELTPEPPMLHGHSVNIDMAFTATIAQLRGYISVEDRNRILGLMSRLGLAIDSPYLTPELLWKATEAITRTRDGLQRAAAPRPIGQCVFMNDLTRSELDKALAVHRAIAQNYPRQGNGEDMYVRLEPALEGAGV

SEQ ID NO: 108 - protein, 409 aa, synthetic construct
MSSVQAKVEVTDQSFHLEGYEKIEFNLDLIEGLFEVGNSGLADNYRTLGRCLAVVDHNVDRLYGDQLRSYFEYYEIDLTVFAIEITEPTKTIDTFLKITDAFCDFNLKRKEPVLVIGGGLVLDVAGFACSAYRRSTNYIRVPSTLIGLIDAGVAIKVAVNHGKLKNRLGAYHPPKQVILDFSFLKTLPVDQIRNGMAELVKIAVVSNEEVFNLLEQHGEELLYNHFGFVGNDAELKQIGHRVNYESIKTMLELEAPNLHELMLDRVIAYGHTWSPTLELAPQIPLLHGHAVNIDMAISATIAEKRGYISALDRDRILGLMSRLGLALDHPLMEIDLMWKATQSIMLTRDGFLRAAMPRPIGTCYFVNDLTREELESAIADHKRLCADYPRAGAGIDAYVGSSELIGSAN

SEQ ID NO: 109 - protein, 415 aa, synthetic construct
MSNPQAVLSATDTEFRVESWERIEFTLSYVDGVFAPHNTELADLYRPWGRCLMVIDETVHEHYGDPIRSYFDHHDIAVTLVPLTIAETAKSLRTLERIVDAYADFGLLRTEPVLVVGGGLTTDVTGFACASYKRGTPYVRIPTTLIGLIDASVAMKVAVNHGRHKNRLGAFHASQQVLLDFSFLATLPEAQVRNGVAEMIKIATVANAGLFDLLEKYGDDLLATRFGHREGTPELRQIAHRCTYDAIHTMLELEHRNLHELDLDRVIAFGHTWSPTLELAPPTPMLHGHAIAIDMAFSATLAARRGDITTGERDRIHRLFSGLGLSVDSTYLTEQLLIDATASIMQTRAGKLRAALPRPIGTCHFANDIEHTELIETLAAHKAVVAGLPTSVEGVEMWSSAKTELTTAPNTEART

SEQ ID NO: 110 - protein, 406 aa, synthetic construct
MTTNLTATVTATENDFRVRAVEERDYLLTYVDGAFSPESSRIADHHRAHGRCLMIVDANVHRLHGDRIRAYFEHHGIALTALPLAIDETQKSLRTVERIVDAFGEFGLIRKEPVLVVGGGLLTDVAGFACAVFRRSTDYVRVPTSLIGLIDASVAIKVAVNHGRTKNRLGAFHASKEVVLDFSFLGTLPTEQVRNGMAELVKIAVVANAEVFRLLEKYGEDLLHTAFGTVDGTPQLRETARKVTHEAIGTMLALEAPNLRELDLDRAIAFGHTWSPALELAPETPYLHGHAISVDMALSCTIAERRGYLATSERDRIFWLLSKVGLSLDSPHLTPELLRAATESIVQTRDGLQRAAMPRPIGTCCFVNDLTESELLDGLAAHRELVARYPRGGAGEDVRVTRSGAA

SEQ ID NO: 111 - protein, 414 aa, synthetic construct
MSTVQAKFEATETAFHVEGYEKIDFSLVFVNGAFDTKNRELADSYRNFGRCLAVVDANVNRLYGSQICEYFKYYNIDLNLFPVTISEPTKNLDTFQSIVDAFADFGLVRKEPVLIVGGGLVTDVAGFACAAYRRSTNYIRIPTTLISLVDAGIAIKVAVNHGKLKNRLGAYHAPKKVMLDFSFLRTLPTPEVRNGMAELVKIAVVSNVEVFELLCEYGADLLTTHFGFDGGTPLLKEVAHRINYESIKTMLALETPNLHELDLDRVIAYGHTWSPTLELAPSVPLLHGHAVNIDMALSATIAEKRGYITVEERDRILGLMSQLGLALDHPLLDIDLLWSATQSITLTRDGLQRAAMPRPIGKCFFVNDLTREELDAALAEHKHACAQYPRAGAGVDAYVGSYQQQNLIEGIANV

SEQ ID NO: 112 - protein, 278 aa, synthetic construct
MSTTIAKPTARPVTPVGILAKKLEAIVQKINQRTDLPADLVDNITQAWQLAAGLDPYLEEYTTSESSALTALAEKTSTEAWQEHFSEGTTVRPLEQEMLSGHVEGQTLKMFVHMTKAKRVLEIGMFTGYSALAMAEALPPDGVLVACEVDPFAAEVGQAAFDKSPDGKKIRVELGPALETLNKLVEAGESFDMVFIDADKKEYITYFQTLLDTNLLAPSGFICVDNTLLQGEVYLPTQQRTANGEAIAQFNRAVALDPRVEQVILPLRDGLTIIRRTA

SEQ ID NO: 113 - protein, 456 aa, synthetic construct
MSLSAPPSRSKIRSTLKTLGTLVLLLLALPLNAAIVLVALLRNLITRPRKRATAANPKTVLISGGKMTKALQLARSFHRAGHRVILVETHKYWLTGHRFSNAVDRFYTVPAPQDDPEGYAQALLDIVQKENVDVYVPVCSPVASYYDALAKETLSPHCEVFHFDADTVKMLDDKYQFAEMARSLGLSVPESHRITSPEQVLDFDFSQSEGRKYILKSIAYDSVRRLDLTKLPCPTPEETAAFVRSLPISPDNPWIMQEFIEGQEYCTHSTVRDGRLRLHCCCESSAFQVNYEHVDNPEIQEWVQRFVKALNLTGQVSFDFIQTDDDGRVYAIECNPRTHSAITMFYNHPGVAEAYLDPDPDLAEPIQPLPSSRPTYWLYHELWRLLTHPRSLQDLRERLKTIFRGKDAIFDWDDPLPFLMVHHWQIPLLLLKNLRQGKDWVRIDFNIGKLVELGGD

SEQ ID NO: 114 - protein, 413 aa, synthetic construct
MVVAENPKNILITGGKMTKALQLARSFHAAGHRVFLVETHKYWLSGHRFSNAVDRFYTVPAPQKDPEGYVQGLLDIVKQENIDVFIPVSSPVASYYDSLAKPVLSPYCEVFHFDAEITKMLDNKFTFSEKARSLGLSAPKSFLITDPEQVLNFDFAADQGSQYILKSIPYDSVHRLDMTKLPCDKEEMAEYVKSLPISEENPWIMQEFITGQEYCTHSTVRDGKIRLHCCSKYPTLFTASSAFQVNYEHVDNPAILQWVTRFVKELNLTGQISFDFIQAEDDGTVYPIECNPRTHSAITMFYNHLPGVVADAYLKDSPDEEEPIQPLPDSKPTYWLYHELWRLTEIRSWSQLQAWINNILKGTDAIFQVNDPLPFLMVHHWQIPLLLLNNLRKLKGWVRIDFNIGKLVELGGD

SEQ ID NO: 115 - protein, 414 aa, synthetic construct
MVVAENPKNILITGGKMTKALQLARSFHAAGHRVFLVETHKYWLSGHRFSNAVDRFYTVPAPQKDPEGYIQALLDIVKQENIDVFVPVSSPVASYYDSLAKPVLSPYCEVFHFDADITKMLDDKFTFSEKARSLGLSAPKSFLITDPEQVLNFDFASDQGSQYILKSIPYDSVHRLDMTKLPCDSKEEMAAYVKSLPISEENPWIMQEFITGQEYCTHSTVRDGKIRLHCCSKYPTLFTASSAFQVNYEHVDNPKILQWVTRFVKELNLTGQISFDFIEAEDDGTVYAIECNPRTHSAITMFYNHLPGVVADAYLGKSPSAEEPIQPLPDSKPTYWLYHEVWRLTEIRSWSQLQTWINNILRGKDAIFQVNDPLPFLMVHHWQIPLLLLNNLRKLKGWVRIDFNIGKLVELGGD

SEQ ID NO: 116 - protein, 413 aa, synthetic construct
MVVAENPKNILLTGGKMTKALQLARSFHAAGHRVILVETHKYWLSGHRFSNAVDRFYTVPAPQKDPEGYTQALLAIAKQENIDVYVPVCSPVASYYDSLAKPVLSGCCEVFHFDADVTKMLDDKFAFSEKARSLGLSVPKSFLITDPEQVLNFDFSNEQKRKYILKSIPYDSVHRLDMTKLPCDSKEEMAAYVKSLPISEENPWIMQEFIPGKEYCTHSTVRNGELRLHCCCEYPTLFTASSAFQVNYENVDNPKILQWVSHFVKELKLTGQISFDFIEAEDDGTVYAIECNPRTHSAITMFYNHLPGVVADAYLGKEPLEEPLQPLPDSKPTYWLYHEVWRLTEIRSFSQLQTWIKNILRGKDAIFSVNDPLPFLMVHHWQIPLLLLNNLRRLKGWIRIDFNIGKLVELGGD

SEQ ID NO: 117 - DNA, 28 bp, synthetic construct
  source 1..28: mol_type = other DNA
catgccatggtgagcattgttcaaacaa

SEQ ID NO: 118 - DNA, 29 bp, synthetic construct
  source 1..29: mol_type = other DNA
catgctgcagtcacgcagttctgcggata

SEQ ID NO: 119 - DNA, 29 bp, synthetic construct
  source 1..29: mol_type = other DNA
cgtcggtaccatggcacaatctatttccg

SEQ ID NO: 120 - DNA, 30 bp, synthetic construct
  source 1..30: mol_type = other DNA
cagactcgagctaatccccacccaattcca

SEQ ID NO: 121 - DNA, 25 bp, synthetic construct
  source 1..25: mol_type = other DNA
catgcatatgccagtacttcgtatc

SEQ ID NO: 122 - DNA, 30 bp, synthetic construct
  source 1..30: mol_type = other DNA
catgctcgagctaaatcatttgtgaaagct

SEQ ID NO: 123 - DNA, 37 bp, synthetic construct
  source 1..37: mol_type = other DNA
taataaggagatataccatggtgaaggtagacacaca

SEQ ID NO: 124 - DNA, 40 bp, synthetic construct
  source 1..40: mol_type = other DNA
gcaagcttgtcgacctgcagtcgatgtacttgaactctag

SEQ ID NO: 125 - DNA, 38 bp, synthetic construct
  source 1..38: mol_type = other DNA
taagaaggagatatacatatggcttctctagaaaatca

SEQ ID NO: 126 - DNA, 40 bp, synthetic construct
  source 1..40: mol_type = other DNA
gtttctttaccagactcgagctaagtgcgccgattaacta

SEQ ID NO: 127 - protein, 180 aa, synthetic construct
GSVEHASSLWLVDADFRALAFESPLPTLAAQVLKSEKLNFLADGFFVKKPKTNGHIGWHNDLPYWPVQGWQCCKIWLPLDTVKQENGRLEYIKGSHQWGKELRERSNPSWFVEPEPHEILSWDMEAGDCLIHHFLTIHHSVTNISSTQRRAIVTNWTGDDVTYYQRPKAWPFKPLEEIDL

SEQ ID NO: 128 - protein, 180 aa, synthetic construct
GNVEHTNSLWLVDADFRALVFESPLATLAAQILKSTKLNFLADGFFVKQPKATSRVGWHNDLPYWPIQGWQCCKIWLALDKVNQQNGRLEYIKGSHRWGKELREDSNPAWFSQPESHELLSWDMEPGDCLVHHLLTIHHSVTNISSTQRRAVVTNWTGDDVTYYPRPKAWPFRPLDEIDI

SEQ ID NO: 129 - protein, 180 aa, synthetic construct
GDVVHDYGLWLKDNDFRDLVFKSPLARVAAQIMESETINFLCDGFFVKKAKADSHVGWHNDLPYWPVKGWKCCKIWLALDPVNQENGRLEYIKGSHLWNKDLRENSNVSWFSEPSYSDILYWDMEPGDALVHHFQTIHHSIGNTTYKSRRAIVTNWTGDDVVYDPSPQTWPFQPIEEIGI

SEQ ID NO: 130 - protein, 180 aa, synthetic construct
GHVEHTSSLWLTDADFRALAFESPLATLTAQVLKSKKLNFLGDGFFVKKPKGETGVGWHNDKSYWPIQGWQCCKIWLALDSVNQENGKLEYIKASHLWGKELREASDPSWFVEPEPHEIISWDMEPGDCLVHHFMTIHHSVRNTSSTRRRAVVINWTGDDVTYERRPNAWPFRPLEEIDI

SEQ ID NO: 131 - protein, 180 aa, synthetic construct
ASAEHTSSLWLVDADFRALAFESPLPKLAVGVLKSEKLNFLADGFFVKRPEANGRIGWHNDLPYWPVQGWQCCKIWLALDTVKQENGRLEYIKGSHQWGRELRERSNPSWFVEPEPHEILSWDMEAGDCLIHHFLTIHHSVTNKSSTQRRAIVTNWTGDDVTYYQRPKAWPFKPLEEIDL

SEQ ID NO: 132 - protein, 180 aa, synthetic construct
ASVEHTSSLWLVDPDFRALVFESPLSTIAAQLLRSEKLNFLADGFFVKKPKATSRVGWHNDLPYWPIQGWQFCKIWLALDNVNEENGRLEYIKGSHQWGKELREDSNPSWFVEPEPHELLSWDMEPGDCLVHHLLTIHHSVTNISSRQRRAVVTNWTGDDVTYYPRLKAWPFRPLEEIDL

SEQ ID NO: 133 - protein, 180 aa, synthetic construct
GSAEHASSIWLIDADFRALAFESPLPTLAAQVLKSKKLNFLADGFFVKKPESNGRIGWHNDLPYWPVQGWQCCKIWLALDTVKQENGRLEYIKGSHQWGKELRERSNPSWFIEPEPHEILSWDMEAGDCLIHHFLTIHHSVTNISSTQRRAIVTNWTGDDVTYYQRPKAWPFKPLEEIDL

SEQ ID NO: 134 - protein, 180 aa, synthetic construct
GNVEHSNSLWLVDTDFRALVFESPLANLAAQFLKSTKLNFLADGFFVKQPKASSRVGWHNDLPYWPIQGWQCCKIWLALDKVNQQNGRLEYIKGSHRWGKELREDSNPSWFSEPEPHELLSWDMEPGDCLVHHLLTIHHSVTNISSTKRRAVVTNWTGDDVTHYPRPKAWPFRPLDEIDI

SEQ ID NO: 135 - protein, 180 aa, synthetic construct
ASVEHTSSLWLVDPDFRALVFESPLSTIAAQLLQSEKLNFLADGFFVKKPKATSRVGWHNDLPYWPIQGWQCCKIWLALDHVNEKNGRLEYIKGSHKWGKELREDSNPLWFVEPEPHELLSWNMEPGDCLVHHLLTIHHSVTNISSTQRRAVVTNWTGDDVTYYPRPKAWPFRSVEEIDL

SEQ ID NO: 136 - protein, 180 aa, synthetic construct
ASAEHTSNLWLVDADFRALAFESPLPTLAVQVLKSKKLNFLADGFFVKKPKSNSRIGWHNDLPYWPIQGWQCCKIWLALDTVNQENGRLEYIKGSHRWGKELRERSNPSWFVEPKPHEILSWDMEAGDCLIHHFLTIHHSVTNISSRQRRAVVTNWTGDDVTYYQRPKAWPFKSIEEIDL

(19) United States
(12) Patent Application Publication
(10) Pub. No.: US 2026/0043057 A1
(43) Pub. Date: Feb. 12, 2026

(54) ENZYMATIC SYNTHESIS OF MYCOSPORINE-LIKE AMINO ACIDS

(71) Applicant: University of Florida Research Foundation, Incorporated, Gainesville, FL (US)

(72) Inventors: Yousong Ding, Gainesville, FL (US); Manyun Chen, Gainesville, FL (US)

(73) Assignee: University of Florida Research Foundation, Incorporated, Gainesville, FL (US)

(21) Appl. No.: 18/998,446

(22) PCT Filed: Jul. 26, 2023
(86) PCT No.: PCT/US2023/071027

§ 371 Date: Jan. 24, 2025

Related U.S. Application Data
(60) Provisional application No. 63/392,467, filed on Jul. 26, 2022.

(51) Int. Cl. C12P 13/04; A61K 8/44; A61Q 17/04; C12N 1/20; C12N 9/00; C12N 9/02; C12N 9/10; C12R 1/19

(52) U.S. Cl. CPC C12P 13/04; A61K 8/44; A61Q 17/04; C12N 1/20; C12N 9/0071; C12N 9/1048; C12N 9/93; C12R 2001/19; C12Y 114/11018; C12Y 603/02004

(57) ABSTRACT

The present invention relates to methods of producing compounds of interest in a recombinant microorganism. In particular, the present invention relates to using a recombinant microorganism comprising a heterologous nucleic acid encoding one or more mycosporine-like amino acid (MAA) biosynthetic enzymes (e.g., MysH) to produce compounds of interest. Compositions comprising compounds produced using such methods are also provided herein. The present disclosure also provides methods of preventing sunburn, cancer, and chronic inflammatory diseases by administering such compositions to subjects in need thereof.

### CROSS-REFERENCE TO RELATED APPLICATIONS

This application claims priority under 35 U.S.C. § 119(e) to U.S. Provisional Application No. 63/392,467, filed Jul. 26, 2022, which is incorporated herein by reference in its entirety.

### GOVERNMENT SUPPORT

This invention was made with government support under Grant No. GM128742 awarded by the National Institutes of Health. The government has certain rights in the invention.

### REFERENCE TO AN ELECTRONIC SEQUENCE LISTING

The contents of the electronic sequence listing (U119570207W000-SEQ-WLC.xml; Size: 170,148 bytes; and Date of Creation: Jul. 26, 2023) is herein incorporated by reference in its entirety.

### BACKGROUND OF THE INVENTION

Skin cancers are among the most common cancer types in the United States with about 1.2 million Americans living with melanoma and 3 million more affected by nonmelanoma skin cancers.^{1,2} Solar radiation, especially ultraviolet (UV) radiation, is an established risk factor of skin cancers,³ as more than 90% of melanoma in some populations are linked to sunlight exposure.⁴ UV rays, mainly UVA (315-400 nm) and UVB (280-315 nm), induce a variety of damages on biomolecules (e.g., DNA and proteins) of living organisms on earth.⁵ In addition to behavioral changes, proper skin protection from excessive sun exposure has proven to be effective in reducing skin cancers.⁶ In this regard, many organic and inorganic compounds have been developed to dissipate the energy of UV rays and/or directly block their reach on the skin, and some have been used as active ingredients of commercial sunscreens.⁷ However, there are increasing concerns regarding the potential negative health impact of synthetic sunscreens (e.g., endocrine disruption, neurotoxicity, and systemic absorption),⁸⁻¹⁰ while multiple organic UV filters are accumulated in almost all water sources globally and may be potential contributors to coral reef bleaching, raising a severe environmental concern over their use.¹¹ Accordingly, there is a need for safer, biodegradable, and environmentally friendly new compounds with UV-modulating, anti-inflammatory, and/or anti-oxidative properties.

### SUMMARY OF THE INVENTION

Natural organisms have developed multiple effective UV mitigation strategies when utilizing solar energy, including the biosynthesis of diverse natural products as photoprotectants.^{12,13} These natural products (e.g., flavonoids, phenols, terpenoids, and polyketides) absorb UV radiation and release energy through thermal de-excitation, similar to synthetic chemical UV filters, while providing additional protection from UV-induced damages with other biological functions, e.g., antioxidants, anti-inflammation, and immunomodulation.¹⁴ These compounds provide important inspiration for the development of new generation sunscreens.¹⁵ One such example, mycosporine-like amino acids (MAAs), are a family of natural, thermally and photochemically stable UV protectants (FIG. 1A).¹⁶ The superior UV protection properties of MAAs has potential to impact the development of next-generation sunscreens for broad cosmetic applications if the low quantity available from natural resources or the lack of efficient synthetic preparation were properly addressed.^{25,26}

Accordingly, in one aspect, the present disclosure provides methods for producing a compound (e.g., an MAA, or a derivative thereof, and any of the compounds delineated herein). The methods of the present invention comprise culturing a recombinant microorganism under conditions suitable for production of the compound and isolating the compound from the recombinant microorganism, wherein the recombinant microorganism comprises a heterologous nucleic acid encoding one or more mycosporine-like amino acid (MAA) biosynthetic enzymes (e.g., a phytanoyl-CoA dioxygenase (MysH), or a homolog thereof). In some embodiments, the one or more MAA biosynthetic enzymes include MysA, MysB, MysC, MysD, and/or MysE.

In certain embodiments, the compound is of Formula (I), or a salt thereof:

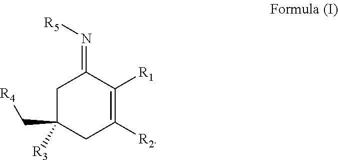

wherein R₁, R₂, R₃, R₄, and R₅ are as defined herein.

In some embodiments, the methods described herein further comprise providing a substrate of the one or more MAA biosynthetic enzymes to the recombinant microorganism. In certain embodiments, the substrate is a compound of Formula (II), or a salt thereof:

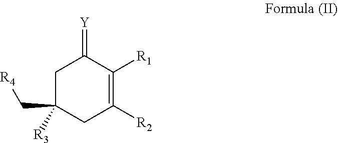

wherein R₁, R₂, R₃, R₄, and Y are as defined herein.

In another aspect, the present disclosure provides a recombinant microorganism comprising a heterologous nucleic acid encoding one or more MAA biosynthetic enzymes. In some embodiments, the one or more MAA biosynthetic enzymes comprise a phytanoyl-CoA dioxygenase (MysH), or a homolog thereof.

In another aspect, the present disclosure provides compositions comprising a compound produced by the methods disclosed herein. In some embodiments, the composition comprises an excipient. The composition may be formulated for topical administration (e.g., for use as a sunscreen or a cosmetic). In certain embodiments, the present disclosure provides methods of making the compositions disclosed herein. Such methods may comprise producing a compound using the methods disclosed herein and adding the compound to one or more excipients to produce the composition.

In another aspect, the present disclosure provides methods of administering a composition (e.g., any of the compositions described herein), comprising applying to composition to a subject. In some embodiments, the composition is applied to the skin of a subject. In certain embodiments, the method is a method of preventing sunburn. In certain embodiments, the method is a method of preventing cancer. In certain embodiments, the method is a method of preventing or treating a chronic inflammatory disease.

In another aspect, the present disclosure provides compounds produced using the methods disclosed herein. In some embodiments, the compounds are of Formula (I), or a salt thereof, as provided herein.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show the structures and biosynthesis of mycosporine-like amino acids. FIG. 1A provides the chemical structures and maximal absorbance of representative mycosporine-like amino acid analogs. FIG. 1B shows the biosynthetic pathway of shinorine, porphyra-334, palythine-Ser, and palythine-Thr.

FIGS. 2A-2B show a sequence similarity network (SSN) and a genome neighborhood network (GNN). FIG. 2A provides an SSN of one cluster with 585 members (shown in FIG. 6) with >45% protein sequence identity. One cluster was formed by 92 MysC homologs including Ava_3856 labeled with an arrow. Dots marked with an asterisk represent homologs from α-proteobacteria and eukaryotes, respectively. FIG. 2B shows that GNN analysis identified enzymes with 8 times or more co-occurrence within ten open reading frames upstream or downstream of 80 MysC homologs. The occurrence times of each enzyme group are labeled. GlyT: glycosyltransferase; Pentap: pentapeptide repeats; Uam2: putative restriction endonuclease.

FIGS. 3A-3C show the enzymes involved in the biosynthesis of mycosporine-like amino acids. FIG. 3A shows the gene organization of the MAA gene cluster from Nostoc linkia NIES-25. FIG. 3B shows representative refactored MAA clusters cloned into pETDuet-1 and pACYCDuet-1. FIG. 3C shows HPLC traces of crude extracts of E. coli cells expressing refactored MAA clusters. I: empty pETDuet-1; II: mysAB; III: mysABC; IV: mysAB2C; V: mysAB2CD; VI: mysABCD-R; VII: mysAB2CDH. All products were detected at 310 nm. ∇ and # indicate shinorine and MG-Ala, respectively.

FIG. 4 provides ¹H-¹H COSY (bold) and selected HMBC (H→C) correlations of isolated palythine-Thr.

FIGS. 5A-5C show analysis of the substrate preference of MysD. FIG. 5A provides HPLC traces of the MysD reactions with MG and L-Thr as substrate. Porphyra-334 was produced in the full reaction but not in the control reaction without MysD or ATP. FIG. 5B provides HPLC analysis showing that MysD accepted L-Ala, L-Arg, L-Cys, L-Gly, L-Ser, and L-Thr as its amino acid substrate. * and ♦ indicate MG-Arg and MG-2-Gly, respectively. The detection wavelength was 334 nm. FIG. 5C shows the relative activities of six amino acid substrates in the MysD reaction. The formation of porphyra-334 in the MysD reaction containing L-Thr after 8 min was determined in HPLC analysis. The corresponding MG consumption level was set as 100% to normalize the relative MG consumption levels in five other reactions that were performed for 30 min to allow the quantitation of corresponding disubstituted MAAs. Data represent mean±s.d. of two independent experiments.

FIG. 6 provides sequence similarity network (SSN) analysis of protein family #02655 in the Pfam database. The analysis identified 22 distinct clusters with a sequence identity of >35% of MysC proteins. The cluster with 92 MysC homologs as a subcluster is circled.

FIG. 7 shows sequence alignment of all phytanoyl-CoA dioxygenases identified in the GNN analysis. The alignment revealed the conserved 2-His-1-carboxylate facial triad (His119, D121 and His198 for A0A367QPY5) (SEQ ID NOs: 127-136).

FIGS. 8A-8B provide mass spectrometry data for porphyra-334 and shinorine. FIG. 8A provides TIC and EIC traces of methanolic extracts of N. linkia NIES-25 cells. Value ranges used to generate EIC traces represent the m/z values of parental ions of porphyra-334 (calculated [M+H]⁺: 347.1449), shinorine (calculated [M+H]⁺: 333.1292), and MG-Ala (calculated [M+H]⁺: 317.1343). Potential peaks for porphyra-334 and shinorine were observed. FIG. 8B provides HRMS and MS/MS spectra of a putative porphyra-334 peak. Proposed structures of fragment ions with m/z values of 186.0995, 200.1155, and 303.1182 are provided.

FIGS. 9A-9B show the maximal UV absorbance and HRMS spectra of 4-DG (FIG. 9A) and MG (FIG. 9B) produced in engineered E. coli.

FIGS. 10A-10B show the maximal UV absorbance and HRMS spectrum (FIG. 10A) and MS/MS spectrum (FIG. 10B) of porphyra-334 produced in engineered E. coli.

FIGS. 11A-11B show the maximal UV absorbance and HRMS spectrum (FIG. 11A) and MS/MS spectrum (FIG. 11B) of MG-Ala produced in engineered E. coli.

FIGS. 12A-12B show the maximal UV absorbance and HRMS spectrum (FIG. 12A) and MS/MS spectrum (FIG. 12B) of shinorine produced in engineered E. coli.

FIG. 13 provides HPLC traces of methanolic extract of E. coli expressing mysABCDH (bottom) and mysABCDH-sdr (top).

FIGS. 14A-14B show the maximal UV absorbance and HRMS spectrum (FIG. 14A) and MS/MS spectrum (FIG. 14B) of palythine-Thr produced in engineered E. coli.

FIG. 15 provides a ¹H NMR spectrum of isolated palythine-Thr (D₂O, 600 MHz). Of note, a chemical shift of formic acid was observed.

FIG. 16 provides a ¹³C NMR spectrum of isolated palythine-Thr (D₂O, 151 MHz). Of note, a chemical shift of formic acid was observed.

FIGS. 17A-17C show 2D NMR spectra of isolated palythine-threonine (D₂O, 600 MHz). FIG. 17A shows ¹H-¹H COSY. FIG. 17B shows HSQC. FIG. 17C shows HMBC.

FIG. 18 provides a proposed pathway for conversion of disubstituted MAAs into palythines by MysH.

FIGS. 19A-19B show the maximal UV absorbance and HRMS spectrum (FIG. 19A) and MS/MS spectrum (FIG. 19B) of palythine-Ser produced in engineered E. coli.

FIGS. 20A-20B provide the HRMS (FIG. 20A) and MS/MS (FIG. 20B) spectra of palythine-Ala produced in engineered E. coli.

FIG. 21 shows SDS-PAGE analysis of recombinant MysD. MysD showed the expected molecular weight at 42.9 kD.

FIG. 22 provides graphs showing the determination of optimal temperature and pH for the MysD reaction. The reaction mixture contained 100 mM buffer (pH 6.5 to 11), 10 mM MgCl₂, 5 mM ATP, 500 nM MysD, 50 μM MG, and 5 mM Thr. The reaction was incubated at 16 to 60° C. for 6 min and then quenched by incubation at 95° C. for 10 min. The highest conversion ratio of MG was set as 100% for normalizing other reactions. Data represent means±s. d. of at least two independent experiments.

FIGS. 23A-23B show analysis of MysD substrate preference. FIG. 23A provides an HPLC trace of the MysD reactions with MG and all 20 amino acids as substrates. The mixtures were separated on a Phenomenex Luna C8 5 um column with mobile phases 0.1 M TEAA (pH 7.0) and 2% methanol. The detection wavelength was 334 nm. All disubstituted MAAs were labeled with ∇ and their traces are shown in gray. FIG. 23B provides LC traces of the MysD reaction with L-Ala as substrate with the detection wavelengths of 334 nm and 310 nm (specific to MG).

FIGS. 24A-24B show the maximal UV absorbance and HRMS spectrum (FIG. 24A) and MS/MS spectrum (FIG. 24B) of MG-Arg produced in the MysD reaction.

FIGS. 25A-25B show the maximal UV absorbance and HRMS spectrum (FIG. 25A) and MS/MS spectrum (FIG. 24B) of MG-Cys produced in the MysD reaction.

FIGS. 26A-26B show the maximal UV absorbance and HRMS spectrum (FIG. 26A) and MS/MS spectrum (FIG. 26B) of mycosporine-2-Gly produced in the MysD reaction.

FIG. 27 shows that MysD accepts L-Ile, L-Met, and L-Val in its reaction. HPLC traces of the MysD reactions with MG and L-Thr, L-Val, L-Met, and L-Ile as substrates. The disubstituted MAA products are indicated by a triangle.

FIGS. 28A-28B show HRMS spectra (FIG. 28A) and MS fragmentation (FIG. 28B) of MG-Ile in the MysD reaction.

FIGS. 29A-29B show HRMS spectra (FIG. 29A) and MS fragmentation (FIG. 29B) of MG-Met in the MysD reaction.

FIGS. 30A-30B show HRMS spectra (FIG. 30A) and MS fragmentation (FIG. 30B) of MG-Val in the MysD reaction.

FIG. 31 shows that mycosporine-amine (M-NH₂) was produced by coexpression of MysH with MysABC in E. coli. Crude extracts of E. coli cells expressing refactored MAA clusters were analyzed by HPLC with a detection wavelength of 320 nm.

FIGS. 32A-32B show HRMS (FIG. 32A) and MS/MS (FIG. 32B) spectra of mycosporine-amine (M-NH₂) produced by coexpression of MysH with MysABC in E. coli. A UV absorbance spectrum is shown as the insert in FIG. 32A.

FIGS. 33A-33B show biochemical characterization of MysH. FIG. 33A shows an SDS-PAGE of purified MysH. Theoretical molecular weight was 31.7 kDa. FIG. 33B shows HPLC traces of the MysH reaction mixtures with a detection wavelength of 320 nm.

FIG. 34 shows a Michaelis-Menten curve of the MysH reaction. The data represent means±s. d. of at least three independent experiments.

FIG. 35 shows LC traces of one-pot MysDH reactions with all 20 amino acid substrates. The reactions were analyzed by HPLC at 320 nm. Palythines and disubstituted MAAs are indicated by triangles and asterisks, respectively. MG-Ile, MG-Met, MG-Val, palythine-Ile, palythine-Met, and palythine-Val were eluted after MG, and their peaks are not shown.

FIGS. 36A-36B show biochemical characterization of recombinant MysC. FIG. 36A shows SDS-PAGE analysis of purified MysC. Theoretical molecular weight was 54.9 kDa.

FIG. 36B shows HPLC traces of selected MysC reactions with 4-DG, L-Ala, L-Gly, and L-Ile as substrates.

FIGS. 37A-37B show that coexpression of a glyT gene led to the production of a new MAA analog in E. coli. FIG. 37A provides a scheme for the MAA BGC in Aphanothece hegewaldii CCALA 016. FIG. 37B shows an HPLC trace for the methanolic extract of E. coli cells co-expressing glyT with mysABCD genes.

FIGS. 38A-38B show HR-MS analysis of the glycosylated MAA analog. HR-MS/MS of parent ion with [M+H]⁺ 523.1761 (FIG. 38A) and HR-MS/MS/MS of the fragment ion with [M+H]⁺ m/z 327.1439 (FIG. 38B).

FIGS. 39A-39B show the in vitro MysD reaction produced 12 di-substituted MAAs. FIG. 39A shows conversion to the MAAs, and FIG. 39B shows the structures of 12 di-substituted MAAs.

FIG. 40 shows additional di-substituted MAA products.

FIG. 41 shows the MysDH coupled reaction produced 12 palythines.

FIG. 42 shows additional palythine products.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”).In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified (e.g., —CH═CHCH₃ or

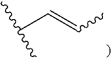

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc}C)₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{c})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, S₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂ wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some embodiments, the leaving group is a brosylate, such asp-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient to provide anti-oxidative or anti-inflammatory effects. In some embodiments, a therapeutically effective amount is an amount sufficient to provide UV-modulating effects (e.g., absorption of UV wavelengths between 310 and 362 nm). In certain embodiments, a therapeutically effective amount is an amount sufficient for preventing sunburn. In certain embodiments, a therapeutically effective amount is an amount sufficient for preventing cancer. In certain embodiments, a therapeutically effective amount is an amount sufficient for preventing or treating a chronic inflammatory disease.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, cancer is skin cancer (e.g., basal-cell skin cancer, squamous-cell skin cancer, or melanoma).

The terms “inflammatory disease” and “inflammatory condition” are used interchangeably herein, and refer to a disease or condition caused by, resulting from, or resulting in inflammation. A “chronic inflammatory disease” is an inflammatory disease that causes symptoms over a prolonged period of time. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent). Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

A “microorganism” refers to a single-celled organism, or a colony of such cells. In some embodiments, the microorganism is a eukaryote. In certain embodiments, the eukaryote is a species of yeast. In some embodiments, the microorganism is a prokaryote. In certain embodiments, the prokaryote is a species of cyanobacteria or a species of bacteria from the human microbiome. In certain embodiments, the prokaryote is E. coli. A “recombinant microorganism” refers to a microorganism that has been genetically altered to express one or more heterologous genes. The genome of the microorganism may be altered, for example, by genetic engineering techniques. In some embodiments, the microorganism is transformed with a vector comprising one or more heterologous genes (e.g., heterologous nucleic acid encoding one or more MAA biosynthetic enzymes, as described herein).

The term “cyanobacteria” refers to members from the group of photoautotrophic prokaryotic microorganisms which can utilize solar energy and fix carbon dioxide. Cyanobacteria are also referred to as blue-green algae. The cyanobacteria species of the present invention can be selected from the group consisting of Synechocystis, Synechococcus, Anabaena, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, and Scytonema.

The term “human microbiome” refers to the aggregate of all the microorganisms that reside on or within human tissues. In some cases, the human microbiome refers specifically to all of the species of bacteria that reside on or within human tissues. Species of human microbiome bacteria for use in the present invention can be selected from the group consisting of, but not limited to, Achromobacter, Acidaminococcus, Acinetobacter, Actinomyces, Aeromonas, Aggregatibacter, Acidaminococcus, Anaerobiospirillum, Alcaligenes, Arachnia, Bacillus, Bacteroides, Bacterionema, Burkholderia, Bifidobacterium, Buchnera, Butyriviberio, Campylobacter, Capnocytophaga, Candida, Clostridium, Chlamydia, Chlamydophila, Citrobacter, Cornybacterium, Cutibacterium, Demodex, Eikenella, Epidermophyton, Enterobacter, Enterococcus, Escherichia, Eubacterium, Faecalibacterium, Flavobacterium, Fusobacterium, Gingiva, Gordonia, Haemophilus, Lactobacillus, Leptotrichia, Malassezia, Methanobrevibacter, Morganella, Mycoplasma, Microbacterium, Micrococcus, Moraxella, Mycobacterium, Mycoplasma, Neisseria, Peptococcus, Peptostreptococcus, Plesiomonas, Porphyromonas, Propionibacterium, Providencia, Pseudomonas, Ruminococcus, Rothia, Ruminococcus, Sarcina, Staphylococcus, Streptococcus, Torulopsis, Treponema, Trichophyton, Veillonella, Vibrio, Wolinella, and Yersinia.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Methods for Producing a Compound

In one aspect, provided herein are methods for producing a compound comprising a) culturing a recombinant microorganism under conditions suitable for production of the compound; and b) isolating the compound from the recombinant microorganism. In some embodiments, the recombinant microorganism comprises a heterologous nucleic acid encoding (e.g., that encodes) one or more mycosporine-like amino acid (MAA) biosynthetic enzymes, wherein the one or more MAA biosynthetic enzymes comprise a phytanoyl-CoA dioxygenase (MysH), or a homolog thereof.

Exemplary MysH enzymes for use in the present invention include, but are not limited to, those of SEQ ID NOs: 1-11, or an amino acid sequence at least 70%, at least 75%, at least 80%, at least 85%, at least 90%, at least 95%, or at least 99% identical to the amino acid sequence of any one of SEQ ID NOs: 1-11:

A0A1Z4LFF0
(SEQ ID NO: 1)
MASLENQIILITGASSGIGTACAKIFAGAGAKLILAARRLERLQQLADILTQDENTEVH
LLELDVRDRSAVESAISNLPASWSDIDILINNAGLSRGLDKLHEGSFTDWEEMIDTNIK
GLLYLSRYVVPGMVSRGRGHVVNLGSIAGHQTYPGGNVYCATKAAVRAISEGLKQ
DLLGTPVRVTSVDPGMVETEFSQVRFHGNAQRANQVYQGVTPLTPDDVADVIFFCV
TRSPHVNINEVVLMPVDQASATLVNRRT
A0A367QPY5
(SEQ ID NO: 2)
MLKVDTLKISSQQVEAFERDGVICVKNALDDIWVERLRTAVDRNISIPGPLEEKNAPR
PEGSVEHASSLWLVDADFRALAFESPLPTLAAQVLKSEKLNFLADGFFVKKPKTNGH
IGWHNDLPYWPVQGWQCCKIWLPLDTVKQENGRLEYIKGSHQWGKELRERSNPSW
FVEPEPHEILSWDMEAGDCLIHHFLTIHHSVTNISSTQRRAIVTNWTGDDVTYYQRPK
AWPFKPLEEIDLPEFNSFKTKKVGEPIDCDIFPRVEVFR
A0A2Z6D3B5
(SEQ ID NO: 3)
MLKLELPKITLQEIEAFEQDGVICVKNVLDNIWVERMRKAVDKNISIAGPLEVKGISK
PEGNVEHTNSLWLVDADFRALVFESPLATLAAQILKSTKLNFLADGFFVKQPKATSR
VGWHNDLPYWPIQGWQCCKIWLALDKVNQQNGRLEYIKGSHRWGKELREDSNPA
WFSQPESHELLSWDMEPGDCLVHHLLTIHHSVTNISSTQRRAVVTNWTGDDVTYYP
RPKAWPFRPLDEIDIPEFDSLKAKKPGEPIDCDMFPKIKWHR
A0A2T1LWM2
(SEQ ID NO: 4)
MLIANSSKISRQEVENFKRDGVICLKNVVDDYWVERMRKAVDRNLLNSNGVRGRK
LKTGDVVHDYGLWLKDNDFRDLVFKSPLARVAAQIMESETINFLCDGFFVKKAKAD
SHVGWHNDLPYWPVKGWKCCKIWLALDPVNQENGRLEYIKGSHLWNKDLRENSN
VSWFSEPSYSDILYWDMEPGDALVHHFQTIHHSIGNTTYKSRRAIVTNWTGDDVVY
DPSPQTWPFQPIEEIGISEFNSLDTLRSGESIDCEIFPKIDLTPSPSPTSRGEQNPNFLKFP
HRL
A0A2L2NS52
(SEQ ID NO: 5)
MLKVDTSKITTQQVEAFERDGVICVKNVLDDIWVERMRRAVDKNVLIPGPLEVKGIP
RAEGHVEHTSSLWLTDADFRALAFESPLATLTAQVLKSKKLNFLGDGFFVKKPKGET
GVGWHNDKSYWPIQGWQCCKIWLALDSVNQENGKLEYIKASHLWGKELREASDPS
WFVEPEPHEIISWDMEPGDCLVHHFMTIHHSVRNTSSTRRRAVVINWTGDDVTYERR
PNAWPFRPLEEIDIPEFESLKAKKSGEPIDCDIFPRVELHR
A0A2C6TQQ8
(SEQ ID NO: 6)
MLKVDTPKISPQQVEAFERDGVICVKNALDDIWIERMRKAVDKNISIPGPLEGKNTPK
KEASAEHTSSLWLVDADFRALAFESPLPKLAVGVLKSEKLNFLADGFFVKRPEANGR
IGWHNDLPYWPVQGWQCCKIWLALDTVKQENGRLEYIKGSHQWGRELRERSNPSW
FVEPEPHEILSWDMEAGDCLIHHFLTIHHSVTNKSSTQRRAIVTNWTGDDVTYYQRP
KAWPFKPLEEIDLPQFNSLKTKKFGEPIDCDIFPRVEVHRHRTHI
A0A252E419
(SEQ ID NO: 7)
MLKIDTLKISLQQIEAFERDGVICLRNVLDESWVERMRTAVDKNVSIPGPLEVKGISR
PEASVEHTSSLWLVDPDFRALVFESPLSTIAAQLLRSEKLNFLADGFFVKKPKATSRV
GWHNDLPYWPIQGWQFCKIWLALDNVNEENGRLEYIKGSHQWGKELREDSNPSWF
VEPEPHELLSWDMEPGDCLVHHLLTIHHSVTNISSRQRRAVVTNWTGDDVTYYPRL
KAWPFRPLEEIDLPEFNSLKTKKTGEQIDCYMFPPIQLHR
A0A1Z4LFC6
(SEQ ID NO: 8)
MLKVDTQKISPQQVEAFERDGVICVKNAVDDIWVERMRTAVDKNISIPGPLEDKNVP
KPQGSAEHASSIWLIDADFRALAFESPLPTLAAQVLKSKKLNFLADGFFVKKPESNGR
IGWHNDLPYWPVQGWQCCKIWLALDTVKQENGRLEYIKGSHQWGKELRERSNPSW
FIEPEPHEILSWDMEAGDCLIHHFLTIHHSVTNISSTQRRAIVTNWTGDDVTYYQRPK
AWPFKPLEEIDLPEFNSLKTKKSGEPIDCDIFPRVQVHR
A0A1Z4IIA4
(SEQ ID NO: 9)
MLKLDLPKITLQEIEAFEQDGVICVKNVLDNIWVERMRKAVDKNLSIAGPLEVKGIT
KPEGNVEHSNSLWLVDTDFRALVFESPLANLAAQFLKSTKLNFLADGFFVKQPKASS
RVGWHNDLPYWPIQGWQCCKIWLALDKVNQQNGRLEYIKGSHRWGKELREDSNPS
WFSEPEPHELLSWDMEPGDCLVHHLLTIHHSVTNISSTKRRAVVTNWTGDDVTHYP
RPKAWPFRPLDEIDIPEFDSLKAKKPGEPIDCDMFPKIKWHR
A0A1Z4HWL1
(SEQ ID NO: 10)
MLKIDTSKISFQQIGAFERDGVICLRNVLDENWVERMRTAVDKNVSINGPLEAKGISR
AEASVEHTSSLWLVDPDFRALVFESPLSTIAAQLLQSEKLNFLADGFFVKKPKATSRV
GWHNDLPYWPIQGWQCCKIWLALDHVNEKNGRLEYIKGSHKWGKELREDSNPLWF
VEPEPHELLSWNMEPGDCLVHHLLTIHHSVTNISSTQRRAVVTNWTGDDVTYYPRPK
AWPFRSVEEIDLPEFNSLKTKKTGEPIDCDMFPQVQLH
A0A1U71924
(SEQ ID NO: 11)
MLKVDTRKISHQQVEAFERDGVICVKNAVDDIWVQRMRTAVDKNVLIPGPLEEKNA
PKPEASAEHTSNLWLVDADFRALAFESPLPTLAVQVLKSKKLNFLADGFFVKKPKSN
SRIGWHNDLPYWPIQGWQCCKIWLALDTVNQENGRLEYIKGSHRWGKELRERSNPS
WFVEPKPHEILSWDMEAGDCLIHHFLTIHHSVTNISSRQRRAVVTNWTGDDVTYYQR
PKAWPFKSIEEIDLPQFNSFKTKKSGEPLDCDIFPRIEVHR

Biosynthetic enzymes other than MysH may also be encoded by the recombinant microorganism used in the methods disclosed herein. In some embodiments, the one or more MAA biosynthetic enzymes further comprise a D-alanine-D-alanine ligase (MysD), or a homolog thereof. Exemplary MysD enzymes for use in the present invention include, but are not limited to, the amino acid sequence of SEQ ID NO: 12, or an amino acid sequence at least 70%, at least 75%, at least 80%, at least 85%, at least 90%, at least 95%, or at least 99% identical to the amino acid sequence of SEQ ID NO: 12:

A0A1Z4LFR3
(SEQ ID NO: 12)
MPVLRILHLVGSAQDDFYCDLSRLYAQDCLAAMAELPYDSAIAYITPDGQWRFPRSL
SREDIAQAKPMPVSEAIEFIAAQNIDIVLPQMFCIPGMTYYRALFDLLEIPYIGNTPDL
MAITAHKARTKAIVEAAGVKVPRGEVLRRGDVPTITPPVVIKPVSSDNSLGVTLVKD
AAEYEAALEKAFEHGDEAIVETFIEGREVRCGIIVKDGELIGLPLEEYLIDSQEKPIRTY
ADKLKKTDDGSLGFAAKGNNKSWILDPNDPITQKVQEVAKKCHQALGCRHYSLFDF
RIDSQGQPWFLEAGLYCSFAPKSVISSMAKAVGIPLNELLTIAIAETLGSNKYSDRISV
VEINEPSKTPRKERELSQMI

In some embodiments, the one or more biosynthetic enzymes comprise an ATP-grasp enzyme (MysC), or a homolog thereof. Exemplary MysC enzymes for use in the present invention include, but are not limited to, the amino acid sequence of any one of SEQ ID NOs: 13-104 and 113-116, or an amino acid sequence at least 70%, at least 75%, at least 80%, at least 85%, at least 90%, at least 95%, or at least 99% identical to the amino acid sequence of any one of SEQ ID NOs: 13-104 and 113-116:

A0A0Q2QHP0
(SEQ ID NO: 13)
MSGVRVHRIWDAGPGRTVAALAALCATLPVDLAVVLVALLVGRQPPRGRLPAEAR
RTVLLNGGKMTKALQLARSFHLAGHRVILVESAKYRWTGHRFSRAVDAFYCVPEPG
TPGYAPALLNIVRYENVDVYVPVSSPAGSVPDAVARELLDGACDVVHSDAKTVQLL
DDKAEFASTAASLSLQVPDSHRITDARQVADFPFPPGRSYILKRIAYNPVGRMNLTRL
SAATPDRNAAYARSLSISEDDPWILQEFIEGREYCTHGTARSGRLQVYGCCESSAAQ
VNYRSVDKPEIRRWVETFVKNLNLSGQVSFDFIEAHDGQVYAIECNPRTHSAITMFH
DHPDLAAAYLNDGHPLITPKHNSRPTYWIYHELWRLLRHPGRLGRLATILRGTDAIFT
GWDPVPYLMVHHLQIPALLWANLRVGKGWSRIDFNIGKLVENGGD
A0A3S0TU06
(SEQ ID NO: 14)
MGRTLATLVVLFGTLPFDLALVLVALLAGRRPSRGRLPAQARRTILLNGGKMTKAL
QLARSFHLAGHRVILVESEKYRWTGHRFSRAVDAFYCVPEPTEPGYALALLDIVRYE
NVDVYVPVSSPAGSVPDAVARELLDGACDVVHSDAKTVQLLDDKAEFASTAASLSL
RVPDSHRITDARQVVDFAFPAGRSYILKRIAYDPVGRMNLTRLSGATPDHNAAYARS
LPISEDDPWILQEFIEGREYCTHGTARSGRLQVYGCCESSSAQVNYRNVDKPEIRRWV
ETFVKNLNLSGQVSFDFIEARDGQVYAIECNPRTHSAITMFHDHPDLAAAYLDDNHP
LITPNDGARPTYWIYHELWRLLRHRGRISRLVTMLRGKDAIFAGWDPMPYLMVHHL
QIPALLWANLRAGKGWSRIDFNIGKLVENGGD
A0A5A7SAT3
(SEQ ID NO: 15)
MREVFQAKTIGTLALLQVVLPLNLALTTFALLRGVFVAPPPVAVAAQRKTILVSGGK
MTKALQLARSFHAAGHRVVLVESSKYRFNGHRFSRAVDRFYTVPAPDSDNYAVALL
AVVRAEEVDVYVPVCSPVASYYDALAKDQLSPHCEVLHCDADMVARLDDKYEFFA
LVASLGLSTPETHRVTAPGQVEEFDFTGTDYILKSIPYDPVHRRDMTTVPRPTATETT
TYARSKPITEATPWIMQEFVRGQEYCTHSLVRDGAVQVFCCCESSAFQINYRMVDKP
EIEEWVGEFAQRLNLTGQVSFDFIQGDDGRLHAIECNPRTHSAITMFYDHPDLARAY
LERGVPVVKPLPHSKPTYWIYHELWRLVTQRGGRAHRLAVIAQGKDAIFDWDDPLP
FLLVHHLQIPSLLLSNLLRRKGWTGIDFNIGKLIEAAGD
A0A0G4HZ53
(SEQ ID NO: 16)
MCRVETRPQVGEHAGMESVPLKAAEGGLVEERKAFLPQSYSLWKDSIEGRLWSLLT
LFGLFISSPFLFAFVALSVLSAVVRKLLRLPAARKLPEGSNKGRGRTALVTGGKMTKS
LDVCRHLKNEGFRVILTETPRYWMSASRFSSAVDKFVVLPVAPETHPEGYVEALRNL
FEKENVSLFAPVCSPFSSLYDAKAAESLPEGAISWSLPAEMVQQLDDKVEFARMAKE
VGLPVPDTLRVESKEEVRRFNSELAEKWRRDSSSAIASGAEKKKTDCRRYILKTLDY
DPMRRLDLFTLPCGPKELEKYLDETTISPDRPWLVQEFLEGREYSSCALSWKGKLLA
FTDNEAVISCYNFKYAGRDKIQEWVRVFCEKYQLSGVICVDFFERADGTQLAIECNP
RFSSNMTAFYNNPRLGAAMADPDLALRSGVTETPLPSSKESNWTLVDLYFHSYTQM
MKNPLAAFTAAAGLLLVSEETKEKQDAYWAPEDPLPSLALHCFHMPALLVRNVWD
GRKWAKIDFCIGKMTEENGD
R1G4T9
(SEQ ID NO: 17)
EVKPNGKVAIVSGGKMTKAYVIARQLKAQGCRVVLLETSKYWMVASRASNCVDRF
AMVPLPEKDLAGYLDAVRALAIEEKADLFIPVTSPAASEYEAQVAPVLPAGCVSWSL
DLETVRDLDDKTAFCSSAERLGLPAPRSHRVASDEEAHAFNEKLLAEAATATAGAET
RYILKSLAYDSMHRLDLFTLPCAPDNPWIIQTFVVGDEYSTCALVKEGRLLAFTDNR
ACLSCFNYTPARSEALRSWVRDFCAARRLSGVVCIDFIVDAQSGTPYAIECNPRFSSN
VLNLFWNPPFGGALFRPHKGGGVEAFFWPPPPPPPLQIWALLSKRPFSLRSAGALLST
VATKKDAYFDVADPLPFIAHLFVHIPALLARNLSTGNKWAKIDPCIGKLTEENGD
A0A433W0B3
(SEQ ID NO: 18)
MLLPQSITPTMQIFAVFQNLGTLLLLAIAFPFNCIVVLTALLWNLVSKPFRDRGILPVH
PKNIMLTGGKMTKALQLARSFHMVGHRVVLVETHKYWLTGHRFSNAVDRFYTVPA
PEKDPEAYSQALLAIAKQENIDVYVPVCSPVASYYDSVAKSVLSGCCEVFHFDAEVT
QMLDDKYEFAEKARSLGLSVPKSFKITNPEQVINFDFSDAERPYILKSIPYDSVRRLNL
TKLPCATPAETAAFVNSLPISPEKPWIMQEFIPGQEYCTHSTVRNGELRMHCCCESSA
FQVNYENVDKPEILAWVRHFVKELGITGQASFDFIQAEDGNVYAIECNPRTHSAITMF
YNHPGVADAFCRDVTCNTSTSRAGLLNSSFINNISGEPAPTIYPLQPLSTSKPTYWTY
HELWRLTGIRSFPQLQTWCKNILRGKDAIFAIDDPLPFLMVHHWQIPLLLLDNLRRLK
GWIRIDFNIGKIVELGGD
A0A139WZN8
(SEQ ID NO: 19)
MTQSISFSSPVPATPPISVKARFIALFQNLGTLTLLLLALPVNAVIVVISLVWNSLTRLF
STQQTTVARSKNILISGGKMTKALQLARSFGAAGHRVVLIETHKYWLSGHRFSNAVS
RFYTTPTPQYDPEAYIQTLIDIVKRENIDVYVPVTSPVASYYDSLAKPALSPYCEVLHF
DADVTKMLDDKFAFSEKARDLGLSVPKSFKITNPEQVLNFDFSQETRKYILKSIPYDS
VRRLDLTKLPCDTLEETAAFVKSLPISPEKPWIMQEFIPGKEFCTHSTVRNGELRLHCC
SESSAFQVNYENVENPEIQAWVKHFVNGLGFTGQVSFDFIQTDDGKVYAIECNPRTH
SAITMFYNHPQVSDAYLGTEPLTEPLQPLPNSKPTYWLYHEVWRLTGIRSFSQLQNW
VRNIFRGTDAIYKLHDPLPFLTVHHWQIPLLLLNNLWQLRGWTKIDFNIGKLVEFGG
D
A0A2Z5X784
(SEQ ID NO: 20)
MLCPYERLVFCLKEKLMTQSIPLSFSQPTTPLTVVKTKIVALFKTLGTLALLLLALPLN
GFVVLISLLWVIVRNPFTKPTAVAAHPQNILVSGAKMTKALQLARSFHAAGNRVILIE
GHKYWLSGHRFSNAVSRFYTVPAPQDDPESYTQALLEIVKKEKIDVYIPVCSPVASY
YDSLAKPVLSEYCEVFHFDADITAMLDDKFAFTDQARSLGLSVPKSFKITDPEQIINFD
FSQETRKYIIKSISYDSVRRLNLTKLPCDTPEETAAFVRSLPISPEKPWIMQEFIPGKEL
CTHSTVRDGELRLHCCSNSSAFQINYENVENPQIREWVQHFVKSLRLTGQVSFDFIQA
EDGTVYAIECNPRTHSAITMFYNHPGVAQAYLGKTPQAAPLEPLADSKPTYWLYHEI
WRLTSIRSWKHLQTWFKNLVRGTDAIYSMDDPIPFLTLHHWQITLLLLQNLQQLKG
WVKIDEN
A0A1Z4GTP3
(SEQ ID NO: 21)
MAQSISLSLPSSTTPSTGVRVKIVALFKTLGTLTLLLIALPFNALIVLIALLWGIARSPF
TKKAVVAANPQTILVSGAKMTKALQLARSFHAAGHRVILIEGHKYWLSGHRFSQAV
SRFYTVPAPQSDPEAYIQALVEIVKKEKVDIYVPVCSPVASYYDSLAKPTLSEYCEVF
HFDADITKMLDDKFAFTDKARSLGLSVPKSFKITDPQQVINFDFSQETRKYILKSIAYD
SVRRLDLTKLPCDSPEETAAFVNSLPISPENPWIMQEFIPGKEFCTHSTVRDGELRLHC
CCHSSAFQINYENVENPQIREWVQQFVKSLRLTGQVSFDFIQAEDGTVYAIECNPRTH
SAITMFYNHPGVAEAYFGKTPLAAPLEPLASSKPTYWIYHEIWRLTNIRSWKQLQTRL
NILFRGTDAIFRLNDPVPFLTLHHWQIPLLLLQNLQKLKGWVKIDFNIGKLVELGGD
A0A1Q4RU46
(SEQ ID NO: 22)
MAQSISLSSPAKTHAPGISASSLKTLGTLTLLLLALPLNASLVLVALLLKSLRPQNVTT
EEPKNILISGGKMTKALQLARSFHEQGHRVILLEAHKYWLTGHRFSFAVNKFYTVEA
PEKDPEGYIQSLVNIVEKENIDVYVPVCSPVASYYDSLAKKALPQCEVIHCDAEMTQ
MLDDKYAFAQTAQSFGLSVPKSFKITEPEQVINFDFSQEKRKYILKSIPYDSVRRLDLT
KLPCDTPEATAAFVRSLPISPEKPWIMQEFIPGKEYCTHSTVRNGVITLHCCCESSAFQ
VNYENVDNPKIFEWVSRFVKELGITGQVSFDFIEAEDGNIYAIECNPRTHSAITMFYN
HPGVADAYLGTGSNLAEPIQPKSTSKPTYWTYHEVWRLITTRSWSDFVYRFKIITHG
KDAIFSWQDPLPFLMNPHWQIFLLLIQNLQKNRGWVRIDFNIGKLVELGGD
A0A0C2R3C6
(SEQ ID NO: 23)
MAQSLPLTSAGGATSPTAFVAQVKALFQNIATLTILLLVLPINAAIVLTSLFWSRVSRF
VRPQTVVAANRKNILISGGKMTKALQIARSFHAAGHRVVLIETHKYWLSGHRFSDAI
SRFYTTPTPQYDPEAYIQALLDIVKKENIDVYVPVTSPVASYYDSLAKPALSPYCEVF
HFDADVTQMLDDKFAFSEKARSFGLSVPKSFKITNPEQVLNFDFSGETRKYILKSIPY
DSVRRLDLTKLPCDTPEETAAFVRSLPISPEKPWIMQEFIPGKEFCTHSTVKNGELRLH
CCAESSAFQVNYENVENPKIQEWVRHFVKELGITGQVSFDFIQAEDGTVYAIECNPRT
HSAITMFYNHPDVADAYLSEEPFTEPLVPLPNSKPTYWTYHEVWRLTGIHSFAQLQT
WIRNFLQGTDAIYQLDDPLPFLMVHHWQIPLLLLNNLRQLKGWTKIDFNIGKLVEIG
GD
A0A2R5FKA4
(SEQ ID NO: 24)
MRKYIFVVFQNLGTLVLLAIAFPLNCIVVLTSLLWNFLKQPFNKSIVVNPNSKNILIAG
ARMTKTLQLARSFHAAGHRVIIIDIEKFWSSGNKYSNSVAGFYTVPDPSKDLEGYVES
LHAIAKTEKIDFFIPVAIFSVIHYDQGQPPLPDFVEFFHFDADVTKILDDKFAFAETARS
FGLSVPKSFKITHPEQVINFDFSHEKRKYILKSIPYDQIRRLNLTKLPCATSAETAAFVN
SLPISEENPWIMQEFIPGKEYCTHTTARDGESRMYCCCESSAFQVNYENVDQQEIMQ
WATHFTKELGKTGQLSFDFIQAEDGTVYAIECNPRTHSAITMFYNHPGVADAYLGKE
PLAESLQPLADSKPTYWLYHEVWRLNEIRNFEQLQTWVRNIRRGKEAIFEVSDPLPFL
MVHHWQIPLLILDNLRRLKGWIRIDFNMGELIE
A0A0M0SH70
(SEQ ID NO: 25)
MTQSISVASPAPKTQSVPLGLRISALWKNVGTLALLLLVLPINAVIVLVSLLLGHQSQ
AIATEPKNILISGAKMTKALQLARSFHAAGHRVVLVETHKYWLTGHRFSKAVSRFYT
VPTPQSDPEAYTQALLDIVKTENIDVYVPVCSPIASYYDSLAKPVLSKFCEVFHCDAD
VTQMLDDKYAFAEKARSLGLSVPKSFKITDPEQILNFDFSQEKRQYILKSIPYDSVRR
LDLTKLPCETPEATADFVNSLPISPQKPWIMQEFIPGKEYCTHSTVRNGELRMHCCCE
SSAFQVNYENVDHPQILEWVRHFVKALGITGQVSFDFIQAEDGTIYAIECNPRTHSAIT
MFYNHPHVADAYLSEIPQLEPIQPLTNSKPTYWTYHEIWRLTGIRSFSQLQTWLKTFF
GGKDAIYCFSDPLPFLTVHHWQIPLLLLQNLQQLKGWIRIDFNIGKLVEFGGD
A0A2T1F866
(SEQ ID NO: 26)
MLLPQSITPTMQIFAVFQNLGTLLLLAIAFPFNCIVVLTALLGNLVSKPFRDRGILPVS
HPKNIMLTGGKMTKALQLARSFHMVGHRVVLVETHKYWLTGHRFSNAVDRFYTVP
APEKDPEGYSQALLAIAKQENIDVYVPVCSPVASYYDSVAKSVLSGCCEVFHFDAEV
TQMLDDKYEFAEKARSLGLSVPKSFKITNPEQVINFDFSDAERPYILKSIPYDSVRRLN
LTKLPCATPAETAAFVNSLPISPEKPWIMQEFIPGQEYCTHSTVRNGELRMHCCCESS
AFQVNYENVDKPEILAWVRHFVKELGITGQASFDFIQAEDGNVYAIECNPRTHSAIT
MFYNHPGVADAFCRDVTCNTSTSRAGLLNSSFINNISGEPAPTIYPLQPLSTSKPTYW
TYHELWRLTGIRSFPQLQTWCKNILRGKDAIFAIDDPLPFLMVHHWQIPLLLLDNLRR
LKGWIRIDFNIGKIVELGGD
A0A367QNV7
(SEQ ID NO: 27)
MAQSISVSSSPAIPSFPSETKIAVIIQNLLTLALLLLALPINATIVLVTLLWHTISRPFQQP
ATKAANPKNILISGGKMTKALQLARSCNAAGHRVVLIETHKYWLSGHRFSQAVDKF
YTVPAPQENPERYTQALIDIIKQENIDVYIPVTSPLGSYYDSLAKPLLSKYCEVFHFDA
DITERLDDKFAFAETARSLGLSVPKSFKITKAEQVLNFDFSQESRKYILKSIPYDSVRR
LDLTKLPCATPEETAAFVRSLPISPEKPWIMQEFIPGKEFCTHSTVRDGELRLHCCCES
SAFQVNYENVENSQIREWVRHFVKELKLTGQVSFDFIQAEDGKVYAIECNPRTHSAIT
TFYDHPQVAQAYLDNEPMAQTLQPLPSSKPTYWTYHEVWRLTGIRSLTQFKKWIANI
WRGTDAIYKSDDPLPFLMVHHWQIPLLLIKNLRQLKGWTRIDFNIGKLVELGGD
A0A2N6JWS5
(SEQ ID NO: 28)
MAQLQSIQASIFAVLQNLGTLALLMIAFPFNCIVVLLSLLLNFLSRPFHKPVILTKNPR
NIMIAGARMTKTLQLARSFHAAGHRVILVDTEKFWLSGNQFSHAVAGFYTVPDPHK
DLEGYTQALRAIAKKENIDFFIPVAIFAVIYYDSMSQHQLFDCCEVFHFNADVTKMLD
DKFAFAEKARSLSLSVPKSFKITAPEQILNFDFSNEKRKYILKSIPYDAVRRLNMTLLP
CDTPEQTAAFVKSLPISEEKPWIMQEFIPGKEYCTHSTVRDGKQTIYCCCESSAFQVN
YENVDKPEILQWVNHFVKELGLTGQISFDFIQAVDGTVYVIECNPRTHSAITMFYNHP
GVADAYLSKQPLAEPLQPLSDSKPTYWLYHEVWRLNEIRSLKQLQTWIKNILRGKDA
IFTVNDPLPFLMVHHWQIPLLLLDNLRRLKGWIRIDENPLLSL
B4VP63
(SEQ ID NO: 29)
MTNSLILAVLQNLGTLTLLAIAFPFNLTVVVVALVWDSLTRPFQNPKVANPNPKTIM
LTGGKMTKSLQLARSFYADGHRVILVESHKYWLVGHRFSRAVDRFYTVPAPNKDPD
GYMEGLLAIAKQENVDVYVPVCSPVASYYDSLAKPVLSGCCEVFHFDPDVTQLLDD
KFAFAQKAREFGLSVPKSFKITDPQQVIDFDFRGEKRKYILKSIPYDSVRRLNLTKLPC
KTPSETAAFVKSLPISEDKPWIMQEFIPGKEYCTHSTVRNGELRLHCCCESSAFQVNY
ENVDQPDILQWVSRFVQGLNLTGQASFDFIKTEDGIVYAIECNPRTHSAITMFYNHPG
VAEAYLSDTPLPEPLQPLPESKPTYWLYHEVWRLNEIRSFGDIRRWFKTVFGGKDAIF
QVNDPLPFLMVHHWQIPLLLLDNLRRMQGWIRIDFNIGKLVELGGD
K9QUQ5
(SEQ ID NO: 30)
MAQSISFDSSPATPSLGLETKIAAIIQNILTLALLLLALPINAIIVCIALVLGTIFRPQTTK
TSNPKNILISGGKMTKALQLARSFHADGHRVVLLETHKYWLTGHRFSQAVDKFYTT
PAPQKKPEDYIKALVDIVKRENIDVYIPVTSPVGSYYDSLAKPELSHHCEVFHFDAEIT
QMLDDKFAMAEKARSLGLSVPKSFKITSGEQVINFDFSRETRKYILKSIAYDSVRRLD
LTKLPCATPEETAAFVRKLPISPEKPWIMQEFIPGKEFCTHSTVRDGEIRLHCCCESSA
FQVNYENIENPQILEWVRHFVKELKLTGQISFDFIQTEDGQVYAIECNPRTHSAITTFY
NHPQVAEAYIGKQPMAETLQPLATSKPTYWTYHEIWRLTGIRSFTQLKTWLKNIWR
GTDAILQLHDPLPFLMVHHWQIPLLLLNNLRQLKGWTRIDFNIGKLVEFGGD
A0A0S3U2V2
(SEQ ID NO: 31)
MLNKLIAALQNLLTLTALLITLPINLAIVLIASLIGLFQRETIPQSNSPKRILITGGKMTK
ALQLARSFHAAGHFVVLVETQKYWLTGHQFSNAVDRFYTVPAPKQDSEAFIQALVD
IVQRENIDFFVPVTSPIESYYCSLAKPELSKYCEVLHFDVGITQLLDDKFELSEKARSL
NLTAPKTYRITDPQQVLDFEFDSSQYILKSIAYNSVHRLDMTKYPLESKAAMKAHLA
TLPISEDNPWILQEFISGQEYCTHSTVRDGKVRLHCCAKSSAFQVNYEQVENSEIQAW
VTTFVKALNLSGQISFDFIESSSGEVYAIECNPRTHSAITMFYNHPDVAKAYLGEPLTV
EPIQPLPTSKPTYWTYHEVWRLITGDRPLYRLQTILHGKDAILQTSDPIPFLMVHHWQI
PLLLLNNLRHLKGWVRIDFNIGKLVELGGD
K9TVZ3
(SEQ ID NO: 32)
MLLPQSITPTMQIFAVFQNLGTLLLLAIAFPFNCIVVLTALLWNLVSKPFRDRGILPVS
HPKNIMLTGGKMTKALQLARSFHMVGHRVVLVETHKYWLTGHRFSNAVDRFYTVP
APEKDPEAYSQALLAIAKQENIDVYVPVCSPVASYYDSVAKSVLSGCCEVFHFDAEV
TQMLDDKYEFAEKARSLGLSVPKSFKITNPEQVINFDFSDAERPYILKSIPYDSVRRLN
LTKLPCATPAETAAFVNSLPISPEKPWIMQEFIPGQEYCTHSTVRNGELRMHCCCESS
AFQVNYENVDKPEIIAWVRHFVKELGITGQASFDFIQAEDGNVYAIECNPRTHSAITM
FYNHPGVADAFCRDVTCNTSTSRAGLLNSSFINNISGEPAPTIYPLQPLSTSKPTYWTY
HELWRLTGIRSFPQLQTWCKNILRGKDAIFAIDDPLPFLMVHHWQIPLLLLDNLRRLK
GWIRIDFNIGKIVELGGD
A0A2N6MZD6
(SEQ ID NO: 33)
MAQLQSIQASIFAVLQNLGTLALLMIAFPFNCIVVLLSLLLNFLSRPFHKPVILTKNPR
NIMIAGARMTKTLQLARSFHAAGHRVILVDTEKFWLSGNQFSHAVAGFYTVPDPHK
DLEGYTQALRAIAKKENIDFFIPVAIFAVIYYDLMSQHPLFDCCEVFHFNADVTKMLD
DKFAFAEKARLLSLSVPKSFKITAPEQILDFDFSNEKRKYILKSIPYDAVRRLNMTLLP
CDTPEQTAAFVKSLPISEEKPWIMQEFIPGKEYCTHSTVRDGKQTIYCCCESSAFQVN
YENVDKPEILQWVNHFVKELGLTGQISFDFIQAVDGTVYAIECNPRTHSAITMFYNHP
GVADAYLSKQPLAEPLQPLSDSKPTYWLYHEVWRLNEIRSLKQLQTWVKNILRGKD
AIFTVNDPLPFLMVHHWQIPLLLLDNLRRLKGWIRIDFNIGELIE
A0A218PXL8
(SEQ ID NO: 34)
MAQSISLSLAKSPGSSTGVWVKLVALFKTLGTLTLLLIALPFNALIVLISLLWGFVRSP
FRQKAVVADHPQTILVSGAKMTKALQLARCFHAAGHRVILIEGHKYWLSGHRFSKA
VSGFYTVPAPELDPLGYIQALVEIVKKEKVDVYVPVCSPVASYYDSLAKPALSEYCE
VFHFDADVTKMLDDKFAFTDQARSLGLSVPKSFKITDHQQVINFDFSQETHKYILKNI
AYDSVRRLNLTKLPCDTPEETAAFVNSLPISEENPWIMQEFIPGKELCTHSTVRDGEL
RLHCCSDSSAFQINYENVENPQIREWVQHFVKSLALTGQVSFDFIQAESGTVYAIECN
PRTHSAITMFYNHPGVAEAYLGKTPLTDLTEPLANSKPTYWIYHEIWRLTGIRSWKQ
LQTSINTLAQGTDAVYQLDDPIPFLTLHHWQIPLLLLKNLQQLKGWVKIDFNIGKLVE
LGGD
A0A1Z4HW63
(SEQ ID NO: 35)
MAQSISLSLPESTTPATSVGVKIAALFKTLGTLTLLLIALPFNALIVLIALLWGIVRSPF
TKKAVVAAHSQTILVSGAKMTKALQLARSFHAAGHRVILIEGHKYWLSGHRFSQAV
SRFYTVPAPQSDSEGYIQALVEIVKQEKVDIYVPVCSPIASYYDSLAKPALSEYCEVFH
FDADITKMLDDKFAFTDKARSLGLSVPKSFKITDPQQVINFDFSQETRKYILKSIAYDS
VRRLDLTKLPCNTSEETAAFVNSLPISPENPWIMQEFIPGKEFCTHSTVRDGELRLHCC
CHSSAFQINYENVENPQICEWVQQFVKSLQLTGQVSFDFIQAEDGSVYAIECNPRTHS
AITMFYNHHGVADAYFGKTPLAAPLEPLASSKPTYWIYHEIWRLTGIRSWKQLQTSV
NTLLRGTDAIYNLNDPVPFLTLHHWQIPLLLLKNLQQLKGWVKIDFNIGKLVELGGD
A0A1Z4LYV8
(SEQ ID NO: 36)
MAQSSVSVSASQPIAPPTSIGMRFFALFQNLATLTLLLLALPINATIVLTTLLLNILTSP
FQKKQTTVVATEKKNILISGGKMTKALQLARFFHSAGHRVILTETHKYWLSGHRFSQ
SVDKFYTTPVPQKDSQAYTQALIDIINKEGIDIYIPVTSPIASYYDSLAKPALSEYCEVF
HIDAATCEMLDDKFAFSEKARSFGLSIPKCFKITNPEQVINFDFSGETRKYILKSIPYDS
VRRLDLTKLPCDTPEETEAFVRSLPISPQKPWIMQEFIPGKEYCTHSTVRDGVMRLHC
CCESSAFQVNYENVENPKIREWVTHFVKELGVTGQLSFDFIEAEDGNVYAIECNPRT
HSAITIFHDQLQQAANAYLSKEPIAAPLQALPNSKPTYWTYHEFWRLNEIRSLSQLGN
WIKNMLRGTDAIYTFDDCLPFLMVHHWQIPVLLLKNLSKLKGWTRIDFNIGKLVELG
GD
A0A654SJH1
(SEQ ID NO: 37)
MAKSVSLSLAKSTTPSTDVRLKLVALFKTLGTLTLLLIALPENGLIVLIALLWGIVQWP
LRKKALVAADPRTVLVSGGKMTKALQLARCFHGAGHRVILIETHKYWLSGHKFSRA
VSAFYTVPSPQSDPEGYIQSLVAIVKKEKVDFYVPVCSPVASYYDSLAKPALSAYCEV
FHFDADITKMLDDKFAFTEQGRSLGLSVPKSFQITDPQQVINFDFSQETRKYILKNIAY
DSVRRLNLTKLPCNTPEETAAFVNSLPISAQNPWIMQEFIPGKELCTHSTVRDGELRL
HCCSNSSAFQINYQNVENPQIRQWVQQFVKSLGLTGQVSFDFIQAEDGTVYAIECNP
RTHSAITMFYNHPGVADAYLGKTPQAAPVEPLANSKPTYWLYHEIWRLTGIRSWKQ
LQTSVNTLVGGTDAIFCFDDPVPFLTLYHWQIPLLLLKNLQDLKGWVKIDFNIGKLVE
LDGD
A0A2C6VZE1
(SEQ ID NO: 38)
MAQSISVSSSPAIPSFPSETKIAVIIQNLLTLALLLLALPFNATIVLVTLLWHTISRPFQ
QATTKTANPKNVLISGAKMTKALQLARSFNAAGHRVVLIETHKYWLSGHRFSQAVD
KFYTVPAPQENPERYTQALIDIIKQENIDVYVPVTSPLGSYYDSLAKPMLSNYCEVFH
FDADITQKLDDKFAFAETARSLGLSVPKSFKITSAEQVLNFDFSQESRKYILKSIPYDS
VRRLDLTKLPCATPEETAAFVKSLPISPEKPWIMQEFIPGKEFCTHSTVRNGELRLHCC
CESSAFQVNYENVENSQIREWVRHFVKEQKLTGQVSFDFIQAEDGRVYAIECNPRTH
SAITTFYDHPQVAQAYLDKEPMAETLQPLPTSKPTYWTYHEVWRLTGIRSFTQLKK
WIANIWRGTDAIYKPDDPLPFLMVHHWQIPLLLLKNLRQLKGWTRIDFNIGKLVELG
GD
A0A2T1EQS1
(SEQ ID NO: 39)
MLALFQNLGTLLLLALAFPFNCIVVLVALLTKPKLPQATVAKAQNILISGGKMTKAL
QLARSFYAAGHRVVLIETDKYWLTGHRFSRAVDAFYTVPAPQKDPEAYIQALVNIA
KKENIDVYIPVCSPISSYYDSLAKPALAGCCEVFHFDADITKMLDDKFAFAQTAQSFG
LSVPKSYKITHPQQVLDFDFSTEQNKYILKSIPYDSVRRLNLTKLPCNTRAETAAFVN
SLPISEEKPWIMQEFITGKEYCTHSTVRDGELRLHCCCESSAFQVNYENVDQPEILQW
VSHFVKQLGVTGQASFDFIRAENGNIYAIECNPRTHSAITMFYNHPGVASAYLSSQPL
KPLQPLTDSKPTYWLYHEVWRLNEIRSLQQLQTWFKNIRRGKESIFAFNDPLPFLMV
HHWQIPLLLLDNLRRLAGWIRIDFNIGKLVEFGGD
A0A1E5QWM1
(SEQ ID NO: 40)
MFSTTFKSLGTLALLKLALPFNLTLVLIASIINIFSTPFKIKKKPNINSKTVLLTGGKMT
KALQLARSFYSAGHRVILVETHKYWLSGHRFSVAVDKFFTIPDPVKDKEGYIDGLLDI
VKRENVDIFIPVSSPVASYYDSVAKMVLSPYCKVLHFDVEMTLVLDDKASLCQKASS
LGLTSPASYLITDVQEILDFDFSKNNHKYILKSIKYDSVYRLNMTQFPFEGMEEYVRS
LPISEENPWVMQQFITGQEYCTHSTVLNGKIRLHCCSMSSHFQVNYEHVDNQKIYEW
VEEFVGKLNLTGQISFDFIQTDDGTVYPIECNPRTHSAISMFYNHPLVADAYLNDGDD
APITPLESSKPTFWTYHELWRLTEVRSPQDLSQWWQKVTKGQDGIFSWQDPLPFLM
VHHWQIPLLLFGNLIKLKPWVKIDFNIGKLVESAGD
A0A218ACV8
(SEQ ID NO: 41)
MAQSISFDSSPATPSLGLETKIAAIIQNILTLTLLLLALPINTAIVFIYLVVGAIFRPQTSK
TSNPKNILISGGKMTKSLQLARSFHAPGHRVVLVETHKYWLTGHRFSQAVDKFYTTP
APQKDPEAYIQALEEIVKRENIDVYIPVTSPVGSYYDSLAKPKLSPHCEVLHFDAEITQ
MLDDKFAMAEKARSLGLSVPKSFKITSSEQVINFDFSGETRKYILKSIPYDSVRRLDLT
KLPCATPEETAAFVRNLPISPEKPWIMQEFIPGKEFCTHSTVRDGEIKLHCCCESSAFQ
VNYENVENPQILEWVKHFVKELKLTGQISFDFIQTEDGQVYAIECNPRTHSAITAFYN
HPLVAEAYIGSVTETLQPLSTSKPTYWTYHEVWRLTGIRSFTQLKTWLHNIWRGTDA
ILKLDDPLPFLMVHHWQIPLLLLNNLRQLKGWTRIDFNIGKLVELGGD
A0A2D3HK59
(SEQ ID NO: 42)
MRKHIFVVFQNLGTLVLLAIAFPLNCIVVLTSLLWSFIKQPFNKSIVVNPNSKNILIAG
ARMTKTLQLARSFHAAGHRVIIIDIEKYWLSGNKYSNSVAGFYTVPDPSKDLEGYVE
TLHAIANTEKIDFFIPVAIFSVIHYDQGKPPLPDCVEFFHFDADVTKILDDKFAFAETA
RSFGLSVPKSFKITDPEQVLNFDFSQEKRKYILKSIPYDQVRRLNLTKLPCDTKSETAA
FVKSLPISEENPWIMQEFIPGKEYCTHTTARDGESRMYCCCESSAFQVNYENVDQREI
MQWASHFTKELGKTGQLSFDFIQAEDGTVYAIECNPRTHSAITMFYNHPGVADAYL
GKEPLAESLQPLPDSKPTYWLYHEVWRLNEIRSFKQLQTWVRNIRRGKEAIFEVSDPL
PFLMVHHWQIPLLILDNLRRLKGWIRIDENMGELIE
A0A2S6VI18
(SEQ ID NO: 43)
MKSRQTPRERTFALLKSLGTLSLLLLAFPFSLSAVVGALLWSSLASLFQKRRVQAEPK
RILLTGAKMTKCLTLARSFHAAGHQVVMVETHKYWLSGNRFSNCVEAFYTVPAPQ
HDAEGYIQGLLNIVKQEKIDMFIPVSSPVASYYDSLAKPALSPYCEVFAFDAETTKLL
DNKFTFNQKAHSVGLSAPKTFLITNPEQVLNFDFAADGSQYILKSIAYDSINRLALLK
LPCAPQKMAEYVRSLPISEENPWIMQEFLKGQEYCTHAVVRDGKLLLYACSKSCDFL
VNYEHDYNPAILDWVTRFVKELNLTGQICLDFIQAEDGTVYPIECNPRTSTCITMFHD
QPKVVADAYLSSGAQASKEPVQPLPDSKPTYWTFHELWRLLTKVKSWKDLQYRLGI
IFNGVDPVFHPRDPLPFLGVNHWQIPLLILNNVRQLKGWERIDFNIGKLVQLGGD
K9X913
(SEQ ID NO: 44)
MQSGQTTSERTFALLKSLGTLTLLLLAFPFSLSVVVGALLWSSLTSLFQKRRVQVEPK
RILLTGAKMTKCLTLARSFHAAGHQVFMVETKKYWLSGNQFSNCVEALYTVPAPQH
DAEGYIQGLLNIVKQEKIDMFIPVSSPVASYYDSLAKPALSPYCEVFAFDAETTKLLD
NKFTFNQKAHSVGLSAPKTFLITNPEQVLNFDFAADGSQYILKSIAYDSINRLALLKLP
CAPEKMAEYVHSLPISAENPWIMQEFLKGQEYCTHAVVRDGKLLLYACSKSCDFLV
NYEHDYNPAILDWVTRFVKELNLTGQICLDFIQAEDGTVYPIECNPRTSTCITMFHDQ
PKVVADAYLSSSAQAPKEPVQPLPESKPTYWTFHELWRLLTKVKSWKDLQYRLGIIF
NGVDPVFHPRDPLPFLGVNHWQIPLLILNNVRQLKGWERIDFNIGKLVQLGGD
A0A1Y0RL91
(SEQ ID NO: 45)
MAHSISLSSRPATPAISIKALLVALFQNLGTLTILLLVLPINAAIVLISLLWSRLSSPWRS
QKAVVATHRKNILISGGKMTKALQLARSFHAAGHRVVLIETHKYWLSGHRFSNAVS
RFYTTPTPQHNPEAYIQALLDIVKREKIDVYVPVTSPVASYYDSLAKPALSPYCEVFH
FDADVTQMLDDKFAFSEKARALGLSVPKSFKITNPEQVINFDFSQETRKYILKSIPYDS
VRRLDLTKLPCDTPEETAAFVRSLPISPEKPWIMQEFIPGKEFCTHSTVKNGELRLHCC
SESSAFQVNYENIENPKIQKWVTHFVKELGITGQISFDFIQAEDGTVYAIECNPRTHSA
ITMFYNHPQVADAYLSQEAFTEPQEPLPNSKPTYWTYHEVWRLTGIRSFAQLQTWIR
NFLRGKDAIYQVDDPLPFLMVHHWQIFLLLLDNLRQFRGWTRIDFNIGKLVELGGD
A0A2P8QMI8
(SEQ ID NO: 46)
MQIFAVFQNLGTLLLLAIAFPFNCIVVLTALFWNLVSKPFRDRGILPVSHPKNIMLTG
GKMTKALQLARSFHMVGHRVVLVETHKYWLTGHRFSNAVDRFYTVPAPEKDPEGY
SQALLAIAKQENIDVYVPVCSPVASYYDSVAKSVLSGCCEVFHFDAEVTQMLDDKY
EFAEKARSLGLSVPKSFKITNPEQVINFDFSDAERPYILKSIPYDSVRRLNLTKLPCATQ
AETAAFVNSLPISPEKPWIMQEFIPGQEYCTHSTVRNGELRMHCCCESSAFQVNYEN
VDKPEILAWVRHFVKELGITGQASFDFIQAEDGNVYAIECNPRTHSAITMFYNHPGV
ADAFCRDVTCNVSTLYPLQPLSTSKPTYWTYHELWRLTGIRSFPQLQTWFKNILRGK
DAIFAIDDPLPFLMVHHWQIPLLLLDNLRRLKGWIRIDFNIGKIVELGGD
A0A6B3P645
(SEQ ID NO: 47)
MALILFVQGRAYALFQNLGTLILLLIVLPFNFLKVIPSLLWNFISQPFQKKVVAENPKN
ILITGAKMTKCLQLARSFHAAGHKVFLLEANKYWLSGNRFSNAVTGFYTLPFPQKD
WEGYSQGLLEIIKKEKIDVFIPVSSPAGSYYESLAKPLISEHCEVLHFDAEITQLLDNKF
TFIEKAKSFGLSVPKSFLITNPEQVLNFDFATDGSKYILKSIPYDSVRRLDMTKLPMNS
KAEMEEFVNSLPISEQRPWIMQEFVKGKEYCTHSTVRKGKVRLYCCCESSEFQVNYH
HVDRPQIYQWVEKFVRELNITGQISFDFIQTEDGRVYPIECNPRTHSAITTFYDHPGVA
DAYLKDSKDENEASLIPLPNSKPTYWTYHELWRLTGIRSLGQLKTWINRIFQGTDGIF
QINDPLPFLMVHHWQIPLLLLGNLQKLKGWVRIDFNIGKLVELGGD
A0A6B3MZW3
(SEQ ID NO: 48)
MGLISGSQKPIYTVLQNLGTLTLLLSVLPFNLLKVLPALLWNFLSKPFQKKLVVENSK
NIILTGAKMTKCLQLARSFQAAGHKVFMLETDKYWLSGNRFSNSVTGFYTVPNPKK
DWNGYCQKLLDIVKKENIDVFIPVSSAVLNYYESLVKPILSEYCEVLHFDVEITKLLD
NKFTFIEKAKSFGLTVPKSFLITKPEQIINFDFATDGSQYILKSIPYDSVRRLNMTKLPM
KSVQEMSNFVKSLPINQEKPWIMQEFVKGKEYCTHSTVRKGQIRLHCCCESSEFQVN
YEHVDHPQIYEWIEKFVKELNLTGQISFDFIQTEDNRVYPIECNPRTHSAITTFYNHPE
VADAYLNDSQNDNESPITPLSNSKPTYWTYHELWRLTAIRSWEQLKAWSKKITAGT
DSIFQFNDPLPFLMVHHWQIPLLLLENLKKLKGWVMIDFNIGKLVELEED
A0A2K8WS68
(SEQ ID NO: 49)
MFLTTFKSLGTLALLKLALPFNLTLVLIASIINIFSNPFKIKKKPNINSKTVLLTGGKMT
KALQLARSFHSAGHRVILVETHKYWLSGHRFSVAVDKFFTMPNPVKDKEGYIDGLL
DIVKRESVDIFIPVSSPVASYYDSVAKMVLSPYCEVLHFDVEMTLVLDDKANLCKKA
SSLGLTSPASYLITNVQEILDFDFSKNNHKYILKSIKYDSVYRLNMTQFPFEGMEEYV
RSLPISEENPWVMQQFITGQEYCTHSTVRNGKIRLHCCSESSHFQVNYKHIDNQKIYE
WVEEFVGKLNLTGQISFDFIQTDDGTVYPIECNPRTHSAISMFYNHPLVADAYLNDG
DDAPITPLESSKPTFWTYHELWRLTEVRSPQDLSQWWQKVTKGQDGIFSWQDPLPFL
MVHHWQIPLLLFGNLMKLKPWVKIDFNIGKLVESAGD
A0A4Q9JE38
(SEQ ID NO: 50)
MTQSISVASVGQTTQSVTLGLRISALFKNLATLALLLLVLPINAAIVLVSLLLGSQSQA
IATEPKNILISGGKMTKALQLARSFHAAGHRVVLVETHKYWLTGHRFSKAVSRFYTL
PTPQSDPEAYTQALLDIVQKENIDVYVPVCSPVASYYDSLAKPVLSKYCEVFHCDAD
VTQMLDDKYAFVEKARSLGLSVPKSFKITDPEQVSNFDFSQEKRKYILKSIPYDSVRR
LDLTKLPCETPEATADFVNSLPISSQKPWIMQEFIPGKEFCTHSTVRNGELRMHCCCE
SSAFQVNYENVDHPQILEWVRHFVKALGITGQVSFDFIEAQDGTIYAIECNPRTHSAIT
MFYNHPDVANAYLSEIPQVEPIQPLINSKPTYWTYHEIWRLTGIRSFSQLQTWLKNFF
GGKDAIYSLSDPLPFLTVHHWQIPLLLLQNLQQLKGWIRIDFNIGKLVEFGGD
Q3M6C5
(SEQ ID NO: 51)
MAQSLPLSSAPATPSLPSQTKIAAIIQNICTLALLLLALPINATIVFISLLVFRPQKVKA
ANPQTILISGGKMTKALQLARSFHAAGHRVVLVETHKYWLTGHRFSQAVDKFYTVP
APQDNPQAYIQALVDIVKQENIDVYIPVTSPVGSYYDSLAKPELSHYCEVFHFDADIT
QMLDDKFALTQKARSLGLSVPKSFKITSPEQVINFDFSGETRKYILKSIPYDSVRRLDL
TKLPCATPEETAAFVRSLPITPEKPWIMQEFIPGKEFCTHSTVRNGELRLHCCCESSAF
QVNYENVNNPQITEWVQHFVKELKLTGQISFDFIQAEDGTVYAIECNPRTHSAITTFY
DHPQVAEAYLSQAPTTETIQPLTTSKPTYWTYHEVWRLTGIRSFTQLQRWLGNIWRG
TDAIYQPDDPLPFLMVHHWQIPLLLLNNLRRLKGWTRIDFNIGKLVELGGD
A0A252E4S5
(SEQ ID NO: 52)
MAQSISLSLPESTTPSTSAGVKIVALFKTLGTLTLLLIALPFNALIVLIALLWGIVRRPF
TKKAAVAAHPQTILVSGAKMTKALQLARSFHAAGHRVILIEGHKYWLSGHRFSKAV
SRFYTVPAPQKDPEGYIQALVEIVKKEKVDVYVPVCSPVASYYDSLAKPALSEYCEV
FHFDADITKMLDDKFAFTDKARSLGLSVPKSFKITDPQQVINFDFSQETRKYILKSIAY
DSVRRLDLTKLPCDTPEETAAFVNSLPISSENPWIMQEFIPGKEFCTHSTVRDGELRLH
CCCNSSAFQINYENVENPQIREWVQQFVKSLRLTGQVSFDFIQAEDGTVYAIECNPRT
HSAITMFYNHPGVADAYLGKTPLAAPLEPLASSKPTYWIYHEIWRLTGIRSWKQLQT
SINTLLRGTDAICCLDDPVPFLTLHHWQIPLLLLKNLQQLKGWVKIDFNIGKLVELGG
D
A0A367RKS4
(SEQ ID NO: 53)
MAQSISLSLPQSTTPSTGVKVKIVALFKTLGTLTLLLIALPFNALIVLISLLWGIGRSPF
TKKAVVATHPQTILVSGAKMTKALQLARSFHAAGHRVILIEGHKYWLSGHRFSKAV
SRFYTVPAPQEDPEGYIQALVEIVKQEKVDVYVPVCSPVASYYDSLAKPALSEYCEV
FHFDADITKMLDDKFAFTDRARSLGLSVPKSFKITDPQQVINFDFSQEIRKYILKSISY
DSVRRLDLTKLPCDTPEQTAAFVNSLPISPEKPWIMQEFIPGKELCTHSTVRNGELRL
HCCSNSSAFQINYENVENPRIREWVQHFVKSLGLTGQVSFDFIQAEDGTTYAIECNPR
THSAITMFYNHSGVANAYFGKTLLDAPLEPLASSKPTYWIYHEIWRLTGIRSWKQLQ
TSVNTIVRGTDAIYCLDDPVPFLTLYHWQIPLLLLKNLQQLKGWVKIDFNIGKLVELG
GD
A0A1E2WNZ8
(SEQ ID NO: 54)
MAQSISLSLPESTTPSTGIRIKIVALFKTLGTLTLLLIALPINALIVLLSLLWSILFTKKPA
VAAHPQTILVSGGKMTKALQLARSFHAAGHRVILVEGHKYWLSGHRFSNAVSRFYT
VPAPQDDPEGYIQALLEIVKKEKVDIYVPVCSPVASYYDSLAKPSLSAYCEVFHFDAE
ITKMLDDKFAFTDQARSLGLSVPKSFKITDAEQVINFDFSKETRKYIIKSISYDSVRRL
NLTKLPCDTPEETAAFVKSLPISPEKPWIMQEFIPGKELCTHSTVRDGELRLHCCSDSS
AFQINYENVENPQIRQWVQHFVKSLGLTGQVSFDFIQAEDGTAYAIECNPRTHSAITM
FYNHPGVAEAYFGKTLLAAPLEPLADSKPTYWIYHEIWRLTGIRSAKQLQTWFQRLV
RGTDAIYQINDPIPFLTLHHWQITLLLLQNLQKLKGWVKIDFNIGKLVELGGD
A0A1B2CWG9
(SEQ ID NO: 55)
MAQSIPFDSASPTPQVSWGVRISALWKTVGTLLLLFLALPVNASIVLISLLWGIFSKPF
EKRVVAAAPKNILISGGKMTKALQLARSFHAAGHRVVLVESHKYWLTGHQFSNAVS
VFYTVSPPEKDPEGYTQQLLDIVKKERIDVYVPVCSPVASYYDSLVKPALSQHCEVF
HCDAEITQMLDDKYAFSEKARSFGLSVPKSFKITNPEQVINFDFSQEKRKYILKSIPYD
SVRRLNLTKLPCDTPEETAAFVRSLPISPEKPWIMQEFIPGKEFCTHSTVRNGELRLHC
CCESSAFQVNYENVNNPQILEWVKHFIKEMGITGQVSFDFIQTEDGTVYAIECNPRTH
SAITMFYNHPGVADAYLGKIPLPEPLQPLADSKPTYWLYHEIWRLTGIRSLSQFWTW
LKNLMRGKDAIYQLNDPLPFLTVPHWQITLLLLQNLRQLRGWVKIDFNIGKLVELGG
D
A0A1U7HY56
(SEQ ID NO: 56)
MQSGQTIRERTFASLKSLGTLTLLLLAFPFSLSVVVGALLWSSLTSLFQKHRVQVKPK
RILLTGAKMTKCLTLARSFHAAGHQVFMVETKKYWLSGNQFSNCVEALYTVPAPQH
DAEGYIQGLLNIVKQEKIDMFIPVSSPVASYYDSLAKPALSPYCEVFAFDAETTKLLD
NKFTFNQKAHSVGLSAPKTFLITNPEQVLNFDFATDGSQYILKSIAYDSINRLALLKLP
CAPATMAKYVHSLPISEENPWIMQEFLKGQEYCTHAVVREGKLMLYACSKSCDFLV
NYEHDYNPAILDWVTRFVKALNLTGQICLDFIQAEDGTVYPIECNPRTSTCITMFHDQ
PKVVADAYLSSSASILKEPVQPLPDSKPTYWTFHELWRLITKVKSWQDLQYRLGIIFN
GVDPVFHPRDPLPFLGVNHWQIPLLILNNVRQLKGWERIDFNIGKLVQLGGD
A0A1L9QXK4
(SEQ ID NO: 57)
MLIILFIQNHAYALFQNLSTFLLLTLLLPFNLLKILPVVLWNILTPIRAKPPGYEKPKNI
LITGAKMSKSLQLARSFNGSGHRVFLLEIHKYWLSGNRFSNAIKGFYTVPNPQKDWD
GYQQAVLEIVQKENINLFIPVSSPAGSYDESRLKPILSPYCEVFHFNLDITELLDNKFTF
IEKAKSLGLSVPQSFLITDSKQILDFDFAQDGSRYILKSIPYDSVRRLDMTKLPMKSEQ
EMEEFVKKLPITEDKPWIMQEFVQGKEYCTHSTVRKGKIRLHCCCESSEFQVNYDHV
EEPEIYQWVETFVRALNLTGQISFDFIKTEDGQVYPIECNPRTHSAITTFHDHPGVADA
YLKDAEDETESPIFPLPDSKPTYWTYHELWRVTEIRSFGQFQAWIKRITEGTDGIFQLN
DPLPFLMVHHWQIPLLLLQNLKKMKGWVRIDFNIGKLVELDGD
A0A2L2NR98
(SEQ ID NO: 58)
MGQSISLSLPQSPTSSTSVRVKIIALFKTLGTLTLLLIALPFNALIVLISLLWGIVRWTLP
RRRRSLFTKNVVAAHPQTILVSGAKMTKALQLARSFHAAGHRVILIEGHKYWLSGH
RFSKAVSRFYTVLAPQSDLEGYIQALVEIVKKEKVDVYVPVSSPVSSYYESLAKAALS
EYCEVFHFDPDITKMLDDKFALTDRARSLGLSVPKSFKITDPQQVINFDFSQETRKYIL
KSIDYDSVRRLNLTKLPCDTPEETAAFVNSLPISPEKPWIMQEFIPGKELCTHSTVRDG
ELRLHCCSDSSAFQINYENVENPQIREWVQHFVKSLALTGQVSFDFIQAQDGTVYAIE
CNPRTHSAITMFYNHPGVADAYLGKTPLAAPLEPLASSKPTYFIYHEIWRLTGIRSWK
QLQTSVNTLVRGTDAIYSLDDPIPFLTLHHWQIPLLLLKNLQQLKGWVKIDFNIGKLV
ELGGD
A0A2H2XFD9
(SEQ ID NO: 59)
MPQSISLTSSPTINQVNNKSVDISSSLKTLGTLTLLLLALPVNATLVLVALLLNSLRPR
NITTAANPKNILISGGKMTKALQLARSFHNAGHRVVLLEAHKYWLTGHRFSFAVNK
FYTVEAPEKDPEGYVQSLVDIVNKENIDVYVPVCSPVASYYDSLAKKALSSQCEVIH
CDALTTQMLDDKYAFTETARGFGLSVPKSFKITDPEQVINFDFSQEKRKYILKSIPYDS
VRRLDLTKLPCDTPEATAAFVRSLPISPEKPWIMQEFIPGKEYCTHSTVRNGEITLHCC
CESSAFQVNYAQVDNPQIFEWVRHFLKQLGITGQVSFDFIEAEDGTVYAIECNPRTHS
AITMFYNHPGVADAYLGTLNNLEEPIQPLPTSKPTYWIYHEMWRLINAGSWSKFVER
LQIITRGTDAIFSWQDPLPFLMNPHWQIFLLLIQNLQKNRGWIRIDFNIGKLVELGGD
A0A533NZW2
(SEQ ID NO: 60)
MFLQAKIWAFFQNIGTLTLLLLALPFNAIVVLPCLLWSWIAKLFQKKVVAANPKNILI
TGGKMTKALQLARCFHAAGHTVFLVETHKYWLSGHRFSRAVKGFFTVPAPEKHAN
GYCQGLLDIVKQEKIDVFIPVSSPVASYYDSIAKSLLSPHCEALTFDAEITEMLDNKFT
FCQKARELGLTAPKAFLITDPEQVLNFDFAADGSRYILKSIAYNSVYRLDLTKLPMSS
KEQMASFVKGLPISESQPWIMQEFISGQEYCTHSTVRNGIVRLHCCSQSSPFQVNYEQ
VDNQNIFQWVQQFVKALNLTGQISLDVIQTKDGKVYPVECNPRTHTAIAMFYNHPG
VADAYILDSKDAREPPIQPLPESKPTYWTYHELWRLTGIRSWGQLKGWFNKIIKGTD
GIFQVNDPLPFLMVHHWQIPLLLLNNMRKFKGWVKIDFNIGKLVELGGD
A0A367RVN3
(SEQ ID NO: 61)
MAQSISLSLPQSPTSSTGIKVKLVALFNTLGTLTLLLIALPFNALIVLISLLWGIVSSPF
TKKAVVAAHPQTILVSGAKMTKALQLARSFHAAGHRVILIEGNKYWLSGHRFSKAV
SRFYTVPAPQEDPEGYIQALVEIVKREKVDVYVPVCSPVASYYDSLAKPLLSEYCEVF
HFDPDITKMLDDKFAFTDRARSLGLSVPKSFKITDPQQVINFDFSQETRKYILKSIDYD
SVRRLNLTKLPCDTPEETAAFVNSLPISAEKPWIMQEFIPGKELCTHSTVRNGELRLH
CCSNSSAFQINYENVENPQIREWVQHFVKSLALTGQVSFDFIQAEDGTAYAIECNPRT
HSAITMFYNHPGVADAYLGKTPLAAPLEPLASSKPTYFLYHEIWRLTGIRSWKQLQT
SVNTLVRGTDAIYSLDDPIPFLTLHHWQIPLLLLKNLQQLKGWVKIDFNIGKLVELGG
D
A0A1Z4TPY4
(SEQ ID NO: 62)
MLMGFFEGEFMTQSISVASPAPKTQSVPLGFRISALWKNVGTLALLLLVLPINAVIVL
VSLLLGHQSQAIATEPKNILISGGKMTKALQLARSFHAAGHRVVLVETHKYWLTGH
RFSKAVSRFYTLPTPQSDPKAYTQALLDIVKKENIDVYVPVCSPVASYYDSLAKPVLS
KYCEVFHCDADVTQMLDDKYAFAEKARSLGLSVPKSFKITDPEQVINFDFSQEKRQY
ILKSIPYDSVRRLDLTKLPCETPEVTADFVNSLPISPQKPWIMQEFIPGKEFCTHSTVRN
GELRMHCCCESSAFQVNYENVDHPQILEWVRHFVKELGITGQVSFDFIQAEDGTIYAI
ECNPRTHSAITMFYNHPSVADAYLSEIPQLEPIQPLFNSKPTYWIYHEIWRLTGIRHWS
QLQTWLKNFFGGKDAIYSFSDPLPFLTVHHWQIPLLLLQNLQQLKGWLRIDFNIGKL
VEFGGD
A0A6B3MAD2
(SEQ ID NO: 63)
MGLISRSQKPVYIALQNLGILTLLLSVLPFNLLKVLPAVLWNFISKPFQKKVVAENSK
NIILTGAKMTKCLQLARSFQVAGHKVFMLETDKYWLSGNRFSNTVTGFYTVPNPKK
NWNGYCQELLDIVKREDIDVFIPVSGAALNYYESLIKPILSEHCEVLHFDIEITKLLDN
KFTFIEKAKSFGLAVPKSFLITNPEQILNFDFPADGGQYILKSIPYDSVRRLDMRKLPM
KSAQEMKDFVNSLPISEEKPWIMQEFVKGKEYCTHSTVRKGQIRLHCCCESSEFQVN
YEHVNHPQIYEWVETFVKELNLTGQISFDFIQTEDNRVYPIECNPRTHSAITTFYNHPE
VADAYLNDSQDDNESPLIPLPNSKPTYWIYHELWRLTAIRSWEQLKDWIKKITAGTD
SIFQFNDPLPFLMVHHWQIPLLLLDNLKKLKGWVMIDFNIGKLVELEED
A0A1Z4IH51
(SEQ ID NO: 64)
MTQSISLSLPESTTPSVGIKVKILALFKTLGTLSLLLVALPFNVLIVLISLLWGIVRVPF
TKNVVATHSQTILVSGAKMTKALQLARSFHADGHRVILIESHKYWLSGHRFSKAVSR
FYTVPSPQKDPESYIQALIEIVKKEKVDVYVPVCSPVASYYDSLAKPALSEYCEVFHF
NADITKMLDDKFAFTQKARALGLSVPKSFKITDPQQVINFDFSQETRKYILKSINYDS
VRRLNLTKLLCDTPEETAAFVKSLPISPETPWIMQEFIPGKEFCTHSTVRDGELRLHCC
CHSSAFQINYENVENPQIREWVQHFVKSLGLTGQVSFDFIQAEDGTVYAIECNPRTHS
AITMFYNHPGVAEAYFGKIPLPAPVEPLATSKPTYWTYHEIWRLTGIRSWKQLQTAIK
TIFQGTDAIYCLDDPLPFLTLHHWQIPLLLLQNLQQLKGWVKIDFNIGKLVELGGD
A0A1Z4IB36
(SEQ ID NO: 65)
MAQSLSLSSSHATPSIPWQTRVAAILQNIGTLTLLLLALPINASIVFISWLIFRPQKVKA
ANPQNILISGGKMTKALQLARSFHAAGHRVVLLETHKYWLTGHRFSVAVDKFYTVP
APQENPQAYIQALVDIVKQENIDVYVPVTSPAGSYYDSLAKPELSRYCEVFHFDADIT
QMLDDKFALVEKARSLGLSVPKSFKITSPEQVINFDFSGESRKYILKSIPYDSVRRLDL
TKLPCATPEETAAFVRTLPISQEKPWIMQEFIPGKEFCTHSTVRDGELRLHCCCESSAF
QVNYENVDNPQIREWVRRFVKELKLTGQISFDFIQAEDGTVYAIECNPRTHSAITTFY
DHPQVAQAYLSKETTAETLQPLATSKPTYWTYHEVWRLTGIRSLTQLGRWLGNIWR
GTDAIYQPGDPLPFLMVHHWQIPLLLLNNLRRLKGWTRIDFNIGKLVELGGD
K9VKW1
(SEQ ID NO: 66)
MLETVSVAAMPSERETNTGNRRFPTAFKTIATLILLLLVMPLNLALTAIALLRSIIIKPF
QSRSTTATPQTILISGGKMTKALQLARSFHQAGHRVILVETEKYWLTGHRYSRAVDR
FYTVPNPQTEEYPQALLKIVRQEGVNVYVPVCSPVASYYDAEVKRVLSGHCTVMHV
DVETLQRLDDKYEFATAAQALGLPVPKSYRITNPQQVIDFDFSDAQRKYIIKSIPYDS
VRRLDLTKLPCETPAETAAFVNSLPISESKPWIMQEYIPGQEFCTHSTVRNGHLQLHC
CCKSSAFQVNYENVDRPDIENWIRQFAKSLNLTGQVSFDFIQAADDGEIYAIECNPRT
HSAITMFYNHPDVAKAYLEPDPLPQTVQPLASSRPTYWIYHEIWRLVTHLSSPKLVSE
RLKIIAQGKDAIFDWDDPLPFLMVHHWQIPLLLWGNLQNPKEWIRIDFNIGKLVEIGG
D
A0A2T1F5R3
(SEQ ID NO: 67)
SRSVDRFYTVPKPQEKDYIDALLEIVQREGVDVYIPVCSPVASYYDALAKQVLSKYC
EVMHFDPELVQKLDDKSEFSAIATSLGLAVPDSYRITDTQQILDFDFAKQAHTYILKSI
PYDSLRRLNLTQLPCETPQQTAAFVEQLPICESNPWIMQAFITGQEYCTHSTVRNGEL
QLHCCCESSAFQINYEMVDKPEIEAWVRKFVSSLKLTGQVSFDFIQTRDGGVYAIEC
NPRTHSAITMFYNHPDVARAYLESDFPLIKPLESSRPTYWIYHEIWRLVTQPTQIGQRL
KIIASGKDAIFDWADPLPFLMVHHAQIPWLLLENLRQLKGWMRIDFNIGKLVEPAGD
K9W0D3
(SEQ ID NO: 68)
MAQVQPIKARIFAVFQNLGTLALLAIAFPINCIVVLASLLWNFCSRPFSKQGVSTLNPK
NILIGGGKMTKTLQLARLFHAAGHRVILFDSEKFRFSGYRFSNAVDRFYTVPDPQTDL
EGYTQALRAIAKQENIDIFIPVGIFAGGYFDSQRQPVLSGCCELFHFDADTMKMLDNK
FTFGEIARSFGLSVPKTFLITDPEQVLQFDFANEKNKYILKSIVYDSVYRLDMTKLPME
SQEKMAAHVNSLPIRKDNPWILQEFISGKEYCTHSTVRNGELTVHCCCESSAFQVNY
ENVDHPEIMQWVSRFVKELKLSGQISFDFMQAEDGTLYAIECNPRTHSAITMYYNHP
DLADAYLSAERRNYALPLQPLPDSKPTYWLYHEVWRLNEIRSLKQLQTWFKNIWRG
KDAIFEVNDPLPFLMVHHCYIPLLLLDSLRKLKGWVRIDFNIGKLVQLEGD
A0A1Z4SWP6
(SEQ ID NO: 69)
MPQSISLTSSPTINQVNNKSVDISSSLKTLGTLTLLLLALPVNATLVLVALLLNSLRPR
NITTAANPKNILISGGKMTKALQLARSFHNAGHRVVLLEAHKYWLTGHRFSFAVNK
FYTVEAPEKDPEGYVQSLVDIVNKENIDVYVPVCSPVASYYDSLAKKALSSQCEVIH
CDALTTQMLDDKYAFTETARGFGLSVPKSFKITDPEQVINFDFSQEKRKYILKSIPYDS
VRRLDLTKLPCDTPEATAAFVRSLPISPEKPWIMQEFIPGKEYCTHSTVRNGEITLHCC
CESSAFQVNYAQVDNPQIFEWVRHFLKQLGITGQVSFDFIEAEDGTVYAIECNPRTHS
AITMFYNHPGVADAYLGTLNNLEEPIQPLPTSKPTYWIYHEMWRLINAGSWSKFVER
LQIITRGTDAIFSWQDPLPFLMNPHWQIFLLLIQNLQKNRGWIRIDFNIGKLVELGGD
A0A1U71932
(SEQ ID NO: 70)
MAQSISVSSSPAMPSLAVETKIAVIIQNILTLALLLLALPINATIVVVTLLWCNISRPFQ
HSATKAANPKNILISGGKMTKALQLARSFNAAGHRVVLIETHKYWLSGHRFSQAVD
KFYTVPAPQENPECYTQALIDIIKQENIDVYIPVTSPLGSYYDSLAKPLLSEYCEVFHF
DADITQKLDDKFAFAETARSLGLSAPKSFKITSAEQVLNFDFSQESRKYILKSIPYDSV
RRLDLTKLPCATPEETAAFVRSLPISPEKPWIMQEFIPGKEFCTHSTVRDGELRLHCCC
ESSAFQVNYENVENSQIREWVRHFVKELKLTGQISFDFIQAEDGRVYAIECNPRTHSA
ITTFYDHPKVAQAYLDKEPMAETLQPLPTSQPTYWTYHEVWRLTGIRSFTQLKKWIA
NIWRGTDAIYKSDDPLPFLMVHHWQIPLLLIDNLRRLKGWTRIDFNIGKLVELGGD
A0A1W5CLX0
(SEQ ID NO: 71)
MAQSLPLSSAPATPSLPSQTKIAAIIQNICTLALLLLALPINATIVFISLLVFRPQKVKAA
NPQTILISGGKMTKALQLARSFHAAGHRVVLVETHKYWLTGHRFSQAVDKFYTVPA
PQDNPQAYIQALVDIVKQENIDVYIPVTSPVGSYYDSLAKPELSHYCEVFHFDADITQ
MLDDKFALTQKARSLGLSVPKSFKITSPEQVINFDFSGETRKYILKSIPYDSVRRLDLT
KLPCATPEETAAFVRSLPITPEKPWIMQEFIPGKEFCTHSTVRNGELRLHCCCESSAFQ
VNYENVNNPQITEWVQHFVKELKLTGQISFDFIQAEDGTVYAIECNPRTHSAITTFYD
HPQVAEAYLSQAPTTETIQPLTTSKPTYWTYHEVWRLTGIRSFTQLQRWLGNIWRGT
DAIYQPDDPLPFLMVHHWQIPLLLLNNLRRLKGWTRIDFNIGKLVELGGD
A0A328IAQ4
(SEQ ID NO: 72)
MTQSISVASVGQTTQSVTLGLRISALFKNLATLALLLLVLPINAVIVLVSVLLGSQSQA
IATEPKNILISGGKMTKALQLARSFHAAGHRVVLVETHKYWLTGHRFSKAVSRFYTL
PTPQSDPQAYTQALLDIVKKESIDVYVPVCSPVASYYDSLAKPVLSKYCEVFHCDAD
VTQMLDDKYAFAEKARSLGLSVPKSFKITDPEQVINFDFSQEKRQYILKSIPYDSVRR
LDLTKLPCETPQATADFVNSLPISPQKPWIMQEFIPGKEYCTHSTVRNGELRMHCCCE
SSAFQVNYENVDHPQILEWVRHFVKALGITGQVSFDFIEAEDGTIYAIECNPRTHSAIT
MFYNHPDVANAYLSEIPQVEPIQPLTNSKPTYWTYHEIWRLTGIRSFSQLQTWVKNFF
GGKDAIYSLSDPLPFLAVHHWQIPLLLLQNLQQLKGWIRIDFNIGKLVEFGGD
A0A533NF66
(SEQ ID NO: 73)
MFLQAKIWAFFQNIGTLTLLLLALPFNAIVVLPCLLWSWIAKLFQKKVVAANPKNILI
TGGKMTKALQLARCFHAAGHTVFLVETHKYWLSGHRFSRAVKGFFTVPAPEKHAN
GYCQGLLDIVKQEKIDVFIPVSSPVASYYDSIAKSLLSPHCEALTFDAEITEMLDNKFT
FCQKARELGLTAPKAFLITDPEQVLNFDFAADGSRYILKSIAYNSVYRLDLTKLPMSS
KEQMASFVKGLPISESQPWIVQEFISGQEYCTHSTVRNGIVRLHCCSQSSPFQVNYEQ
VDNQKIFQWVQQFVKALNLTGQISLDVIQTKDGKVYPVECNPRTHTAIAMFYNHPG
VADAYLLDSKDAREPPIQPLPESKPTYWTYHELWRLTGIRSWGQLKGWFNKIIKGTD
GIFQVNDPLPFLMVHHWQIPLLLLNNMRKFKGWVKIDFNIGKLVELGGD
A0A479ZZ55
(SEQ ID NO: 74)
MFPINLTLVITAFLTNLITLPFPKKITYENSKNILLTGGKMTKSLQLARSFHRAGHKVF
MVETHKYWLSGHQYSKAVKKFLTVPAPEKDPEGYCQSLLDIVKREKIDVFIPVSSPV
ASYYDSLAKPILSPYCEVFHFDTEMTKTLDDKFSLCEQARVLGLTAPKVFLITSPGEII
NFDFSQEQNPYIIKSIQYDSVTRLDMTKFPFEGMKEYVKKLPISKERPWVMQEFIKGQ
EYCTHSTVRDGEIRLHCCSKSSPFQVNYEQVDNPEIFQWVQKFVKELNLTGQISFDF
MQTEDGKVYPIECNPRTHTAITMFYDHPGLADAYLEPGKNQPHIEPLPTSKPTYWLY
HELWRITGIRSFNDLTNWLNKVIKGKDAMLDKDDPLPFLMVHHWQIVLLLLQNMV
KLKGWVRIDFNIGKLVEIGGD
A0A357A498
(SEQ ID NO: 75)
MLIILFIQNRAYALFQNLSTFLLLTLLLPFNLLKILPALLWNILTSIRAKLPGDEKPKNI
LITGAKMSKSLQLARSFNGAGHRVFLLETHKYWLSGNRFSNAIKDFYTVPNSEKNW
DGYQQAVLEIVQKENINLFIPVSSAAGSYDESRLKAILSPYCEVFHFDLDITELLDNKF
TFIEKAKNLGLSVPKSFLMTDSKQILDFDFVQDGSRYILKSIPYDSVRRLDMTKLPMK
SEQEMEEFVKELPITEDKPWIMQEFVQGKEYCTHSTVRKGKIRLYCCCESSEFQVNY
NHVEEPEIYQWVKTFVRALNLTGQISFDFIKTEDGQVYPIECNPRTHSAITTFHDHPGV
ADAYLKDVEDETKSPIFPLPDSKPTYWTYHELWRLTQIRSFGQFKAWIKRMIEGTDGI
FQPHDPLPFLMVHHWQIPLLILQNLKTMKGWVRIDFNIGKLVELDGD
A0A1Z4QDW0
(SEQ ID NO: 76)
MAQSISVDSSPAIPSLASETKIAVIIQNILTLALLLLALPINATIVLVTLFWGTILRPFQHS
ATKTANPKNILISGGKMTKALQLARSFHAAGHKVVLLETHKYWLTGHRFSQAVDKF
YTVPAPQENPESYTQALIDIIKQENIDVYIPVTSPLGSYYDSLAKPLLSRHCEVFHFDV
DITQNLDDKFEFAQKARSLNLSAPKSFKITSAEQVLNFDFSQESRKYILKSIPYDSVRR
LDLTKLPCATPEETAAFVRSLPISPEKPWIMQEFIPGKEFCTHSTVRDGELRLHCCCES
SAFQVNYENVENSQIREWVRHFVKELKLTGQISFDFIQAEDGAVYAIECNPRTHSAIT
TFYDHPKVAQAYLDQEPMAETLQPLPTSKPTYWTYHEVWRLTGIRSFTQLQKWLAN
IGRGTDAIYKLDDPLPFLMVHHWQIPLLLLNNLLRLKGWTRIDFNIGKLVELGGD
K9R4C7
(SEQ ID NO: 77)
MAQSSIPVLSSQTATHTISLGRRFVALVQNLATLTALLLALPINATIVFISLVLKILISP
FQKEQTTVTTAERKNILISGGKMTKALQLARFFHAAGHRVVLTETHKYWLSGHRFS
QAVDKFYTTPVPQKDSQIYTQALIDIVNKENIDIYIPVTSPIASYYDALAKQTLSEYCE
VFHIDAATCEMLDDKFAFSEKARSFGLSVPKSFKITNPEQVLNFDFSGETRKYILKSIP
YDSVRRLDLTKLPCDTPEETEAFVRSLPISPQKPWIMQEFIPGKEYCTHSTIRDGVVRL
HCCCESSAFQVNYENVENAKIREWVTHFVKELGVTGQLSFDFIEAEDGNVYAIECNP
RTHSAITIFHDQLQPAANAYLSKEPIKEPLQALINSKPTYWTYHEFWRLNEIRSFSQLG
NWIKNMLQGTDAIYTFDDSLPFLMVHHWQIPLLLLKNLFKLKGWTRIDFNIGKLVES
GGD
A0A3S0ZZ73
(SEQ ID NO: 78)
MAQSISLTESQTTVKPLAVWGKINALLKNLGTLVLLLVALPINATIVLVSLLWNLLAK
PFQKEQTVAGDRKNILISGAKMTKALQLARSFHAAGHRVVLLETHKYWLSGHRFSK
AVDNFYTTPVPQRDPQAYTQALIDIIEKENIDVYIPVTSPIASYYDSLAKPVLSQYCEV
FHFDAAVTQMLDDKFAFSEKARSLGLSVPKSFKITSPEQVLNFDFSQETRKYILKSIPY
DSVRRLDLTKLPCDTPEQTEAFVRSLPISAQKPWIMQEFIPGKEFCTHSTVRDGEIRLH
CCCESSAFQVNYEHVEHPQISEWIARFVKGLGITGQISFDFIQAEDGSVYAIECNPRTH
SAITTFHDRPEVAQAYLGKEAMTEPLQPLPSSKPTYWLYHEVWRLTSIRSLAQLRTWI
RNIWRGTDAIYKLDDPLPFLMLHHWQIPLLLLNNLWRLKGWTRIDFNIGKLVELGGD
A0A3C0NJT8
(SEQ ID NO: 79)
MAQLLFVRTPSFTMLKSLGTLTLLLIAFPINSIVVLTSLLWGLLSRPFQKQPLPADNQK
TAMFTGGKMTKALQLARSFHAAGHRVILVETHKYWLTGHRFSNAVDRFYTIPAPQK
DPEGYTQALLNIAKQENVDIYIPVCSPVSSYYDSLAKPALSGCCEVFHFDADITKMLD
DKFAFSEKARALGLSVPKSFKITNPEQVLNFDFSNETRKYILKSIPYDSVRRLNLTKLP
CDTPEETAAFVKSLPISEEKPWIMQEFIPGQEYCTHSTVRDGELRLHCCCESSAFQVN
YENVDQPEIMKWVSHFVKELKLTGQASFDFIQAEDGAIYAIECNPRTHSAITMFYNHP
GVADAYLGKEPLAEPLQPLPDSKPTYWLYHEIWRLNEIRSWSQLQTWMNNLLRGTD
AIFDVNDPLPFLTVHHWQIPVLLLDNLRKLRGWVRIDFNIGKLVESGGD
B2J6X7
(SEQ ID NO: 80)
MAQSISLSLPQSTTPSKGVRLKIAALLKTIGTLILLLIALPLNALIVLISLMCRPFTKKPA
VATHPQNILVSGGKMTKALQLARSFHAAGHRVILIEGHKYWLSGHRFSNSVSRFYTV
PAPQDDPEGYTQALLEIVKREKIDVYVPVCSPVASYYDSLAKSALSEYCEVFHFDADI
TKMLDDKFAFTDRARSLGLSAPKSFKITDPEQVINFDFSKETRKYILKSISYDSVRRLN
LTKLPCDTPEETAAFVKSLPISPEKPWIMQEFIPGKELCTHSTVRDGELRLHCCSNSSA
FQINYENVENPQIQEWVQHFVKSLRLTGQISLDFIQAEDGTAYAIECNPRTHSAITMF
YNHPGVAEAYLGKTPLAAPLEPLADSKPTYWIYHEIWRLTGIRSGQQLQTWFGRLVR
GTDAIYRLDDPIPFLTLHHWQITLLLLQNLQRLKGWVKIDFNIGKLVELGGD
A0A0CINCV3
(SEQ ID NO: 81)
MTKLQPIKARIIAVFQNLGTLLLLAIAFPINCSVVLVSLLWNFFSRPSHKQVVLTENPK
NILIGGGRMTKTLQLARSFHAAGHRVILVDIDKYWLSGHRFSRAVAGYYTVPAPQK
DLEGYTQALRAIAKKENIDFFIPVAIFAVSYFDSKGEPVLSGCCEIFHFDADITKMLDD
KFAFAEKARSLGLSVPKSFKITDPEQVLNFDFSQEKRKYILKSIPYDCLRRLNMTKLP
CDTFDMTAEFVKSLPISEEKPWIMQEFIPGKEYCTHSTVRDGELRLYCCCESSAFQVN
YENVDRPEIRQWVQQFVQEVGLTGEISFDIIQADDGTVYPIECNPRTHSAITMFYNHP
GVANAYLNKEPLVEPLQPLADSKPTYWLYHEVWRLTGIRSLKQLQTWIRNILRGKE
AIFSVSDPLPFMMVHHWQIPLLLLDNLRRLKGWVRIDFNLGELIESEEY
A0A1Z4S904
(SEQ ID NO: 82)
MAQSISFSSAPATPSVPSTSKIAAIFPNIGTLTLLLLALPINASIVLITLLLRAILRPFQPSA
VKAANPKNILISGGKMTKALQLARSFHAAGHRVVLLETHKYWLTGHQYSQAVDKF
YTVSAPQENPERYTQALVDIIKQENIDVYIPVTSPLGSYYDSLAKPELSRYCEVFHFDA
DITQMLDDKYELAQTARSLGLSVPKSFKITSAEQVLNFDFSGETRKYILKSIPYDSVRR
LDLTKLPCATPEETAAFVRSLPISPEKPWIMQEFIPGKEFCTHSTVRNGELRLHCCCES
SAFQVNYENVENPQILEWVKHFVKELKLTGQISFDFIQAEDGKVYAIECNPRTHSAIT
TFYDHPKVAEAYLSQEATTETLQPLPTSKPTYWTYHEVWRLTGIRSFKQLKTWIVNI
WRGTDAIYKFDDPLPFLMVHHWQIPLLLLKNLRQLKGWTRIDFNIGKLVELGGD
A0A2K8SZ63
(SEQ ID NO: 83)
MFQNLGTLVLLAIAFPLNCIVVLTSLLWSFIKQPFNKSIVVNPNSKNILIAGARMTKTL
QLARSFHAAGHRVIIIDIEKYWLSGNKYSNSVAGFYTVPDPSKDLEGYVETLHAIANT
EKIDFFIPVAIFSVIHYDQGKPPLPDCVEFFHFDADVTKILDDKFAFAETARSFGLSVPK
SFKITDPEQVLNFDFSQEKRKYILKSIPYDQVRRLNLTKLPCDTKSETAAFVKSLPISEE
NPWIMQEFIPGKEYCTHTTARDGESRMYCCCESSAFQVNYENVDQREIMQWASHFT
KELGKTGQLSFDFIQAEDGTVYAIECNPRTHSAITMFYNHPGVADAYLGKEPLAESL
QPLPDSKPTYWLYHEVWRLNEIRSFKQLQTWVRNIRRGKEAIFEVSDPLPFLMVHHW
QIPLLILDNLRRLKGWIRIDFNMGELIE
A0A3N6PGG7
(SEQ ID NO: 84)
MALILFVQGRAYALFQNLGTLILLLIVLPFNFLKVIPSLLWNFISQPFQKKVVAENPKN
ILITGAKMTKCLQLARSFHAAGHKVFLLEANKYWLSGNRFSNAVTGFYTLPFPQKD
WEGYSQGLLEIIKKEKIDVFIPVSSPAGSYYESLAKPLISEHCEVLHFDAEITQLLDNKF
TFIEKAKSFGLSVPKSFLITNPEQVLNFDFATDGSKYILKSIPYDSVRRLDMTKLPMNS
KAEMEEFVNSLPISEQRPWIMQEFVKGKEYCTHSTVRKGKVRLYCCCESSEFQVNYH
HVDRPQIYQWVEKFVRELNITGQISFDFIQTEDGRVYPIECNPRTHSAITTFYDHPGVA
DAYLKDSKDENEASLIPLPNSKPTYWTYHELWRLTGIRSLGQLKTWINRIFQGTDGIF
QINDPLPFLMVHHWQIPLLLLGNLQKLKGWVRIDFNIGKLVELGGD
A0A0C2QMV0
(SEQ ID NO: 85)
MKEQIFIVFQNLGTLVLLAIAFPFNCIVVLTSLVWNFIKQPFSQSIVVNPNSKNILIAGA
RMTKTLQLARSFHAAGHRVIIIDIEKFWSSGNKYSNSVAGFYTVPDPSKDLEGYVESL
HAIAKKEKIDFFIPVAIFSVIHYDSQGKPPLPDDVEFFHFDADVTKILDDKFAFAETAR
SFGLSVPKSFKITDPEQVLNFDFSQEKRKYILKSIPYDQVRRLNLTKLPCDTPSQTAAF
VKTLPISEEKPWIMQEFIPGKEYCTHTTARDGESRMYCCCESSAFQVNYENVDQPEI
MQWASHFTKELGKTGQLSFDFIQAEDGTVYAIECNPRTHSAITMFYNHPGVADAYL
GKEPLAESLQPLSDSKPTYWLYHEVWRLNEIRSFKQLQTWVRNIRRGKEAIFEVSDPL
PFLMVHHWQIPLLILDNLRRLKGWIRIDFNMGELID
Q3M6C5
(SEQ ID NO: 86)
MAQSLPLSSAPATPSLPSQTKIAAIIQNICTLALLLLALPINATIVFISLLVFRPQKVKA
ANPQTILISGGKMTKALQLARSFHAAGHRVVLVETHKYWLTGHRFSQAVDKFYTVP
APQDNPQAYIQALVDIVKQENIDVYIPVTSPVGSYYDSLAKPELSHYCEVFHFDADIT
QMLDDKFALTQKARSLGLSVPKSFKITSPEQVINFDFSGETRKYILKSIPYDSVRRLDL
TKLPCATPEETAAFVRSLPITPEKPWIMQEFIPGKEFCTHSTVRNGELRLHCCCESSAF
QVNYENVNNPQITEWVQHFVKELKLTGQISFDFIQAEDGTVYAIECNPRTHSAITTFY
DHPQVAEAYLSQAPTTETIQPLTTSKPTYWTYHEVWRLTGIRSFTQLQRWLGNIWRG
TDAIYQPDDPLPFLMVHHWQIPLLLLNNLRRLKGWTRIDFNIGKLVELGGD
A0A1Z4ND62
(SEQ ID NO: 87)
MIDTVSLNKSLAEKGFGRREIGVIGRNLATLGLLLLVLPINLLLTGVGLISRVSLRNPIS
QKTILISGGKMTKALLIARRFHAAGHRVILIESHKYWLTGHRFSNAVNKFYTVPAPEK
NPSAYIQALLDIIKREKVDLYVPVCSPVASYYDALVKSEMGFLTQVFHCDPEMVKML
DDKFTFAETARKLGLSVPKSFLITHPHQVINFDFQKETRPYILKSIRYDSVRRLDLTKL
PCETPEATERFVRSLPISPENPWIMQEFIPGQEYCTHSTVKNGELRMHCTSKSSAFQV
NYENIDHPRIQSWVSKFVKELGITGQVSFDFIETEDGEVYAIECNPRTHSAITMFYNHP
RVADAYLDEGVWEQPIQPLPDSKPTYWLYHEIWRLTGIRSWKDLQYRWKVLSTGV
DAIYSLDDPLPFLMVHHWQIPLLLWQNLLQLRGWVRIDFNIGKLVELGGD
A0A0D8ZR72
(SEQ ID NO: 88)
MQKMFAIFQNLGTLTLLAIAFPFNCIVVLSALVWNLISQPFQKQVVFNPDAKNILIGG
GRMTKTLQLARSFHAAGHRVILFDIDKNWFSGYRFSNAVAGFYTVPDPIKDLEGYTI
ALRAIAKQENIDFFVPVGIFANDYFDSKRQPVLSGCCETFHFDADTMKMLDNKFTFT
QKARSLSLSVPKAYLITDPEQVLKFDFSNEKNKYILKSIVYDPVFRLDLTKLPMESLE
KMAIHVRNLPISKDNPWILQEFITGQEYCTHSTVRNGELTVHCCCESSAFQVNYENV
DKPEILQWVSHFVKELQLTGQISFDFIQAEDGTIYAIECNPRTHSAITMYYNHPGLAD
AYLGQKPLAELLQPLPDSKPTYWLYHEVWRLNEIRSLKQLQTWFKNILRGKDAIFDV
NDPLPFLMVHHWHIPLLLLDNLQKLKGWVRIDFNIGKIVQVSD
A0A2T1LWM6
(SEQ ID NO: 89)
MDNLFNSSADSSSLSKGWLRSIQGSSLKTLGTLLLLLLMLPFNLALTLTALVWSWVW
PFRKRVIASNPKTVMISGGKMTKALQLARSFYMAGHRVILVETHKYWLVGHRYSW
AVDRFYTIPDPKQDTEGYLQGLLDIAQKEQVDLYVPVCSPVASYYDALAKELLAQQ
CDVFHEDAKTVQQLDDKYQFAQAATNLGLTVPKSFKITHPQQVLDFDFSKETHPYII
KSIPYDSVNRLNLTKLPCASRQDTEMFVNSLPISETKPWVMQEFITGQEYCVHSTVK
NGELRVYCCCESSAFQVNYEAVDIPEIKQWVTQFVQGMKLTGQMSFDFIRTPTGEVY
AIECNPRTHSAITLFYNHPDLAKAYLDPEPFSEPLEPLASARPTYWTYHEFWRLVTHL
SSLQEVAYRLGILFKGKDAIFSWNDPLPFLMVHGWQIPLLLLKSLRQGKDWIRIDFNI
GKLVQMGGD
K9XU47
(SEQ ID NO: 90)
MTQIFFVSGRGSAVLQNLGTLVLLLFLLPFNLIAVAFSAVINIFSGSKQRLTKTDVPKR
ILITGAKMTKALQLARSFHQRGHEVYLVETHKYWLSGHRFSRAVKGFFTVPTPEKEP
DAYCQRLLEIVQQKNIDVFIPVSSPIASYYDSLAKKILEPDCEAIHFDPEITAMLDDKY
AFCTKAKELGLSAPKVFCFTSPQQVIDFDFESDGSQYIVKSIPYDSVRRLDLTKLPFEG
MESYLRSLPISSEKPWVMQEFIRGQEYCFHATVRKGKIRLHCCSQSSPFQVNYEQVD
NPAIYQWVEKFVRELNLTGQICFDMIQTPDGTVYPIECNPRLHSAITMFHDHPGVAD
AYLLDGEQAITPLPDSKPTYWTYHELWRLLQVRSLSELQAWWHKVSRGTDAILQGD
DPLPFLMLHNWQIPLLLLDNLRRLKGWIRIDFNIGKLVELEGD
A0A2Z6D2K3
(SEQ ID NO: 91)
MTQSISLSLPESTTPSTGIKVKIVALFKTLGTLTLLLIALPENVLIVLISLLWGIVRVPF
TKNVVATHPQTILVSGAKMTKALQLARSFHADGHRVILIEGHKYWLSGHRFSKAVS
RFYTVPAPQSDPEGYIQALIEIVKKEKVDVYVPVCSPVASYYDSLAKPALSEYCEVFH
FDADITKMLDDKFAFTEKARSLGLSVPKSFKITDPQQVINFDFSQETRKYILKSINYDS
VRRLNLTKLPCDTPEQTAAFVKSLPISPETPWIMQEFIPGKEFCTHSTVRDGELRLHCC
CHSSAFQINYENVENPQIQAWIQHFVKSLRLTGQVSFDFIQAEDGQVYAIECNPRTHS
AITMFYNHPGVAEAYFGKTPLAAPLEPLPSSKPTYWTYHEIWRLTGVRSWKQLQTRL
NILLRGTDAIYCLDDPIPFLTLHHWQIPLLLLQNLQQLKAWVKIDFNIGKLVELGGD
A0A5P8W9G9
(SEQ ID NO: 92)
MAQSISLSVPKSTTPSTGVSIKIVALFKTLGTLTLLLIALPINAFIVLLSLLWGILFTKK
PAVAAHPQNILVSGGKMTKALQLARSFHAAGHRVILIEGHKYWLSGHRFSNAVSRF
YTVPAPQDDPQGYTQALLEIVKQEKIDIYVPVCSPVASYYDSLAKPALSEYCEVFHFD
ADITKMLDDKFAFTDQARSLGLSVPKSFKITDPEQVINFDFSKETRKYILKSISYDSVR
RLNLTKLPCDTPEETAAFVNSLPISPEKPWIMQEFIPGKELCTHSTVRDGELRLHCCSD
SSAFQINYENVENPQIREWVQHFVKSLGLTGQVSFDFIQAEDGTAYAIECNPRTHSAI
TMFYNHPGVAEAYFGKTPLAAPLEPLADSKPTYWVYHEIWRLTGIRSGKQLQTWFA
RLVRGTDAIYKIDDPLPFLTLHHWQIALLLLQNLQQLKGWVKIDFNIGKLVELGGD
A0A1S6LXZ0
(SEQ ID NO: 93)
MRKHIFVVFQNLGTLVLLAIAFPLNCIVVLTSLLWSFIKQPFNKSIVVNPNSKNILIAG
ARMTKTLQLARSFHAAGHRVIIIDIEKYWLSGNKYSNSVAGFYTVPDPSKDLEGYVE
TLHAIANTEKIDFFIPVAIFSVIHYDQGKPPLPDCVEFFHFDADVTKILDDKFAFAETA
RSFGLSVPKSFKITDPEQVLNFDFSQEKRKYILKSIPYDQVRRLNLTKLPCDTKSETAA
FVKSLPISEENPWIMQEFIPGKEYCTHTTARDGESRMYCCCESSAFQVNYENVDQREI
MQWASHFTKELGKTGQLSFDFIQAEDGTVYAIECNPRTHSAITMFYNHPGVADAYL
GKEPLAESLQPLPDSKPTYWLYHEVWRLNEIRSFKQLQTWVRNIRRGKEAIFEVSDPL
PFLMVHHWQIPLLILDNLRRLKGWIRIDFNMGELIE
A0A1Z4LFB5
(SEQ ID NO: 94)
MAQSISVSSSPAIPSFPSETKIAVIIQNLLTLALLLLALPINAAIVLVTLLWHTISRPFQQP
ATKAANPKNILISGGKMTKALQLARSCAAAGHRVILIETHKYWLSGHRFSQAVDKFY
TVPAPQENPERYTQALIDIIKQENIDVYIPVTSPLGSYYDSLAKPLLSEYCEVFHFDIDI
TEKLDDKFAFAETARSLGLSVPKSFKITSAEQVLNFDFSQESRKYILKSIPYDSVRRLD
LTKLPCATPEETAAFVRSLPISPDKPWIMQEFIPGKEFCTHSTVRDGELRLHCCCESSA
FQVNYENVENSQIREWVRHFVKELKLTGQVSFDFIQAEDGRVYAIECNPRTHSAITTF
YDHPQVAQAYLDNEPMAETLQPLPSSKPTYWTYHEVWRLTGIRSFTQLKKWIANIW
RGTDAIYKPDDPLPFLMVHHWQIPLLLLKNLRQIKGWTRIDFNIGKLVELGGD
A0A4D9CF37
(SEQ ID NO: 95)
MTQSISVASVGQTTQSVTLGLRISALFKNLATLALLLLVLPINAAIVLVSLLLGSQSQA
IATEPKNILISGGKMTKALQLARSFHAAGHRVVLVETHKYWLTGHRFSKAVSRFYTL
PTPQSDPEAYTQALLDIVQKESINVYVPVCSPVSSYYDSLAKPVLSKYCEVFHCDAD
VTQMLDDKYAFAEKARSLGLSVPKSFKITDPKQVINFDFSQEKRKYILKSIPYDSVRR
LDLTKLPCESPEATADFVNSLPISSQKPWIMQEFIPGKEFCTHSTVRNGELRMHCCCE
SSAFQVNYENVDHPQILEWVRHFVKALGITGQVSFDFIEAQDGTIYAIECNPRTHSAIT
MFYNHPDVANAYLSEIPQVEPIQPLINSKPTYWTYHEIWRLTGIRSFSQLQTWVKNFF
GGKDAIYSLSDPLPFLTVHHWQIPLLLLQNLQQLKGWIRIDFNIGKLVEFGGD
A0A1B2CWF7
(SEQ ID NO: 96)
MAQSIPFDSASPTPQVSWGVRISALWKTVGTLLLLFLALPVNASIVLISLLWGIFSKPF
EKRVVAAAPKNILISGGKMTKALQLARSFHAAGHRVVLVESHKYWLTGHQFSNAVS
VFYTVSPPEKDPEGYTQQLLDIVKKERIDVYVPVCSPVASYYDSLVKPALSQHCEVF
HCDAEITQMLDDKYAFSEKARSFGLSVPKSFKITNPEQVINFDFSQEKRKYILKSIPYD
SVRRLNLTKLPCDTPEETAAFVRSLPISPEKPWIMQEFIPGKEFCTHSTVRNGELRLHC
CCESSAFQVNYENVNNPQILEWVKHFIKEMGITGQVSFDFIQTEDGTVYAIECNPRTH
SAITMFYNHPGVADAYLGKIPLPEPLQPLADSKPTYWLYHEIWRLTGIRSLSQFWTW
LKNLMRGKDAIYQLNDPLPFLTVPHWQITLLLLQNLRQLRGWVKIDFNIGKLVELGG
D
A0A0C1N3Z4
(SEQ ID NO: 97)
MTQSISFSSPVPATPPFCVKTRFIALFQNLGALTLLLLALPINVAIVLISLIWSFLSRLFS
TQETTVAGAKNILISGGKMTKALQLARFFSAAGHRVVLIETHKYWLSGHRFSNAVSR
FYTTPTPQDEPEEYIQTLVDIVKRENIDVYVPVTSPVASYYDSLAKPALSPYCEVLHF
DADVTKMLDDKFAFSEKARALGLSVPKSFKITNPEQVLNFDFSQETRKYILKSLPYDS
VRRLDLTKLPCNTPEETAAFVKSLPISLEKPWIMQEFIPGKEFCTHSTVRNGDLKLHC
CSESSAFQVNYENVKNPKIQEWVRHFVKGLGLTGQVSFDFIQADDGKVYAIECNPRT
HSAITMFYNHPQVADAYLGTEPLAEPLAPVPNSKPTYWLYHEVWRLTGIRSFAQLQS
WIRNILRGTDAIYELHDPLPFLMVHHWQIALLLLNNLRQLKGWTKIDFNIGKLVELG
GD
A0A2L2N6B5
(SEQ ID NO: 98)
MRKHIFVVFQNLGTLVLLALAFPLNSIVVLTSLLWNFLKQPFSKSIVVNPNSKNILIAG
ARMTKTLQLARSFHAAGHRVIIIDIEKFWSSGNKYSNSVAGFYTVPDPSKDLEGYVE
TLHAIAKTEKIDFFIPVAIFSVIHYDRGKPPLPDFCEFFHFDADVTKSLDDKFAFAETA
RSFGLSVPKSFKITNPEQVLNFDFSQEKRKYILKSIPYDQIRRLNLTKLPCDTQSETAAF
VKSLPISEENPWIMQEFIPGKEYCTHTTARDGESRMYCCCESSAFQVNYENVDRLEIM
EWASHFTKQLGKTGQLSFDFIQAEDGTVYAIECNPRTHSAITMFYNHPGVADAYLGK
NPLAESLQPLGDSKPTYWLYHEVWRLNEIRSFKQLQTWLRNIRRGKEAMFEVSDPLP
FLMVHHWQIPLLILDNLRRLKGWIRIDFNMGELIE
A0A1Z4Q915
(SEQ ID NO: 99)
MVELQFIKARIFAVFRNLGTLALLAIAFPFNCIVVLAALLWNFFTRPFQKQVVLSENP
KNILIGGGRMTKTLQLARSFHAAGHRVILVDIHKYWLSGHRFSKAVAGYYTVPEPQK
DLEGYTQALRAIAKKENIDFFIPVAIFAVSYFDPQNKPVLAGCCEIFHEDGEVTKMLD
DKFAFAEKARSFGLSVPKSFKITAPEQVLNFDFSQEKNKYILKSIPYDSVRRLNMTKL
PCDTTEQTAAFVKSLPISEENPWIMQEFIPGQEYCTHSSLRNGELRLHCCCESSAFQV
NYENVDKPEIMQWVSHFVKELGLTGEASFDIIQAVDGTVYPIECNPRTHSAITMFYN
HPGVADAYLGKEPLAEPLQPLPDSKPTHWLYHEVWRLTGIRSLKQLQTWVRNILRG
KDAIFEVHDPLPFLMVHHWQIPLLLLDNLRRLKGWIRIDFNLGELIE
A0A2Z5VN68
(SEQ ID NO: 100)
MHFNCGAEKLMAQSISLSLPKSTTPSTGVRIKIVALFKTLGTLTLLLIALPINAFIVLLS
LLWSIPFTKKPAVAAHPQNILVSGGKMTKALQLARSFHAAGHRVILVEGHKYWLSG
HRFSKAVSRFYTVPAPQDDPEGYTQALLEIVKQEKIDIYVPVCSPIASYYDSLAKPALS
EYCEVFHFDADITKMLDDKFAFTDQARSLGLSVPKSFKITDPEQVINFDFSKETRKYIL
KSISYDSVRRLNLTKLPCDTPEETAAFVNSLPISPEKPWIMQEFIPGKELCTHSTVRDG
ELRLHCCSDSSAFQINYENVENPQIREWVQHFVKSLGLTGQVSFDFIQAEDGTAYAIE
CNPRTHSAITMFYNHPSVAEAYFGKTPLAAPLEPLADSKPTYWVYHEIWRLTGIRSG
KQLQTWFTRLVRGTDAIYKIDDPLPFLTLHHWQIALLLLQNLQQLKGWVKIDFNIGK
LVELGGD
A0A1Z4UKN2
(SEQ ID NO: 101)
MFPINLTLVITAFLTNLITLPFQKKITYENPKNILLTGGKMTKSLQLARSFHRAGHKVF
MVETHKYWLSGHQYSKAVKKFLTVPAPEKDPEGYCQSLLDIVKREKIDVFIPVSSPV
ASYYDSLAKPILSPYCEVFHFDTEMTKTLDDKFSLCEQARVLGLTAPKVFLITSPGEII
NFDFSQEQNPYIIKSIQYDSVTRLDMTKFPFEGMKEYVKKLPISKERPWVMQEFIKGQ
EYCTHSTVRDGEIRLHCCSKSSPFQVNYEQVDNPEIFQWVQKFVKELNLTGQISFDF
MQTEDGKVYPIECNPRTHTAITMFYDHPGLADAYLEPGKNQPHIEPLPTSKPTYWLY
HELWRITGIRSFNDLTNWLNKVIKGKDAMLDKDDPLPFLMVHHWQIVLLLLQNMV
KLKGWVRIDFNIGKLVEIGGD
A0A5Q0GJK5
(SEQ ID NO: 102)
MAQSLPLSSAPATPSLPSQTKIAAIIQNICTLALLLLALPINATIVFISLLVFRPQKVKA
ANPQTILISGGKMTKALQLARSFHAAGHRVVLVETHKYWLTGHRFSQAVDKFYTVP
APQDNPQAYIQALVDIVKQENIDVYIPVTSPVGSYYDSLAKPELSHYCEVFHFDADIT
QMLDDKFALTQKARSLGLSVPKSFKITSPEQVINFDFSGETRKYILKSIPYDSVRRLDL
TKLPCATPEETAAFVRSLPITPEKPWIMQEFIPGKEFCTHSTVRNGELRLHCCCESSAF
QVNYENVNNPQITEWVQHFVKELKLTGQISFDFIQAEDGTVYAIECNPRTHSAITTFY
DHPQVAEAYLSQAPTTETIQPLTTSKPTYWTYHEVWRLTGIRSFTQLQRWLGNIWRG
TDAIYQPDDPLPFLMVHHWQIPLLLLNNLRRLKGWTRIDFNIGKLVELGGD
A0ZIV3
(SEQ ID NO: 103)
MAQSISLSLGNSPTSSTGVWVKLVALFKTLGTLTLLLIALPFNALIVLISLLWGFVRSP
FRQKAVVAEHPQTILVSGAKMTKALQLARCFHAAGHRVILIEGHKYWLSGHRFSKA
VSGFYTVPAPQLDPEAYIQALVDIVEKEQVDVYVPVCSPVASYYDSLAKPALSEYCE
VFHFDADVTKMLDDKFAFTAQARSLGLSVPKSFKITDTQQVINFDFSQETHKYILKNI
AYDSVRRLNLTKLPCDTPEETAAFVNSLPISEENPWIMQEFIPGKELCTHSTVRDGEL
RLHCCSDSSAFQINYENVENTQIREWVQHFVKSLALTGQISFDFIQAESGTVYAIECNP
RTHSAITMFYNHPGVAEAYLGKTTLDAPLEPLTNSKPTYWIYHEIWRLTGIRSWKQL
QTAVNTLLRGTDAIFQLNDPVPFLTLHHWQIPLLLLKNLQQLKGWVKIDFNIGKLVE
LDGD
A0A3S1ANM2
(SEQ ID NO: 104)
MIIHMAQSISLSSPAKTHAPGISASSLKTLGTLTLLLLALPLNASLVLVALLLKSLRPQ
NFTTEKPKNILISGGKMTKALQLARSFHNAGHRVILLEAHKYWLTGHRFSSAVNKFY
TVEAPEKDPEGYIQSLVDIVEKENIDVYVPVCSPVASYYDSLAKKALPQCEVIHCDAE
MTQMLDDKHAFAQTAQSFGLSVPKSFKITDPEQVINFDFSQEKRKYILKSIPYDSVRR
LDLTRLPCDTPEATAAFVRSLPISSEKPWIMQEFIPGKEYCTHSTVRNGVITLHCCCES
SAFQVNYENVDNPKIFEWVSRFVKELGITGQVSFDFIEAEDGNIYAIECNPRTHSAITM
FYNHPGVADAYLGTGNNLAEPIQPKFTSKPTYWTYHEIWRLFNTRSWSDFVYRFKII
KHGKDAIFSWQDPLPFLMNPHWQIFLLLIQNLQKNRGWIRIDFNIGKLVELGGD
MysC-158
(SEQ ID NO: 113)
MSLSAPPSRSKIRSTLKTLGTLVLLLLALPLNAAIVLVALLRNLITRPRKRATAANPKT
VLISGGKMTKALQLARSFHRAGHRVILVETHKYWLTGHRFSNAVDRFYTVPAPQDD
PEGYAQALLDIVQKENVDVYVPVCSPVASYYDALAKETLSPHCEVFHFDADTVKML
DDKYQFAEMARSLGLSVPESHRITSPEQVLDFDFSQSEGRKYILKSIAYDSVRRLDLT
KLPCPTPEETAAFVRSLPISPDNPWIMQEFIEGQEYCTHSTVRDGRLRLHCCCESSAFQ
VNYEHVDNPEIQEWVQRFVKALNLTGQVSFDFIQTDDDGRVYAIECNPRTHSAITMF
YNHPGVAEAYLDPDPDLAEPIQPLPSSRPTYWLYHELWRLLTHPRSLQDLRERLKTIF
RGKDAIFDWDDPLPFLMVHHWQIPLLLLKNLRQGKDWVRIDFNIGKLVELGGD
MysC-175
(SEQ ID NO: 114)
MVVAENPKNILITGGKMTKALQLARSFHAAGHRVFLVETHKYWLSGHRFSNAVDRF
YTVPAPQKDPEGYVQGLLDIVKQENIDVFIPVSSPVASYYDSLAKPVLSPYCEVFHFD
AEITKMLDNKFTFSEKARSLGLSAPKSFLITDPEQVLNFDFAADQGSQYILKSIPYDSV
HRLDMTKLPCDKEEMAEYVKSLPISEENPWIMQEFITGQEYCTHSTVRDGKIRLHCC
SKYPTLFTASSAFQVNYEHVDNPAILQWVTRFVKELNLTGQISFDFIQAEDDGTVYPI
ECNPRTHSAITMFYNHLPGVVADAYLKDSPDEEEPIQPLPDSKPTYWLYHELWRLTEI
RSWSQLQAWINNILKGTDAIFQVNDPLPFLMVHHWQIPLLLLNNLRKLKGWVRIDEN
IGKLVELGGD
MysC-225
(SEQ ID NO: 115)
MVVAENPKNILITGGKMTKALQLARSFHAAGHRVFLVETHKYWLSGHRFSNAVDRF
YTVPAPQKDPEGYIQALLDIVKQENIDVFVPVSSPVASYYDSLAKPVLSPYCEVFHFD
ADITKMLDDKFTFSEKARSLGLSAPKSFLITDPEQVLNFDFASDQGSQYILKSIPYDSV
HRLDMTKLPCDSKEEMAAYVKSLPISEENPWIMQEFITGQEYCTHSTVRDGKIRLHC
CSKYPTLFTASSAFQVNYEHVDNPKILQWVTRFVKELNLTGQISFDFIEAEDDGTVYA
IECNPRTHSAITMFYNHLPGVVADAYLGKSPSAEEPIQPLPDSKPTYWLYHEVWRLTE
IRSWSQLQTWINNILRGKDAIFQVNDPLPFLMVHHWQIPLLLLNNLRKLKGWVRIDF
NIGKLVELGGD
MysC-230
(SEQ ID NO: 116)
MVVAENPKNILLTGGKMTKALQLARSFHAAGHRVILVETHKYWLSGHRFSNAVDRF
YTVPAPQKDPEGYTQALLAIAKQENIDVYVPVCSPVASYYDSLAKPVLSGCCEVFHF
DADVTKMLDDKFAFSEKARSLGLSVPKSFLITDPEQVLNFDFSNEQKRKYILKSIPYD
SVHRLDMTKLPCDSKEEMAAYVKSLPISEENPWIMQEFIPGKEYCTHSTVRNGELRL
HCCCEYPTLFTASSAFQVNYENVDNPKILQWVSHFVKELKLTGQISFDFIEAEDDGTV
YAIECNPRTHSAITMFYNHLPGVVADAYLGKEPLEEPLQPLPDSKPTYWLYHEVWRL
TEIRSFSQLQTWIKNILRGKDAIFSVNDPLPFLMVHHWQIPLLLLNNLRRLKGWIRIDF
NIGKLVELGGD

In some embodiments, the one or more biosynthetic enzymes comprise a dimethyl-4-deoxygadusol synthase (MysA), or a homolog thereof. Exemplary MysA enzymes for use in the present invention include, but are not limited to, the amino acid sequence of any one of SEQ ID NOs: 105-111, or an amino acid sequence at least 70%, at least 75%, at least 80%, at least 85%, at least 90%, at least 95%, or at least 99% identical to the amino acid sequence of any one of SEQ ID NOs: 105-111:

A0A2K8WSM2
(SEQ ID NO: 105)
MGNGALAENLKEDDKTVIWRPHEEKYRTSEWYTGSGQITTADEGLSFEVTAVYQLK
SEVKVVKDIFAISNHTLANIYRPRSRCIAVVDQTVAELYGEKIEGYFQAQEIPLELMVI
RAWESDKTPETVHRILAFLGKDGCDVSRNEPVLVIGGGVLSDVAGLACALQHRRTP
YIMIGTTIIAAIDAGPSPRTCTNGTQFKNSIGVYHPPVLTLVDRQFFSTLDMGHIRNGM
AEIIKMAVTDDKELFELLEQYGQELIKTRFATIDASEELEKIADLIIYKALYAYMKHEG
TNMFETYQDRPHAYGHTWSPRFEPAVKLMHGHAVTIGMAFGATLAQELGWLSQEE
CQRIINLSSKLGLSVFHPILEDVQIMVDGQKNMRRKRGDGGLWAPLPTTIGACDYVQ
EVEPELLNQAVVAHKKYCSQLPHEGAGEQMYLSDLGLE
A0AOD5ACA9
(SEQ ID NO: 106)
MSNLQAQVVAGDRSFRVEGYERIEYDLIYVDGVFAIENTELADSYRPYGRALMVVD
EAVHDIYGDRISAYFDHHEIALTVVPVHIAETAKSLETFERIVGEFDAFGLVRTEPVLV
VGGGLTTDVAGFACASYRRNTPYIRIPTTLIGLIDASVSIKVAVNYGKHKNRLGAYH
ASQKVLLDFSFLGTLPEDQVRNGMAELIKISVVGNLEIFEMLEQYGPELLRTRFGHLD
GTAELRSVADKLTYSAIATMLELEAPNLHEIDLDRVIAFGHTWSPTLELTPPAPFFHG
HAINIDMALSTTVAEQRGHLSTADRDRVLGVMSSIGLALDSPYLTPELLSEATASILK
TRDGILRAAVPDPIGTCRFLNDLDAAELADVLTLHKKICLDFPRAGEGLDMFTAPTP
A0A0K1S781
(SEQ ID NO: 107)
MAGIKATFTSTDCAFHIQGYEKIDFSLLYVNGAFKIGNPEIAESYAPFRRCLMVIDQT
VYGLYRQQIDQYFAHYQIDLTVFQVSIKEPEKTLRTFEKIVDAFADFGLVRKEPVLVV
GGGLTTDVAGFACSAYRRKTNYIRVPTSLIGLIDASVAIKVAVNHGKLKNRLGAYHA
SQKVILDFSFLGTLPIDQIRNGMAELIKIAVVGNQEIFELLEEHGAALLHSRFGYLNGT
PELQAVGHRLTYKAIQAMLELEVPNLHELDLDRVIAYGHTWSPTLELTPEPPMLHGH
SVNIDMAFTATIAQLRGYISVEDRNRILGLMSRLGLAIDSPYLTPELLWKATEAITRTR
DGLQRAAAPRPIGQCVFMNDLTRSELDKALAVHRAIAQNYPRQGNGEDMYVRLEP
ALEGAGV
A0A0P4UW20
(SEQ ID NO: 108)
MSSVQAKVEVTDQSFHLEGYEKIEFNLDLIEGLFEVGNSGLADNYRTLGRCLAVVDH
NVDRLYGDQLRSYFEYYEIDLTVFAIEITEPTKTIDTFLKITDAFCDFNLKRKEPVLVIG
GGLVLDVAGFACSAYRRSTNYIRVPSTLIGLIDAGVAIKVAVNHGKLKNRLGAYHPP
KQVILDFSFLKTLPVDQIRNGMAELVKIAVVSNEEVENLLEQHGEELLYNHFGFVGN
DAELKQIGHRVNYESIKTMLELEAPNLHELMLDRVIAYGHTWSPTLELAPQIPLLHG
HAVNIDMAISATIAEKRGYISALDRDRILGLMSRLGLALDHPLMEIDLMWKATQSIM
LTRDGFLRAAMPRPIGTCYFVNDLTREELESAIADHKRLCADYPRAGAGIDAYVGSS
ELIGSAN
A0A1Q8JXW2
(SEQ ID NO: 109)
MSNPQAVLSATDTEFRVESWERIEFTLSYVDGVFAPHNTELADLYRPWGRCLMVIDE
TVHEHYGDPIRSYFDHHDIAVTLVPLTIAETAKSLRTLERIVDAYADFGLLRTEPVLV
VGGGLTTDVTGFACASYKRGTPYVRIPTTLIGLIDASVAMKVAVNHGRHKNRLGAF
HASQQVLLDFSFLATLPEAQVRNGVAEMIKIATVANAGLFDLLEKYGDDLLATRFGH
REGTPELRQIAHRCTYDAIHTMLELEHRNLHELDLDRVIAFGHTWSPTLELAPPTPML
HGHAIAIDMAFSATLAARRGDITTGERDRIHRLFSGLGLSVDSTYLTEQLLIDATASIM
QTRAGKLRAALPRPIGTCHFANDIEHTELIETLAAHKAVVAGLPTSVEGVEMWSSAK
TELTTAPNTEART
A0A347Q3N8
(SEQ ID NO: 110)
MTTNLTATVTATENDERVRAVEERDYLLTYVDGAFSPESSRIADHHRAHGRCLMIV
DANVHRLHGDRIRAYFEHHGIALTALPLAIDETQKSLRTVERIVDAFGEFGLIRKEPV
LVVGGGLLTDVAGFACAVFRRSTDYVRVPTSLIGLIDASVAIKVAVNHGRTKNRLGA
FHASKEVVLDFSFLGTLPTEQVRNGMAELVKIAVVANAEVFRLLEKYGEDLLHTAFG
TVDGTPQLRETARKVTHEAIGTMLALEAPNLRELDLDRAIAFGHTWSPALELAPETP
YLHGHAISVDMALSCTIAERRGYLATSERDRIFWLLSKVGLSLDSPHLTPELLRAATE
SIVQTRDGLQRAAMPRPIGTCCFVNDLTESELLDGLAAHRELVARYPRGGAGEDVRV
TRSGAA
A0A6J4VHE9
(SEQ ID NO: 111)
MSTVQAKFEATETAFHVEGYEKIDFSLVFVNGAFDTKNRELADSYRNFGRCLAVVD
ANVNRLYGSQICEYFKYYNIDLNLFPVTISEPTKNLDTFQSIVDAFADFGLVRKEPVLI
VGGGLVTDVAGFACAAYRRSTNYIRIPTTLISLVDAGIAIKVAVNHGKLKNRLGAYH
APKKVMLDFSFLRTLPTPEVRNGMAELVKIAVVSNVEVFELLCEYGADLLTTHFGFD
GGTPLLKEVAHRINYESIKTMLALETPNLHELDLDRVIAYGHTWSPTLELAPSVPLLH
GHAVNIDMALSATIAEKRGYITVEERDRILGLMSQLGLALDHPLLDIDLLWSATQSIT
LTRDGLQRAAMPRPIGKCFFVNDLTREELDAALAEHKHACAQYPRAGAGVDAYVG
SYQQQNLIEGIANV

In some embodiments, the one or more biosynthetic enzymes comprise an O-methyltransferase (MysB), or a homolog thereof. Exemplary MysB enzymes for use in the present invention include, but are not limited to, the amino acid sequence of SEQ ID NO: 112, or an amino acid sequence at least 70%, at least 75%, at least 80%, at least 85%, at least 90%, at least 95%, or at least 99% identical to the amino acid sequence of SEQ ID NO: 112:

A0A1Z4LFB8
(SEQ ID NO: 112)
MSTTIAKPTARPVTPVGILAKKLEAIVQKINQRTDLPADLVDNITQAWQLAAGLDPY
LEEYTTSESSALTALAEKTSTEAWQEHFSEGTTVRPLEQEMLSGHVEGQTLKMFVH
MTKAKRVLEIGMFTGYSALAMAEALPPDGVLVACEVDPFAAEVGQAAFDKSPDGK
KIRVELGPALETLNKLVEAGESFDMVFIDADKKEYITYFQTLLDTNLLAPSGFICVDN
TLLQGEVYLPTQQRTANGEAIAQFNRAVALDPRVEQVILPLRDGLTIIRRTA

In some embodiments, the one or more biosynthetic enzymes comprise a non-ribosomal peptide synthetase (NRPS)-like enzyme (MysE), or a homolog thereof. In certain embodiments, the one or more biosynthetic enzymes comprises an enzyme with an amino acid sequence at least 70%, at least 75%, at least 80%, at least 85%, at least 90%, at least 95%, or at least 99% identical to the amino acid sequence of a MysE enzyme, or a homolog thereof.

Compounds of varying structures can be produced using the methods of the present invention. In some embodiments, the compound is a palythine analog. In certain embodiments, the compound has UV-modulating activity. For example, the compounds of the present invention may absorb UV wavelengths between 310 nm and 362 nm. In certain embodiments, the compound is a compound of Formula (I), or a salt thereof:

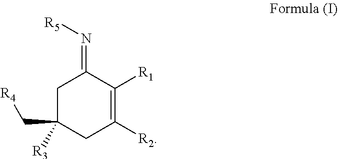

In the compounds of Formula (I) described herein, each of R₁, R₂, R₃, and R₄ may independently be selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl. In some embodiments, R₁ is —OR^{a}, wherein R^{a} is optionally substituted C₁₋₆ alkyl. In certain embodiments, R₁ is —OCH₃. In some embodiments, R₂ is —NH₂. In certain embodiments, R₃ is —OH. In some embodiments, R₄ is —OH. In some embodiments, R₁ is —OCH₃, R₂ is —NH₂, R₃ is —OH, and R₄ is —OH.

The compounds of Formula (I) described herein also include a moiety R₅. R₅ may be any natural or non-natural amino acid, or a derivative thereof. In certain embodiments, R₅ is threonine. In certain embodiments, R₅ is serine. In certain embodiments, R₅ is isoleucine. In certain embodiments, R₅ is methionine. In certain embodiments, R₅ is valine. In some embodiments, R₁ is —OCH₃, R₂ is —NH₂, R₃ is —OH, R₄ is —OH, and R₅ is threonine. In some embodiments, R₁ is —OCH₃, R₂ is —NH₂, R₃ is —OH, R₄ is —OH, and R₅ is serine. In some embodiments, R₁ is —OCH₃, R₂ is —NH₂, R₃ is —OH, R₄ is —OH, and R₅ is isoleucine. In some embodiments, R₁ is —OCH₃, R₂ is —NH₂, R₃ is —OH, R₄ is —OH, and R₅ is methionine. In some embodiments, R₁ is —OCH₃, R₂ is —NH₂, R₃ is —OH, R₄ is —OH, and R₅ is valine.

In some embodiments, the compound of Formula (I) is of the formula:

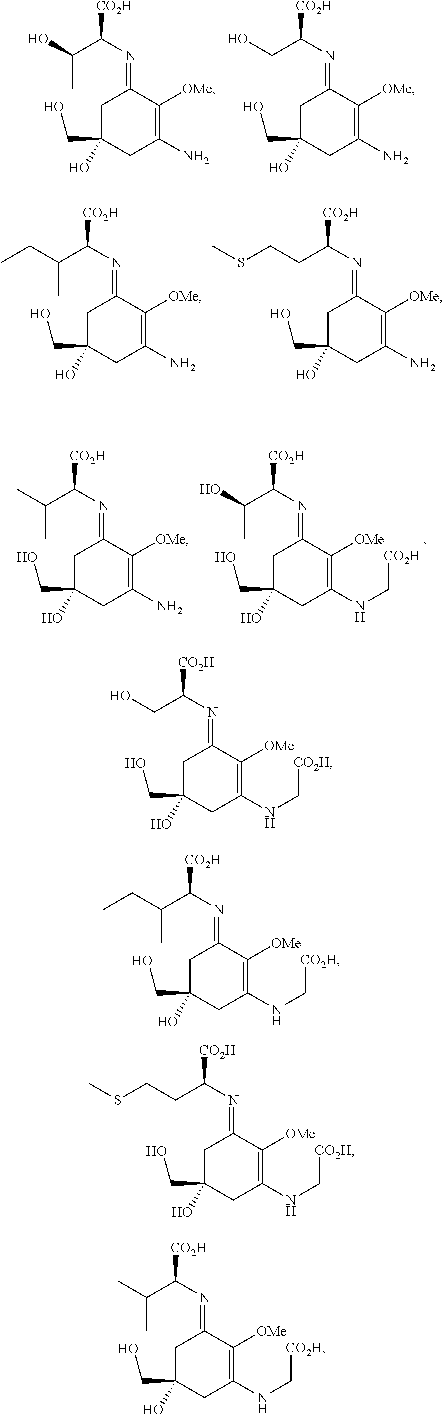

or a salt thereof.
In certain embodiments, the compound of Formula (I) is not

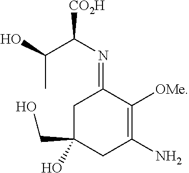 [SMILES: COC1=C(N)C[C@@](O)(CO)C/C1=N\[C@H](C(=O)O)[C@@H](C)O]

In certain embodiments, the compound of Formula (I) is not

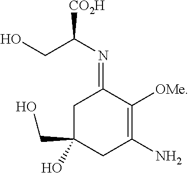 [SMILES: COC1=C(N)C[C@@](O)(CO)C/C1=N\[C@@H](CO)C(=O)O]

In certain embodiments, the compound of Formula (I) is not

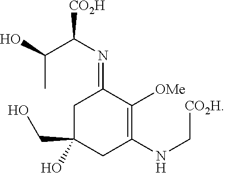 [SMILES: COC1=C(NCC(=O)O)C[C@@](O)(CO)C/C1=N\[C@H](C(=O)O)[C@@H](C)O]

In certain embodiments, the compound of Formula (I) is not

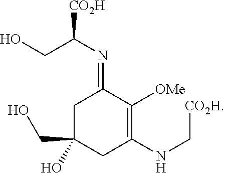 [SMILES: COC1=C(NCC(=O)O)C[C@@](O)(CO)C/C1=N\[C@@H](CO)C(=O)O]

In certain embodiments, the compound of Formula (I) is not

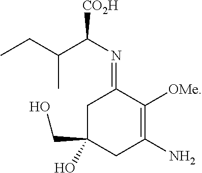 [SMILES: CCC(C)[C@H](/N=C1\C[C@](O)(CO)CC(N)=C1OC)C(=O)O]

In certain embodiments, the compound of Formula (I) is not

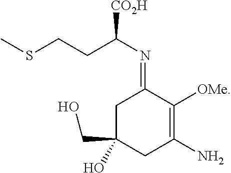 [SMILES: COC1=C(N)C[C@@](O)(CO)C/C1=N\[C@@H](CCSC)C(=O)O]

In certain embodiments, the compound of Formula (I) is not

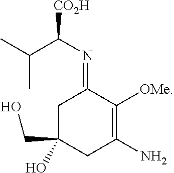 [SMILES: COC1=C(N)C[C@@](O)(CO)C/C1=N\[C@H](C(=O)O)C(C)C]

In certain embodiments, the compound of Formula (I) is not

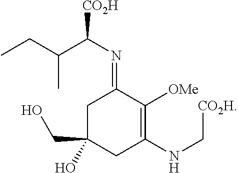 [SMILES: CCC(C)[C@H](/N=C1\C[C@](O)(CO)CC(NCC(=O)O)=C1OC)C(=O)O]

In certain embodiments, the compound of Formula (I) is not

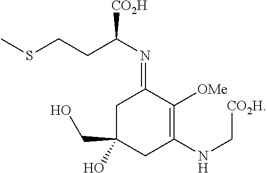 [SMILES: COC1=C(NCC(=O)O)C[C@@](O)(CO)C/C1=N\[C@@H](CCSC)C(=O)O]

In certain embodiments, the compound of Formula (I) is not

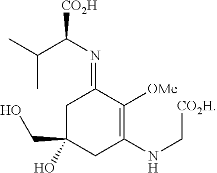 [SMILES: COC1=C(NCC(=O)O)C[C@@](O)(CO)C/C1=N\[C@H](C(=O)O)C(C)C]

In some embodiments, the compound produced by the methods described herein is of the formula:

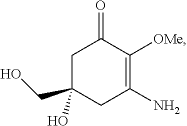 [SMILES: COC1=C(N)C[C@@](O)(CO)CC1=O]

or a salt thereof.

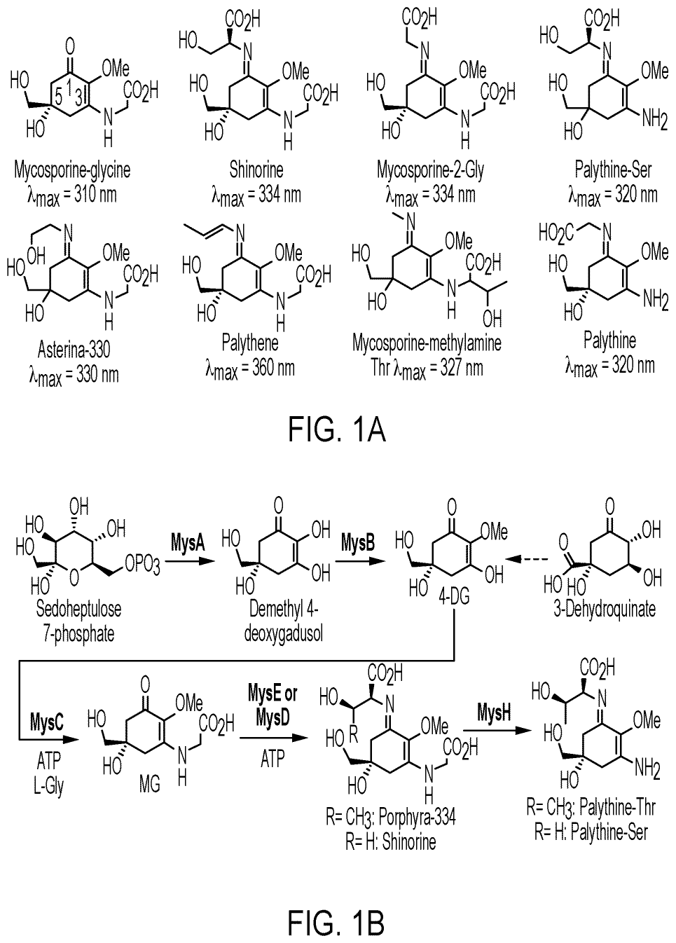

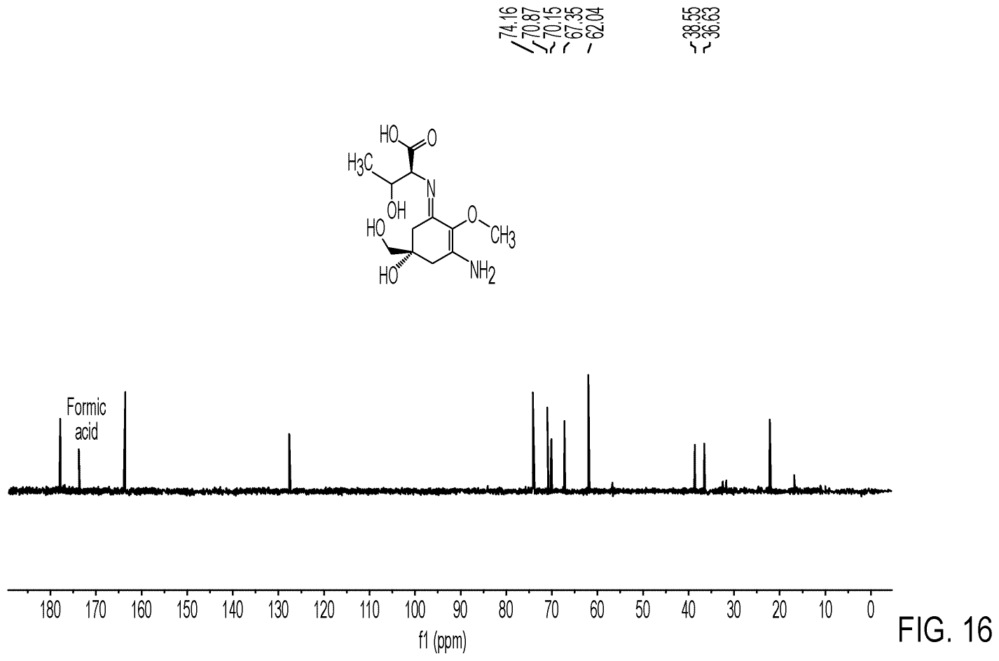

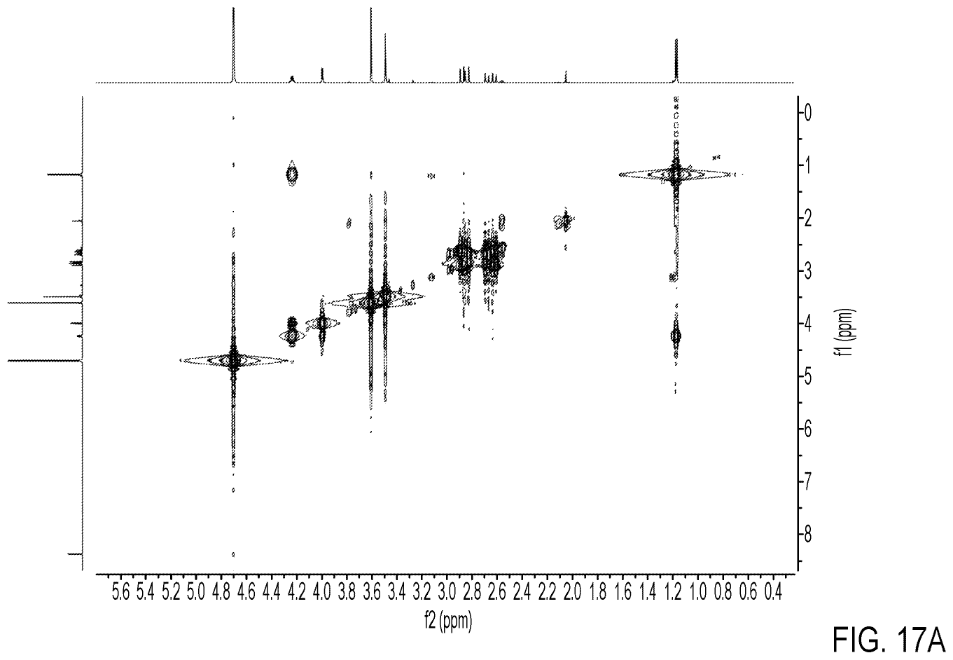

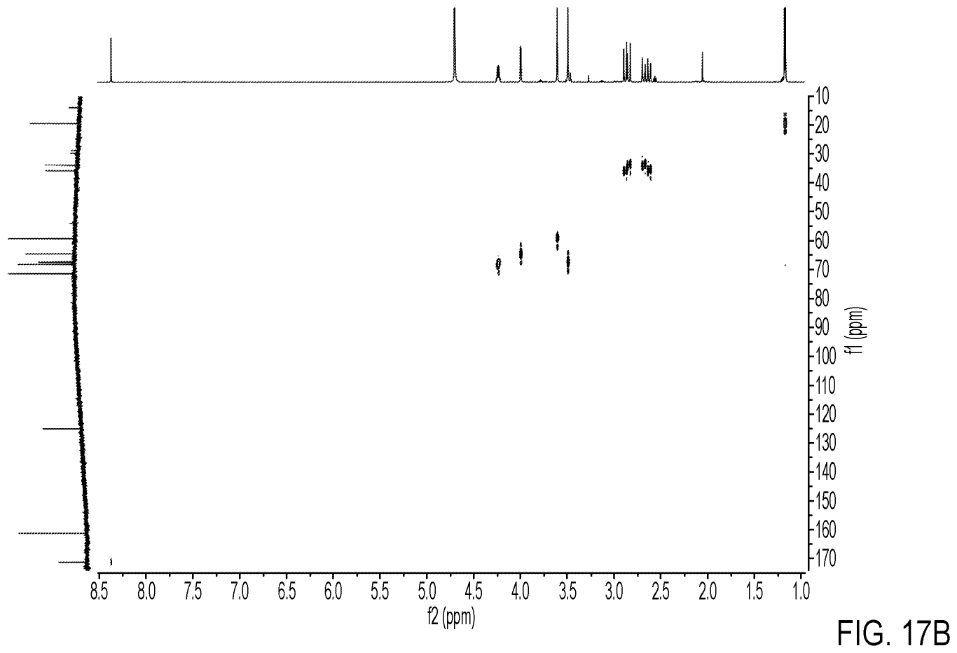

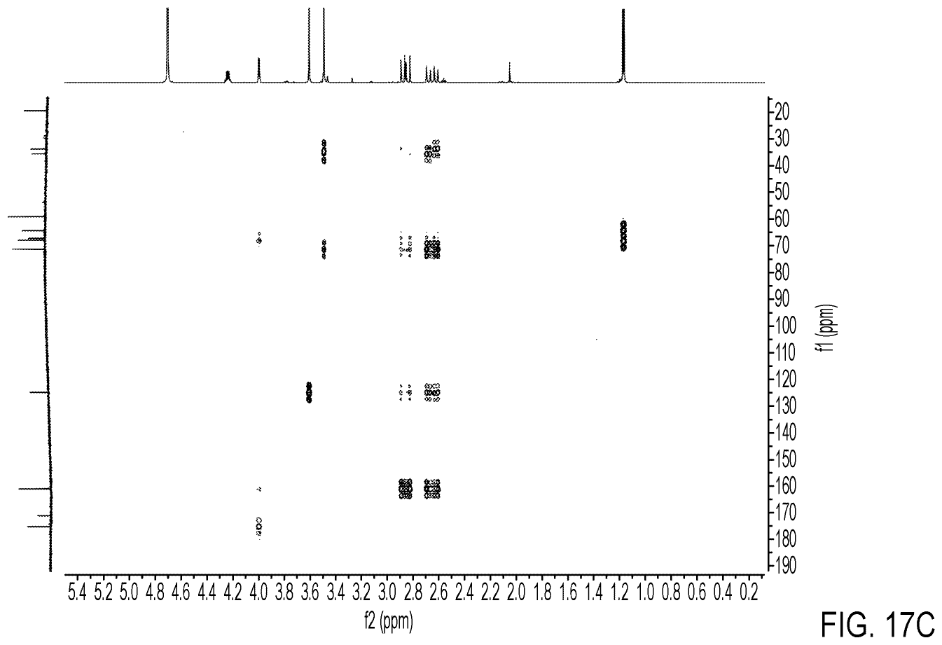

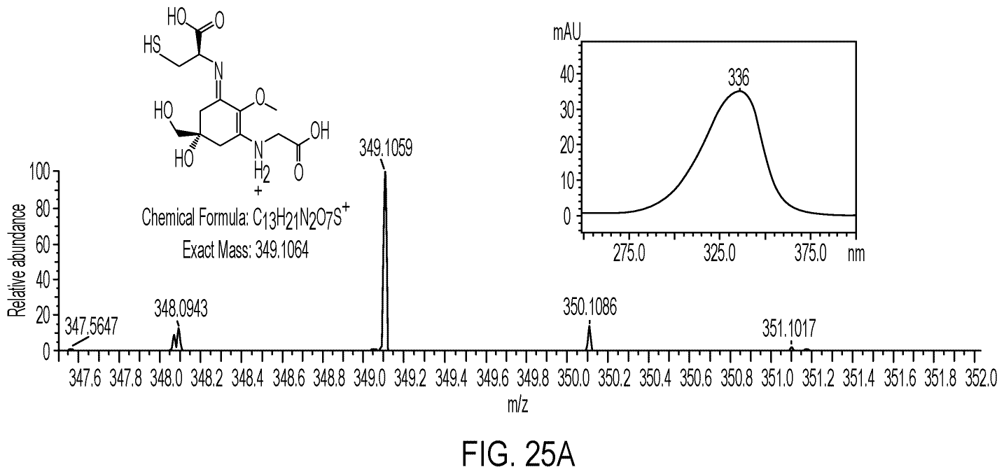

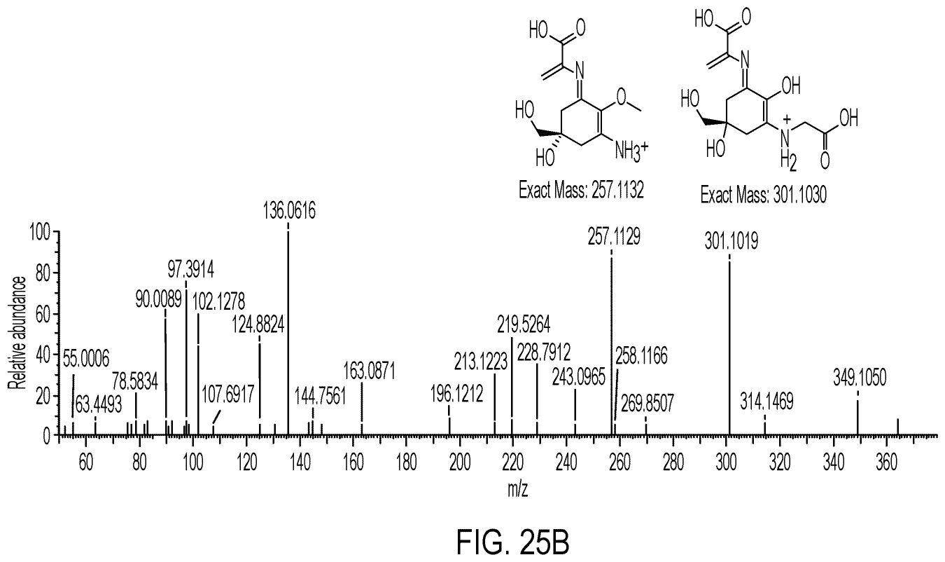

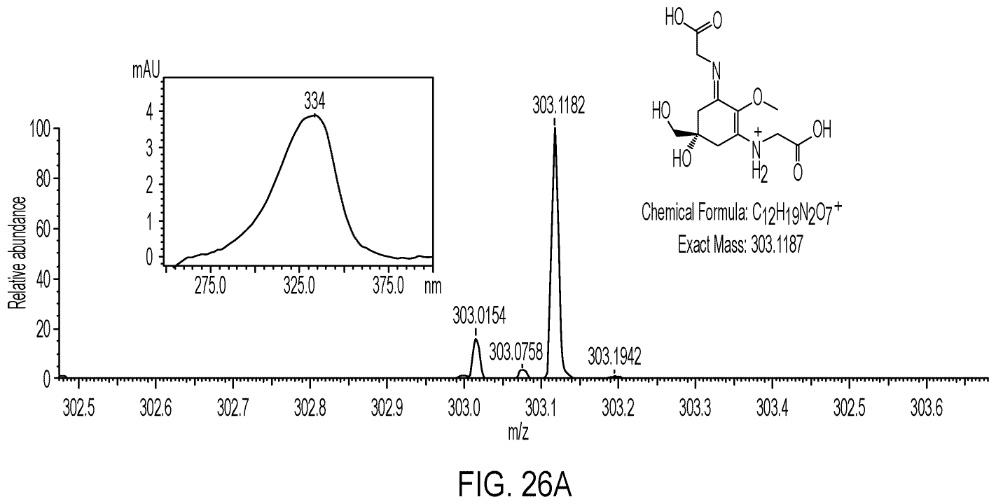

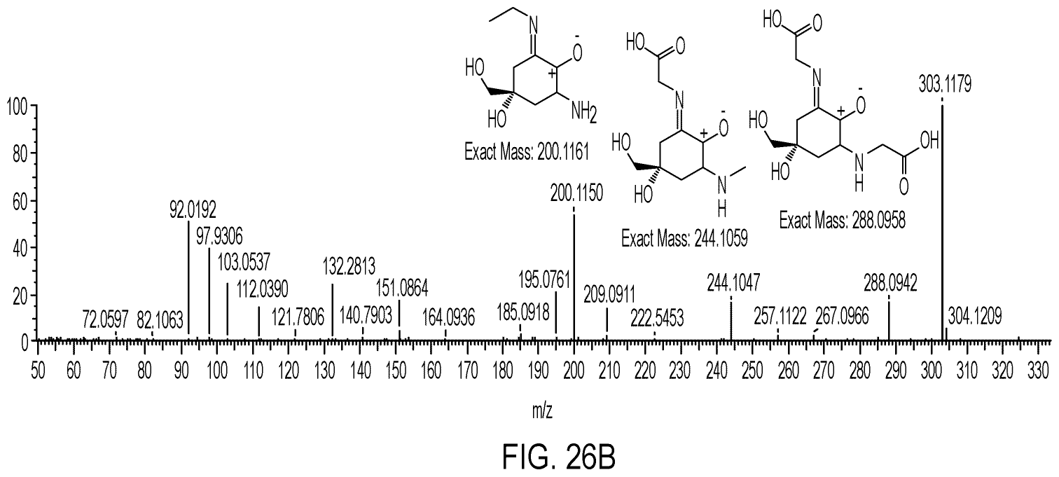

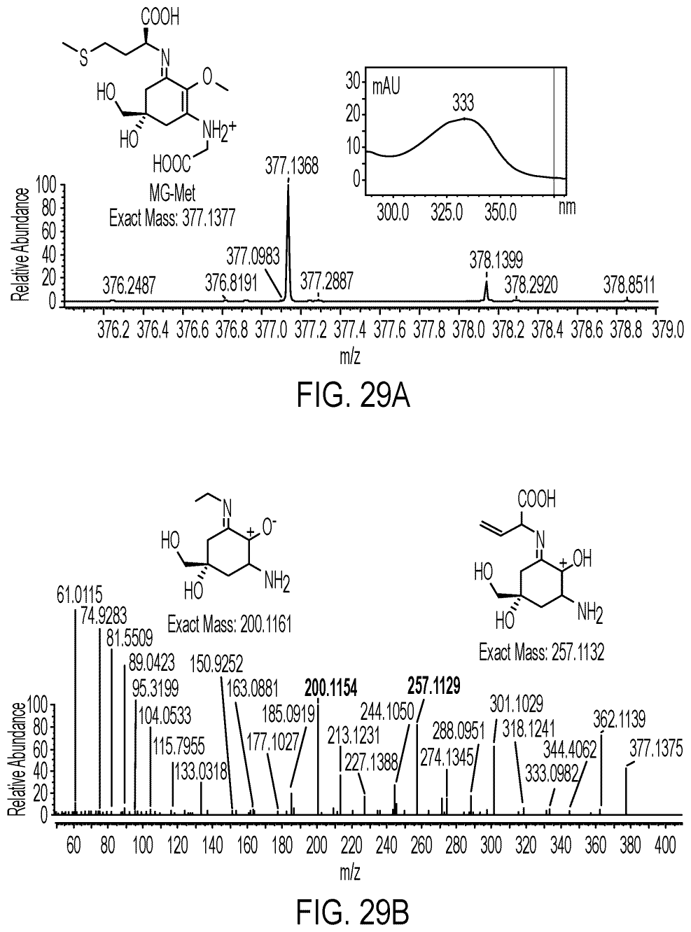

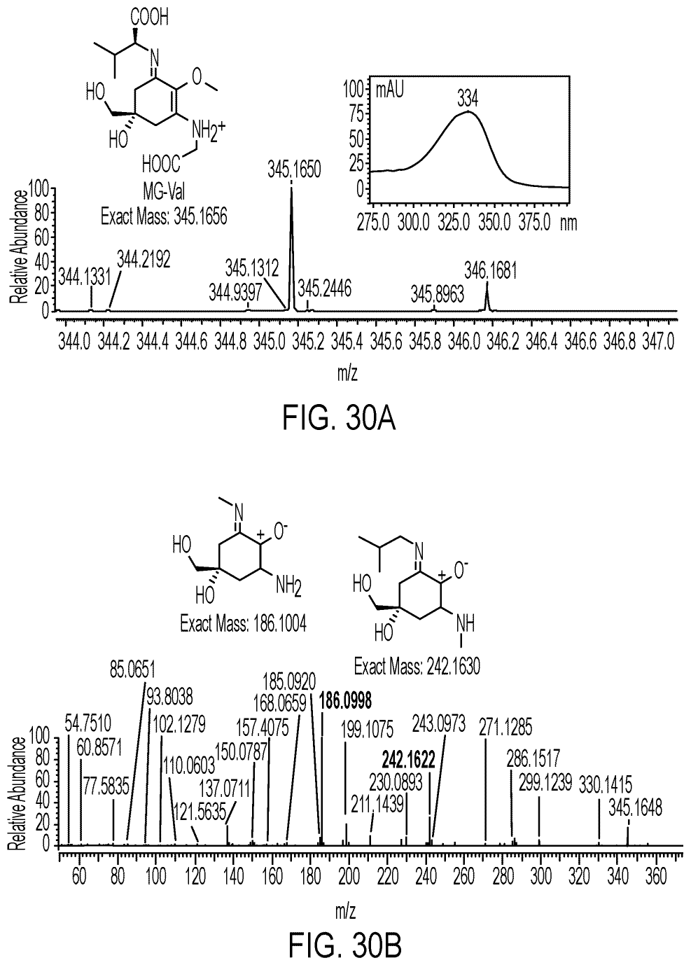

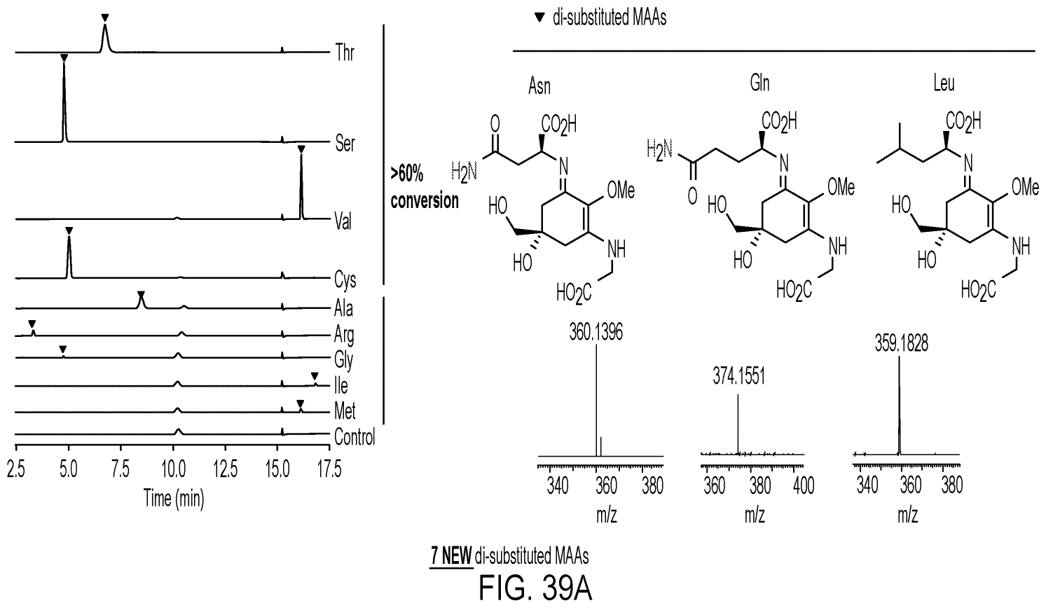

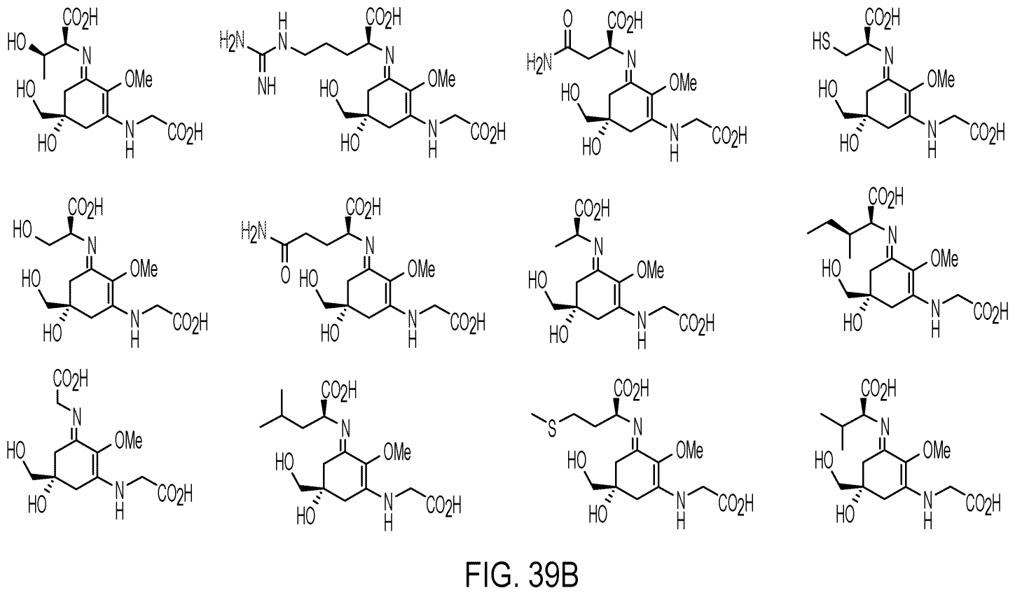

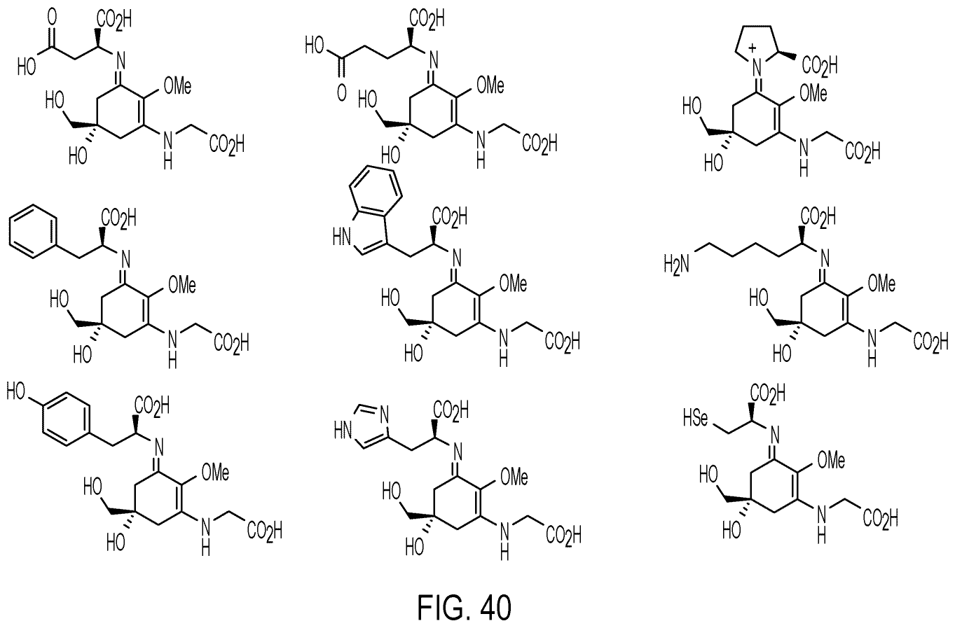

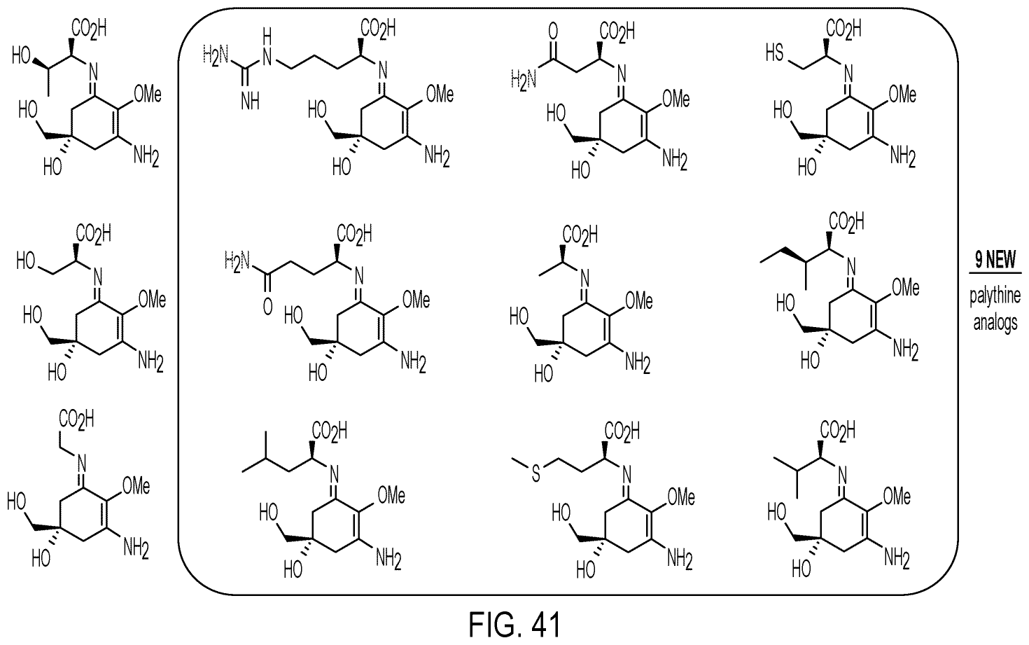

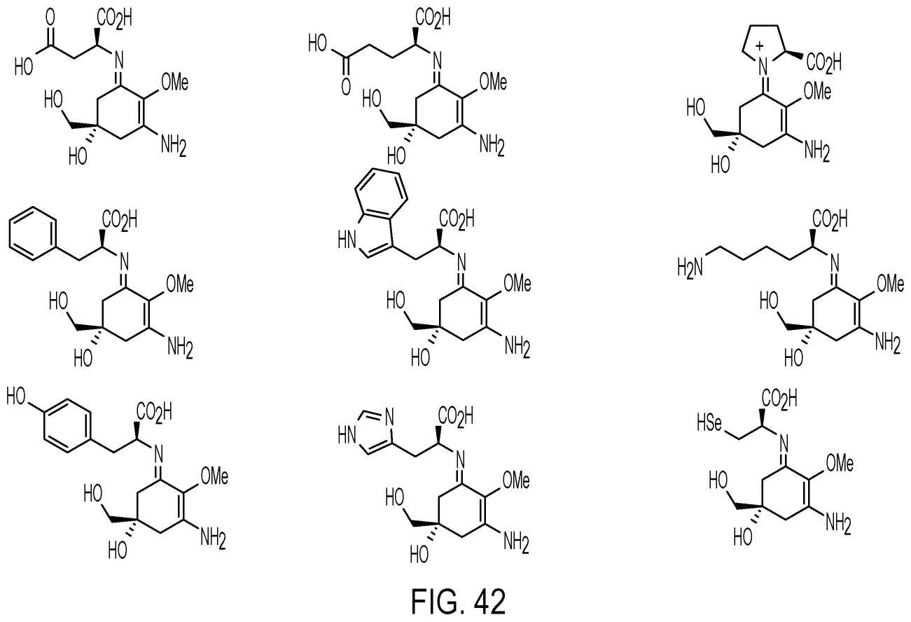

In certain embodiments, the compound of Formula (I) is a compound delineated herein, including in any of the figures herein. In certain embodiments, the compound of Formula (I) is a compound of any of FIGS. 1-42. In certain embodiments, the compound of Formula (I) is a compound of any of FIGS. 39-42.

The methods disclosed herein may further comprise providing a substrate of one of the MAA biosynthetic enzymes to the recombinant microorganism. In some embodiments, the substrate is a compound of Formula (II), or a salt thereof:

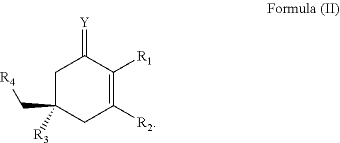

In the compounds of Formula (II) described herein, each of R₁, R₂, R₃, and R₄ may independently be selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl. In certain embodiments, R₁ is —OH. In certain embodiments, R₁ is —OCH₃. In some embodiments, R₂ is —OH. In certain embodiments, R² is —NH₂. In some embodiments, R₂ is —(NH)R^{b}, wherein R^{b} is optionally substituted alkyl. In certain embodiments, R₂ is —NHCH₂CO₂H. In some embodiments, R₃ is —OH. In some embodiments, R₄ is —OH. In some embodiments, R₁ is —OCH₃, R₂ is —(NH)R^{b}, R₃ is —OH, and R₄ is —OH. In some embodiments, R₁ is —OCH₃, R₂ is —NH₂, R₃ is —OH, and R₄ is —OH. In some embodiments, R₁ is —OH, R₂ is —OH, R₃ is —OH, and R₄ is —OH. In some embodiments, R₁ is —OCH₃, R₂ is —OH, R₃ is —OH, and R₄ is —OH.

The compounds of Formula (II) described herein also include a moiety Y. Y may be O or NR₅, wherein R₅ is optionally substituted C₁₋₆ alkyl, optionally substituted C₁₋₆ alkenyl, or an amino acid (e.g., any natural or non-natural amino acid, or a derivative thereof). In certain embodiments, Y is O. In some embodiments, Y is NR₅. In certain embodiments, Y is NR₅ and R₅ is threonine. In certain embodiments, Y is NR₅ and R₅ is serine. In certain embodiments, Y is NR₅ and R₅ is isoleucine. In certain embodiments, Y is NR₅ and R₅ is methionine. In certain embodiments, Y is NR₅ and R₅ is valine.

In some embodiments, the substrate is a compound of the formula:

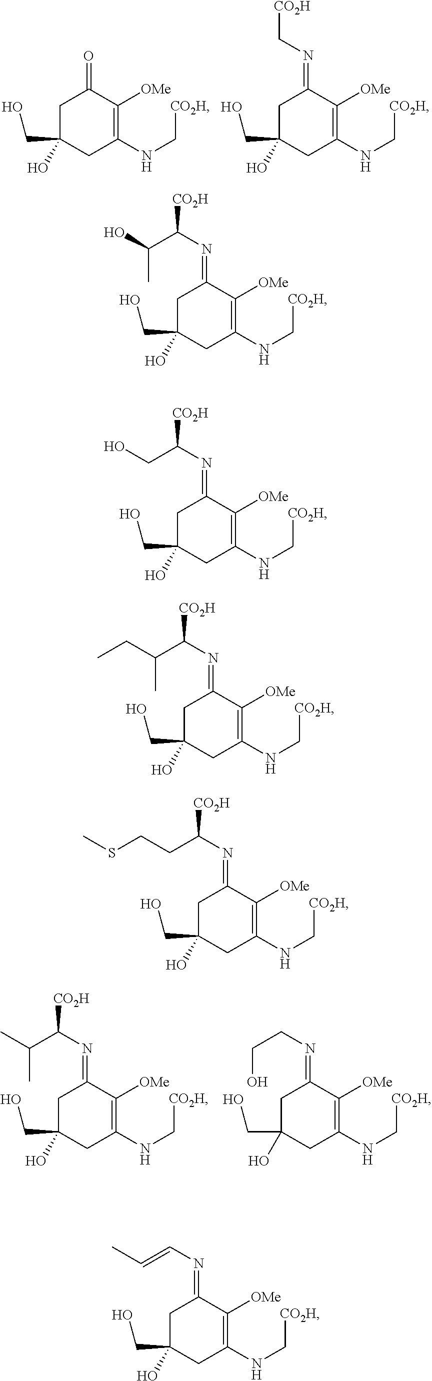

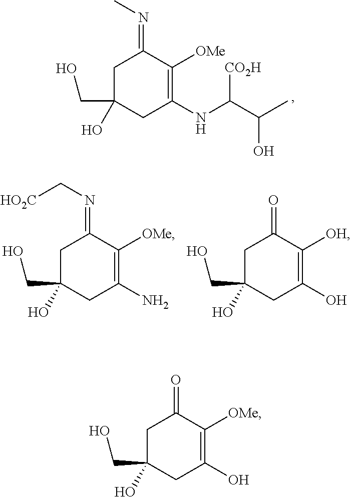 [SMILES: C/N=C1\CC(O)(CO)CC(NC(C(=O)O)C(C)O)=C1OC ; "," ; COC1=C(N)C[C@@](O)(CO)C/C1=N\CC(=O)O ; O=C1C[C@](O)(CO)CC(O)=C1O ; COC1=C(O)C[C@@](O)(CO)CC1=O]

or a salt thereof. In certain embodiments, the substrate is not a compound of the formula

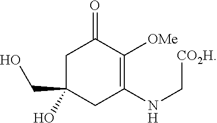 [SMILES: COC1=C(NCC(=O)O)C[C@@](O)(CO)CC1=O]

In certain embodiments, the substrate is not a compound of the formula

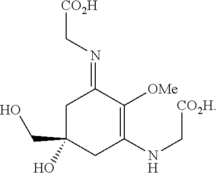 [SMILES: COC1=C(NCC(=O)O)C[C@@](O)(CO)C/C1=N\CC(=O)O]

In certain embodiments, the substrate is not a compound of the formula

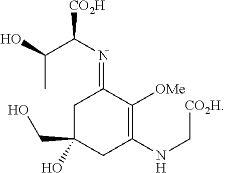 [SMILES: COC1=C(NCC(=O)O)C[C@@](O)(CO)C/C1=N\[C@H](C(=O)O)[C@@H](C)O]

In certain embodiments, the substrate is not a compound of the formula

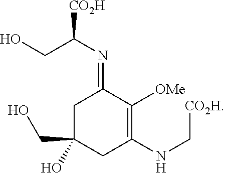 [SMILES: COC1=C(NCC(=O)O)C[C@@](O)(CO)C/C1=N\[C@@H](CO)C(=O)O]

In certain embodiments, the substrate is not a compound of the formula

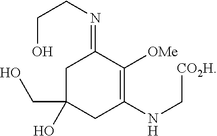 [SMILES: COC1=C(NCC(=O)O)CC(O)(CO)C/C1=N\CCO]

In certain embodiments, the substrate is not a compound of the formula

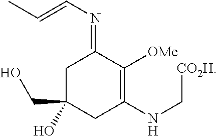 [SMILES: C/C=C/N=C1\C[C@](O)(CO)CC(NCC(=O)O)=C1OC]

In certain embodiments, the substrate is not a compound of the formula

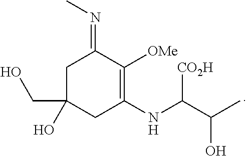 [SMILES: C/N=C1\CC(O)(CO)CC(NC(C(=O)O)C(C)O)=C1OC]

In certain embodiments, the substrate is not a compound of the formula

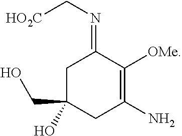 [SMILES: COC1=C(N)C[C@@](O)(CO)C/C1=N\CC(=O)O]

In certain embodiments, the substrate is not a compound of the formula

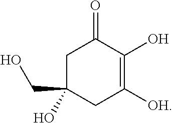 [SMILES: O=C1C[C@](O)(CO)CC(O)=C1O]

In certain embodiments, the substrate is not a compound of the formula

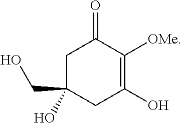 [SMILES: COC1=C(O)C[C@@](O)(CO)CC1=O]

In certain embodiments, the substrate is not a compound of the formula

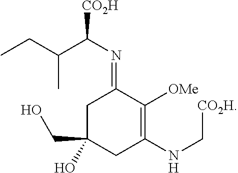 [SMILES: CCC(C)[C@H](/N=C1\C[C@](O)(CO)CC(NCC(=O)O)=C1OC)C(=O)O]

In certain embodiments, the substrate is not a compound of the formula

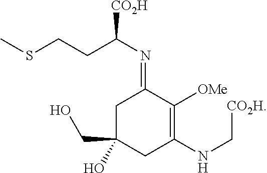 [SMILES: COC1=C(NCC(=O)O)C[C@@](O)(CO)C/C1=N\[C@@H](CCSC)C(=O)O]

In certain embodiments, the substrate is not a compound of the formula

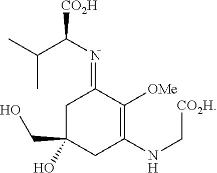 [SMILES: COC1=C(NCC(=O)O)C[C@@](O)(CO)C/C1=N\[C@H](C(=O)O)C(C)C]

In certain embodiments, the substrate is a compound delineated herein, including in any of the figures herein. In certain embodiments, the substrate is a compound of any of FIGS. 1-42. In certain embodiments, the substrate is a compound of any of FIGS. 39-42.

In some embodiments, the methods described herein further comprise producing a glycosylated MAA. In certain embodiments, the one or more MAA biosynthetic enzymes encoded by the microorganism further comprise a glycosyltransferase (GlyT), or a homolog thereof.

Any suitable microorganism that can be genetically manipulated (e.g., genomically engineered, or transformed with a suitable vector to express a heterologous gene) may be used in the methods of the present invention. For example, the recombinant microorganism may be a species of bacteria or yeast. In some embodiments, the recombinant microorganism is a species of cyanobacteria. In some embodiments, the recombinant microorganism is a species of bacteria from the human microbiome (e.g., including, but not limited to, any of the species listed herein). In certain embodiments, the recombinant microorganism is E. coli.

The present disclosure also encompasses recombinant microorganisms for use in performing the methods of the present invention. For instance, in one aspect the present disclosure includes recombinant microorganisms comprising a heterologous nucleic acid encoding one or more MAA biosynthetic enzymes, wherein the one or more MAA biosynthetic enzymes comprise a phytanoyl-CoA dioxygenase (MysH), or a homolog thereof. In another aspect, the present disclosure provides methods of producing a compound, comprising culturing such a recombinant microorganism under conditions suitable for production of the compound and isolating the compound from the recombinant microorganism.

### Compositions

In one aspect, the present disclosure provides compositions comprising a compound produced by the methods of the present invention (e.g., a compound of Formula (I), or a salt thereof). In some embodiments, the composition optionally comprises one or more suitable excipients. In certain embodiments, the compositions described herein comprise a compound of Formula (I), or a salt thereof, and an excipient.

In certain embodiments, the compound described herein is provided in an effective amount in the composition. In certain embodiments, the effective amount is a therapeutically effective amount. In certain embodiments, the effective amount is a prophylactically effective amount. In certain embodiments, the compound is provided in an amount effective for preventing sunburn in a subject. In certain embodiments, the compound is provided in an amount effective for preventing cancer (e.g., skin cancer) in the subject. In certain embodiments, the compound is provided in an amount effective for treating or preventing a chronic inflammatory disease or condition in a subject in need thereof. In certain embodiments, the effective amount is an amount effective for reducing symptoms (e.g., symptoms of sunburn) by at least about 10%, at least about 20%, at least about 30%, at least about 40%, at least about 50%, at least about 60%, at least about 70%, at least about 80%, at least about 90%, at least about 95%, or at least about 98%.

Compositions described herein can be prepared by any method known in the art. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit, or into a formulation for topical administration.

Relative amounts of the active ingredient, the excipient, and/or any additional ingredients in a composition described herein will vary. The composition may comprise between 0.1% and 100% (w/w) active ingredient.

Excipients used in the manufacture of the provided compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Dosage forms for topical and/or transdermal administration of a compound produced by the methods described herein may include ointments, pastes, creams, lotions, gels, powders, solutions, sprays, inhalants, and/or patches. Generally, the active ingredient is admixed under sterile conditions with an acceptable carrier or excipient and/or any needed preservatives and/or buffers as can be required. In some embodiments, the composition for topical administration is formulated as a sunscreen. In certain embodiments, the composition for topical administration is formulated as a cosmetic.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

The compositions described herein may also comprise one or more additional active ingredients (e.g., additional compounds with UV-modulating, anti-inflammatory, and/or anti-oxidative activity). In certain embodiments, a composition described herein including a compound described herein and an additional active ingredient shows a synergistic effect (e.g., improved prevention of sunburn in a subject) that is absent in a composition including either the compound or the additional active ingredient, but not both.

Thus, in one aspect, the present disclosure contemplates compositions comprising a compound produced by any of the methods of the present invention and optionally an excipient. In some embodiments, the composition is for topical administration. In certain embodiments, the composition is formulated as a sunscreen. In certain embodiments, the composition is formulated as a cosmetic (e.g., make-up, concealer, a moisturizer, etc.). In another aspect, the present disclosure provides methods of making a composition as described herein, comprising culturing a recombinant microorganism under conditions suitable for production of a compound, as described herein, and isolating the compound from the recombinant microorganism, wherein the recombinant microorganism comprises a heterologous nucleic acid encoding one or more MAA biosynthetic enzymes, wherein the one or more MAA biosynthetic enzymes comprise a phytanoyl-CoA dioxygenase (MysH), or a homolog thereof, and adding the compound to one or more excipients to produce the composition.

### Methods of Prevention and Treatment

In another aspect, the present disclosure includes methods of administering a compound (e.g., any of the compounds disclosed herein). In some embodiments, a method of administering a compound comprises applying any of the compositions disclosed herein to a subject. In certain embodiments, the composition is applied on the skin of a subject in need thereof. In some embodiments, the method is a method preventing sunburn in a subject in need thereof.

In certain embodiments, the method is a method of preventing cancer in a subject in need thereof (e.g., skin cancers such as melanoma, basal cell carcinoma, or squamous cell carcinoma as described herein). MAAs and related compounds have utility as anti-cancer agents through their antioxidant and anti-proliferative activities (Mar. Drugs 2017, 15(10), 326). For example, the compounds of the present disclosure have UV-modulating activity and may prevent DNA damage in skin cells caused by UV radiation from the sun when applied to the skin in any of the compositions disclosed herein.

In certain embodiments, the method is a method of preventing or treating a chronic inflammatory disease in a subject in need thereof. For example, compounds of the present disclosure have anti-oxidative and anti-inflammatory activities and may prevent or alleviate symptoms of an inflammatory disease when applied to the skin in any of the compositions disclosed herein.

### Compounds

In another aspect, the present disclosure provides compounds produced by the methods of the present invention. In some embodiments, the present disclosure provides compounds produced by culturing a recombinant microorganism under conditions suitable for production of the compound and isolating the compound from the recombinant microorganism. In certain embodiments, the recombinant microorganism comprises a heterologous nucleic acid encoding one or more MAA biosynthetic enzymes, wherein the one or more MAA biosynthetic enzymes comprise a phytanoyl-CoA dioxygenase (MysH), or a homolog thereof. In some embodiments, the heterologous nucleic acid encodes additional MAA biosynthetic enzymes (e.g., MysA, MysB, MysC, MysD, and/or MysE, or homologs or variants thereof).

In some embodiments, the compound is a compound of Formula (I), or a salt thereof:

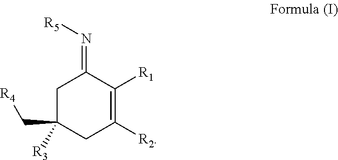

In the compounds of Formula (I) described herein, each of R₁, R₂, R₃, and R₄ may independently be selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl. In some embodiments, R₁ is —OR^{a}, wherein R^{a} is optionally substituted C₁₋₆ alkyl. In certain embodiments, R₁ is —OCH₃. In some embodiments, R₂ is —NH₂. In certain embodiments, R₃ is —OH. In some embodiments, R₄ is —OH.

The compounds of Formula (I) described herein also include a moiety R₅. R₅ may be any natural or non-natural amino acid, or a derivative thereof. In certain embodiments, R₅ is threonine. In certain embodiments, R₅ is serine. In certain embodiments, R₅ is isoleucine. In certain embodiments, R₅ is methionine. In certain embodiments, R₅ is valine.

In some embodiments, the compound of Formula (I) is of the formula:

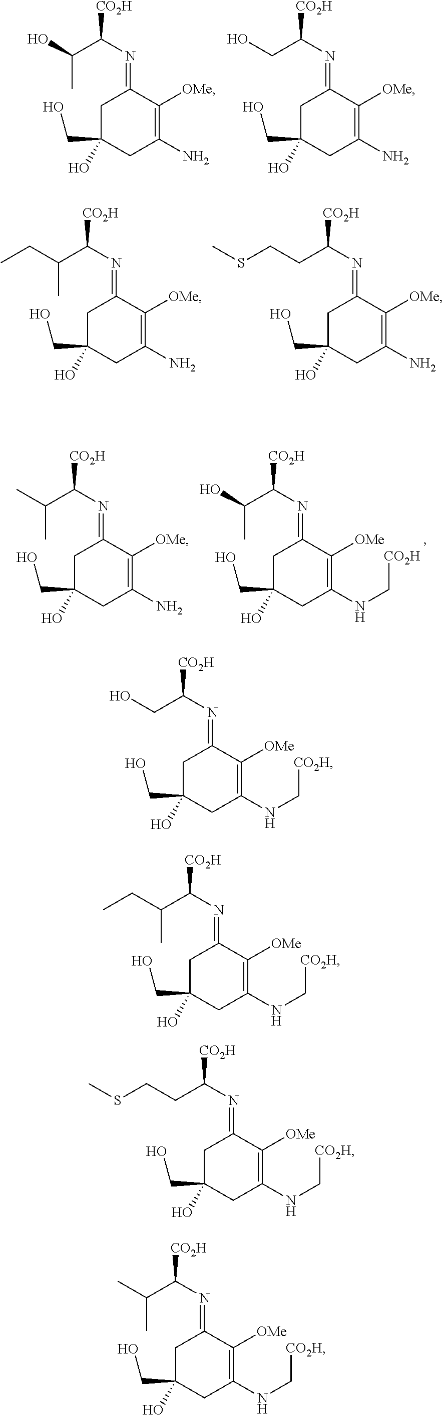

or a salt thereof. In certain embodiments, the compound of Formula (I) is not

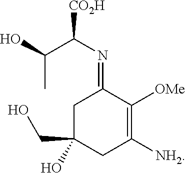 [SMILES: COC1=C(N)C[C@@](O)(CO)C/C1=N\[C@H](C(=O)O)[C@@H](C)O]

In certain embodiments, the compound of Formula (I) is not

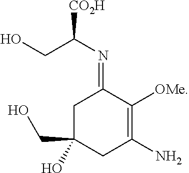 [SMILES: COC1=C(N)C[C@@](O)(CO)C/C1=N\[C@@H](CO)C(=O)O]

In certain embodiments, the compound of Formula (I) is not

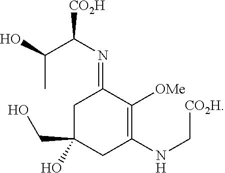 [SMILES: COC1=C(NCC(=O)O)C[C@@](O)(CO)C/C1=N\[C@H](C(=O)O)[C@@H](C)O]

In certain embodiments, the compound of Formula (I) is not

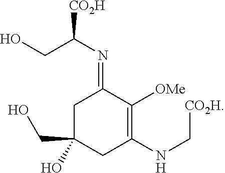 [SMILES: COC1=C(NCC(=O)O)C[C@@](O)(CO)C/C1=N\[C@@H](CO)C(=O)O]

In certain embodiments, the compound of Formula (I) is not

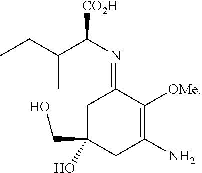 [SMILES: CCC(C)[C@H](/N=C1\C[C@](O)(CO)CC(N)=C1OC)C(=O)O]

In certain embodiments, the compound of Formula (I) is not

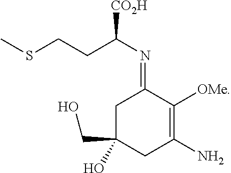 [SMILES: COC1=C(N)C[C@@](O)(CO)C/C1=N\[C@@H](CCSC)C(=O)O]

In certain embodiments, the compound of Formula (I) is not

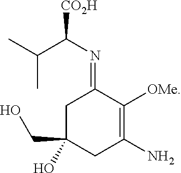 [SMILES: COC1=C(N)C[C@@](O)(CO)C/C1=N\[C@H](C(=O)O)C(C)C]

In certain embodiments, the compound of Formula (I) is not

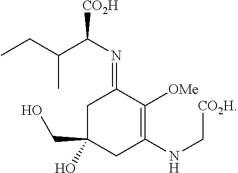 [SMILES: CCC(C)[C@H](/N=C1\C[C@](O)(CO)CC(NCC(=O)O)=C1OC)C(=O)O]

In certain embodiments, the compound of Formula (I) is not

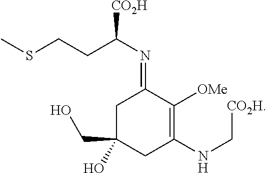 [SMILES: COC1=C(NCC(=O)O)C[C@@](O)(CO)C/C1=N\[C@@H](CCSC)C(=O)O]

In certain embodiments, the compound of Formula (I) is not

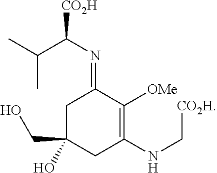 [SMILES: COC1=C(NCC(=O)O)C[C@@](O)(CO)C/C1=N\[C@H](C(=O)O)C(C)C]

In some embodiments the compound produced by the methods of the present disclosure is of the formula:

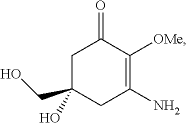 [SMILES: COC1=C(N)C[C@@](O)(CO)CC1=O]

or a salt thereof.

In certain embodiments, the compound of Formula (I) is a compound delineated herein, including in any of the figures herein. In certain embodiments, the compound of Formula (I) is a compound of any of FIGS. 1-42. In certain embodiments, the compound of Formula (I) is a compound of any of FIGS. 39-42.

In some embodiments, a compound of the present invention, or a salt thereof, is provided in a composition (e.g., in any of the forms disclosed herein). In some embodiments, the composition is for topical administration. In certain embodiments, the composition is formulated as a sunscreen. In certain embodiments, the composition is formulated as a cosmetic.

In one aspect, the present disclosure provides methods of administering the compounds of the present invention comprising applying any of the compositions disclosed herein to a subject. In some embodiments, the composition is applied on the skin of a subject. In certain embodiments, the composition is applied on the skin of a subject in need thereof as a method of preventing sunburn (e.g., when the composition is formulated as a sunscreen). In certain embodiments, the composition is applied on the skin of a subject in need thereof as a method of preventing cancer. In certain embodiments, the composition is applied on the skin of a subject in need thereof as a method of treating or preventing a chronic inflammatory disease.

### EXAMPLES

Mycosporine-like amino acids (MAAs) are a family of natural, thermally and photochemically stable UV protectants (FIG. 1A).¹⁶ Originally isolated from terrestrial fungal species, over 30 MAA analogs have been identified from taxonomically diverse marine and terrestrial organisms (e.g., cyanobacteria, eukaryotic algae, corals, plants, and vertebrates) and possess various functional groups at the C1 and, to a lesser extent, the C3 of the characteristic cyclohexenimine core (FIG. 1A).¹⁶⁻¹⁸ Indeed, the majority of MAAs carry a C3-L-Gly moiety, though L-Ala, L-Glu, and other amine-containing components also appear. Common amino acid building blocks at the C1 include L-Ser (shinorine), L-Thr (porphyra-334), L-Gly (mycosporine-2-Gly) and L-Ala.¹⁶⁻¹⁸ These moieties at the C1 and C3 can likely be converted into other functional groups, including amino alcohol (e.g., asterina-330), enaminone (e.g., palythene), methyl amine (e.g., mycosporine-methylamine-Thr), or an amine group (e.g., palythine and palythine-Ser),^{17,19} while glycosylated MAAs have been produced in a variety of organisms.^{20,21} Of note, except a few analogs (e.g., mycosporine-glycine, porphyra-334, palythene and palythine),²²⁻²⁵ the absolute configuration of the majority of MAAs, particularly the C5, has not been fully elucidated. Despite notable structural diversity, these MAA analogs display absorption maxima between 310 and 362 nm and possess extinction coefficients of up to 50,000 M⁻¹·cm⁻¹.¹⁶⁻¹⁷ They are among the strongest UV absorbing compounds, and the cyclohexenimine core is critical for the dissemination of UV energy. Furthermore, accumulated evidence demonstrates the antioxidative, anti-inflammatory and antiaging properties of MAAs, providing another mechanism of photoprotection.¹⁴

Recently, several initial biosynthetic steps of MAAs have been elucidated through biochemical and genetic studies. Their biosynthesis starts from the production of 4-deoxygudasol (4-DG) from sedoheptulose 7-phosphate, an intermediate of the pentose phosphate pathway, by a dimethyl 4-degadusol synthase (DDGS; MysA) and an O-methyltransferase (O-MT; MysB) (FIG. 1B).²⁷ In some microbes, 4-DG may also be produced from 3-dehydroquinate of the shikimate pathway through incompletely defined enzymatic steps.²⁸ Next, an ATP-grasp enzyme MysC converts 4-DG into mycosporine-glycine (MG) by introducing an amino acid moiety, primarily L-Gly, at the C3 of 4-DG (FIG. 1B). It has recently been discovered that MysC from the cyanobacterium Anabaena variabilis ATCC 29413 phosphorylates 4-DG rather than L-Gly, typical to other ATP grasp enzymes.²⁷ MG is the direct biosynthetic precursor of disubstituted MAAs (e.g., porphyra-334) with an amino acid moiety at the C1 (FIG. 1B). It was biochemically confirmed that a non-ribosomal peptide synthetase (NRPS)-like enzyme MysE, which contains an adenylation (A), a thiolation (T), and a thioesterase (TE) domain, catalyzes this step in the biosynthesis of shinorine.²⁷ On the other hand, an MAA biosynthetic gene cluster (BGC) from the cyanobacterium Nostoc punctiforme ATCC 29133 has no NRPS gene but a D-Ala-D-Ala ligase-like enzyme gene mysD.²⁹ The heterologous expression of this BGC in E. coli produces three MAA analogs, shinorine (the major product), porphyra-334, and mycosporine-2-Gly, confirming MysD's involvement in the MAA biosynthesis. However, the following biosynthetic route from disubstituted MAAs to other MAA analogs remains completely unknown.

The heterologous production of serial MAA analogs, including palythines, in E. coli is described herein. Sequence similarity network (SSN) and genome neighborhood network (GNN) analyses of known MAA biosynthetic enzymes were used to identify a putative mysD-containing BGC in the genome of Nostoc linckia NIES-25 that is adjacent to a short-chain dehydrogenase/reductase (SDR) gene and a nonheme iron(II)- and 2-oxoglutarate-dependent (Fe/2OG) oxygenase gene MysH.³⁰ Heterologous expression of multiple refactored MAA BGCs in E. coli produced MAA analogs and demonstrated the direct conversion of disubstituted MAAs into palythines by the Fe/2OG enzyme MysH. Furthermore, biochemical characterization of its recombinant MysD supported its role in the formation of porphyra-334, shinorine, and other MAA analogs. Such enzymes are useful for the development of next-generation sunscreens via synthetic biology and biocatalysis approaches.

### Example 1: Distribution of MAA BGCs in Microbial Genomes

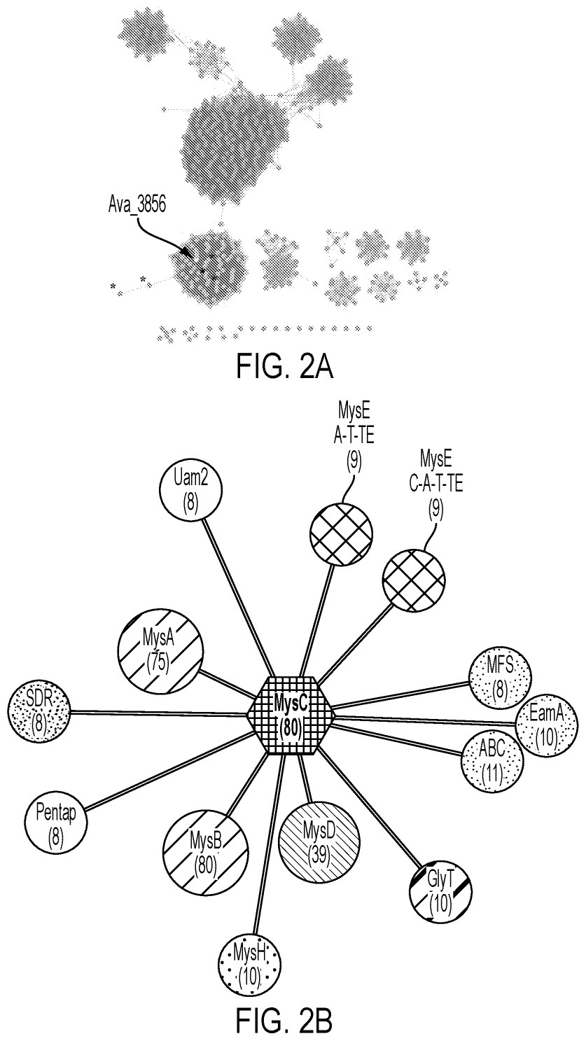

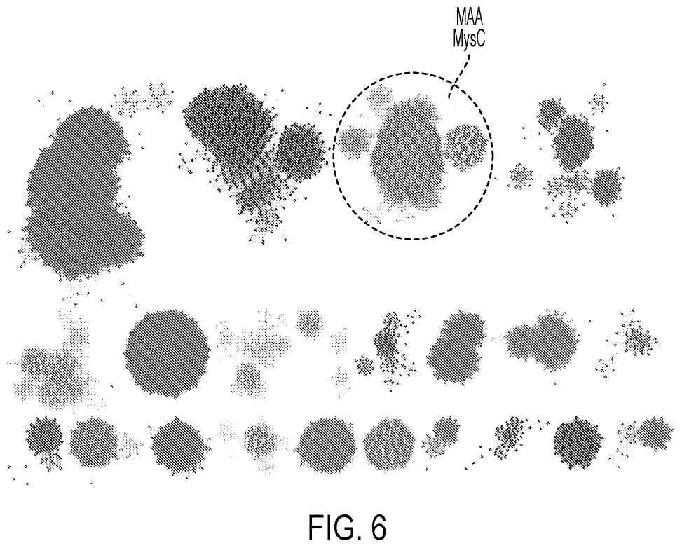

Genome mining has become a powerful approach for the discovery of new natural products and enzymology,³¹ supported by the exponential growth of genomic sequence data. To probe the distribution of MAA BGCs, MysC (Ava_3856) from A. variabilis ATCC 29413 was first used as the query to mine its homologs in the UniRef50 database that includes all proteins with at least 50% sequence identity to and 80% overlap with the longest sequence in the family.^{27,32} This analysis revealed that MysC belongs to the protein family #02655 (ATP_Grasp_3, PF02655) in the Pfam database,³³ which includes 8,435 ATP grasp enzyme homologs (October 2020). Subsequent SSN analysis of this family identified 22 distinct clusters with a sequence identity of >35P (FIG. 6). One cluster of 585 members was reanalyzed to separate homologs with >45% protein sequence identity into 15 clusters and 11 singletons, including one cluster formed by 92 MysC homologs (FIG. 2A, Table 1). Except for three MysC homologs from α-proteobacteria (e.g., Mycobacterium sp.) and two from eukaryotes (e.g., Chromera velia), the rest all are from cyanobacteria. This result suggested that several microbial phyla can use MAAs for photoprotection. The increasing availability of eukaryotic genomes (e.g., fungi, corals and macroalgae) will lead to more complete understanding of the MAA genomic distribution. Furthermore, this study indicated the use of SSN analysis for genome-based natural product research.

TABLE 1
Accession numbers for MysC homologs shown in FIG. 2A.

| Uniprot ID | Gene Name | Species | Phylum |
|---|---|---|---|
| A0A0Q2QHP0 | AO501_14480 | Mycobacterium gordonae | Actinobacteria |
| A0A3S0TU06 | EKK34_29475 | Mycobacterium sp. | Actinobacteria |
| A0A5A7SAT3 | FOY51_14930 | Rhodococcus sp. C1-24 | Actinobacteria |
| A0A0G4HZ53 | Cvel_9647 | Chromera velia CCMP2878 | Chromerida |
| R1G4T9 | EMIHUDRAFT_52960 | Emiliania huxleyi | Haptista |
| A0A433W0B3 | DSM107010_29350 | Chroococcidiopsis cubana | Cyanobacteria |
| | | SAG 39.79 | |
| A0A139WZN8 | WA1_05090 | Scytonema hofmannii PCC | Cyanobacteria |
| | | 7110 | |
| A0A2Z5X784 | mysC | Nostoc verrucosum | Cyanobacteria |
| A0A1Z4GTP3 | NIES2100_06370 | Calothrix sp. NIES-2100 | Cyanobacteria |
| A0A1Q4RU46 | NIES2101_15200 | Calothrix sp. HK-06 | Cyanobacteria |
| A0A0C2R3C6 | SD80_01670 | Scytonema tolypothrichoides | Cyanobacteria |
| | | VB-61278 | |
| A0A2R5FKA4 | NIES4072_28690 | Nostoc commune NIES-4072 | Cyanobacteria |
| A0A0M0SH70 | AMR41_24200 | Hapalosiphon sp. MRB220 | Cyanobacteria |
| A0A2T1F866 | C7B80_31420 | Cyanosarcina cf. burmensis | Cyanobacteria |
| | | CCALA 770 | |
| A0A367QNV7 | A6S26_15830 | Nostoc sp. ATCC 43529 | Cyanobacteria |
| A0A2N6JWS5 | CEN44_24325 | Fischerella muscicola | Cyanobacteria |
| | | CCMEE 5323 | |
| B4VP63 | MC7420_4633 | Coleofasciculus | Cyanobacteria |
| | | chthonoplastes PCC 7420 | |
| K9QUQ5 | Nos7524_3368 | Nostoc sp. PCC 7524 | Cyanobacteria |
| A0A0S3U2V2 | LEP3755_23100 | Leptolyngbya sp. NIES-3755 | Cyanobacteria |
| K9TVZ3 | Chro_0780 | Chroococcidiopsis thermalis | Cyanobacteria |
| | | PCC 7203 | |
| A0A2N6MZD6 | CEN39_11340 | Fischerella thermalis | Cyanobacteria |
| | | CCMEE 5201 | |
| A0A218PXL8 | NIES3585_03720 | Nodularia sp. NIES-3585 | Cyanobacteria |
| A0A1Z4HW63 | NIES2107_59490 | Nostoc carneum NIES-2107 | Cyanobacteria |
| A0A1Z4LYV8 | NIES267_58470 | Calothrix parasitica NIES- | Cyanobacteria |
| | | 267 | |
| A0A654SJH1 | apha_01438 | Chrysosporum ovalisporum | Cyanobacteria |
| A0A2C6VZE1 | VF13_24910 | Nostoc linckia z16 | Cyanobacteria |
| A0A2T1EQS1 | C7B70_02210 | Chlorogloea sp. CCALA 695 | Cyanobacteria |
| A0A1E5QWM1 | A5482_11085 | Cyanobacterium sp. IPPAS | Cyanobacteria |
| | | B-1200 | |
| A0A2I8ACV8 | CLI64_23890 | Nostoc sp. CENA543 | Cyanobacteria |
| A0A2D3HK59 | mylE | Nostoc flagelliforme | Cyanobacteria |
| A0A367QJH7 | A6V25_22315 | Nostoc sp. ATCC 53789 | Cyanobacteria |
| A0A2S6VI18 | B1A85_06375 | Chroococcidiopsis sp. TS- | Cyanobacteria |
| | | 821 | |
| K9X913 | Glo7428_0523 | Gloeocapsa sp. PCC 7428 | Cyanobacteria |
| A0A1Y0RL91 | BZZ01_16725 | Nostocales cyanobacterium | Cyanobacteria |
| | | HT-58-2 | |
| A0A2P8QMI8 | C7Y66_19855 | Chroococcidiopsis sp. | Cyanobacteria |
| | | CCALA 051 | |
| A0A6B3P645 | F6K60_05300 | Okeania sp. SIO1F9 | Cyanobacteria |
| A0A6B3MZW3 | F6J89_01825 | Symploca sp. SIO1C4 | Cyanobacteria |
| A0A2K8WS68 | AA637_12615 | Cyanobacterium sp. HL-69 | Cyanobacteria |
| A0A4Q9JE38 | B4U84_12935 | Westiellopsis prolifica IICB1 | Cyanobacteria |
| Q3M6C5 | Ava_3856 | Anabaena variabilis ATCC | Cyanobacteria |
| | | 29413 | |
| A0A252E4S5 | BV372_13530 | Nostoc sp. T09 | Cyanobacteria |
| A0A367RKS4 | A6770_15820 | Nostoc minutum NIES-26 | Cyanobacteria |
| A0A1E2WNZ8 | A4S05_34795 | Nostoc sp. KVJ20 | Cyanobacteria |
| A0A1B2CWG9 | UCFS15_00407 | Heteroscytonema crispum | Cyanobacteria |
| | | UCFS15 | |
| A0A1U7HY56 | NIES1031_04760 | Chroogloeocystis siderophila | Cyanobacteria |
| | | 5.2 s.c.1 | |
| A0A1L9QXK4 | BI308_00105 | Roseofilum reptotaenium | Cyanobacteria |
| | | AO1-A | |
| A0A2L2NR98 | NLP_2817 | Nostoc sp. ‘Lobaria | Cyanobacteria |
| | | pulmonaria (5183) | |
| | | cyanobiont’ | |
| A0A2H2XFD9 | NIES4071_48500 | Calothrix sp. NIES-4071 | Cyanobacteria |
| A0A533NZW2 | EBE86_16905 | Hormoscilla sp. GUM202 | Cyanobacteria |
| A0A367RVN3 | A6769_04950 | Nostoc punctiforme NIES- | Cyanobacteria |
| | | 2108 | |
| A0A1Z4TPY4 | NIES4106_37630 | Fischerella sp. NIES-4106 | Cyanobacteria |
| A0A6B3MAD2 | F6K58_17255 | Symploca sp. SIO2E9 | Cyanobacteria |
| A0A1Z4IH51 | NIES2111_57410 | Nostoc sp. NIES-2111 | Cyanobacteria |
| A0A1Z4IB36 | NIES2111_35850 | Nostoc sp. NIES-2111 | Cyanobacteria |
| K9VKW1 | Osc7112_3784 | Oscillatoria nigro-viridis | Cyanobacteria |
| | | PCC 7112 | |
| A0A2T1F5R3 | C7B77_28500 | Chamaesiphon polymorphus | Cyanobacteria |
| | | CCALA 037 | |
| K9W0D3 | Cri9333_2377 | Crinalium epipsammum PCC | Cyanobacteria |
| | | 9333 | |
| A0A1Z4SWP6 | NIES4105_48440 | Calothrix sp. NIES-4105 | Cyanobacteria |
| A0A1U7I932 | FACHB389_18875 | Nostoc calcicola FACHB- | Cyanobacteria |
| | | 389 | |
| A0A1W5CLX0 | AN489_06955 | Anabaena sp. 39858 | Cyanobacteria |
| A0A328IAQ4 | C6Y22_26065 | Hapalosiphonaceae | Cyanobacteria |
| | | cyanobacterium JJU2 | |
| A0A533NF66 | EBE85_21135 | Hormoscilla sp. GUM007 | Cyanobacteria |
| A0A479ZZ55 | SR1949_29190 | Sphaerospermopsis | Cyanobacteria |
| | | reniformis | |
| A0A357A498 | DD761_02610 | Cyanobacteria bacterium | Cyanobacteria |
| | | UBA11691 | |
| A0A1Z4QDW0 | NIES4074_07940 | Cylindrospermum sp. NIES- | Cyanobacteria |
| | | 4074 | |
| K9R4C7 | Riv7116_0136 | Rivularia sp. PCC 7116 | Cyanobacteria |
| A0A3S0ZZ73 | PCC6912_44900 | Chlorogloeopsis fritschii | Cyanobacteria |
| | | PCC 6912 | |
| A0A3C0NJT8 | DCP31_40620 | Cyanobacteria bacterium | Cyanobacteria |
| | | UBA8543 | |
| B2J6X7 | Npun_R5598 | Nostoc punctiforme PCC | Cyanobacteria |
| | | 73102 | |
| A0A0C1NCV3 | DA73_0218765 | Tolypothrix bouteillei | Cyanobacteria |
| | | VB521301 | |
| A0A1Z4S904 | NIES4103_38540 | Nostoc sp. NIES-4103 | Cyanobacteria |
| A0A2K8SZ63 | COO91_06032 | Nostoc flagelliforme | Cyanobacteria |
| | | CCNUN1 | |
| A0A3N6PGG7 | D5R40_05450 | Okeania hirsuta | Cyanobacteria |
| A0A0C2QMV0 | SD80_01695 | Scytonema tolypothrichoides | Cyanobacteria |
| | | VB-61278 | |
| Q3M6C5 | Ava_3856 | Trichormus variabilis strain | Cyanobacteria |
| | | ATCC 29413 | |
| A0A1Z4ND62 | NIES3974_02980 | Calothrix sp. NIES-3974 | Cyanobacteria |
| A0A0D8ZR72 | UH38_14315 | Aliterella atlantica | Cyanobacteria |
| | | CENA595 | |
| A0A2T1LWM6 | C7H19_13915 | Aphanothece hegewaldii | Cyanobacteria |
| | | CCALA 016 | |
| K9XU47 | Sta7437_1637 | Stanieria cyanosphaera PCC | Cyanobacteria |
| | | 7437 | |
| A0A2Z6D2K3 | NIES2109_59170 | Nostoc sp. HK-01 | Cyanobacteria |
| A0A5P8W9G9 | GXM_06696 | Nostoc sphaeroides | Cyanobacteria |
| | | CCNUC1 | |
| A0A1S6LXZ0 | mylE | Nostoc commune var. | Cyanobacteria |
| | | flagelliforme QSY 1 | |
| A0A1Z4LFB5 | NIES25_64150 | Nostoc linckia NIES-25 | Cyanobacteria |
| A0A4D9CF37 | BLD44_013555 | Mastigocladus laminosus | Cyanobacteria |
| | | UU774 | |
| A0A1B2CWF7 | mysC | Heteroscytonema crispum | Cyanobacteria |
| | | UCFS10 | |
| A0A0C1N3Z4 | DA73_0239150 | Tolypothrix bouteillei | Cyanobacteria |
| | | VB521301 | |
| A0A2L2N6B5 | NPM_2790 | Nostoc sp. ‘Peltigera | Cyanobacteria |
| | | membranacea cyanobiont’ | |
| | | N6 | |
| A0A1Z4Q915 | NIES4073_76020 | Scytonema sp. NIES-4073 | Cyanobacteria |
| A0A2Z5VN68 | mysC | Nostoc commune KU002 | Cyanobacteria |
| A0A1Z4UKN2 | NIES73_09950 | Sphaerospermopsis | Cyanobacteria |
| | | kisseleviana NIES-73 | |
| A0A5Q0GJK5 | EH233_17470 | Anabaena sp. YBS01 | Cyanobacteria |
| A0ZIV3 | NSP_23010 | Nodularia spumigena | Cyanobacteria |
| | | CCY9414 | |
| A0A3S1ANM2 | DSM106972_036000 | Calothrix desertica PCC | Cyanobacteria |
| | | 7102 | |

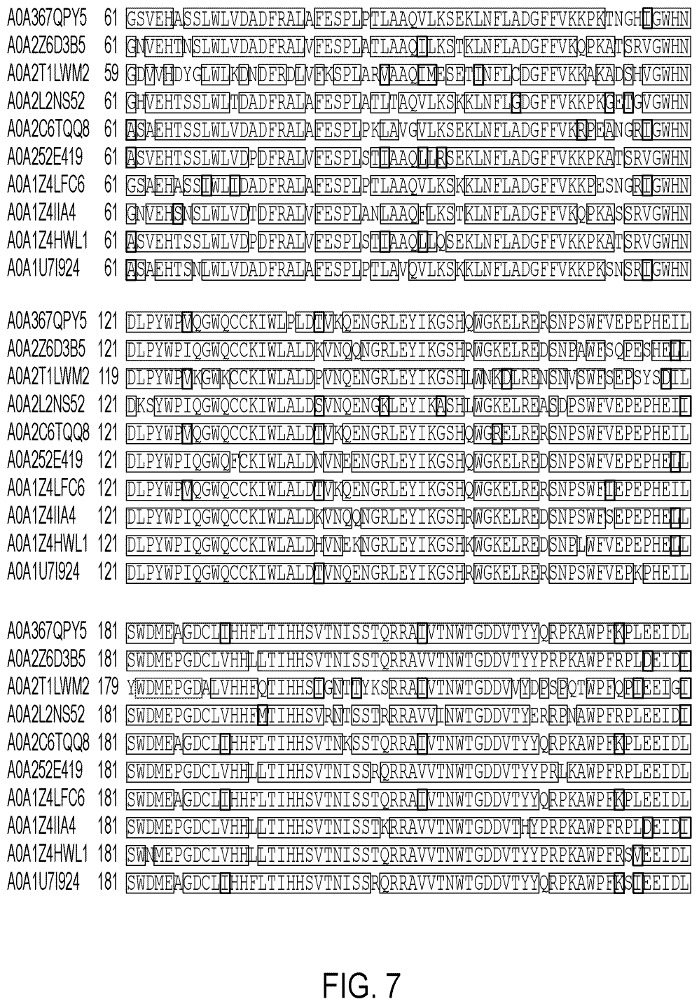

Next, GNN analysis of the MysC cluster was performed to identify enzymes with high co-occurrence frequency within ten open reading frames upstream or downstream of MysC (FIG. 2B). A total of 12 MysC homologs had no nearby open reading frames in the GNN analysis and were removed from further analysis. These homologs are all predicted from unassembled whole-genome shotgun sequencing projects. As expected, homologs of MysA (75), MysB (80), NRPS MysE (18), and MysD (39) were frequently colocalized with 80 MysC homologs to form the MAA BGC (FIG. 2B). In addition to MysEs with the A-T-TE domain organization,²⁷ nine enzymes carry an additional condensation (C) domain.²¹ Furthermore, high occurrence of transporters (29) was observed, including ABC, EamA-like,³⁴ and major facilitator superfamily (MFS) transporters, though almost all known MAAs have been extracted only from biomasses and might be located in the extracellular matrix.^{17,21,26,35-36} A recent study also found the frequent presence of a transporter gene within the MAA BGCs in the microbial mat communities of Shark Bay, Australia.³⁷ Importantly, the GNN analysis revealed three enzyme groups that may contribute to the structural diversity of MAAs, including glycosyltransferases (10), phytanoyl-CoA dioxygenases (10), and short-chain dehydrogenases/reductases (SDRs, 8). Although many glycosylated MAA analogs have been reported, the corresponding glycosyltransferases remain unidentified.²¹ Phytanoyl-CoA dioxygenases belong to the Fe(II)/2OG enzyme family and the 10 enzymes colocalized with MysCs all carry the catalytically essential 2-His-1-carboxylate facial triad for coordinating Fe(II) (FIG. 7).³⁸ Phytanoyl-CoA dioxygenases catalyze the α-hydroxylation of phytanoyl-CoA in the degradation of phytanic acid.³⁹ On the other hand, members of the Fe(II)/2OG enzyme family are known to catalyze a wide range of reactions, e.g., hydroxylation, decarboxylation, dehydration, oxidation, reduction, isomerization, ring formation, and expansion,⁴⁰ some of which may lead to the production of MAA analogs (FIG. 1A). These phytanoyl-CoA dioxygenases related to the MAA biosynthesis are referred to herein as MysHs. Similar to Fe(II)/2OG enzymes, SDRs form a large protein superfamily that demonstrates a broad substrate range and rich function diversity.⁴¹ Two other protein groups that are frequently co-occurred with MysC are restriction endonucleases and pentapeptide repeats, whose roles in the biosynthesis of MAAs are unclear.

### Example 2: Heterologous Expression of Refactored MAA BGCs from Nostoc linkia NIES-25 in E. coli

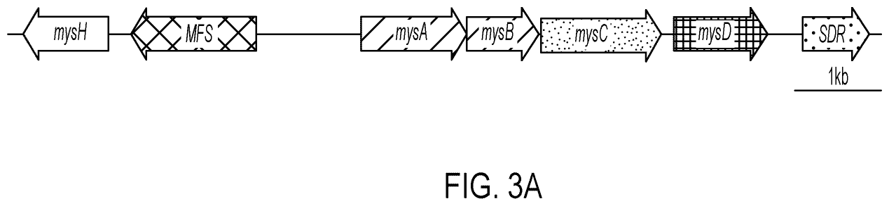

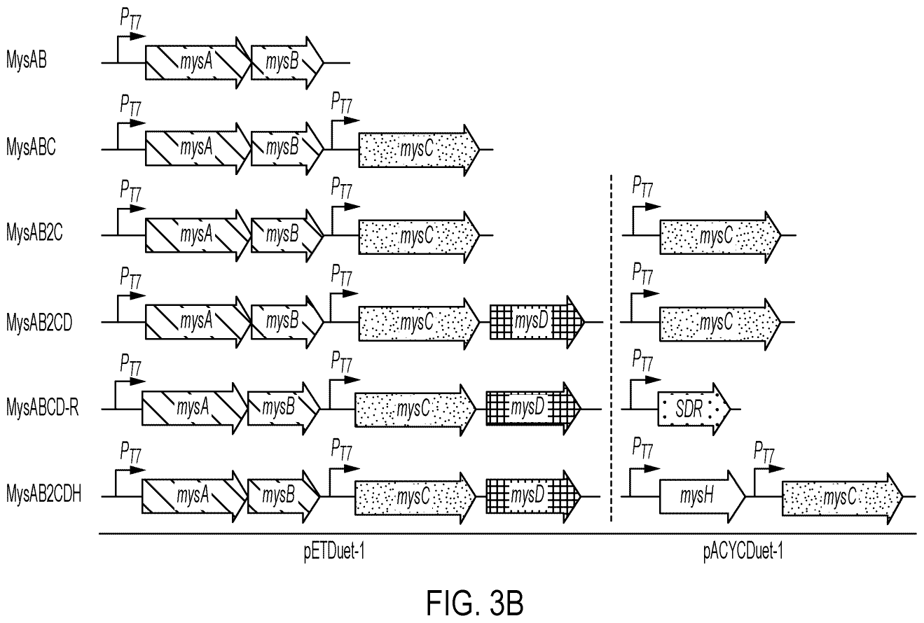

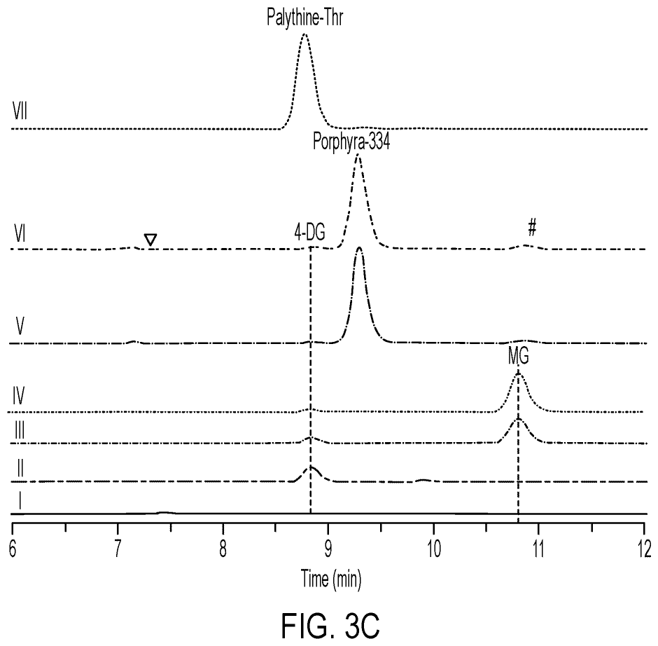

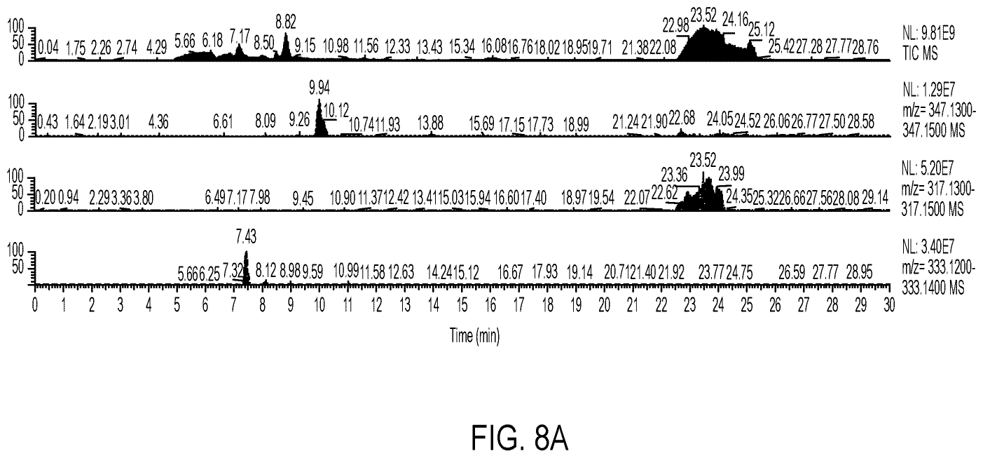

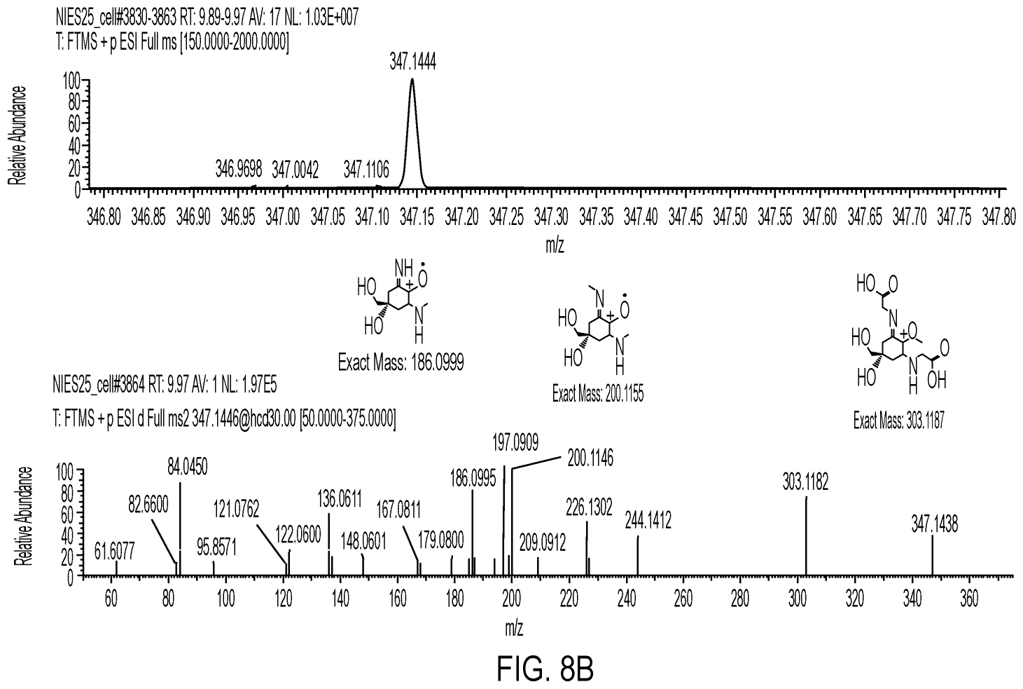

Based on the results of the above bioinformatics studies, new MAA biosynthetic enzymes were characterized. Specifically, a putative 9.6-kb MAA BGC was selected from a 1.78-Mb plasmid (GenBank: AP018223.1) in Nostoc linkia NIES-25, which encodes MysA-D (NIES25_64130 to NIES25_64160), a phytanoyl-CoA dioxygenase (MysH, NIES25_64110), an MFS transporter (NIES25_64120), and a SDR (NIES25_64170) (FIG. 3A, Table 2). To examine the expression of this cluster in N. linkia NIES-25, this strain was cultured in BG-11 medium at 26° C. for 21 days. However, HPLC analysis of methanolic extracts of pelleted cells and lyophilized culture medium failed to identify any peak with maximal absorbance between 310 to 360 nm. On the other hand, extracted ion chromatogram (EIC) extraction of LC-high resolution (HR) MS data of methanolic extracts of pelleted cells revealed a peak corresponding to the parental ions of porphyra-334 (observed M+H]⁺: 347.1444; calculated [M+H]⁺: 347.1449, FIG. 8), whose selective MS/MS fragmentation ions further suggested the production of porphyra-334. EIC analysis suggested a putative peak corresponding to shinorine (observed M+H]⁺: 333.1400; calculated [M+H]⁺: 333.1292, FIG. 8A), but its low abundance yielded only a low quality MS/MS spectrum, preventing a reliable structural identification. A peak for putative MG-Ala (calculated [M+H]⁺: 317.1343) was not observed in EIC analysis (FIG. 8A). Nonetheless, this study suggested that the MAA cluster in N. linkia NIES-25 is active under the culturing conditions.

TABLE 2
Bioinformatic analysis of MAA gene cluster from Nostoc linkia NIES-25.

| | Protein | | | | |
|---|---|---|---|---|---|
| Gene name | accession | Size¹ | Homolog, origin | ID/SI² | Predicted function |
| NIES25_64110 | BAY79923.1 | 267 | WP_190955827.1, | 98/99 | Phytanoyl-CoA |
| | | | Nostoc | | dioxygenase |
| NIES25_64120 | BAY79924.1 | 485 | WP_190955828.1, | 94/97 | Major facilitator |
| | | | Nostoc | | transporter |
| NIES25_64130 | BAY79925.1 | 410 | RCJ25793.1, | 98/99 | Sedoheptulose 7- |
| | | | Nostoc sp. | | phosphate cyclase |
| | | | ATCC 43529 | | |
| NIES25_64140 | BAY79926.1 | 278 | WP_190955830.1, | 95/97 | Class I SAM- |
| | | | Nostoc | | dependent |
| | | | | | methyltransferase |
| NIES25_64150 | BAY79927.1 | 464 | WP_190955831.1, | 97/98 | ATP-grasp ligase |
| | | | Nostoc | | |
| NIES25_64160 | BAY79928.1 | 368 | WP_190955832.1, | 93/96 | D-alanine-D-alanine |
| | | | Nostoc | | ligase |
| NIES25_64170 | BAY79929.1 | 257 | RCJ25797.1, | 98/98 | short-chain |
| | | | Nostoc sp. | | dehydrogenase/ |
| | | | ATCC 43529 | | reductase |

Note:
¹amino acid;
²identities/similarities (%).

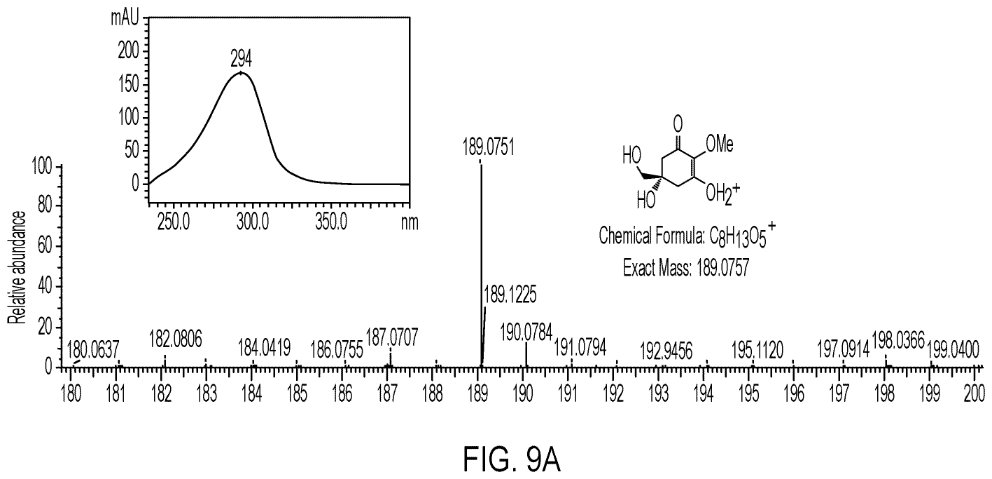

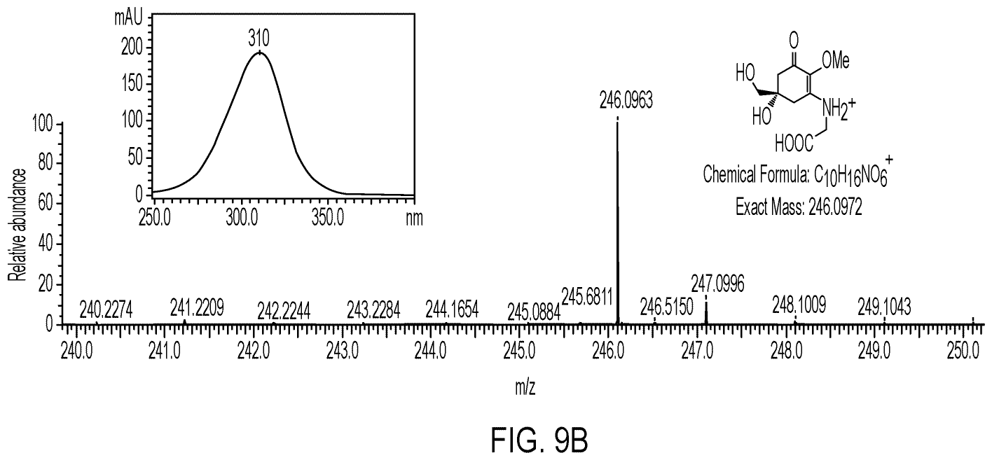

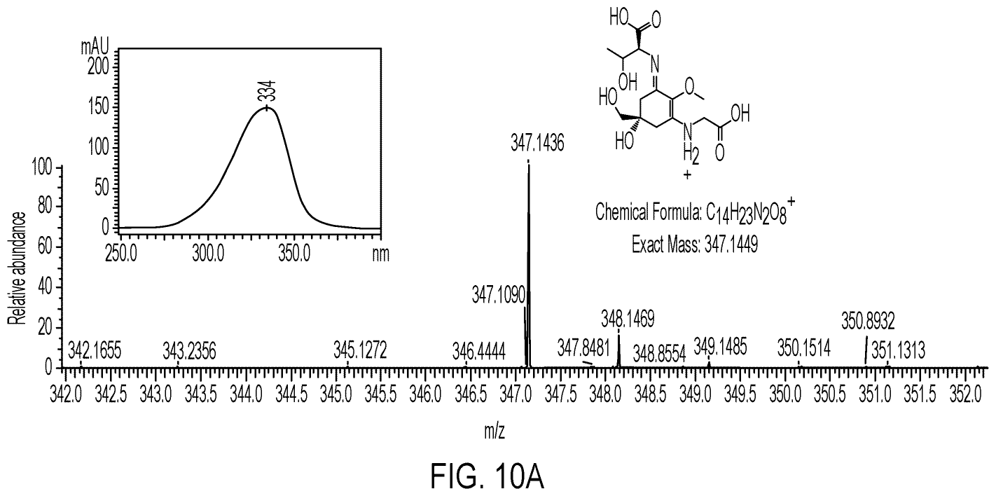

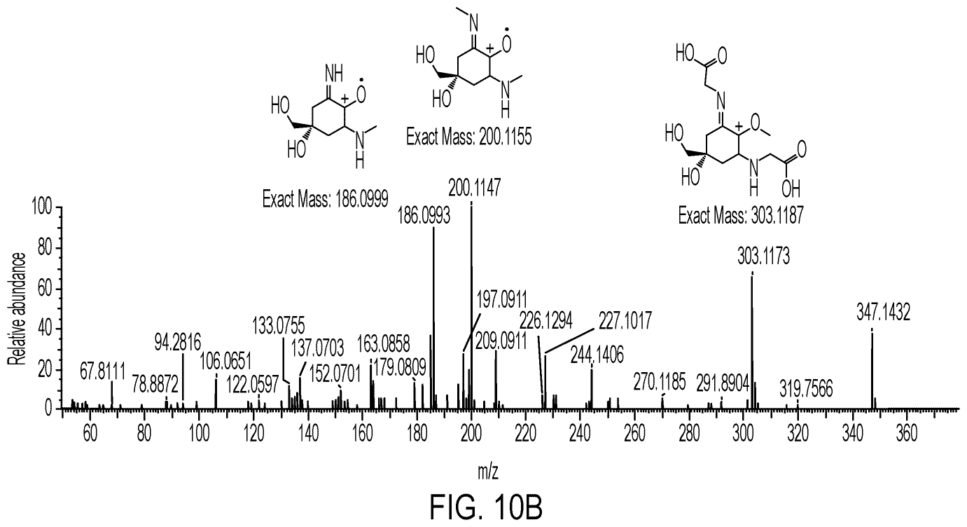

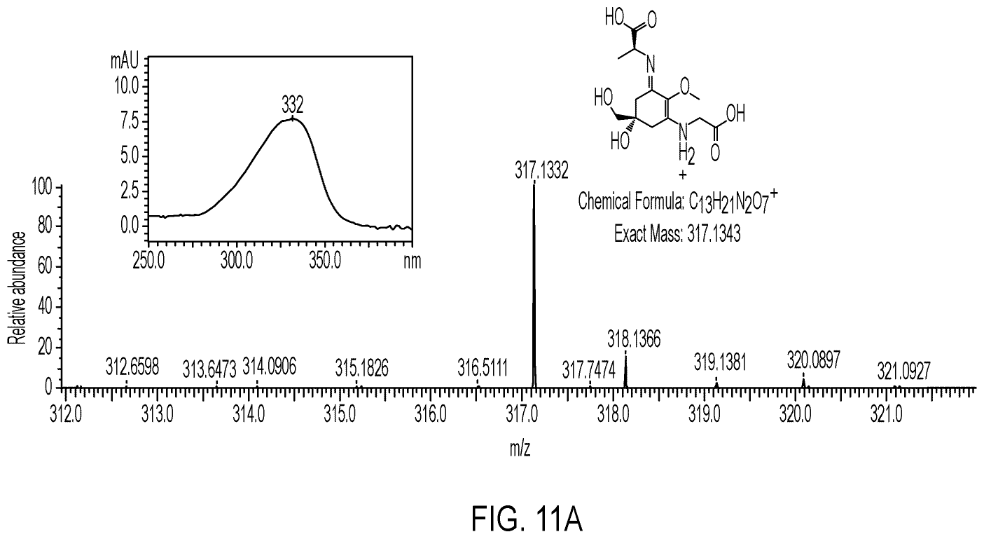

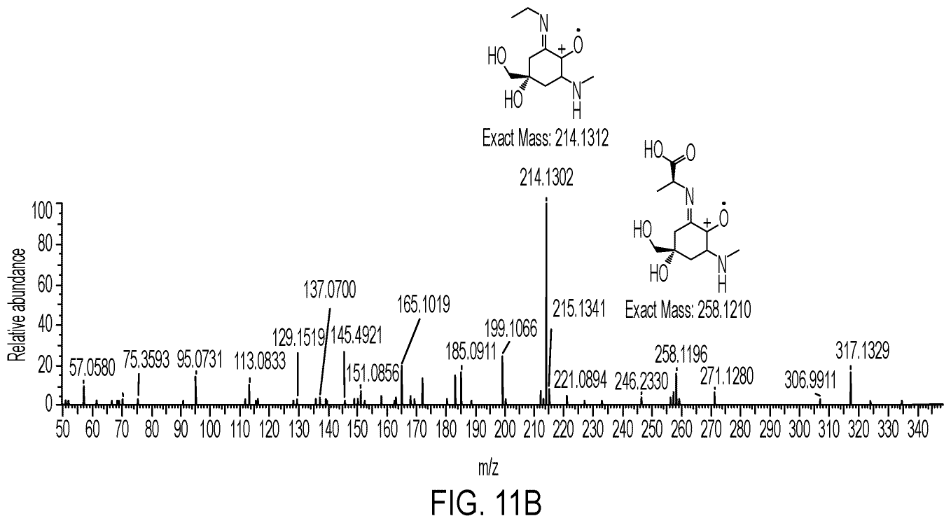

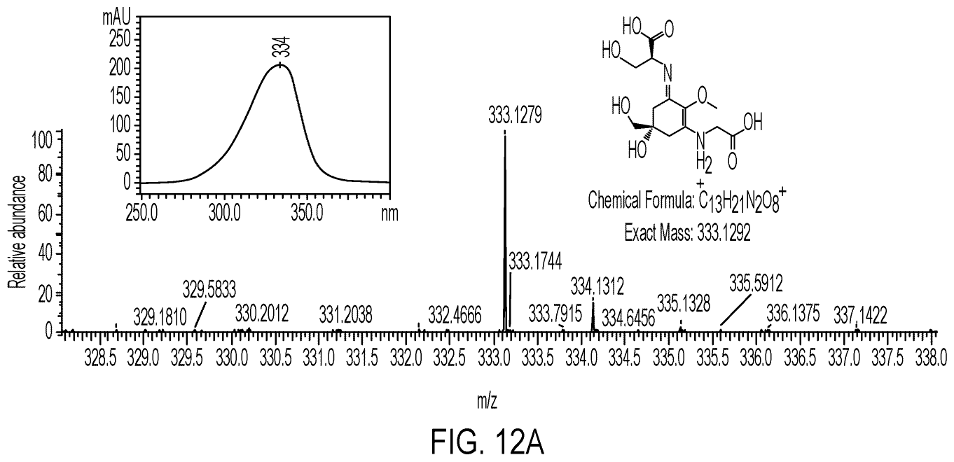

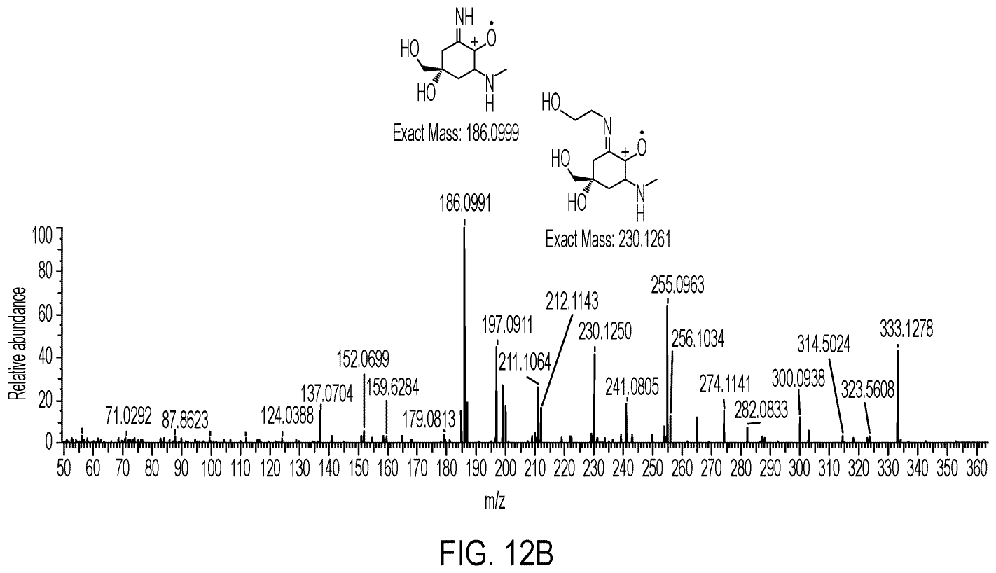

To further characterize MAA biosynthesis in N. linkia NIES-25, multiple refactored BGCs were designed for heterologous expression in E. coli BL21-Gold (DE3) (FIG. 3B). The co-expression of mysAB under the control of the T7 promoter in pETDuet-1 led to the production of 4-DG (FIG. 3C, II), which showed maximal absorbance at 294 nm and a protonated ion of m/z 189.0751 (calculated [M+H]⁺: 189.0757, FIG. 9), agreeing with reported data.²⁶ 4-DG was only detected from the methanolic extract of cell pellets, the same for all other MAAs described below. No 4-DG was detected in the control transformed with the empty pETDuet-1 (FIG. 3C, I). When mysC was expressed along with mysAB in pETDuet-1, the production of MG was observed in E. coli (FIG. 3C, III) as confirmed by its maximal absorbance at 310 nm and protonated ion of m/z 246.0963 (calculated [M+H]⁺: 246.0972, FIG. 9). A small quantity of 4-DG was still observed (FIG. 3C, III), suggesting the imbalanced catalytic activity of MysC compared with MysAB. Indeed, when one additional copy of mysC was coexpressed in a middle-copy number vector pACYCDuet-1 (FIG. 3B), the peak area of MG was improved by about 1.5 times while that of 4-DG was decreased by about 50% (FIG. 3C, IV). Next, the catalytic function of MysD in the production of disubstituted MAAs was examined by coexpressing its gene with mycAB2C in E. coli (FIG. 3B). HPLC analysis of the methanolic extract of E. coli pellets expressing mysAB2CD revealed one new major peak with the retention time of 9.3 min and one new minor peak at 10.8 min, while 4-DG was still found (FIG. 3C, V). These new peaks showed the same maximal absorbance at around 334 nm (FIGS. 10 and 11). HRMS and MS/MS analysis indicated the production of porphyra-334 as the major peak (observed [M+H]⁺: 347.1436; calculated [M+H]⁺: 347.1449, FIG. 10). The minor peak showed the protonated ion of m/z 317.1332 (calculated [M+H]⁺: 317.1343, FIG. 11), and HRMS/MS analysis indicated it to be MG-Ala.³⁵ As shinorine is commonly isolated along with porphyra-334, a careful search of the LC and LC-MS spectra led to the identification of shinorine with a retention time of 7.3 min (FIG. 3C, V), a protonated ion of m/z 333.1279 (calculated [M+H]⁺: 333.1292) and an expected MS/MS fragmentation (FIG. 12). The production of these three disubstituted MAAs demonstrates that MysD from N. linkia NIES-25 functionalizes the C1 of MG using multiple amino acids as substrate, with a strong preference to L-Thr. Substrate promiscuity of MysD has previously been observed in the heterologous expression of the MAA BGC from N. punctiforme ATCC 29133 and Actinosynnema mirum DSM 43827 in E. coli and Streptomyces avermitilis SUKA22, respectively.^{29,35} In both cases, shinorine was the dominant product, suggesting different substrate preferences of MysD of different origins.

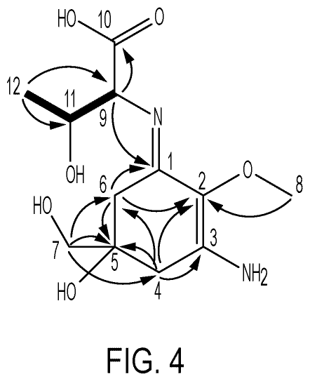

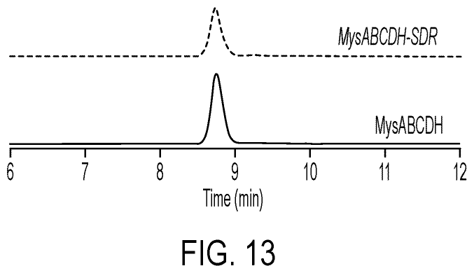

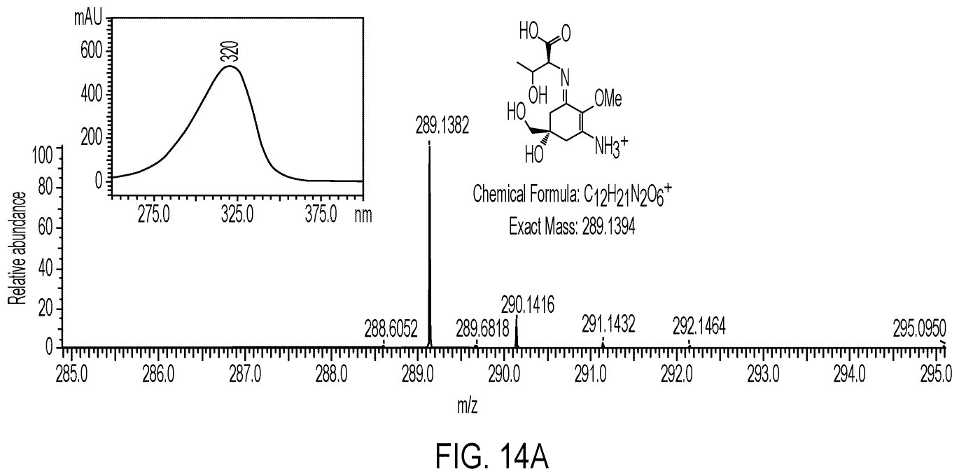

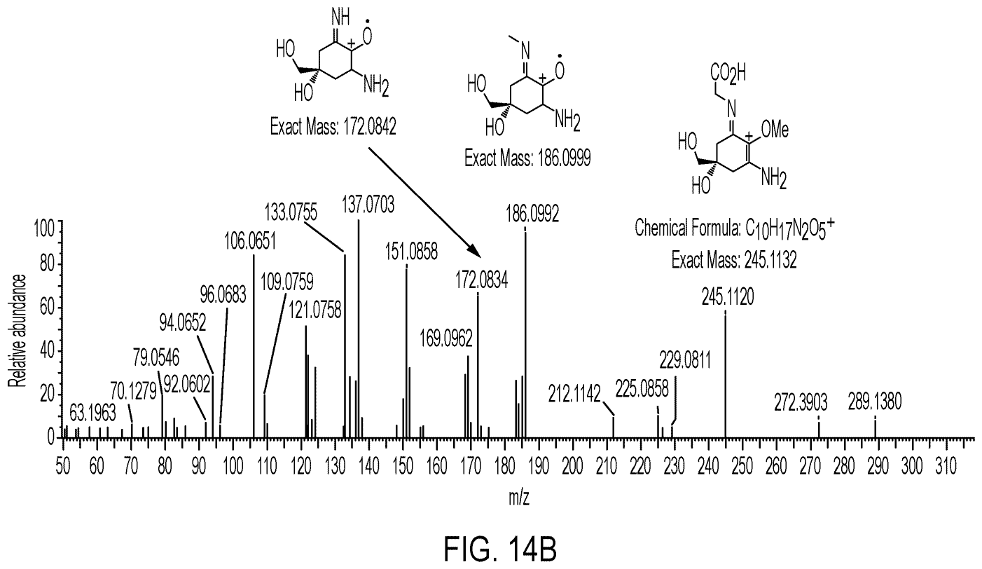

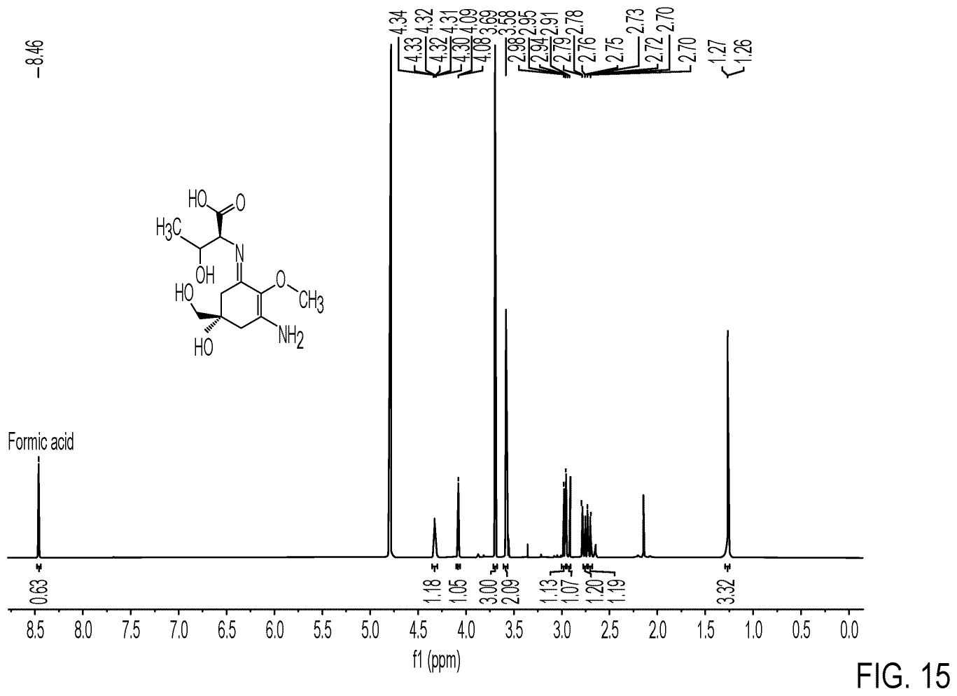

The successful production of disubstituted MAAs by expressing mysA-D from N. linkia NIES-25 in E. coli prompted characterization of the functions of two other biosynthetic genes in the cluster. Co-expression of sdr on pACYCDuet-1 (FIG. 3B) had no obvious change on the product profile of E. coli expressing mysABCD on pETDuet-1 (FIG. 3C, VI), suggesting the unclear enzymatic function of SDR for the MAA biosynthesis. Similarly, the sdr gene is adjacent to the MAA BGC in Scytonema cf. crispum UCFS15 and its coexpression with the cluster produces only shinorine in E. coli.²¹ In contrast, when mysH was cloned alone or with the second copy of mysC in pACYCDuet-1 (FIG. 3B) and expressed in E. coli transformed with mysABCD, a new major peak with the retention time of close to 8.8 min was observed concurrently with the almost complete disappearance of porphyra-334 (FIG. 3C, VII, FIG. 13). The content of the new peak showed maximal absorbance at 320 nm and its molecular formula was established as C₁₂H₂₀N₂O₆ based on a protonated ion of m/z 289.1382 (calculated [M+H]⁺: 289.1394, FIG. 14). HRMS/MS analysis of the parent molecular ion generated multiple fragment ions (e.g., m/z 245.112, 186.099, and 172.083) suggesting the peak content as palythine-Thr (FIG. 14).^{20,42} To further elucidate its structure, about 1 mg of this compound was purified for 1D and 2D NMR analysis (Table 3, FIGS. 15, 16, and 17). Comparison of its ¹H and ¹³C chemical shifts to those of palythine-Thr in a recent report allowed the assignment of 3-aminocyclohexenimine (C1, 2, 3, 4, 5, and 6) and Thr (C9, 10, 11, and 12, Table 3).⁴³ Furthermore, the assignment of the Thr moiety was supported by C12-H/C11-H/C9-H COSY correlations and HMBC correlations from C12-H to C9/C11 and from C9-H to C10 (FIG. 4). The presence of 3-aminocyclohexenimine moiety was supported by the HMBC correlations from C4-H to C2/C3/C5/C6, from C6-H to C1/C2/C5, and from C7-H to C4/C5/C6. The connectivity of the Thr and 3-aminocyclohexenimine moieties was further confirmed by the HMBC correlation from C9-H to C1 (FIG. 4). Additionally, the HMBC correlation from C8-H to C2 supported the presence of a methoxy group at the C2 (FIG. 4). Collectively, the combination of HRMS and NMR analyses indicates the production of palythine-Thr in E. coli expressing mysAB2CDH from N. linkia NIES-25. Importantly, these results support the direct conversion of porphyra-334 into palythine-Thr catalyzed by MysH (FIGS. 3 and 4), an advance in understanding MAA biosynthesis. Given the same biosynthetic origin, palythine-Thr likely share the same C5-S configuration as porphyra-334.

TABLE 3
Comparison of ¹H and ¹³C NMR chemical shifts of palythine-Thr
determined in the current work and a recent report.⁵⁰

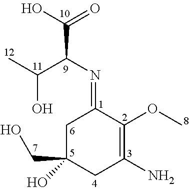 [SMILES: COC1=C(N)C[C@@](O)(CO)C/C1=N\[C@H](C(=O)O)C(C)O]

| | palythine-Thr^{a} | | literature^{a} | |
|---|---|---|---|---|
| Position | δ_{C}, type | δ_{H} (J in Hz) | δ_{C}, type | δ_{H} (J in Hz) |
| 1 | 163.8. C | | 163.8. C | |
| 2 | 127.7, C | | 127.7, C | |
| 3 | 163.8, C | | 163.8, C | |
| 4 | 38.6, | 2.97 (17.1, d) 2.71 (17.1, 1.4, | 38.6, | 2.96 (17.4, d) 2.71 (17.4, |
| 5 | 74.2, C | | 74.1, C | |
| 6 | 36.6, | 2.93 (17.5, d) 2.77 (17.5, 1.3, | 36.7, | 2.92 (17.4, d) 2.77 (17.4, |
| 7 | 70.2, | 3.58, s | 70.2, | 3.58, s |
| 8 | 62.0, | 3.69, s | 62.1, | 3.69, s |
| 9 | 67.4, CH | 4.08 (4.6, d) | 67.4, CH | 4.08 (4.8, d) |
| 10 | 177.9, C | | 177.9, C | |
| 11 | 70.9, CH | 4.32, m | 70.9, CH | 4.32, m |
| 12 | 22.2, | 1.26 (6.5, d) | 22.2, | 1.26, (6.6 d) |

^{a}D₂O

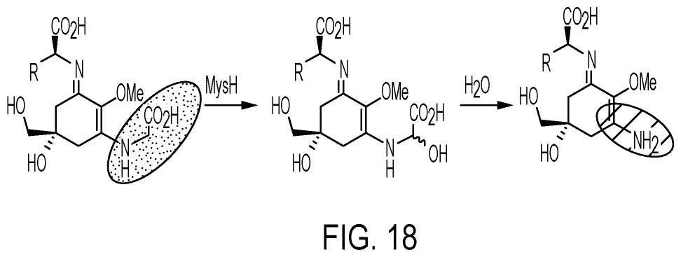

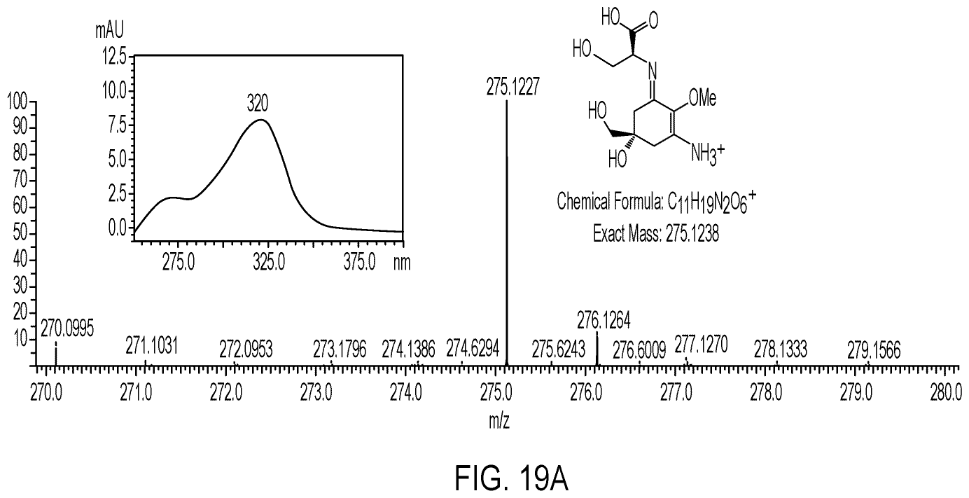

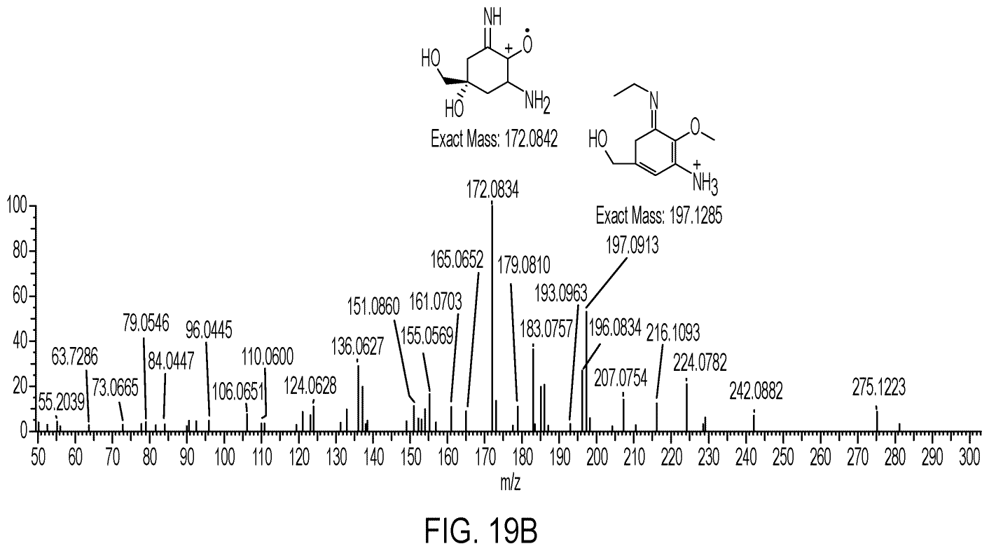

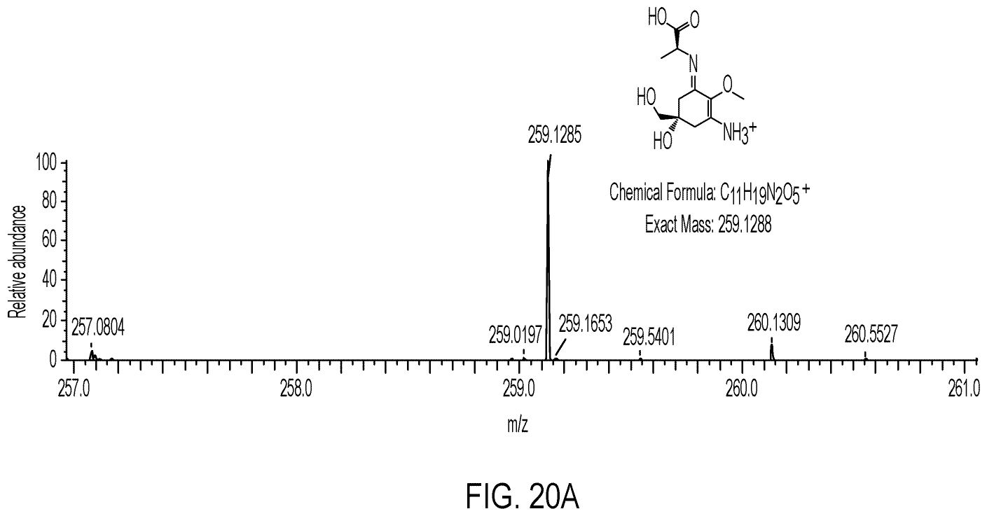

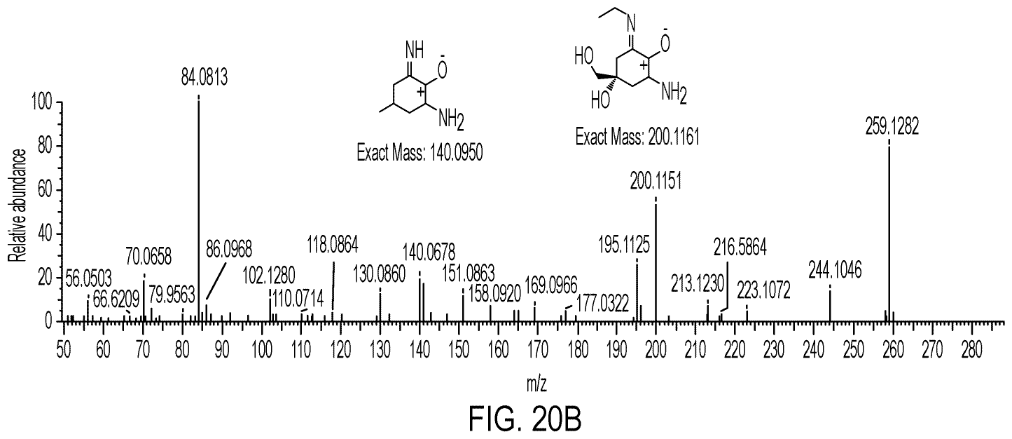

Current known palythines include palythine, palythine-Ser, palythine-Thr and their derivatives produced by corals, cyanobacteria, and other organisms (FIG. 1A).^{16,44} Similar to the biosynthesis of palythine-Thr, palythine and palythine-Ser may be converted directly from corresponding mycosporine-2-Gly and shinorine by MysH homologs (FIG. 18) and retain the same C5-S configuration (FIG. 1A). The direct conversion of the L-Gly moiety into the amine is a new reaction to the Fe(II)/2OG enzyme family.⁴⁰ One potential reaction path is that MysH catalyzes an α-hydroxylation on the C3-L-Gly moiety, followed by automatic hydrolysis to release palythines and glyoxylic acid (FIG. 18). The C3-amine of palythines can be further methylated by an N-methyltransferase to produce MAA analogs carrying a C3-methylamine (e.g., mycosporine-methylamine-Thr, FIG. 1A).¹⁶ Since E. coli expressing mycAB2CD produced porphyra-334, shinorine and MG-Ala (FIG. 3C, V), formation palythine-Ser and palythine-Ala in the crude extract of E. coli cell pellets expressing mysAB2CDH was investigated. Expected m/z values 275.1227 and 259.1288 for these two palythines were identified (calculated [M+H]⁺: 275.1238 for palythine-Ser; 259.1288 for palythine-Ala, FIGS. 19 and 20), indicating the substrate promiscuity of MysH. Palythine-Ser showed maximal absorbance at 320 nm and HRMS/MS fragmentations of both compounds suggested their structure assignment (FIGS. 19 and 20). Finally, both mysH and SDR were coexpressed with mysABCD in E. coli, and the same product profile as that of the coexpression of mysABCDH were observed (FIG. 13), indicating that SDR may not take any palythines as substrate.

### Example 3: Biochemical Characterization of Recombinant MysD

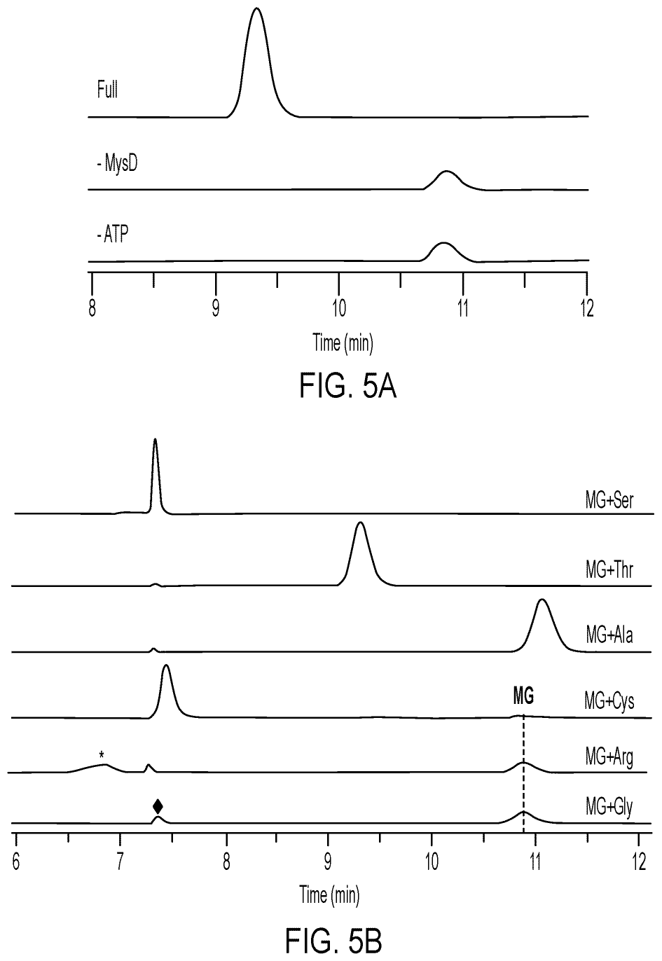

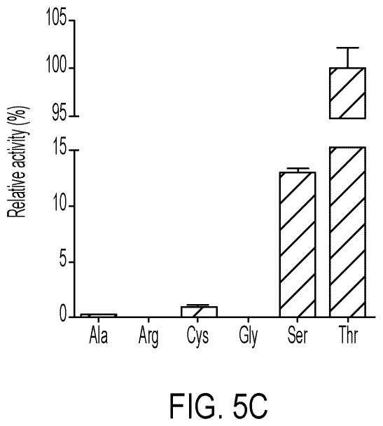

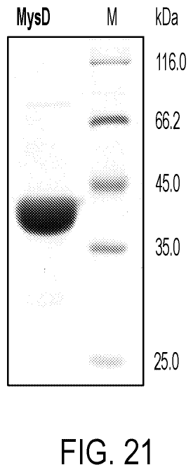

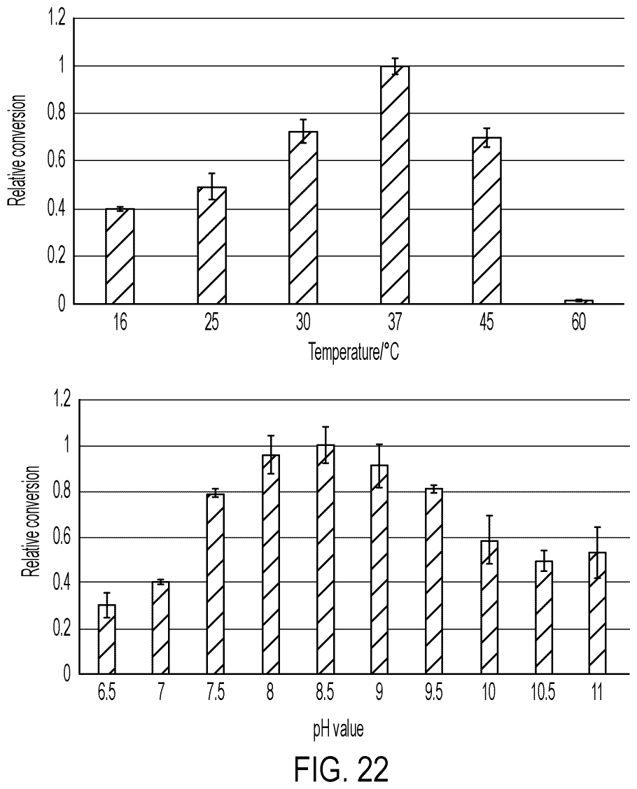

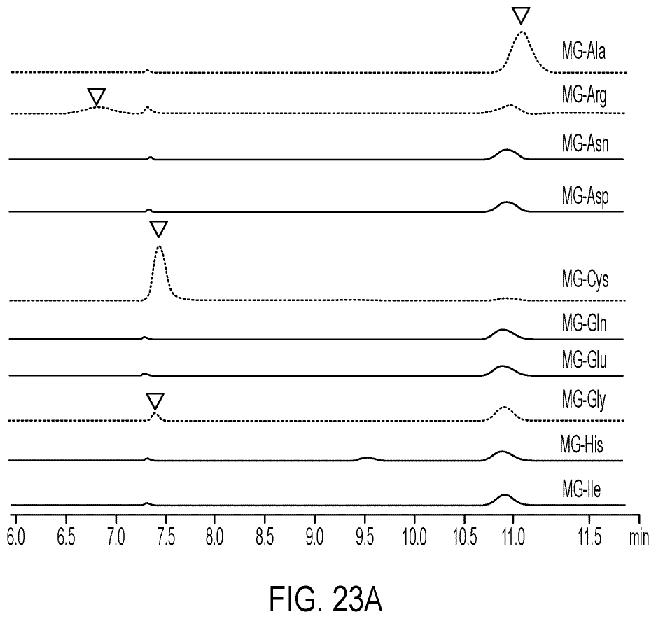

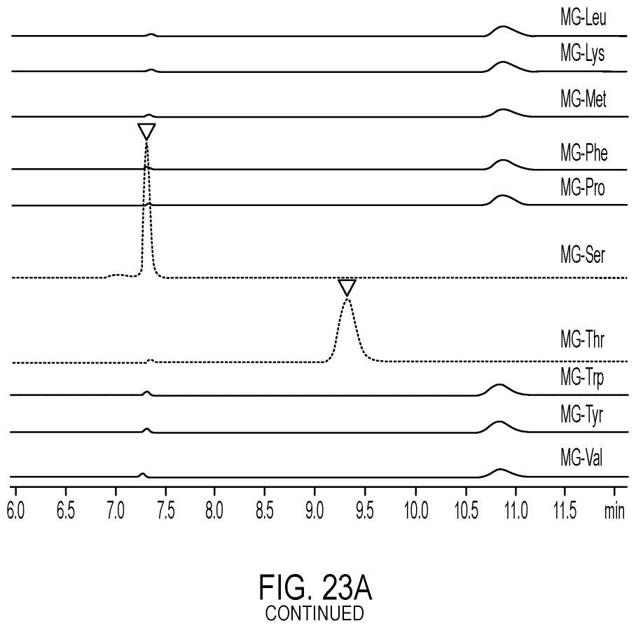

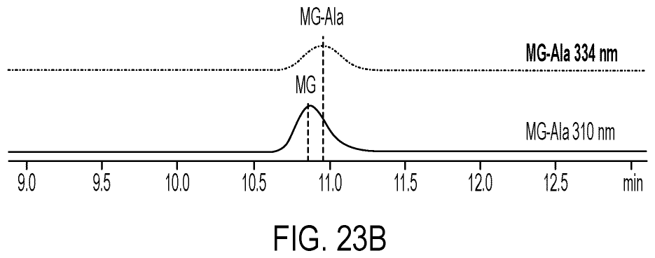

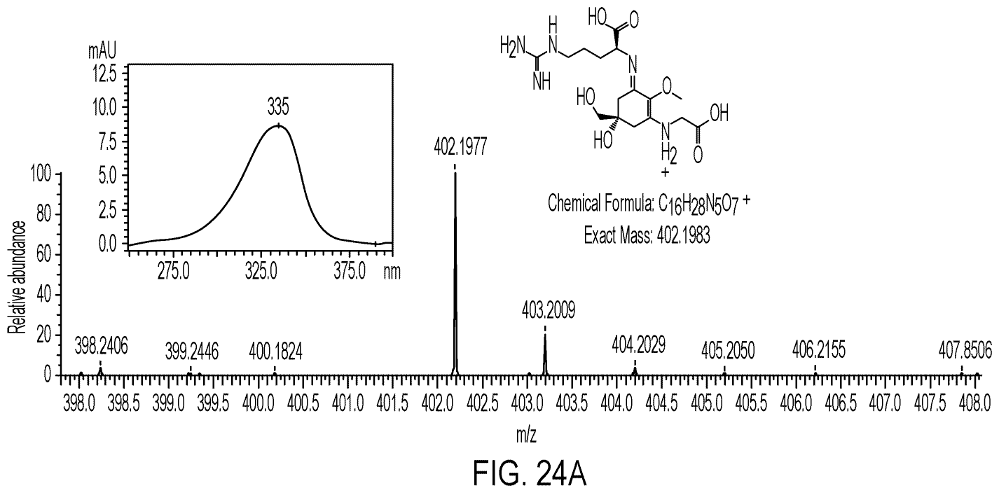

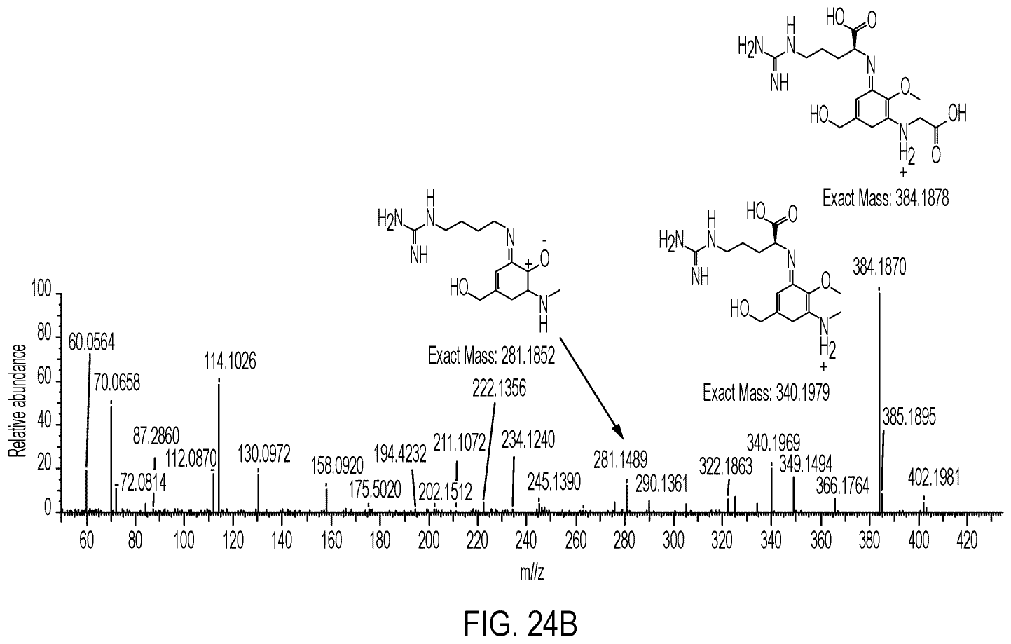

The current and previous heterologous expression studies supported the function of MysD in the biosynthesis of disubstituted MAAs (FIG. 3).^{29,35} To further characterize its catalytic properties, recombinant His₆-tagged MysD of N. linkia NIES-25 was prepared from E. coli after a single affinity purification (FIG. 21). The enzyme reaction was performed with MysD (0.5 μM), MG (50 μM), and L-Thr (1 mM) in the presence of ATP (1 mM) and Mg²⁺ (10 mM) at room temperature for 2 h. HPLC analysis of the reaction mixture identified the formation of porphyra-334 (FIG. 5A), which showed the same maximal absorbance and MS spectrum as that from the heterologous production (FIG. 3C, FIG. 10). No product was formed in the control reactions without enzyme or ATP (FIG. 5A). The requirement of ATP for the MysD reaction supports its prediction as the D-Ala-D-Ala ligase-like enzyme of the ATP grasp superfamily.²⁹ The optimal temperature and pH of its reaction were determined at 37° C. and pH=8.5 (FIG. 22). Under these optimal reaction conditions, all 20 natural amino acids were screened (5 mM) along with MG (50 μM) in the MysD reaction. HPLC analysis found that MysD was able to accept six amino acids as its substrate, including L-Ala, L-Arg, L-Cys, L-Gly, L-Ser and L-Thr (FIG. 5B, FIG. 23A). LC-HRMS and MS/MS analysis indicated the formation of their corresponding disubstituted MAAs, MG-Ala, MG-Arg (observed [M+H]⁺: 402.1977; calculated [M+H]⁺: 402.1983), MG-Cys (observed [M+H]⁺: 349.1059; calculated [M+H]⁺: 349.1064), mycosporine-2-Gly (observed [M+H]⁺: 303.1182; calculated [M+H]⁺: 303.1187), shinorine, and porphyra-334 (FIGS. 24, 25, and 26). L-Ser and L-Thr led to the complete consumption of MG in the MysD reactions after 3 h, followed by L-Cys. The retention times of MG-Ala and MG were very close and the left shoulder of the MG-Ala peak at 310 nm suggested a small amount of MG left in the reaction (FIG. 23B). Nonetheless, the result of this biochemical study well agreed with the production of porphyra-334 along with small amounts of shinorine and MG-Ala in the above heterologous expression study (FIG. 3C). To further understand MysD's substrate preference, the enzyme concentration was lowered to 0.25 μM. Under these conditions, MysD showed the highest activity toward L-Thr, converting about 40% MG into porphyra-334 in 8 min. The consumed MG level in this reaction was set as 100% to normalize its level in the five other reactions, which was determined from the concentrations of produced disubstituted MAAs in the reactions (FIG. 5C). This quantitative analysis showed that the consumption level of MG in the MysD reaction containing L-Ser was about 12.7% to that with L-Thr, followed by L-Cys (0.9%) and L-Ala (0.4%), and two other amino acids (about 0.06%). Together, the results of these biochemical studies highlight the broad substrate scope of MysD and its strong preference toward L-Thr in the MAA biosynthesis.

Recent advances in bioinformatics and synthetic biology tools have unleashed the potential of all organisms for the discovery of new natural products and new enzymology for a variety of applications.⁴⁷ In the search for new MAA analogs, a group of Fe(II)/2OG enzymes that are frequently co-occurred with the known MAA biosynthetic enzymes was identified. Refactoring such an MAA BGC from N. linkia NIES-25 for the heterologous expression in E. coli interrogated the catalytic functions of MysA, MysB, MysC, MysD, MysH, and one SDR for the biosynthesis of MAA analogs. The direct conversion of disubstituted MAAs into corresponding palythines by MysH filled a critical gap in the biosynthetic understanding of many MAA analogs produced by a variety of prokaryotic and eukaryotic organisms. Furthermore, this work provided the first biochemical insights into the substrate preference of MysD.

### Experimental Procedures

General Experimental Procedures. Molecular biology reagents and chemicals were purchased from Thermo Scientific, NEB, Fisher Scientific or Sigma-Aldrich. GeneJET Plasmid Miniprep Kit and GeneJETGel Extraction Kit (Thermo Scientific) were used for plasmid preparation and DNA purification, respectively. E. coli DH5α (Agilent) was used for routine cloning studies and E. coli BL21-gold(DE3) (Agilent) was used for protein expression and heterologous production. The cyanobacterial strain Nostoc linkia NIES-25 was obtained from National Institute for Environmental Studies, Japan. DNA sequencing was performed with GENEWIZ or Eurofins. A Shimadzu Prominence UHPLC system (Kyoto, Japan) coupled with a PDA detector was used for HPLC analysis. NMR spectra were recorded in D₂O on a Bruker 600 MHz spectrometer located in the AMRIS facility at the University of Florida, Gainesville, FL, USA. Spectroscopy data were collected using Topspin 3.5 software. HRMS data were generated on a Thermo Fisher Q Exactive Focus mass spectrometer equipped with an electrospray probe on Universal Ion Max API source.

Bioinformatics Analysis. The SSN of ATP-grasp ligases (ATP_Grasp_3, PF02655) was generated by EFI-Enzyme Similarity Tool (efi.igb.illinois.edu) with ˜35% cut-off threshold.³⁰ The identified MysC containing cluster (585 homologs) was further re-analyzed with ˜45% cut-off threshold. The resultant MysC-containing cluster was submitted for GNN analysis (efi.igb.illinois.edu) with a neighborhood size set at 10 and a co-occurrence lower limit set at 10%. All the SSNs and GNN were visualized in Cytoscape.⁴⁸ The amino acid sequences of mined MysH homologs were aligned by ClustalW algorithm.⁴⁹

Construction of Refactored BGCs. The MAA biosynthetic genes were amplified from isolated genomic DNA of Nostoc linkia NIES-25. The mysAB together were amplified and cloned into pETDuet-1 NcoI/PstI sites to give pETDuet-1-mysAB. The mysC or mysCD were then cloned into the KpnI/XhoI site of pETDuet-1-mysAB to give pETDuet-1-mysABC and pETDuet-1-mysABCD. The sdr was cloned into the NdeI/XhoI site of pACYCDuet-1, and the mysH was cloned into the NcoI/PstI site of pACYCDuet-1 or pACYCDuet-1-sdr. The mysC was then cloned into the KpnI/XhoI site of pACYCDuet-1 or pACYCDuet-1-mysH. All oligonucleotide primers (Table 4) used were ordered from Sigma-Aldrich. The resultant constructs were transformed or co-transformed into E. coli BL21-gold(DE3). After appropriate antibiotics selection, positive clones were used for fermentation.

TABLE 4
Primers used to construct refactored BGCs and express MysD.

| Primer | Sequence (5′-3′) |
|---|---|
| MysA-NcoI-F | CATGCCATGGTGAGCATTGTTCAAACAA (SEQ ID NO: 117) |
| MysB-PstI-R | CATGCTGCAGTCACGCAGTTCTGCGGATA (SEQ ID NO: 118) |
| MysC-KpnI-F | CGTCGGTACCATGGCACAATCTATTTCCG (SEQ ID NO: 119) |
| MysC-XhoI-R | CAGACTCGAGCTAATCCCCACCCAATTCCA (SEQ ID NO: 120) |
| MysD-NdeI-F | CATGCATATGCCAGTACTTCGTATC (SEQ ID NO: 121) |
| MysD-XhoI-R | CATGCTCGAGCTAAATCATTTGTGAAAGCT (SEQ ID NO: 122) |
| MysH-NcoI-F | TAATAAGGAGATATACCATGGTGAAGGTAGACACACA (SEQ |
| | ID NO: 123) |
| MysH-PstI- R | GCAAGCTTGTCGACCTGCAGTCGATGTACTTGAACTCTAG |
| | (SEQ ID NO: 124) |
| SDR-NdeI-F | TAAGAAGGAGATATACATATGGCTTCTCTAGAAAATCA |
| | (SEQ ID NO: 125) |
| SDR-XhoI-R | GTTTCTTTACCAGACTCGAGCTAAGTGCGCCGATTAACTA |
| | (SEQ ID NO: 126) |

Fermentation, Extraction, and Isolation. To characterize MAA production in its native producer, Nostoc linkia NIES-25 was cultured in 300 mL BG-11 medium (Sigma-Aldrich) at 26° C. The culture was air bubbled and received a lighting cycle of 16 h/8 h (light/dark) with the illumination of 2000-2500 lux. After 21 days, the cells were pelleted down by centrifugation (4500 rpm, 15 min). The cyanobacterial cell pellet was lysed by sonication in ice-cold methanol (10 s pulse and 20 s rest, 2 min pulse in total). After centrifugation (4500 rpm, 30 min), the clear supernatants of lysates were collected and evaporated under reduced pressure. The dried extracts were resuspended in water (1 mL) for HPLC and LC-HRMS analysis. Following the same procedure, the expensed culture medium was lyophilized and re-dissolved in water (1 mL) for HPLC and LC-MS analysis.

To characterize the heterologous expression of the MAA BGC from Nostoc linkia NIES-25, E. coli strains carrying refactored gene clusters were cultured in 2×50 mL in Luria-Bertani broth supplemented with 50 μg/mL ampicillin and/or chloramphenicol (37° C., 225 rpm).

When the cell culture OD₆₀₀ reached 0.5, IPTG (final concentration 0.1 mM) was added to the culture to induce gene expression (18° C., 180 rpm, 20 h). The cells were harvested by centrifugation (4500 rpm, 10 min), and collected cell pellets were extracted twice by 1 mL methanol. The methanolic extracts were dried in the speed vacuum concentrator and resuspended in water (300 μL) for HPLC and LC-MS analysis.

For the large-scale production of palythine-Thr, E. coli expressing mysAB2CDH was cultured in 8×1 L Luria-Bertani broth using the same expression conditions as described above. After expression, the cells were harvested by centrifugation (6000 rpm, 20 min), and lysed by sonication in 2×30 mL ice-cold methanol (10 s pulse and 20 s rest, 8 min pulse in total). The cell lysates were centrifuged (4500 rpm, 10 min) and the clear supernatants were evaporated under reduced pressure. The dried methanolic extracts were resuspended in 1 mL water and were first purified on an Agilent Zorbax SB-C18 column (9.4×250 mm, 5 μm) using 0.1% formic acid in water and 2% methanol as mobile phases. Corresponding fractions were collected (maximal absorption at 320 nm), combined, evaporated to remove organic solvents, and then lyophilized. The residues were resuspended in water (200 μL) and further purified on a Phenomenex Luna C8 column (4.6×250 mm, 5 μm) using the same mobile phases above. Palythine-Thr fractions were collected, combined, evaporated to remove organic solvents, and lyophilized. About 1 mg of palythine-Thr was purified for NMR analysis.

Palythine-Thr: white solid; ¹H NMR (600 MHz, D₂O) δ 4.32 (m, 1H), 4.08 (d, J=4.6 Hz, 1H), 3.69 (s, 3H), 3.58 (s, 2H), 2.97 (d, J=17.1 Hz, 1H), 2.93 (d, J=17.5 Hz, 1H), 2.77 (dd, J=17.5, 1.3 Hz, 1H), 2.71 (dd, J=17.1, 1.4 Hz, 1H), 1.26 (d, J=6.5 Hz, 3H); ¹³C NMR (151 MHz, D₂O) δ 177.90, 163.8, 163.8, 127.7, 74.2, 70.9, 70.2, 67.4, 62.0, 38.6, 36.6, 22.2.

MysD Expression and Purification. The mysD gene was amplified from the isolated genomic DNA of Nostoc linkia NIES-25 and inserted into the NdeI/XhoI sites of pET28b, and the resultant construct pET28b-mysD was transformed into E. coli BL21-gold(DE3) for the expression of recombinant N-His₆-tagged MysD. Protein expression was carried out in 500 mL Luria-Bertani broth supplemented with 50 g/mL kanamycin (37° C., 225 rpm).

When the cell culture OD₆₀₀ reached 0.5, IPTG (final concentration 0.1 mM) was added to the culture to induce gene expression (18° C., 180 rpm, 20 h). The cells were harvested by centrifugation (6000 rpm, 20 min), and collected cell pellets were resuspended in the lysis buffer (25 mM Tris-Cl, pH 8.0, 100 mM NaCl, 1 mM β-mercaptoethanol and 10 mM imidazole) and lysed by sonication on ice (10 s pulse and 20 s rest, 1 min in total).

Following centrifugation (15000 rpm, 4° C., 30 min), recombinant MysD was purified by the HisTrap Ni-NTA affinity column (GE Healthcare). N-His₆-tagged MysD was eluted using a 0-100% B gradient in 15 min at the flow rate of 2 mL/min, using A buffer (25 mM Tris-Cl, pH 8.0, 250 mM NaCl, 1 mM β-mercaptoethanol and 30 mM imidazole) and B buffer (25 mM Tris-Cl, pH 8.0, 250 mM NaCl, 1 mM β-mercaptoethanol and 300 mM imidazole). Fractions with recombinant MysD were collected, concentrated, and buffer-exchanged into storage buffer (50 mM Tris-Cl, pH 8.0, 10% glycerol). The purity of the recombinant protein was analyzed on SDS-PAGE and the concentration was determined by NanoDrop.

MysD Reaction. MG was purified from extracts of E. coli expressing MysAB2C by HPLC and used as the substrate for the MysD reactions. The quality of MG was calculated based on its reported extinction coefficient (28,100 M⁻¹cm⁻¹). The initial MysD reactions included MG (50 μM), L-Thr (1 mM), Mg²⁺ (10 mM), and ATP (1 mM) in 100 mM Tris-Cl, pH 7.5. The reactions were initiated by adding MysD (0.5 μM) and then incubated at room temperature for 2 h. The control reactions omitted MysD or ATP. All reactions were quenched by heat inactivation at 95° C. for 10 min. After centrifugation at 20,000×g for 15 min, the clear supernatants were collected for HPLC and LC-HRMS analysis. To determine the optimal reaction conditions, the MysD reaction was performed in 100 mM buffer with a pH of 6.5 to 11 at 16 to 60° C. for 6 min. To explore the substrate scope of MysD, all 20 natural amino acids (5 mM) were screened in the above reaction mixtures under the optimal conditions for 3 h. The reactions were terminated and then analyzed in the HPLC and/or LC-MS analysis. To determine the relative activity of six identified amino acids as MysD's substrates, a two-step strategy was used. First, the reactions were performed with 0.25 μM MysD for 8 min, which led to no more than 50% consumption of MG into porphyra-334 with the best substrate L-Thr and into shinorine with L-Ser. For the other four amino acids, the levels of their corresponding disubstituted MAAs were determined after the reaction time was elongated to 30 min. All reactions were performed in at least two independent replicates.

HPLC and LC-MS Analysis. Samples were analyzed on a Shimadzu Prominence UHPLC system (Kyoto, Japan) coupled with a PDA detector. The compounds were separated on a Phenomenex Luna C8 column (4.6×250 mm, 5 μm) using the following HPLC program: 2% B for 15 min, 2-90% B gradient in 2 min, 90% B for 2 min, 90-2% in 2 min, and re-equilibration in 2% B for 6 min. The A phase was 0.1 M triethylammonium acetate pH 7.0 and the B phase was methanol. The flow rate was set at 0.5 mL/min. In the quantitative analysis of relative activity of MysD with different amino acid substrates, water containing 0.1% formic acid was used as phase A to fully separate MG with MG-Ala. LC-HRMS and HRMS/MS experiments were conducted on Thermo Scientific™ Q Exactive Focus mass spectrometer with Dionex™ Ultimate™ RSLC 3000 uHPLC system, equipped with H-ESI II probe on Ion Max API Source. Methanol (B)/Water (A) containing 0.1% formic acid were used as mobile phases, and the same LC program was used as in the HPLC analysis. The eluents from the first 3 min were diverted to waste by a diverting valve. MS1 signals were acquired under the Full MS positive ion mode covering a mass range of m/z 150-2000, with resolution at 35,000 and AGC target at 1e6. Fragmentation was obtained with the Parallel Reaction Monitoring (PRM) mode using an inclusion list of calculated parental ions. The AGC target was set at 5e4 for MS2. Precursor ions were selected in the quadrapole typically with an isolation width of 3.0 m/z and fragmented in the HCD cell at a collision energy (CE) of 30. For some ions, the isolation width was 2.0 m/z and step-wise CE of 15, 20, and 25 were used.

### Example 4: MysD Accepts Additional Substrates L-Ile, L-Met, and L-Val to Produce New MAA Analogs

Previously, different bioinformatic approaches were taken to assess the distribution of the MAA biosynthesis, and a putative gene cluster was identified from Nostoc linckia NIES-25 that encodes a short-chain dehydrogenase/reductase (SDR) and a nonheme iron(II)- and 2-oxoglutarate-dependent oxygenase (MysH) as potential new biosynthetic enzymes. Heterologous expression of refactored gene clusters in E. coli produced two known biosynthetic intermediates, 4-deoxygadusol (4-DG) and mycosporine-glycine (MG), and three disubstituted MAA analogs, porphyra-334, shinorine, and mycosporine-glycine-alanine. Importantly, the disubstituted MAAs were converted into palythines by MysH in E. coli. Furthermore, biochemical characterization revealed the substrate preference of recombinant MysD, an ATP-grasp ligase, in the formation of disubstituted MAAs. This study advances the biosynthetic understanding of an important family of natural UV photoprotectants and opens new opportunities to the development of next-generation sunscreens.

The use of two ATP-grasp ligases MysC and MysD and MysH has now been further expanded to generate a library of mono- and di-substituted MAA analogs and palythines. In addition, a glycosyltransferase was identified that could contribute to the synthesis of glycosylated MAA analogs.

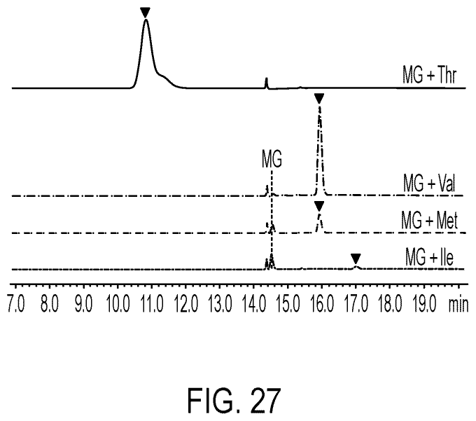

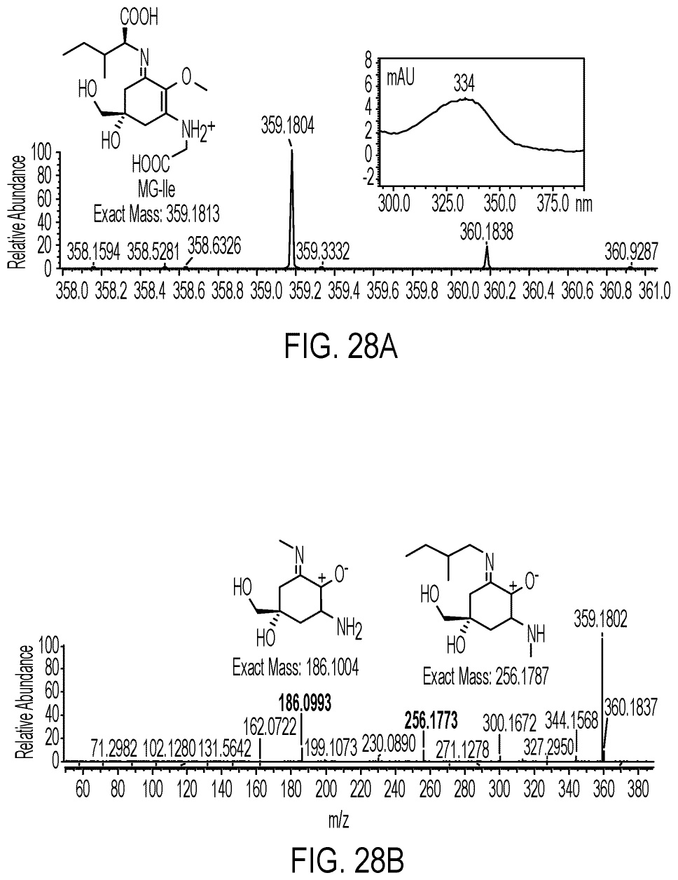

Previously, it was demonstrated that the recombinant MysD of Nostoc linckia NIES-25 accepts six natural amino acids (l-Thr, l-Ser, l-Cys, l-Ala, l-Arg, and l-Gly) as its substrates to synthesize MAA analogs. It was recently found that three other natural amino acids are also utilized in the MysD reaction, including l-Ile, l-Met, and l-Val (FIG. 27). The reaction solutions contained 50 μM mycosporine-glycine (MG) and 5 mM amino acid substrates. After adding 0.5 μM MysD, the reactions were initiated and carried out at 37° C. for 24 hours. Their corresponding di-substituted MAAs showed the expected m/z values in the LC-HRMS analysis (MG-Ile, observed [M+H]⁺ m/z 359.1804, calculated [M+H]⁺ 359.1813; MG-Met, observed [M+H]⁺ m/z 377.1368, calculated [M+H]⁺ 377.1377; MG-Val, observed [M+H]⁺ m/z 345.1650, calculated [M+H]+ 345.1656). Furthermore, their structures were validated by HR-MS/MS analysis (FIGS. 28A-28B, 29A-29B, and 30A-30B). Of note, these three new di-substituted MAAs were eluted after MG, suggesting a higher hydrophobicity.

### Example 5: MysH Cleaves the Glycine Side Chain of MG In Vivo

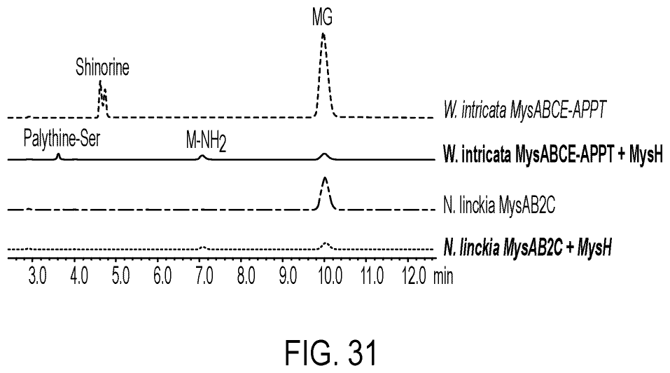

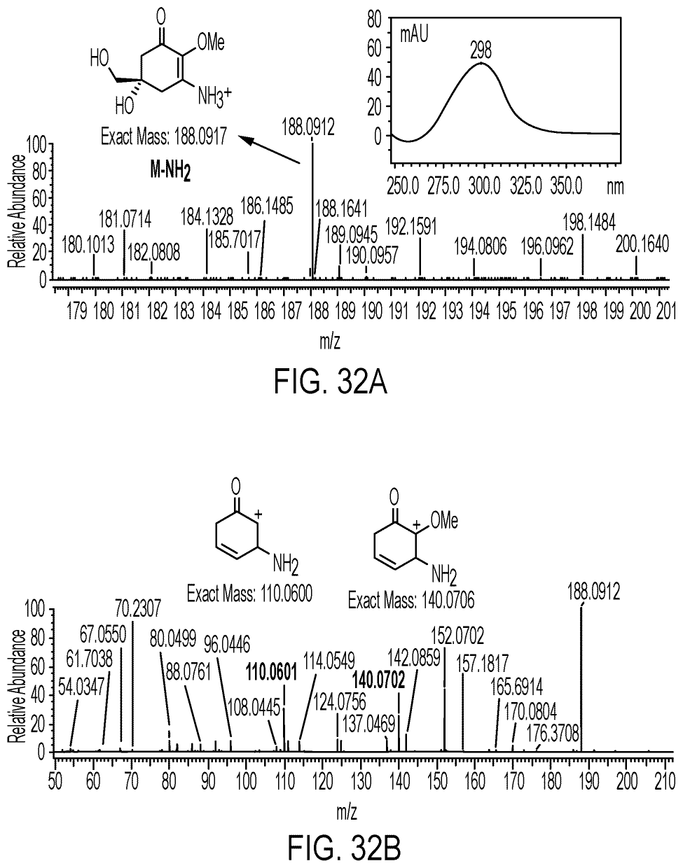

It was previously reported that MysH from Nostoc linckia NIES-25 converts disubstituted MAAs into palythine-Thr, palythine-Ser, and palythine-Ala when expressed in E. coli, indicating the substrate flexibility of MysH. MysH was coexpressed with MysA, MysB, and MysC, all from Nostoc linckia NIES-25 in E. coli. MysA, MysB, and MysC together produce MG. Interestingly, in addition to the reduced amount of MG, a novel metabolite with a retention time of 7.05 min was observed (FIG. 31). Its maximal UV absorbance was at 298 nm (FIGS. 32A-32B), which is close to that of 4-DG. Based on the HRMS and MS/MS spectra, this molecule was predicted to be mycosporine-amine (M-NH₂, observed [M+H]⁺ m/z 188.0912, calculated [M+H]⁺ 188.0923), which is produced from MG by MysH (FIGS. 32A-32B). To further characterize the substrate flexibility of MysH, MysH was coexpressed with an MAA biosynthetic gene cluster (BGC) from Westiella intricata UH strain HT-29-1 in E. coli. This BGC encodes MysA, MysB, MysC, and MysE (a nonribosomal peptide synthetase-like enzyme) (doi.org/10.1186/si2864-015-1855-z). MysE requires a posttranslational phosphopantetheinylation modification to become a catalytically functional enzyme, which can be catalyzed by a phosphopantetheinyltransferase from the cyanobacterium Anabaena sp. PCC 7102, APPT (doi.org/10.1038/s41598-017-12244-3). When expressed along with APPT in E. coli, the MAA BGC from W. intricata UH strain HT-29-1 produced shinorine and a large amount of MG (FIG. 31). Remarkably, coexpressed MysH completely converted shinorine into palythine-Ser, while the majority of MG was converted into M-NH₂ (FIG. 31). This result suggested that MysH can use both mono- and di-substituted MAAs as its substrates.

### Example 6: Biochemical Characterization of MysH

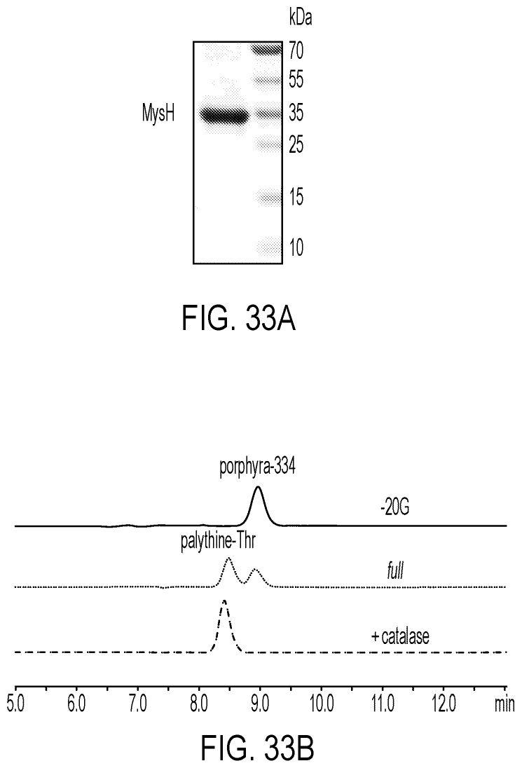

To further characterize the catalytic properties of MysH, the recombinant MysH of Nostoc linckia NIES-25 was prepared with a C-terminal His₆-tag in E. coli after a single Ni-NTA affinity purification (FIG. 33A). The MysH reaction contained 50 mM Tris-Cl at pH 7.5, 0.5 uM MysH, 50 uM porphyra-334, 1 mM 2-oxoglutarate (2OG), 1 mM Fe(NH₄)₂(SO₄)₂, 10 mM ascorbate, and the reaction was performed at room temperature overnight. MysH successfully converted porphyra-334 into palythine-Thr in the HPLC analysis, while the complete conversion was not achieved with a higher enzyme concentration or a longer reaction time. No consumption of porphyra-334 was observed in the control reaction lacking 2OG (FIG. 33B). To improve the reaction conversion, different concentrations of 2OG, Fe(NH₄)₂(SO₄)₂, ascorbate, and shaking speed were tested, but no significant improvement was observed. On the other hand, the inclusion of catalase led to the full conversion of porphyra-334 by MysH (FIG. 33B). The Fe(III)-O—O· species is likely one key intermediate in the MysH reaction. Hydrogen peroxide may be released via hydroxylation of Fe(III)-O—O· and inhibit the enzyme reaction.

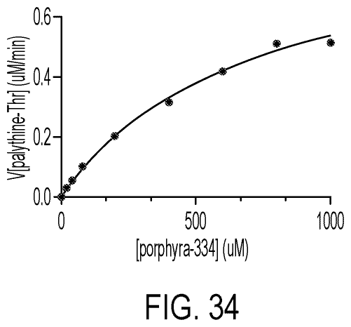

The optimal MysH reaction conditions were determined to be 50 mM HEPES, pH 7.5, 0.5 uM MysH, 1 mM α-KG, 1 mM ascorbate, 10 uM Fe(NH₄)₂(SO₄)₂, and 8 μg/mL catalase. Steady-state kinetic studies were performed with 20 to 1000 uM porphyra-334, and the reactions were carried out at room temperature for 30 min. The reactions followed Michaelis-Menten kinetics (FIG. 34). The kinetic parameters were Kₘ: 385 μM, Vₘₐₓ: 0.62 uM/min, k_{cat}: 1.24 min⁻¹.

### Example 7: One-Pot Reaction with MysD and MysH Produce 12 Palythines

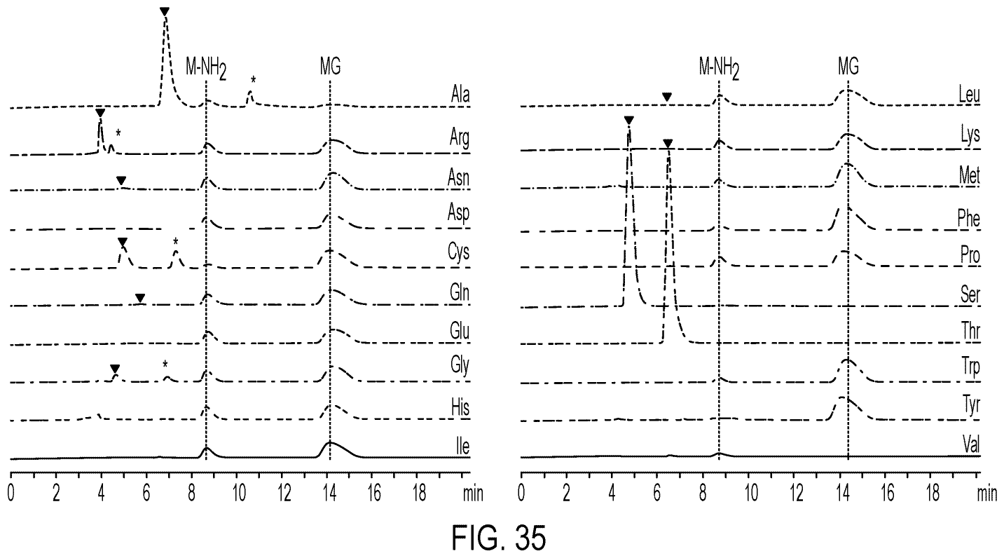

Given the notable substrate flexibility of MysD and MysH, their one-pot reactions to produce palythines were examined next. The optimal conditions were first determined. Temperatures ranging from 20 to 37° C. showed a minimal effect on the reaction turnover. The optimal pH was determined to be 8.0, while the optimal molar ratio of MysD to MysH was determined to be 1:3. The following conditions were then used for the MysD and MysH coupled reaction: 50 mM HEPES, pH 8.0, 10 mM MgCl₂, 40 uM MG, 5 mM amino acid, 5 mM ATP, 0.5 uM MysD, 1.5 uM MysH, 1 mM 20G, 1 mM ascorbate, 10 uM Fe(NH₄)₂(SO₄)₂, and 8 μg/mL catalase. All twenty natural amino acids were screened in the overnight reaction at room temperature. In the one-pot reaction, MysD still accepted l-Thr, l-Ser, l-Cys, l-Ala, l-Arg, and l-Gly as its substrates, and MysH then converted the disubstituted MAA analogs into corresponding palythines (FIG. 35). In addition, palythine-Gln and palythine-Leu were also synthesized in the one-pot reactions, although MG-Gln and MG-Leu were not observed in the reactions with MysD alone. Furthermore, disubstituted MAA analogs with L-Ile, L-Met, and L-Val moieties were also produced by MysD and then converted into corresponding palythines by MysH. Palythine-Ile, palythine-Met, and palythine-Val were eluted after 22 min with the current HPLC program and were not shown in the LC trace. Their corresponding molecular weights and those of all other palythines were confirmed in HR-MS analysis (palythine-Ala, observed [M+H]⁺ m/z 259.1284, calculated [M+H]⁺ 259.1288; palythine-Arg, observed [M+H]⁺ m/z 344.2060, calculated [M+H]⁺ 344.1928; palythine-Asn, observed [M+H]⁺ m/z 302.1349, calculated [M+H]⁺ 302.1347; palythine-Cys, observed [M+H]⁺ m/z 291.1088, calculated [M+H]⁺ 291.1099; palythine-Gln, observed [M+H]⁺ m/z 316.1497, calculated [M+H]⁺ 316.1503; palythine-Gly, observed [M+H]⁺ m/z 245.1124, calculated [M+H]⁺ 245.1132; palythine-Ile, observed [M+H]⁺ m/z 301.1750, calculated [M+H]⁺ 301.1758; palythine-Leu, observed [M+H]⁺ m/z 301.1750, calculated [M+H]⁺ 301.1758; palythine-Ser, observed [M+H]⁺ m/z 275.1238, calculated [M+H]⁺ 275.1227; palythine-Thr, observed [M+H]⁺ m/z 289.1382, calculated [M+H]⁺ 289.1394; palythine-Met, observed [M+H]⁺ m/z 319.1312, calculated [M+H]⁺ 319.1322; palythine-Val, observed [M+H]⁺ m/z 287.1594, calculated [M+H]⁺ 287.1600). M-NH₂ was observed in almost all reactions except for those with l-Thr and l-Ser.

### Example 8: MysC Accepts L-Ala as its Substrate

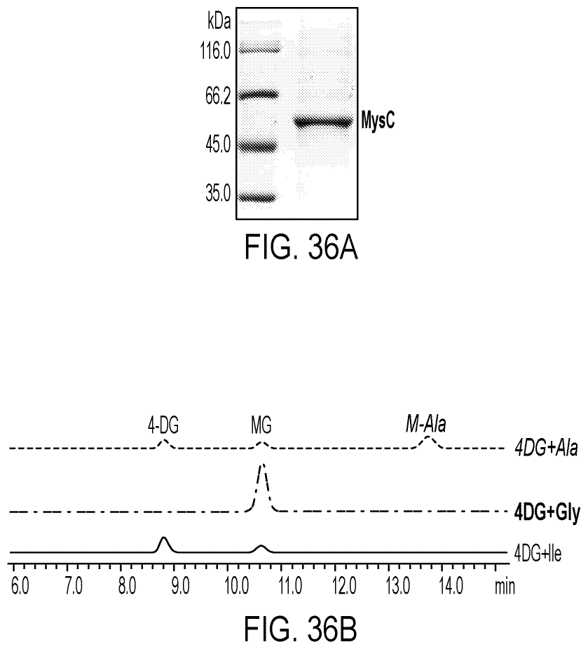

Natural MAAs are dominant with a C3-glycine, but some analogs carry a different C3 moiety, including alanine, serine, glutamic acid, glutamicol, lysine, ornithine, GABA, etc. (doi: 10.3390/antiox4030603; doi: 10.1128/AEM.01632-16; doi: 10.3390/md17060356). To further characterize the catalytic properties of MysC from Nostoc linckia NIES-25, its recombinant protein was prepared with an N-terminal His₆-tag from E. coli after a single Ni-NTA affinity purification (FIG. 36A). The MysC reaction was then prepared in 50 mM HEPES pH 7.5 with 50 uM 4-DG, 5 mM ATP, 5 mM glycine, and 0.5 uM MysC. The recombinant MysC converted 4-DG and Glycine into MG (FIG. 36B). Among all 20 natural amino acids, alanine was another amino acid to be accepted by MysC to form mycosporine-alanine (M-Ala) (FIG. 36B). Note that there was glycine contamination in the protein purification process, leading to the formation of MG in all reactions.

### Example 9: Ancestral Construction of MysC

Compared with MysD, the substrate scope of MysC is more stringent. As the ancestral MysC homologs may possess a broader substrate scope, the ancestral sequences of MysC homologs using the webserver FireProt^{ASR} (doi: 10.1093/bib/bbaa337). Four computed ancestor MysC homologs (Table 5) were synthesized and heterologously expressed in E. coli. They can be used to synthesize new MAA analogs.

TABLE 5
Sequences of MysC ancestors

| MysC homolog | Sequence |
|---|---|
| MysC-158 | MSLSAPPSRSKIRSTLKTLGTLVLLLLALPLNAAIVLV |
| (computed ancestor) | ALLRNLITRPRKRATAANPKTVLISGGKMTKALQLAR |
| | SFHRAGHRVILVETHKYWLTGHRFSNAVDRFYTVPA |
| | PQDDPEGYAQALLDIVQKENVDVYVPVCSPVASYYD |
| | ALAKETLSPHCEVFHFDADTVKMLDDKYQFAEMAR |
| | SLGLSVPESHRITSPEQVLDFDFSQSEGRKYILKSIAYD |
| | SVRRLDLTKLPCPTPEETAAFVRSLPISPDNPWIMQEFI |
| | EGQEYCTHSTVRDGRLRLHCCCESSAFQVNYEHVDN |
| | PEIQEWVQRFVKALNLTGQVSFDFIQTDDDGRVYAIE |
| | CNPRTHSAITMFYNHPGVAEAYLDPDPDLAEPIQPLP |
| | SSRPTYWLYHELWRLLTHPRSLQDLRERLKTIFRGKD |
| | AIFDWDDPLPFLMVHHWQIPLLLLKNLRQGKDWVRI |
| | DFNIGKLVELGGD (SEQ ID NO: 113) |
| MysC-175 | MVVAENPKNILITGGKMTKALQLARSFHAAGHRVFL |
| (computed ancestor) | VETHKYWLSGHRFSNAVDRFYTVPAPQKDPEGYVQ |
| | GLLDIVKQENIDVFIPVSSPVASYYDSLAKPVLSPYCE |
| | VFHFDAEITKMLDNKFTFSEKARSLGLSAPKSFLITDP |
| | EQVLNFDFAADQGSQYILKSIPYDSVHRLDMTKLPCD |
| | KEEMAEYVKSLPISEENPWIMQEFITGQEYCTHSTVR |
| | DGKIRLHCCSKYPTLFTASSAFQVNYEHVDNPAILQW |
| | VTRFVKELNLTGQISFDFIQAEDDGTVYPIECNPRTHS |
| | AITMFYNHLPGVVADAYLKDSPDEEEPIQPLPDSKPT |
| | YWLYHELWRLTEIRSWSQLQAWINNILKGTDAIFQV |
| | NDPLPFLMVHHWQIPLLLLNNLRKLKGWVRIDFNIG |
| | KLVELGGD (SEQ ID NO: 114) |
| MysC-225 | MVVAENPKNILITGGKMTKALQLARSFHAAGHRVFL |
| (computed ancestor) | VETHKYWLSGHRFSNAVDRFYTVPAPQKDPEGYIQA |
| | LLDIVKQENIDVFVPVSSPVASYYDSLAKPVLSPYCE |
| | VFHFDADITKMLDDKFTFSEKARSLGLSAPKSFLITDP |
| | EQVLNFDFASDQGSQYILKSIPYDSVHRLDMTKLPCD |
| | SKEEMAAYVKSLPISEENPWIMQEFITGQEYCTHSTV |
| | RDGKIRLHCCSKYPTLFTASSAFQVNYEHVDNPKILQ |
| | WVTRFVKELNLTGQISFDFIEAEDDGTVYAIECNPRT |
| | HSAITMFYNHLPGVVADAYLGKSPSAEEPIQPLPDSK |
| | PTYWLYHEVWRLTEIRSWSQLQTWINNILRGKDAIFQ |
| | VNDPLPFLMVHHWQIPLLLLNNLRKLKGWVRIDFNI |
| | GKLVELGGD (SEQ ID NO: 115) |
| MysC-230 | MVVAENPKNILLTGGKMTKALQLARSFHAAGHRVIL |
| (computed ancestor) | VETHKYWLSGHRFSNAVDRFYTVPAPQKDPEGYTQ |
| | ALLAIAKQENIDVYVPVCSPVASYYDSLAKPVLSGCC |
| | EVFHFDADVTKMLDDKFAFSEKARSLGLSVPKSFLIT |
| | DPEQVLNFDFSNEQKRKYILKSIPYDSVHRLDMTKLP |
| | CDSKEEMAAYVKSLPISEENPWIMQEFIPGKEYCTHS |
| | TVRNGELRLHCCCEYPTLFTASSAFQVNYENVDNPKI |
| | LQWVSHFVKELKLTGQISFDFIEAEDDGTVYAIECNP |
| | RTHSAITMFYNHLPGVVADAYLGKEPLEEPLQPLPDS |
| | KPTYWLYHEVWRLTEIRSFSQLQTWIKNILRGKDAIF |
| | SVNDPLPFLMVHHWQIPLLLLNNLRRLKGWIRIDFNI |
| | GKLVELGGD (SEQ ID NO: 116) |

### Example 10: Co-Expression of a Glycosyltransferase with MysABCD

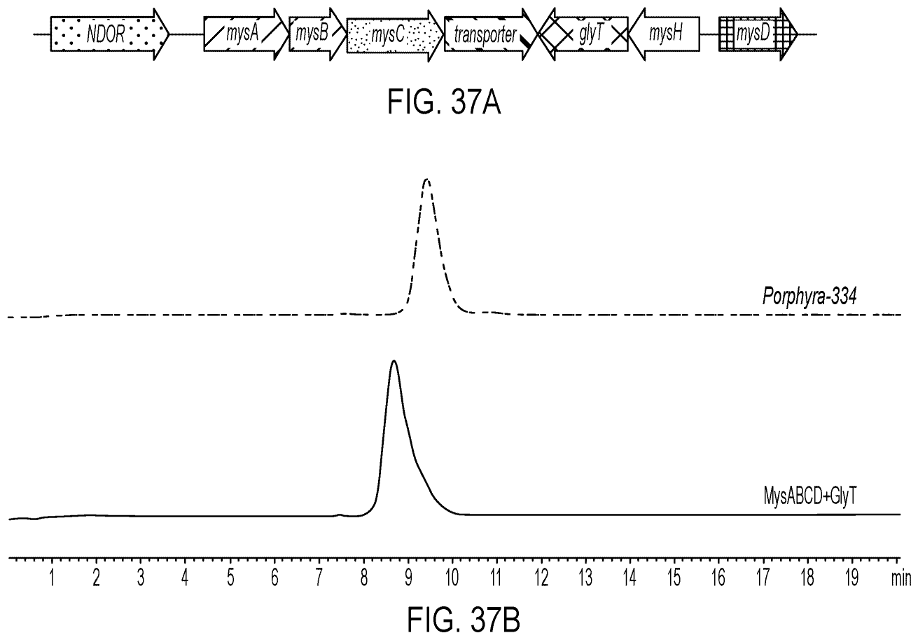

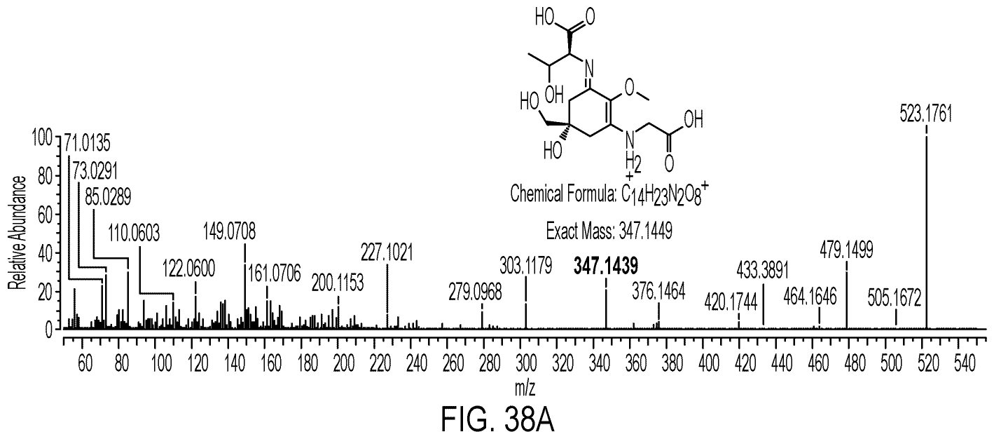

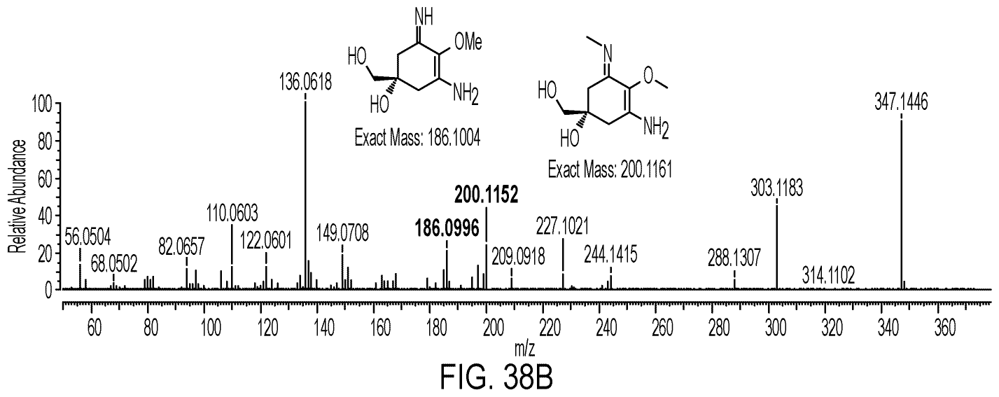

In the previous studies, the frequent occurrence of glycosyltransferase (GlyT) genes in the MAA BGCs was observed (1000 co-occurrence frequency). Many glycosylated MAA analogs have been reported, but the corresponding GlyTs remain uncharacterized. Here, the GlyT gene from Aphanothece hegewaldii CCALA 016 (Genbank accession: WP_106457502.1) was synthesized and cloned into the expression vector pET28a. The glyT gene sits in the same operon as mysH in Aphanothece hegewaldii MAA BGC (FIG. 37A). The pET28a-g/glyT was co-transformed with pETduet-mysAB-mysCD into E. coli cells. The HPLC analysis of the methanolic extracts showed that the MAA analog isolated from cells co-expressing GlyT was eluted earlier than porphyra-334 (FIG. 37B). The LC-HRMS analysis revealed that this analog has an observed [M+H]⁺ m/z 523.1761, which corresponds to the porphyra-334 derivatized with a seven-carbon sugar moiety. Further, MS/MS and MS/MS/MS analysis confirmed the presence of the porphyra-334 moiety (FIG. 38).

### Methods

General experimental procedures. Molecular biology reagents and chemicals were purchased from Thermo Scientific, NEB, Fisher Scientific or Sigma-Aldrich. GeneJET Plasmid Miniprep Kit and GeneJETGel Extraction Kit (Thermo Scientific) were used for plasmid preparation and DNA purification, respectively. E. coli DH5α (Agilent) was used for routine cloning studies and E. coli BL21-gold(DE3) (Agilent) was used for protein expression and heterologous production. DNA sequencing was performed with GENEWIZ or Eurofins. A Shimadzu Prominence UHPLC system (Kyoto, Japan) coupled with a PDA detector was used for HPLC analysis. HRMS data were generated on a Thermo Fisher Q Exactive Focus mass spectrometer equipped with an electrospray probe on Universal Ion Max API source.

Protein expression and purification. The mysD and mysC gene were amplified from the isolated genomic DNA of Nostoc linckia NIES-25 and inserted into the NdeI/XhoI sites of pET28b, and the resultant constructs pET28b-mysD or pET28b-mysC were transformed into E. coli BL21-gold(DE3) for the expression of the recombinant protein. The mysC ancestor genes were codon optimized and synthesized with Twist Bioscience for expression in E. coli. The genes were inserted into NdeI/XhoI sites of pET28a, and the resultant construct pET28a-mysC was transformed into E. coli BL21-gold(DE3) for the expression of a recombinant protein. The mysH gene was amplified from the isolated genomic DNA of Nostoc linckia NIES-25 and inserted into the NcoI/XhoI sites of pET28b, and the resultant construct pET28b-mysH was transformed into E. coli BL21-gold(DE3) for the expression of the recombinant protein with a C-His₆ tag.

Protein expression was carried out in 500 mL Luria-Bertani broth supplemented with 50 μg/mL kanamycin (37° C., 225 rpm). When the cell culture OD₆₀₀ reached 0.5, IPTG (final concentration 0.1 mM) was added to the culture to induce protein expression (18° C., 180 rpm, 20 h). The cells were harvested by centrifugation (6000 rpm, 20 min), and collected cell pellets were resuspended in the lysis buffer (25 mM Tris-Cl, pH 8.0, 100 mM NaCl, 1 mM β-mercaptoethanol and 10 mM imidazole) and lysed by sonication on ice (10 s pulse and 20 s rest, 1 min in total). Following centrifugation (15000 rpm, 4° C., 30 min), recombinant N-His₆-tagged MysD, N-His₆-tagged MysC or C-His₆-tagged MysH were purified by the HisTrap Ni-NTA affinity column (GE Healthcare). Recombinant proteins were eluted using a 0-100% B gradient in 15 min at the flow rate of 2 mL/min, using A buffer (25 mM Tris-Cl, pH 8.0, 250 mM NaCl, 1 mM β-mercaptoethanol and 30 mM imidazole) and B buffer (25 mM Tris-Cl, pH 8.0, 250 mM NaCl, 1 mM β-mercaptoethanol and 300 mM imidazole). Fractions with recombinant proteins were collected, concentrated, and buffer-exchanged into storage buffer (50 mM Tris-Cl, pH 8.0, 10% glycerol). The purity of the recombinant proteins was analyzed on SDS-PAGE, and the concentration was determined by NanoDrop.

In vitro enzymatic reactions. 4-DG, MG, and porphyra-334 were purified from extracts of E. coli expressing MysAB, MysAB2C or MysAB2CD by HPLC and used as the substrate for the enzymatic reactions. The quality of MG was calculated based on its extinction coefficient (28,100 M⁻¹cm⁻¹). The detailed reaction condition are discussed above. All reactions were quenched by heat inactivation at 95° C. for 10 min. After centrifugation at 20,000×g for 15 min, the clear supernatants were collected for LC-HRMS analysis.

HPLC and LC-HRMS analysis. Samples were analyzed on a Shimadzu Prominence UHPLC system (Kyoto, Japan) coupled with a PDA detector. Unless stated elsewhere, the following HPLC procedure was performed. The compounds were separated on a Phenomenex Luna C8 column (4.6×250 mm, 5 μm) using the following HPLC program: 2% B for 15 min, 2-90% B gradient in 2 min, 90% B in 2 min, 90-2% in 2 min, and re-equilibration in 2% B for 6 min. The A phase was water with 0.1 M triethylamine acetate (TEAA) at pH 7 and the B phase was methanol. The flow rate was set at 0.5 mL/min. LC-HRMS and HRMS/MS experiments were conducted on a Thermo Scientific Q Exactive Focus mass spectrometer with a Dionex Ultimate RSLC 3000 uHPLC system, equipped with the H-ESI II probe on an Ion Max API Source. Methanol (B)/water (A) containing 0.1% formic acid were used as mobile phases. The eluents from the first 3 min were diverted to waste by a diverting valve. MS1 signals were acquired under the Full MS positive ion mode, covering a mass range of m/z 150-2000, with resolution at 35 000 and AGC target at 1×10⁶.

Bioinformatic analysis of MysC. Protein sequences from 595 cyanobacteria genomes were obtained by protein BLAST search against the NCBI non-redundant protein database (E-value<1e-5) using query sequences for Nostoc linckia NIES-25 MysC (accession: WP_096541779.1). After filtering sequence length to obtain proteins with 350-550 amino acids, 464 MysD homologs were retrieved. After removing redundant protein at 95% identity, 163 MysC homologs were aligned in Mega Align using the Clustalw, and the phylogenic tree was computed with 1000 bootstraps. The MysC homolog sequences were submitted for ancestral construction using Fireprot^{ASR} (loschmidt.chemi.muni.cz/fireprotasr/).

### REFERENCES

1. Rogers, H. W.; Weinstock, M. A.; Feldman, S. R.; Coldiron, B. M., Incidence estimate of nonmelanoma skin cancer (keratinocyte carcinomas) in the U.S. population, 2012. JAMA Dermatol. 2015, 151 (10), 1081-1086.
2. Siegel, R. L.; Miller, K. D.; Fuchs, H. E.; Jemal, A., Cancer statistics, 2021. CA: Cancer J. Clin. 2021, 71 (1), 7-33.
3. Moan, J.; Grigalavicius, M.; Baturaite, Z.; Dahlback, A.; Juzeniene, A., The relationship between UV exposure and incidence of skin cancer. Photodermatol. Photoimmunol. Photomed. 2015, 31 (1), 26-35.
4. Armstrong, B. K.; Kricker, A., How much melanoma is caused by sun exposure. Melanoma Res. 1993, 3 (6), 395-401.
5. Holick, M. F., Biological effects of sunlight, ultraviolet radiation, visible light, infrared radiation and vitamin D for health. Anticancer Res. 2016, 36 (3), 1345-1356.
6. Ghiasvand, R.; Weiderpass, E.; Green, A. C.; Lund, E.; Veierod, M. B., Sunscreen use and subsequent melanoma risk: A population-based cohort study. J. Clin. Oncol. 2016, 34 (33), 3976-3983.
7. Latha, M. S.; Martis, J.; Shobha, V.; Sham Shinde, R.; Bangera, S.; Krishnankutty, B.; Bellary, S.; Varughese, S.; Rao, P.; Naveen Kumar, B. R., Sunscreening agents: a review. J. Clin. Aesthet. Dermatol. 2013, 6 (1), 16-26.
8. Krause, M.; Klit, A.; Jensen, M. B.; Soeborg, T.; Frederiksen, H.; Schlumpf, M.; Lichtensteiger, W.; Skakkebaek, N. E.; Drzewiecki, K. T., Sunscreens: are they beneficial for health? An overview of endocrine disrupting properties of UV-filters. Int. J Androl. 2012, 35 (3), 424-436.
9. Ruszkiewicz, J. A.; Pinkas, A.; Ferrer, B.; Peres, T. V.; Tsatsakis, A.; Aschner, M., Neurotoxic effect of active ingredients in sunscreen products, a contemporary review. Toxicol. Rep. 2017, 4, 245-259.
10. Matta, M. K.; Zusterzeel, R.; Pilli, N. R.; Patel, V.; Volpe, D. A.; Florian, J.; Oh, L.; Bashaw, E.; Zineh, I.; Sanabria, C.; Kemp, S.; Godfrey, A.; Adah, S.; Coelho, S.; Wang, J.; Furlong, L. A.; Ganley, C.; Michele, T.; Strauss, D. G., Effect of sunscreen application under maximal use conditions on plasma concentration of sunscreen active ingredients a randomized clinical trial. JAMA 2019, 321 (21), 2082-2091.
11. Schneider, S. L.; Lim, H. W., Review of environmental effects of oxybenzone and other sunscreen active ingredients. J. Am. Acad. Dermatol. 2019, 80 (1), 266-271.
12. Pandika, M., Looking to nature for new sunscreens. ACS Cent. Sci. 2018, 4 (7), 788-790.
13. Saewan, N.; Jimtaisong, A., Natural products as photoprotection. J. Cosmet. Dermatol. 2015, 14 (1), 47-63.
14. Kageyama, H.; Waditee-Sirisattha, R., Antioxidative, anti-inflammatory, and anti-aging properties of mycosporine-like amino acids: Molecular and cellular mechanisms in the protection of skin-aging. Mar. Drugs 2019, 17 (4), 222. doi: 10.3390/md17040222.
15. Losantos, R.; Funes-Ardoiz, I.; Aguilera, J.; Herrera-Ceballos, E.; Garcia-Iriepa, C.; Campos, P. J.; Sampedro, D., Rational design and synthesis of efficient sunscreens to boost the solar protection factor. Angew. Chem. Int. Ed. Engl. 2017, 56 (10), 2632-2635.
16. Carreto, J. I.; Carignan, M. O., Mycosporine-like amino acids: Relevant secondary metabolites. Chemical and ecological aspects. Mar. Drugs 2011, 9 (3), 387-446.
17. M. Bandaranayake, W., Mycosporines: are they nature's sunscreens? Nat. Prod. Rep. 1998, 15 (2), 159-172.
18. Sinha, R. P.; Singh, S. P.; Hader, D. P., Database on mycosporines and mycosporine-like amino acids (MAAs) in fungi, cyanobacteria, macroalgae, phytoplankton and animals. J. Photochem. Photobiol. B 2007, 89 (1), 29-35.
19. Kicklighter, C. E.; Kamio, M.; Nguyen, L.; Germann, M. W.; Derby, C. D., Mycosporine-like amino acids are multifunctional molecules in sea hares and their marine community. Proc Natl Acad Sci USA 2011, 108 (28), 11494-11499.
20. Nazifi, E.; Wada, N.; Yamaba, M.; Asano, T.; Nishiuchi, T.; Matsugo, S.; Sakamoto, T., Glycosylated porphyra-334 and palythine-threonine from the terrestrial cyanobacterium Nostoc commune. Mar. Drugs 2013, 11 (9), 3124-3154.
21. D'Agostino, P. M.; Javalkote, V. S.; Mazmouz, R.; Pickford, R.; Puranik, P. R.; Neilan, B. A., Comparative profiling and discovery of novel glycosylated mycosporine-like amino acids in two strains of the cyanobacterium Scytonema cf crispum. Appl. Environ. Microbiol. 2016, 82 (19), 5951-5959.
22. Akio, F.; Takeshi, M.; Isami, T.; Isao, S., The crystal and molecular structure of palythine trihydrate. Bull. Chem. Soc. Jpn. 1980, 53 (2), 319-323.
23. Daisuke, U.; Chuji, K.; Akio, W.; Yoshimasa, H., Crystal and molecule structure of palythiene possessing a novel 360 nm chromophore. Chem. Lett. 1980, 9 (6), 755-756.
24. Klisch, M.; Richter, P.; Puchta, R.; Hader, D.-P.; Bauer, W., The stereostructure of porphyra-334: An experimental and calculational NMR investigation. Evidence for an efficient ‘proton sponge’. Helv. Chim. Acta 2007, 90 (3), 488-511.
25. White, J. D.; Cammack, J. H.; Sakuma, K.; Rewcastle, G. W.; Widener, R. K., Transformations of quinic acid. Asymmetric synthesis and absolute configuration of mycosporin I and mycosporin-gly. J. Org. Chem. 1995, 60 (12), 3600-3611.
26. Yang, G.; Cozad, M. A.; Holland, D. A.; Zhang, Y.; Luesch, H.; Ding, Y., Photosynthetic production of sunscreen shinorine using an engineered cyanobacterium. ACS Synth. Biol. 2018, 7 (2), 664-671.
27. Balskus, E. P.; Walsh, C. T., The genetic and molecular basis for sunscreen biosynthesis in cyanobacteria. Science 2010, 329 (5999), 1653-1656.
28. Pope, M. A.; Spence, E.; Seralvo, V.; Gacesa, R.; Heidelberger, S.; Weston, A. J.; Dunlap, W. C.; Shick, J. M.; Long, P. F., O-Methyltransferase is shared between the pentose phosphate and shikimate pathways and is essential for mycosporine-like amino acid biosynthesis in Anabaena variabilis ATCC 29413. Chembiochem 2015, 16 (2), 320-327.
29. Gao, Q.; Garcia-Pichel, F., An ATP-grasp ligase involved in the last biosynthetic step of the iminomycosporine shinorine in Nostoc punctiforme ATCC 29133. J. Bacteriol. 2011, 193 (21), 5923-5928.
30. Zallot, R.; Oberg, N.; Gerlt, J. A., The EFI web resource for genomic enzymology tools: Leveraging protein, genome, and metagenome databases to discover novel enzymes and metabolic pathways. Biochemistry 2019, 58 (41), 4169-4182.
31. Challis, G. L., Genome mining for novel natural product discovery. J. Med. Chem. 2008, 51 (9), 2618-2628.
32. Suzek, B. E.; Wang, Y.; Huang, H.; McGarvey, P. B.; Wu, C. H.; UniProt, C., UniRef clusters: a comprehensive and scalable alternative for improving sequence similarity searches. Bioinformatics 2015, 31 (6), 926-932.
33. El-Gebali, S.; Mistry, J.; Bateman, A.; Eddy, S. R.; Luciani, A.; Potter, S. C.; Qureshi, M.; Richardson, L. J.; Salazar, G. A.; Smart, A.; Sonnhammer, E. L. L.; Hirsh, L.; Paladin, L.; Piovesan, D.; Tosatto, S. C. E.; Finn, R. D., The Pfam protein families database in 2019. Nucleic Acids Res. 2019, 47 (D1), D427-D432.
34. Franke, I.; Resch, A.; Dassler, T.; Maier, T.; Bock, A., YfiK from Escherichia coli promotes export of O-acetylserine and cysteine. J. Bacteriol. 2003, 185 (4), 1161-1166.
35. Miyamoto, K. T.; Komatsu, M.; Ikeda, H., Discovery of gene cluster for mycosporine-like amino acid biosynthesis from Actinomycetales microorganisms and production of a novel mycosporine-like amino acid by heterologous expression. Appl. Environ. Microbiol. 2014, 80 (16), 5028-5036.
36. Hu, C.; Voller, G.; Sussmuth, R.; Dittmann, E.; Kehr, J. C., Functional assessment of mycosporine-like amino acids in Microcystis aeruginosa strain PCC 7806. Environ. Microbiol. 2015, 17 (5), 1548-1559.
37. D'Agostino, P. M.; Woodhouse, J. N.; Liew, H. T.; Sehnal, L.; Pickford, R.; Wong, H. L.; Burns, B. P.; Neilan, B. A., Bioinformatic, phylogenetic and chemical analysis of the UV-absorbing compounds scytonemin and mycosporine-like amino acids from the microbial mat communities of Shark Bay, Australia. Environ. Microbiol. 2019, 21 (2), 702-715.
38. Hegg, E. L.; Que, L., Jr., The 2-His-1-carboxylate facial triad—an emerging structural motif in mononuclear non-heme iron(II) enzymes. Eur. J. Biochem. 1997, 250 (3), 625-629.
39. Mihalik, S. J.; Morrell, J. C.; Kim, D.; Sacksteder, K. A.; Watkins, P. A.; Gould, S. J., Identification of PAHX, a Refsum disease gene. Nat. Genet. 1997, 17 (2), 185-189.
40. Islam, M. S.; Leissing, T. M.; Chowdhury, R.; Hopkinson, R. J.; Schofield, C. J., 2-Oxoglutarate-dependent oxygenases. Annu. Rev. Biochem. 2018, 87, 585-620.
41. Kavanagh, K. L.; Jornvall, H.; Persson, B.; Oppermann, U., Medium- and short-chain dehydrogenase/reductase gene and protein families: the SDR superfamily: functional and structural diversity within a family of metabolic and regulatory enzymes. Cell Mol. Life Sci. 2008, 65 (24), 3895-3906.
42. Carignan, M. O.; Cardozo, K. H.; Oliveira-Silva, D.; Colepicolo, P.; Carreto, J. I., Palythine-threonine, a major novel mycosporine-like amino acid (MAA) isolated from the hermatypic coral Pocillopora capitata. J. Photochem. Photobiol. B 2009, 94 (3), 191-200.
43. Orfanoudaki, M.; Hartmann, A.; Ngoc, H. N.; Gelbrich, T.; West, J.; Karsten, U.; Ganzera, M., Mycosporine-like amino acids, brominated and sulphated phenols: Suitable chemotaxonomic markers for the reassessment of classification of Bostrychia calliptera (Ceramiales, Rhodophyta). Phytochemistry 2020, 174, 112344. doi: 10.1016/j.phytochem.2020.
44. Geraldes, V.; Jacinavicius, F. R.; Genuario, D. B.; Pinto, E., Identification and distribution of mycosporine-like amino acids in Brazilian cyanobacteria using ultrahigh-performance liquid chromatography with diode array detection coupled to quadrupole time-of-flight mass spectrometry. Rapid Commun. Mass Spectrom. 2020, 34 Suppl 3, e8634. doi: 10.1002/rem.8634.
45. Pederick, J. L.; Thompson, A. P.; Bell, S. G.; Bruning, J. B., D-Alanine-D-alanine ligase as a model for the activation of ATP-grasp enzymes by monovalent cations. J. Biol. Chem. 2020, 295 (23), 7894-7904.
46. Lessard, I. A.; Healy, V. L.; Park, I. S.; Walsh, C. T., Determinants for differential effects on D-Ala-D-lactate vs D-Ala-D-Ala formation by the VanA ligase from vancomycin-resistant enterococci. Biochemistry 1999, 38 (42), 14006-14022.
47. Harvey, A. L.; Edrada-Ebel, R.; Quinn, R. J., The re-emergence of natural products for drug discovery in the genomics era. Nat. Rev. Drug Discov. 2015, 14 (2), 111-129.
48. Shannon, P.; Markiel, A.; Ozier, O.; Baliga, N. S.; Wang, J. T.; Ramage, D.; Amin, N.; Schwikowski, B.; Ideker, T., Cytoscape: a software environment for integrated models of biomolecular interaction networks. Genome Res 2003, 13 (11), 2498-2504.
49. Thompson, J. D.; Higgins, D. G.; Gibson, T. J., Clustal-W—Improving the sensitivity of progressive multiple sequence alignment through sequence weighting, position-specific gap penalties and weight matrix choice. Nucleic Acids Res. 1994, 22 (22), 4673-4680.
50. Orfanoudaki, M.; Hartmann, A.; Ngoc, H. N.; Gelbrich, T.; West, J.; Karsten, U.; Ganzera, M., Mycosporine-like amino acids, brominated and sulphated phenols: Suitable chemotaxonomic markers for the reassessment of classification of Bostrychia calliptera (Ceramiales, Rhodophyta). Phytochemistry 2020, 174, 112344.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A method for producing a compound, comprising:
a) culturing a recombinant microorganism under conditions suitable for production of the compound; and
b) isolating the compound from the recombinant microorganism,
wherein the recombinant microorganism comprises a heterologous nucleic acid encoding one or more mycosporine-like amino acid (MAA) biosynthetic enzymes, wherein the one or more MAA biosynthetic enzymes comprise a phytanoyl-CoA dioxygenase (MysH), or a homolog thereof.

2. The method of claim 1, wherein the phytanoyl-CoA dioxygenase comprises an amino acid sequence of any one of SEQ ID NOs: 1-11, or an amino acid sequence at least 70%, at least 75%, at least 80%, at least 85%, at least 90%, at least 95%, or at least 99% identical to the amino acid sequence of any one of SEQ ID NOs: 1-11.

3. The method of claim 1 or 2, wherein the one or more MAA biosynthetic enzymes further comprise a D-alanine-D-alanine ligase (MysD), or a homolog thereof.

4. The method of claim 3, wherein the D-alanine-D-alanine ligase comprises an amino acid sequence of SEQ ID NO: 12, or an amino acid sequence at least 70%, at least 75%, at least 80%, at least 85%, at least 90%, at least 95%, or at least 99% identical to the amino acid sequence of SEQ ID NO: 12.

5. The method of any one of claims 1-4, wherein the one or more MAA biosynthetic enzymes further comprise an ATP-grasp enzyme (MysC), or a homolog thereof.

6. The method of claim 5, wherein the ATP-grasp enzyme comprises an amino acid sequence of any one of SEQ ID NOs: 13-104 and 113-116, or an amino acid sequence at least 70%, at least 75%, at least 80%, at least 85%, at least 90%, at least 95%, or at least 99% identical to the amino acid sequence of any one of SEQ ID NOs: 13-104 and 113-116.

7. The method of any one of claims 1-6, wherein the one or more biosynthetic enzymes further comprise one or more enzymes selected from the group consisting of a dimethyl-4-deoxygadusol synthase (MysA), an O-methyltransferase (MysB), and a non-ribosomal peptide synthetase (NRPS)-like enzyme (MysE).

8. The method of any one of claims 1-7, wherein the compound is a palythine analog.

9. The method of any one of claims 1-8, wherein the compound has UV-modulating activity.

10. The method of claim 9, wherein the UV-modulating activity comprises absorption of UV wavelengths between 310 and 362 nm.

11. The method of any one of claims 1-10, wherein the compound is of Formula (I), or a salt thereof: 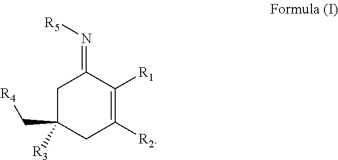
wherein:
each of R₁, R₂, R₃, and R₄ is independently selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl; and
R₅ is an amino acid.

12. The method of claim 11, wherein R₁ is —OR^{a}, wherein R^{a} is optionally substituted C₁₋₆ alkyl.

13. The method of claim 12, wherein R₁ is —OCH₃.

14. The method of any one of claims 11-13, wherein R₂ is —NH₂.

15. The method of any one of claims 11-14, wherein R₃ is —OH.

16. The method of any one of claims 11-15, wherein R₄ is —OH.

17. The method of any one of claims 11-16, wherein R₅ is threonine.

18. The method of any one of claims 11-16, wherein R₅ is serine.

19. The method of any one of claims 11-16, wherein R₅ is isoleucine.

20. The method of any one of claims 11-16, wherein R₅ is methionine.

21. The method of any one of claims 11-16, wherein R₅ is valine.

22. The method of any one of claims 1-11, wherein the compound is of the formula: 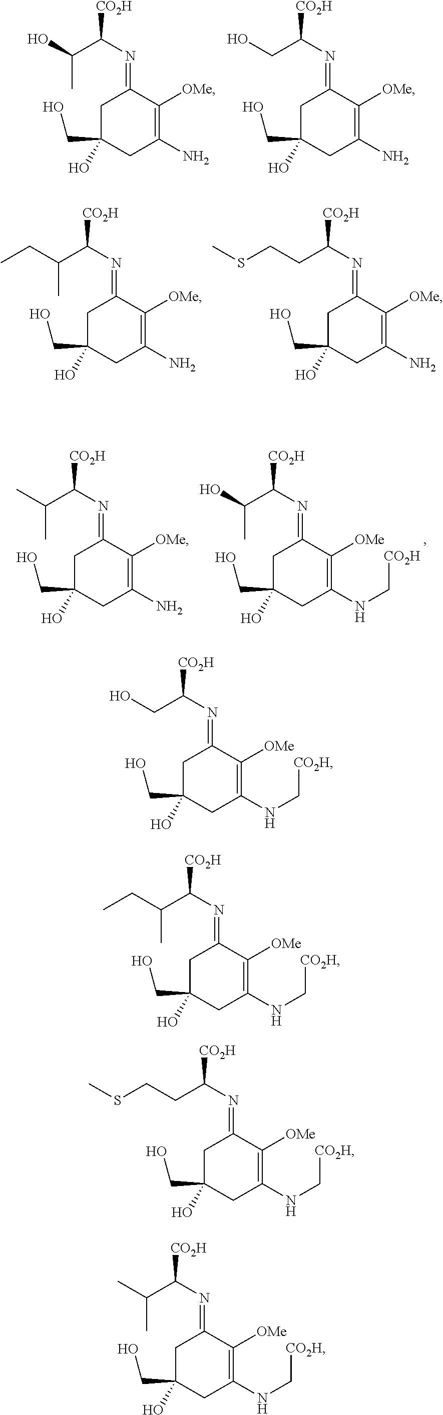
or a salt thereof.

23. The method of any one of claims 1-10, wherein the compound is of the formula: 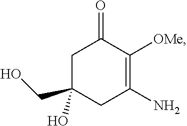 [SMILES: COC1=C(N)C[C@@](O)(CO)CC1=O]
or a salt thereof.

24. The method of any one of claims 1-23, further comprising providing a substrate of the one or more mycosporine-like amino acid (MAA) biosynthetic enzymes to the recombinant microorganism.

25. The method of claim 24, wherein the substrate is a compound of Formula (II), or a salt thereof: 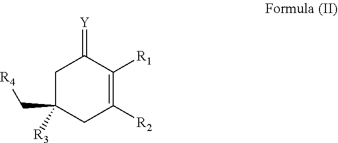
wherein:
each of R₁, R₂, R₃, and R₄ is independently selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl; and
Y is O or NR₅, wherein R₅ is optionally substituted C₁₋₆ alkyl, optionally substituted C₁₋₆ alkenyl, or an amino acid.

26. The method of claim 25, wherein R₁ is —OH.

27. The method of claim 25, wherein R₁ is —OCH₃.

28. The method of any one of claims 25-27, wherein R₂ is —OH.

29. The method of any one of claims 25-27, wherein R² is —NH₂.

30. The method of any one of claims 25-27, wherein R₂ is —(NH)R^{b}, wherein R^{b} is optionally substituted alkyl.

31. The method of claim 30, wherein R₂ is —NHCH₂CO₂H.

32. The method of any one of claims 25-31, wherein R₃ is —OH.

33. The method of any one of claims 25-32, wherein R₄ is —OH.

34. The method of any one of claims 25-33, wherein Y is O.

35. The method of any one of claims 25-33, wherein Y is NR₅.

36. The method of claim 35, wherein R₅ is threonine.

37. The method of claim 35, wherein R₅ is serine.

38. The method of claim 35, wherein R₅ is isoleucine.

39. The method of claim 35, wherein R₅ is methionine.

40. The method claim 35, wherein R₅ is valine.

41. The method of claim 25, wherein the substrate is of the formula: 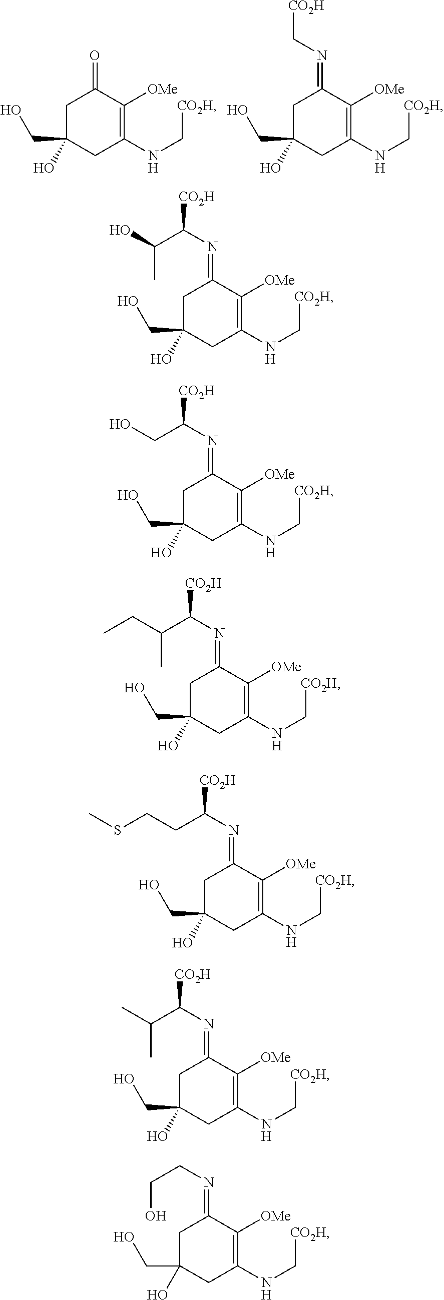 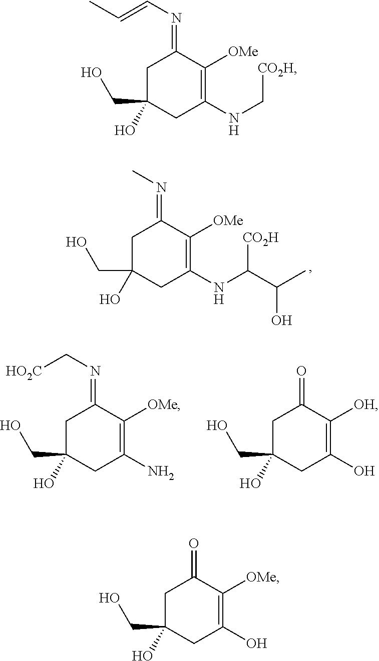
or a salt thereof.

42. The method of any one of claims 1-41, wherein the one or more MAA biosynthetic enzymes further comprise a glycosyltransferase (GlyT), or a homolog thereof.

43. The method of any one of claims 1-42, wherein the recombinant microorganism is a species of bacteria or yeast.

44. The method of claim 43, wherein the bacteria is a species of cyanobacteria.

45. The method of claim 43, wherein the bacteria is a species from the human microbiome.

46. The method of claim 43, wherein the bacteria is E. coli.

47. A recombinant microorganism comprising a heterologous nucleic acid encoding one or more mycosporine-like amino acid (MAA) biosynthetic enzymes, wherein the one or more MAA biosynthetic enzymes comprise a phytanoyl-CoA dioxygenase (MysH), or a homolog thereof.

48. A method of producing a compound, comprising:
a) culturing the recombinant microorganism of claim 47 under conditions suitable for production of the compound; and
b) isolating the compound from the recombinant microorganism.

49. A composition comprising a compound produced by the method of any one of claims 1-46 or 48 and optionally an excipient.

50. The composition of claim 49, wherein the composition is for topical administration.

51. The composition of claim 49 or 50, wherein the composition is formulated as a sunscreen.

52. The composition of claim 49 or 50, wherein the composition is formulated as a cosmetic.

53. A method of making the composition of any one of claims 49-52, comprising:
a) culturing a recombinant microorganism under conditions suitable for production of the compound;
b) isolating the compound from the recombinant microorganism,
wherein the recombinant microorganism comprises a heterologous nucleic acid encoding one or more mycosporine-like amino acid (MAA) biosynthetic enzymes, wherein the one or more MAA biosynthetic enzymes comprise a phytanoyl-CoA dioxygenase (MysH), or a homolog thereof, and
c) adding the compound to one or more excipients to produce the composition.

54. A method of administering a compound, comprising applying the composition of any one of claims 49-52 to a subject.

55. A method of preventing sunburn, comprising applying the composition of any one of claims 49-52 on the skin of a subject in need thereof.

56. A method of preventing cancer, comprising applying the composition of any one of claims 49-52 on the skin of a subject in need thereof.

57. A method of preventing or treating a chronic inflammatory disease, comprising administering the composition of any one of claims 49-52 to a subject in need thereof.

58. A compound produced by:
a) culturing a recombinant microorganism under conditions suitable for production of the compound; and
b) isolating the compound from the recombinant microorganism,
wherein the recombinant microorganism comprises a heterologous nucleic acid encoding one or more mycosporine-like amino acid (MAA) biosynthetic enzymes, wherein the one or more MAA biosynthetic enzymes comprise a phytanoyl-CoA dioxygenase (MysH), or a homolog thereof.

59. The compound of claim 58, wherein the compound is of Formula (I), or a salt thereof: 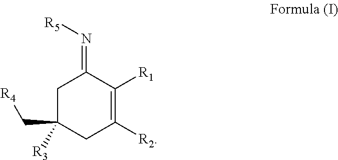
wherein:
each of R₁, R₂, R₃, and R₄ is independently selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl; and
R₅ is an amino acid.

60. The compound of claim 59, wherein R₁ is —OR^{a}, wherein R^{a} is optionally substituted C₁₋₆ alkyl.

61. The compound of claim 60, wherein R₁ is —OCH₃.

62. The compound of any one of claims 59-61, wherein R₂ is —NH₂.

63. The compound of any one of claims 59-62, wherein R₃ is —OH.

64. The compound of any one of claims 59-63, wherein R₄ is —OH.

65. The compound of any one of claims 59-64, wherein R₅ is threonine.

66. The compound of any one of claims 59-65, wherein R₅ is serine.

67. The compound of any one of claims 59-65, wherein R₅ is isoleucine.

68. The compound of any one of claims 59-65, wherein R₅ is methionine.

69. The compound of any one of claims 59-65, wherein, wherein R₅ is valine.

70. The compound of any one of claims 59-69, wherein the compound is of the formula: 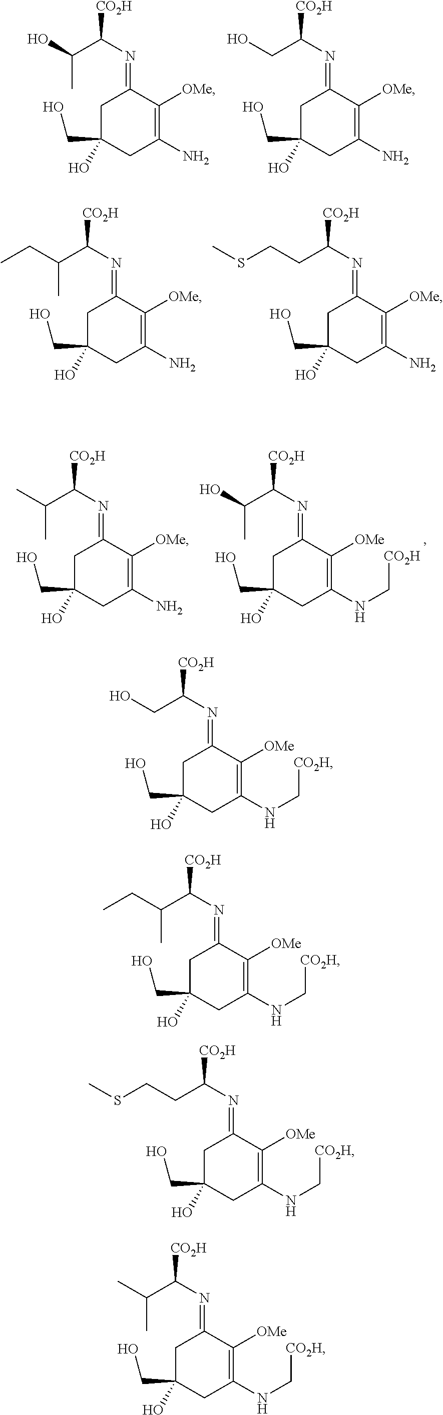
or a salt thereof.

71. The compound of claim 58, wherein the compound is of the formula: 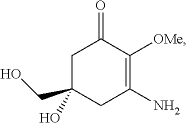 [SMILES: COC1=C(N)C[C@@](O)(CO)CC1=O]
or a salt thereof.

72. A composition comprising the compound of any one of claims 58-71, or a salt thereof.

73. The composition of claim 72, wherein the composition is for topical administration.

74. The composition of claim 72 or 73, wherein the composition is formulated as a sunscreen.

75. The composition of claim 72 or 73, wherein the composition is formulated as a cosmetic.

76. A method of administering a compound, comprising applying the composition of any one of claims 72-75 to a subject.

77. A method of preventing sunburn, comprising applying the composition of any one of claims 72-75 on the skin of a subject in need thereof.

78. A method of preventing cancer, comprising applying the composition of any one of claims 72-75 on the skin of a subject in need thereof.

79. A method of treating or preventing a chronic inflammatory disease, comprising applying the composition of any one of claims 72-75 on the skin of a subject in need thereof.
